(19) United States
(12) Patent Application Publication
(10) Pub. No.: US 2023/0127839 A1
(43) Pub. Date: Apr. 27, 2023

(54) COMPOUNDS AND COMPOSITIONS FOR TREATING CONDITIONS ASSOCIATED WITH STING ACTIVITY

(71) Applicant: IFM Due, Inc., Boston, MA (US)

(72) Inventors: Hans Martin Seidel, Concord, MA (US); William R. Roush, Boston, MA (US); Shankar Venkatraman, Lansdale, PA (US); Jason Katz, Newton, MA (US)

(21) Appl. No.: 17/789,694

(22) PCT Filed: Dec. 30, 2020
(86) PCT No.: PCT/US2020/067483

§ 371 Date: Jun. 28, 2022

Related U.S. Application Data
(60) Provisional application No. 62/955,839, filed on Dec. 31, 2019.
(60) Provisional application No. 63/090,538, filed on Oct. 12, 2020.

(51) Int. Cl. C07D 401/14; C07D 401/12; C07D 209/40; C07D 413/14; C07D 491/107; C07D 405/14; C07D 403/12; C07D 498/08

(52) U.S. Cl. CPC C07D 401/14; C07D 401/12; C07D 209/40; C07D 413/14; C07D 491/107; C07D 405/14; C07D 403/12; C07D 498/08

(57) ABSTRACT

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

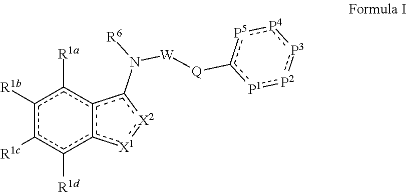

### CROSS REFERENCE TO RELATED APPLICATIONS

This application claims the benefit of U.S. Provisional Application Ser. No. 62/955,839, filed on Dec. 31, 2019; and U.S. Provisional Application Ser. No. 63/090,538, filed on Oct. 12, 2020; each of which is incorporated herein by reference in its entirety.

### TECHNICAL FIELD

This disclosure features chemical entities (e.g., a compound or a pharmaceutically acceptable salt, and/or hydrate, and/or cocrystal, and/or prodrug, and/or tautomer, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

### BACKGROUND

STING, also known as transmembrane protein 173 (TMEM173) and MPYS/MITA/ERIS, is a protein that in humans is encoded by the TMEM173 gene. STING has been shown to play a role in innate immunity. STING induces type I interferon production when cells are infected with intracellular pathogens, such as viruses, mycobacteria and intracellular parasites. Type I interferon, mediated by STING, protects infected cells and nearby cells from local infection in an autocrine and paracrine manner.

The STING pathway is pivotal in mediating the recognition of cytosolic DNA. In this context, STING, a transmembrane protein localized to the endoplasmic reticulum (ER), acts as a second messenger receptor for 2′, 3′ cyclic GMP-AMP (hereafter cGAMP), which is produced by cGAS after dsDNA binding. In addition, STING can also function as a primary pattern recognition receptor for bacterial cyclic dinucleotides (CDNs) and small molecule agonists. The recognition of endogenous or prokaryotic CDNs proceeds through the carboxy-terminal domain of STING, which faces into the cytosol and creates a V-shaped binding pocket formed by a STING homodimer. Ligand-induced activation of STING triggers its re-localization to the Golgi, a process essential to promote the interaction of STING with TBK1. This protein complex, in turn, signals through the transcription factors IRF-3 to induce type I interferons (IFNs) and other co-regulated antiviral factors. In addition, STING was shown to trigger NF-κB and MAP kinase activation. Following the initiation of signal transduction, STING is rapidly degraded, a step considered important in terminating the inflammatory response.

Excessive activation of STING is associated with a subset of monogenic autoinflammatory conditions, the so-called type I interferonopathies. Examples of these diseases include a clinical syndrome referred to as STING-associated vasculopathy with onset in infancy (SAVI), which is caused by gain-of-function mutations in TMEM173 (the gene name of STING). Moreover, STING is implicated in the pathogenesis of Aicardi-Goutières Syndrome (AGS) and genetic forms of lupus. As opposed to SAVI, it is the dysregulation of nucleic acid metabolism that underlies continuous innate immune activation in AGS. Apart from these genetic disorders, emerging evidence points to a more general pathogenic role for STING in a range of inflammation-associated disorders such as systemic lupus erythematosus, rheumatoid arthritis and cancer. Thus, small molecule-based pharmacological interventions into the STING signaling pathway hold significant potential for the treatment of a wide spectrum of diseases

### SUMMARY

This disclosure features chemical entities (e.g., a compound or a pharmaceutically acceptable salt, and/or hydrate, and/or cocrystal, and/or prodrug, and/or tautomer, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

An “antagonist” of STING includes compounds that, at the protein level, directly bind or modify STING such that an activity of STING is decreased, e.g., by inhibition, blocking or dampening agonist-mediated responses, altered distribution, or otherwise. STING antagonists include chemical entities, which interfere or inhibit STING signaling.

In one aspect, compounds of Formula (I), or a pharmaceutically acceptable salt thereof, are featured.

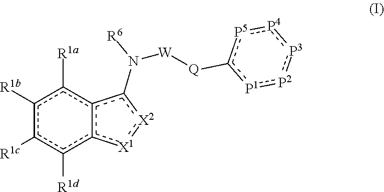

in which R^{1a}, R^{1b}, R^{1c}, R^{1d}, X¹, X², R⁶, W, Q, P¹, P², P³, P⁴, and P⁵ can be as defined anywhere herein.

In one aspect, compounds of Formula (I), or a pharmaceutically acceptable salt thereof, or a prodrug thereof, or a tautomer thereof, or any combination of the foregoing, are featured. “Prodrug” is meant to indicate a compound that may be converted under physiological conditions or by solvolysis to a biologically active compound described herein (e.g., compound of Formula (I)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for inhibiting (e.g., antagonizing) STING activity are featured that include contacting STING with a chemical entity described herein (e.g., a compound described generically or specifically herein or a pharmaceutically acceptable salt thereof or compositions containing the same). Methods include in vitro methods, e.g., contacting a sample that includes one or more cells comprising STING (e.g., innate immune cells, e.g., mast cells, macrophages, dendritic cells (DCs), and natural killer cells) with the chemical entity. Methods can also include in vivo methods; e.g., administering the chemical entity to a subject (e.g., a human) having a disease in which increased (e.g., excessive) STING signaling contributes to the pathology and/or symptoms and/or progression of the disease.

In one aspect, methods of treating a condition, disease or disorder ameliorated by antagonizing STING are featured, e.g., treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). The methods include administering to a subject in need of such treatment an effective amount of a chemical entity described herein (e.g., a compound described generically or specifically herein or a pharmaceutically acceptable salt thereof or compositions containing the same).

In another aspect, methods of treating cancer are featured that include administering to a subject in need of such treatment an effective amount of a chemical entity described herein (e.g., a compound described generically or specifically herein or a pharmaceutically acceptable salt thereof or compositions containing the same).

In a further aspect, methods of treating other STING-associated conditions are featured, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis. The methods include administering to a subject in need of such treatment an effective amount of a chemical entity described herein (e.g., a compound described generically or specifically herein or a pharmaceutically acceptable salt thereof or compositions containing the same).

In another aspect, methods of suppressing STING-dependent type I interferon production in a subject in need thereof are featured that include administering to the subject an effective amount of a chemical entity described herein (e.g., a compound described generically or specifically herein or a pharmaceutically acceptable salt thereof or compositions containing the same).

In a further aspect, methods of treating a disease in which increased (e.g., excessive) STING activation (e.g., STING signaling) contributes to the pathology and/or symptoms and/or progression of the disease are featured. The methods include administering to a subject in need of such treatment an effective amount of a chemical entity described herein (e.g., a compound described generically or specifically herein or a pharmaceutically acceptable salt thereof or compositions containing the same).

In another aspect, methods of treatment are featured that include administering an effective amount of a chemical entity described herein (e.g., a compound described generically or specifically herein or a pharmaceutically acceptable salt thereof or compositions containing the same) to a subject; wherein the subject has (or is predisposed to have) a disease in which increased (e.g., excessive) STING activation (e.g., STING signaling) contributes to the pathology and/or symptoms and/or progression of the disease.

In a further aspect, methods of treatment that include administering to a subject a chemical entity described herein (e.g., a compound described generically or specifically herein or a pharmaceutically acceptable salt thereof or compositions containing the same), wherein the chemical entity is administered in an amount effective to treat a disease in which increased (e.g., excessive) STING activation (e.g., STING signaling) contributes to the pathology and/or symptoms and/or progression of the disease, thereby treating the disease.

In another aspect, is a compound, or a pharmaceutically acceptable salt or tautomer thereof, described herein, for use in the treatment of a disease, condition or disorder modulated by STING inhibition.

In another aspect, is a compound, or a pharmaceutically acceptable salt or tautomer thereof, described herein for use in the treatment of a condition, disease or disorder associated with increased (e.g., excessive) STING activation.

In another aspect, is a compound, or a pharmaceutically acceptable salt or tautomer thereof, described herein for use in the treatment of cancer.

In another aspect, is a compound, or a pharmaceutically acceptable salt or tautomer thereof, described herein for use in the treatment of cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

In another aspect, is a compound, or a pharmaceutically acceptable salt or tautomer thereof, described herein for use in the treatment of type I interferonopathies.

In another aspect, is a compound, or a pharmaceutically acceptable salt or tautomer thereof, described herein for use in the treatment of type I interferonopathies selected from STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis.

In another aspect, is the use of a compound, or a pharmaceutically acceptable salt or tautomer thereof, described herein for use in the manufacture of a medicament for the treatment of a condition, disease or disorder associated with increased (e.g., excessive) STING activation.

In another aspect, is the use of a compound, or a pharmaceutically acceptable salt or tautomer thereof, described herein for use in the manufacture of a medicament for the treatment of cancer.

In another aspect, is the use of a compound, or a pharmaceutically acceptable salt or tautomer thereof, described herein for use in the manufacture of a medicament for the treatment of cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

In another aspect, is the use of a compound, or a pharmaceutically acceptable salt or tautomer thereof, described herein for use in the manufacture of a medicament for the treatment of type I interferonopathies.

In another aspect, is the use of a compound, or a pharmaceutically acceptable salt or tautomer thereof, described herein for use in the manufacture of a medicament for the treatment of type I interferonopathies selected from STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis.

In another aspect, is the use of a compound, or a pharmaceutically acceptable salt or tautomer thereof, described herein, for the treatment of a disease, condition or disorder modulated by STING inhibition.

In another aspect, is the use of a compound, or a pharmaceutically acceptable salt or tautomer thereof, described herein for the treatment of a condition, disease or disorder associated with increased (e.g., excessive) STING activation.

In another aspect, is the use of a compound, or a pharmaceutically acceptable salt or tautomer thereof, described herein for the treatment of cancer.

In another aspect, is the use of a compound, or a pharmaceutically acceptable salt or tautomer thereof, described herein for the treatment of cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

In another aspect, is the use of a compound, or a pharmaceutically acceptable salt or tautomer thereof, described herein for the treatment of type I interferonopathies.

In another aspect, is the use of a compound, or a pharmaceutically acceptable salt or tautomer thereof, described herein for the treatment of type I interferonopathies selected from STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis.

Embodiments can include one or more of the following features.

The chemical entity can be administered in combination with one or more additional therapeutic agents and/or regimens. For examples, methods can further include administering one or more (e.g., two, three, four, five, six, or more) additional agents.

The chemical entity can be administered in combination with one or more additional therapeutic agents and/or regimens that are useful for treating other STING-associated conditions, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis.

The chemical entity can be administered in combination with one or more additional cancer therapies (e.g., surgery, radiotherapy, chemotherapy, toxin therapy, immunotherapy, cryotherapy or gene therapy, or a combination thereof; e.g., chemotherapy that includes administering one or more (e.g., two, three, four, five, six, or more) additional chemotherapeutic agents. Non-limiting examples of additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

The subject can have cancer; e.g., the subject has undergone and/or is undergoing and/or will undergo one or more cancer therapies.

Non-limiting examples of cancer include melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma. In certain embodiments, the cancer can be a refractory cancer.

The chemical entity can be administered intratumorally.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “STING” is meant to include, without limitation, nucleic acids, polynucleotides, oligonucleotides, sense and antisense polynucleotide strands, complementary sequences, peptides, polypeptides, proteins, homologous and/or orthologous STING molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The term “halo” refers to fluoro (F), chloro (Cl), bromo (Br), or iodo (I).

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “alkoxy” refers to an —O-alkyl radical (e.g., —OCH₃).

The term “alkylene” refers to a divalent alkyl (e.g., —CH₂—).

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, dihydro-1H-indenyl and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butanyl, bicyclo[2.1.0]pentanyl, bicyclo[1.1.1]pentanyl, bicyclo[3.1.0]hexanyl, bicyclo[2.1.1]hexanyl, bicyclo[3.2.0]heptanyl, bicyclo[4.1.0]heptanyl, bicyclo[2.2.1]heptanyl, bicyclo[3.1.1]heptanyl, bicyclo[4.2.0]octanyl, bicyclo[3.2.1]octanyl, bicyclo[2.2.2]octanyl, and the like. Cycloalkyl also includes spirocyclic rings (e.g., spirocyclic bicycle wherein two rings are connected through just one atom). Non-limiting examples of spirocyclic cycloalkyls include spiro[2.2]pentanyl, spiro[2.5]octanyl, spiro[3.5]nonanyl, spiro[3.5]nonanyl, spiro[3.5]nonanyl, spiro[4.4]nonanyl, spiro[2.6]nonanyl, spiro[4.5]decanyl, spiro[3.6]decanyl, spiro[5.5]undecanyl, and the like. The term “saturated” as used in this context means only single bonds present between constituent carbon atoms.

The term “cycloalkenyl” as used herein means partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl.

As partially unsaturated cyclic hydrocarbon groups, cycloalkenyl groups may have any degree of unsaturation provided that one or more double bonds is present in the ring, none of the rings in the ring system are aromatic, and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic, and at least one ring in the system contains one or more heteroatoms independently selected from the group consisting of N, O, and S (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, benzo[d]thiazolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl.

The term “heterocyclyl” refers to a mon-, bi-, tri-, or polycyclic saturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butanyl, 2-azabicyclo[2.1.0]pentanyl, 2-azabicyclo[1.1.1]pentanyl, 3-azabicyclo[3.1.0]hexanyl, 5-azabicyclo[2.1.1]hexanyl, 3-azabicyclo[3.2.0]heptanyl, octahydrocyclopenta[c]pyrrolyl, 3-azabicyclo[4.1.0]heptanyl, 7-azabicyclo[2.2.1]heptanyl, 6-azabicyclo[3.1.1]heptanyl, 7-azabicyclo[4.2.0]octanyl, 2-azabicyclo[2.2.2]octanyl, 3-azabicyclo[3.2.1]octanyl, 2-oxabicyclo[1.1.0]butanyl, 2-oxabicyclo[2.1.0]pentanyl, 2-oxabicyclo[1.1.1]pentanyl, 3-oxabicyclo[3.1.0]hexanyl, 5-oxabicyclo[2.1.1]hexanyl, 3-oxabicyclo[3.2.0]heptanyl, 3-oxabicyclo[4.1.0]heptanyl, 7-oxabicyclo[2.2.1]heptanyl, 6-oxabicyclo[3.1.1]heptanyl, 7-oxabicyclo[4.2.0]octanyl, 2-oxabicyclo[2.2.2]octanyl, 3-oxabicyclo[3.2.1]octanyl, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentanyl, 4-azaspiro[2.5]octanyl, 1-azaspiro[3.5]nonanyl, 2-azaspiro[3.5]nonanyl, 7-azaspiro[3.5]nonanyl, 2-azaspiro[4.4]nonanyl, 6-azaspiro[2.6]nonanyl, 1,7-diazaspiro[4.5]decanyl, 7-azaspiro[4.5]decanyl, 2,5-diazaspiro[3.6]decanyl, 3-azaspiro[5.5]undecanyl, 2-oxaspiro[2.2]pentanyl, 4-oxaspiro[2.5]octanyl, 1-oxaspiro[3.5]nonanyl, 2-oxaspiro[3.5]nonanyl, 7-oxaspiro[3.5]nonanyl, 2-oxaspiro[4.4]nonanyl, 6-oxaspiro[2.6]nonanyl, 1,7-dioxaspiro[4.5]decanyl, 2,5-dioxaspiro[3.6]decanyl, 1-oxaspiro[5.5]undecanyl, 3-oxaspiro[5.5]undecane, 3-oxa-9-azaspiro[5.5]undecanyl and the like. The term “saturated” as used in this context means only single bonds present between constituent ring atoms and other available valences occupied by hydrogen and/or other substituents as defined herein.

The term “heterocycloalkenyl” as used herein means partially unsaturated cyclic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkenyl groups include, without limitation, tetrahydropyridyl, dihydropyrazinyl, dihydropyridyl, dihydropyrrolyl, dihydrofuranyl, dihydrothiophenyl. As partially unsaturated cyclic groups, heterocycloalkenyl groups may have any degree of unsaturation provided that one or more double bonds is present in the ring, none of the rings in the ring system are aromatic, and the heterocycloalkenyl group is not fully saturated overall. Heterocycloalkenyl may include multiple fused and/or bridged and/or spirocyclic rings.

As used herein, when a ring is described as being “aromatic”, it means said ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

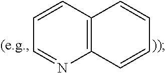

(ii) a single ring atom (spiro-fused ring systems)

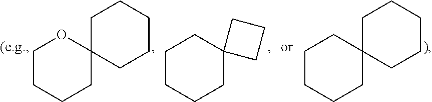

or (iii) a contiguous array of ring atoms (bridged ring systems having all bridge lengths >0)

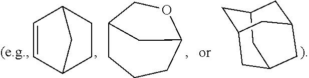

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

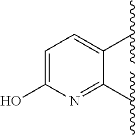 [SMILES: *c1ccc(O)nc1*]

encompasses the tautomeric form containing the moiety:

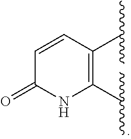 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The details of one or more embodiments of the invention are set forth in the accompanying drawings and the description below. Other features and advantages of the invention will be apparent from the description and drawings, and from the claims.

### DETAILED DESCRIPTION

This disclosure features chemical entities (e.g., a compound or a pharmaceutically acceptable salt, and/or hydrate, and/or cocrystal, and/or prodrug, and/or tautomer, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

Formula I Compounds

In one aspect, this disclosure features compounds of Formula (I):

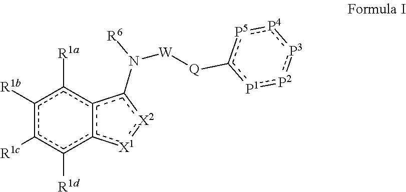

or a pharmaceutically acceptable salt thereof or a tautomer thereof, wherein:

X¹ is selected from the group consisting of O, S, N, NR², and CR¹;

X² is selected from the group consisting of O, S, N, NR⁴, and CR⁵;

each [symbol] is independently a single bond or a double bond, provided that:

the five-membered ring comprising X¹ and X² is heteroaryl;

the 6-membered ring

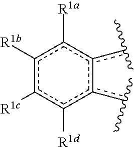

is aromatic; and

and the ring comprising P¹, P², P³, P⁴, and P⁵ is aromatic; P¹, P², P³, P⁴, and P⁵ are defined according to (AA) or (BB):

### AA

each of P¹, P², P³, P⁴, and P⁵ is independently selected from the group consisting of: N, CH, CR⁷, and CR^{c}, provided that 1-2 of P¹, P², P³, P⁴, and P⁵ is an independently selected CR⁷; or

### BB

P¹ is absent, thereby providing a 5-membered ring,

each of P², P³, P⁴, and P⁵ is independently selected from the group consisting of O, S, N, NH, NR^{d}, NR⁷, CH, CR⁷, and CR^{c}, provided that 1-3 of P², P³, P⁴, and P⁵ is O, S, N, NH, NR^{d}, or NR⁷; and 1-2 of P², P³, P⁴, and P⁵ is an independently selected NR⁷ or CR⁷;

each R⁷ is independently selected from the group consisting of: —R⁸ and -L³-R⁹;

R⁸ and R⁹ are independently selected from the group consisting of:

(a) C₃₋₁₂ cycloalkyl or C₃₋₁₂ cycloalkenyl, each of which is optionally substituted with 1-4 independently selected R⁷′;

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R⁷′;

(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R⁷′; and

(d) C₆₋₁₀ aryl optionally substituted with 1-4 independently selected R⁷′;

-L³ is selected from the group consisting of —O—, —C₁₋₄ alkylene, —S—, —NH—, S(O)₁₋₂, C(═O)NH, NHC(═O), C(═O)O, OC(═O), C(═O), NHS(O)₂, and S(O)₂NH;

each occurrence of R⁷′ is independently selected from the group consisting of: halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),

W is selected from the group consisting of:

(i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);

Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents point of attachment to W;

each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);

each occurrence of R² is independently selected from the group consisting of:

(i) H;

(ii) C₁₋₆ alkyl, which is optionally substituted with 1-3 independently selected R^{a};

(iii) —C(O)(C₁₋₆ alkyl) optionally substituted with 1-3 independently selected R^{a};

(iv) —C(O)O(C₁₋₄ alkyl) optionally substituted with 1-3 independently R^{a};

(v) —CON(R′)(R″);

(vi) —S(O)₁₋₂(NR′R″);

(vii) —S(O)₁₋₂(C₁₋₄ alkyl) optionally substituted with 1-3 independently selected R^{a};

(viii) —OH;

(ix) C₁₋₄ alkoxy; and

(x) -L⁴-L⁵-Rⁱ;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; —C(═O)N(R′)(R″); and -L¹-L²-R^{h};

R^{d} is selected from the group consisting of: C₁₋₆ alkyl optionally substituted with 1-3 substituents each independently selected from the group consisting of: halo, C₁₋₃ alkoxy, C₁₋₃ haloalkoxy, OH, and C₃₋₆ cycloalkyl; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂N(R′)(R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂N(R′)(R″); —S(O)₁₋₂(C₁₋₄ alkyl); —OH; and C₁₋₄ alkoxy; or

R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene; -L² is —O—, —N(H)—, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};

-L⁵- is selected from the group consisting of a bond and C₁₋₄ alkylene;

Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and

each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl;

or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

In one aspect, this disclosure features compounds of Formula (I):

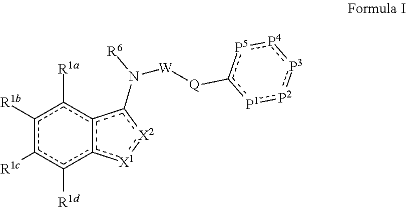

or a pharmaceutically acceptable salt thereof or a tautomer thereof, wherein:

X¹ is selected from the group consisting of O, S, N, NR², and CR¹;

X² is selected from the group consisting of O, S, N, NR⁴, and CR⁵;

each [symbol] is independently a single bond or a double bond, provided that:

the five-membered ring comprising X¹ and X² is heteroaryl;

the 6-membered ring

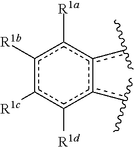

is aromatic; and

and the ring comprising P¹, P², P³, P⁴, and P⁵ is aromatic;

P¹, P², P³, P⁴, and P⁵ are defined according to (AA) or (BB):

### AA

each of P¹, P², P³, P⁴, and P⁵ is independently selected from the group consisting of: N, CH, CR⁷, and CR^{c}, provided that 1-2 of P¹, P², P³, P⁴, and P⁵ is an independently selected CR⁷; or

### BB

P¹ is absent, thereby providing a 5-membered ring,

each of P², P³, P⁴, and P⁵ is independently selected from the group consisting of O, S, N, NH, NR^{d}, NR⁷, CH, CR⁷, and CR^{c}, provided that 1-3 of P², P³, P⁴, and P⁵ is O, S, N, NH, NR^{d}, or NR⁷; and 1-2 of P², P³, P⁴, and P⁵ is an independently selected NR⁷ or CR⁷;

each R⁷ is independently selected from the group consisting of: —R⁸ and -L³-R⁹

R⁸ and R⁹ are independently selected from the group consisting of:

(a) C₃₋₁₂ cycloalkyl or C₃₋₁₂ cycloalkenyl, each of which is optionally substituted with 1-4 independently selected R⁷′;

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R⁷′;

(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R⁷′; and

(d) C₆₋₁₀ aryl optionally substituted with 1-4 independently selected R⁷′;

-L³ is selected from the group consisting of —O—, —CH₂—, —S—, —NH—, S(O)₁₋₂, C(═O)NH, NHC(═O), C(═O)O, OC(═O), C(═O), NHS(O)₂, and S(O)₂NH;

each occurrence of R⁷′ is independently selected from the group consisting of:

halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),

W is selected from the group consisting of:

(i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);

Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents point of attachment to W;

each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);

each occurrence of R² is independently selected from the group consisting of:

(i) H;

(ii) C₁₋₆ alkyl, which is optionally substituted with 1-3 independently selected R^{a};

(iii) —C(O)(C₁₋₆ alkyl) optionally substituted with 1-3 independently selected R^{a};

(iv) —C(O)O(C₁₋₄ alkyl) optionally substituted with 1-3 independently R^{a};

(v) —CON(R′)(R″);

(vi) —S(O)₁₋₂(NR′R″);

(vii) —S(O)₁₋₂(C₁₋₄ alkyl) optionally substituted with 1-3 independently selected R^{a};

(viii) —OH;

(ix) C₁₋₄ alkoxy; and

(x) -L⁴-L⁵-Rⁱ;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{e} is independently selected from the group consisting of:

halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; —C(═O)N(R′)(R″); and -L¹-L²-R^{h};

R^{d} is selected from the group consisting of: C₁₋₆ alkyl optionally substituted with 1-3 substituents each independently selected from the group consisting of halo, C₁₋₃ alkoxy, C₁₋₃ haloalkoxy, and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or

R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene; -L² is —O—, —N(H)—, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};

-L⁵- is selected from the group consisting of a bond and C₁₋₄ alkylene;

Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and

each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

In one aspect, this disclosure features compounds of Formula (I):

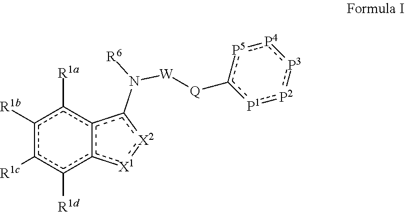

or a pharmaceutically acceptable salt thereof or a tautomer thereof,

X¹ is selected from the group consisting of O, S, N, NR², and CR¹;

X² is selected from the group consisting of O, S, N, NR⁴, and CR⁵;

each [symbol] is independently a single bond or a double bond, provided that: the five-membered ring comprising X¹ and X² is heteroaryl;

the 6-membered ring is aromatic:

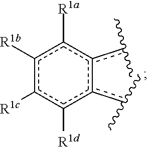

and

the ring comprising P¹, P², P³, P⁴, and P⁵ is aromatic; P¹, P², P³, P⁴, and P⁵ are defined according to (AA) or (BB):

### AA

each of P¹, P², P³, P⁴, and P⁵ is independently selected from the group consisting of: N, CH, CR⁷, and CR^{c} provided that:

1-2 of P¹, P², P³, P⁴, and P⁵ is an independently selected CR⁷; or

### BB

P¹ is absent (thereby providing a 5-membered ring),

each of P², P³, P⁴, and P⁵ is independently selected from the group consisting of O, S, N, NH, NR^{d}, NR⁷, CH, CR⁷, and CR^{c};

provided that 1-3 of P², P³, P⁴, and P⁵ is O, S, N, NH, NR^{d}, or NR⁷; and

1-2 of P², P³, P⁴, and P⁵ is an independently selected NR⁷ or CR⁷;

each R⁷ is independently selected from the group consisting of: —R⁸ and -L³-R⁹

R⁸ and R⁹ are independently selected from the group consisting of:

(a) C₃₋₁₂ cycloalkyl or C₃₋₁₂ cycloalkenyl, each of which is optionally substituted with 1-4 independently selected R⁷′,

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R⁷′;

(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R⁷′; and

(d) C₆₋₁₀ aryl optionally substituted with 1-4 independently selected R⁷′;

-L³ is selected from the group consisting of —O—, —CH₂—, —S—, —NH—, S(O)₁₋₂, C(═O)NH, NHC(═O), C(═O)O, OC(═O), C(═O), NHS(O)₂, and S(O)₂NH;

each occurrence of R⁷′ is independently selected from the group consisting of:

halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),

W is selected from the group consisting of:

(i) C(═O); (ii) C(═S); (iii) S(O)₁₋₂; (iv) C(═NR^{d}) or C(═N—CN); (v) C(═NH); (vi) C(═C—NO₂); (vii) S(O)N(R^{d}); and (viii) S(O)NH;

Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents point of attachment to W;

each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);

each occurrence of R² is independently selected from the group consisting of:

(i) H;

(ii) C₁₋₆ alkyl, which is optionally substituted with 1-3 independently selected R^{a};

(iii) —C(O)(C₁₋₆ alkyl) optionally substituted with 1-3 independently selected R^{a};

(iv) —C(O)O(C₁₋₄ alkyl) optionally substituted with 1-3 independently R^{a};

(v) —CON(R′)(R″);

(vi) —S(O)₁₋₂(NR′R″);

(vii) —S(O)₁₋₂(C₁₋₄ alkyl) optionally substituted with 1-3 independently selected R^{a};

(viii) —OH;

(ix) C₁₋₄ alkoxy; and

(x) -L⁴-L⁵-Rⁱ;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo; (b) cyano; (c) C₁₋₁₀ alkyl which is optionally substituted with 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (1) —S(O)₁₋₂(NR′R″); (m) —C₁₋₄ thioalkoxy; (n) —NO₂; (o) —C(═O)(C₁₋₁₀ alkyl); (p) —C(═O)O(C₁₋₄ alkyl); (q) —C(═O)OH; (r) —C(═O)N(R′)(R″); and (s) -L¹-L²-R^{h};

R^{d} is selected from the group consisting of: C₁₋₆ alkyl optionally substituted with 1-3 substituents each independently selected from the group consisting of halo and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene; -L² is —O—, —N(H)—, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and Ci-4 haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};

-L⁵- is selected from the group consisting of a bond and C₁₋₄ alkylene;

Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy; and

each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

In some embodiments, it is provided that:

(a) when X¹ is NR²; X² is CH; each of R^{1a}, R^{1b}, R^{1c}, R^{1d}, and R⁶ is H; W is C(═O); Q is NH; and P¹, P², P³, P⁴, and P⁵ are defined according to (AA); then:
R² cannot be CH₂CH₂OCH₃, CH₃, CH₂CH₃, or SO₂-(p-tolyl) when the

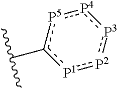

moiety is

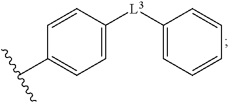

and -L³ is —O—, —NH—, or C(═O), and
R² cannot be CH₂CH₂CH₂N(CH₃)₂ or CH₂CH₂CH₂N(CH₂CH₃)₂ when the

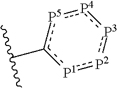

moiety is pyrimidinyl or pyridyl, R⁷ is R⁸, and R⁸ is unsubstituted phenyl; and

(b) the compound is not:

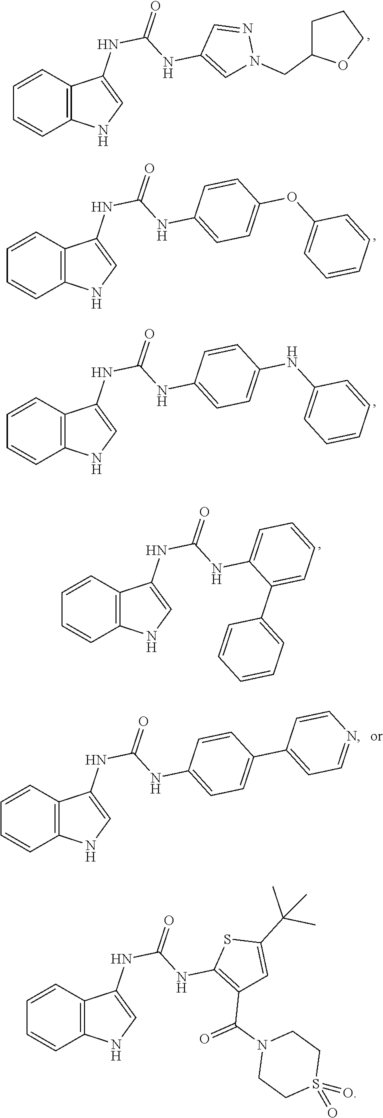 [SMILES: O=C(Nc1cnn(CC2CCCO2)c1)Nc1c[nH]c2ccccc12 ; O=C(Nc1ccc(Oc2ccccc2)cc1)Nc1c[nH]c2ccccc12 ; O=C(Nc1ccc(Nc2ccccc2)cc1)Nc1c[nH]c2ccccc12 ; O=C(Nc1ccccc1-c1ccccc1)Nc1c[nH]c2ccccc12 ; O=C(Nc1ccc(-c2ccncc2)cc1)Nc1c[nH]c2ccccc12 ; CC(C)(C)c1cc(C(=O)N2CCS(=O)(=O)CC2)c(NC(=O)Nc2c[nH]c3ccccc23)s1]

The Variables P¹, P², P³, P⁴, and P⁵
Embodiments when P¹, P², P³, P⁴, and P⁵ are as Defined According to (AA)

In some embodiments, P¹, P², P³, P⁴, and P⁵ are defined according to (AA).

In some embodiments, one of P¹, P², P³, P⁴, and P⁵ is N.

In some embodiments, two of P¹, P², P³, P⁴, and P⁵ are N.

In some embodiments, each one of P¹, P², P³, P⁴, and P⁵ is independently selected from the group consisting of CH, CR⁷, and, CR^{c}.

In some embodiments, one of P¹, P², P³, P⁴, and P⁵ is CR⁷.

In certain of these embodiments, P³ is CR⁷.

In some embodiments, P⁴ is N. In certain embodiments, P³ is CR⁷; and P⁴ is N.

In some embodiments, each of P¹, P², and P⁵ is independently selected from the group consisting of CH and CR^{c}. In certain embodiments, P³ is CR⁷; P⁴ is N; and each of P¹, P², and P⁵ is independently selected from the group consisting of CH and CR^{c}.

In some embodiments, one of P¹, P², and P⁵ is N; and each remaining of P¹, P², and P⁵ is independently selected from the group consisting of CH and CR^{c}. In certain embodiments, P³ is CR⁷; P⁴ is N; and one of P¹, P², and P⁵ is N; and each remaining of P¹, P², and P⁵ is independently selected from the group consisting of CH and CR^{c}.

In some embodiments, P¹ is N.

In certain of these embodiments, each of P², P⁴, and P⁵ is independently selected from the group consisting of CH and CR^{c}.

In certain other embodiments, one of P², P⁴, and P⁵ is N; and each remaining of P², P⁴, and P⁵ is independently selected from the group consisting of CH and CR^{c}.

In certain embodiments, P³ is CR⁷; P⁴ is N; and each of P¹, P², and P⁵ is independently selected from the group consisting of CH and CR^{c}.

In certain embodiments, P³ is CR⁷; P⁴ is N; P¹ is N; and each of P² and P⁵ is independently selected from the group consisting of CH and CR^{c}.

In certain embodiments, P³ is CR⁷; P⁴ is N; P⁵ is N; and each of P² and P¹ is independently selected from the group consisting of CH and CR^{c}.

In certain embodiments, P³ is CR⁷; and each of P¹, P², P⁴ and P⁵ is independently selected from the group consisting of CH and CR^{c}.

In certain embodiments, P³ is CR⁷; P¹ is N; and each of P², P⁴, and P⁵ is independently selected from the group consisting of CH and CR^{c}.

In certain embodiments, P³ is CR⁷; P⁴ and P² are N; and each of P¹ and P⁵ is independently selected from the group consisting of CH and CR^{c}.

In some embodiments, P⁴ is CR⁷.

In certain of these embodiments, each of P¹, P², P³, and P⁵ is independently selected from the group consisting of N, CH, and CR^{c}. As a non-limiting example, each of P¹, P², P³, and P⁵ can be independently selected from the group consisting of CH and CR^{c}.

In certain other embodiments, one of P¹, P², P³, and P⁵ is N; and each remaining of P¹, P², P³, and P⁵ is independently selected from the group consisting of CH and CR^{c}.

In certain embodiments, P⁴ is CR⁷; P³ is N; and each of P¹, P², and P⁵ is independently selected from the group consisting of CH and CR^{c}.

In certain embodiments, P⁴ is CR⁷; P² is N; and each of P¹, P³, and P⁵ is independently selected from the group consisting of CH and CR^{c}.

Embodiments when P¹, P², P³, P⁴, and P⁵ are as Defined According to (BB)

In some embodiments, P¹, P², P³, P⁴, and P⁵ are as defined according to (BB).

In some embodiments, one of P², P³, P⁴, and P⁵ is CR⁷ or NR⁷. For example, P³ is CR⁷ or NR⁷. In certain of these embodiments, each remaining P², P³, P⁴, and P⁵ is independently selected from the group consisting of: CH, CR^{c}, S, N, NH, and NR^{d}, provided that 1-3 (e.g., 1-2) of P², P³, P⁴, and P⁵ is S, N, NH, or NR^{d}.

In certain embodiments, P³ is CR⁷ or NR⁷; and each of P², P⁴, and P⁵ is independently selected from the group consisting of: O, S, N, NH, NR^{d}, CH, and CR^{c}, provided that 1-3 of P², P³, P⁴, and P⁵ is O, S, N, NH, NR^{d}, or NR⁷.

In certain of these embodiments, P³ is NR⁷; and each of P², P⁴, and P⁵ is independently selected from the group consisting of: O, S, N, NH, NR^{d}, CH, and CR^{c}.

In certain of the foregoing embodiments, P³ is NR⁷; and each of P², P⁴, and P⁵ is independently selected from the group consisting of: N, CH, and CR^{c}.

In certain embodiments, P³ is NR⁷; P² is CH or CR^{c} (e.g., CH); P⁴ is N; and P⁵ is CH or CR^{c} (e.g., CH).

In certain embodiments, P³ is NR⁷; P² is N; P⁴ is CH or CR^{c}, such as CH; and P⁵ is CH or CR^{c}, such as CH.

In certain embodiments, P³ is NR⁷; P² is CH or CR^{c}, such as C; P⁴ is CH or CR^{c}, such as CH; and P⁵ is N.

In certain embodiments, P³ is CR⁷; and each of P², P⁴, and P⁵ is independently selected from the group consisting of: CH, CR^{c}, S, N, NH, and NR^{d}, provided that 1-2 (e.g., 2) of P², P⁴, and P⁵ is S, N, NH, or NR^{d}.

In certain embodiments, P³ is CR⁷; P² is NH, NR^{d}, or S (e.g., S); P⁵ is N; and P⁴ is CH or CR^{c} (e.g., CH).

In certain embodiments, P³ is CR⁷; P² is NH, NR^{d}, or S (e.g., S); P⁵ is CH or CR^{c}; and P⁴ is N.

Non-Limiting Combinations of P¹, P², P³, P⁴, and P⁵

In some embodiments, the

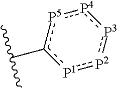

moiety has the formula:

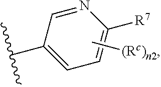

wherein n2 is 0, 1, or 2.

In certain embodiments, the

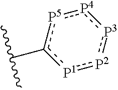

moiety has the formula:

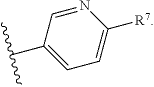

In certain embodiments, the

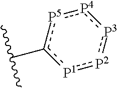

moiety has the formula:

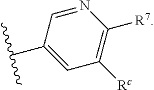

In some embodiments, the

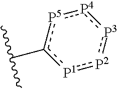

moiety has the formula:

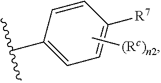

wherein n2 is 0, 1, or 2.

In certain of these embodiments, the

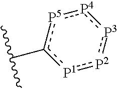

moiety has the formula:

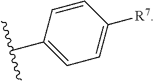

In certain embodiments, the

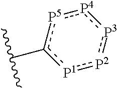

moiety has the formula:

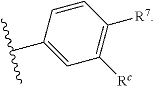

In some embodiments, the

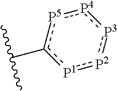

moiety has the formula:

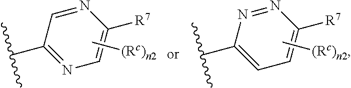

herein n2 is 0, 1, or 2.

In some embodiments, the

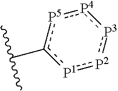

moiety has the formula:

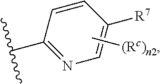

wherein n2 is 0, 1, or 2.

In certain of these embodiments, the

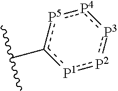

moiety has the formula:

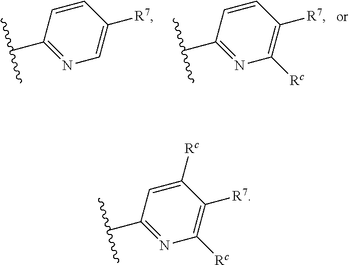

In some embodiments, the

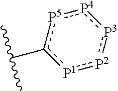

moiety has the formula:

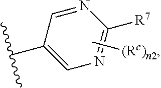

wherein n2 is 0, 1, or 2.

In some embodiments, the

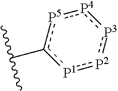

moiety has the formula:

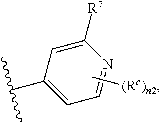

wherein n2 is 0, 1, or 2.

In certain of these embodiments, the

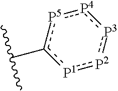

moiety has the formula:

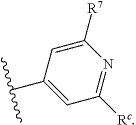

In certain embodiments, the

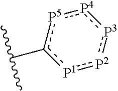

moiety has the formula:

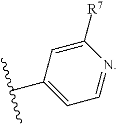

In some embodiments, the

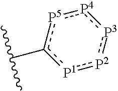

moiety has the formula:

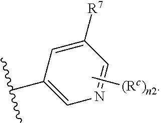

In certain of these embodiments, the

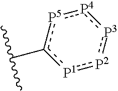

moiety has the formula:

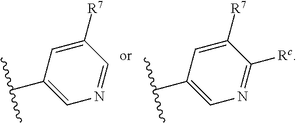

In some embodiments, the

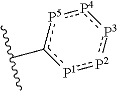

moiety has the formula:

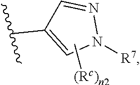

wherein n2 is 0 or 1, such as 0.

In certain of these embodiments, the

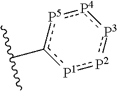

moiety has the formula:

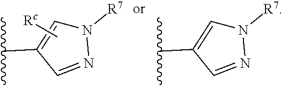

In some embodiments, the

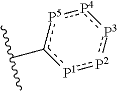

moiety has the formula:

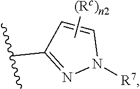

wherein n2 is 0 or 1, such as 0.

In some embodiments, the

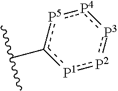

moiety has the formula:

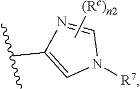

wherein n2 is 0 or 1, such as 0.

### The Variable R⁷

In some embodiments, R⁷ is R⁸.

In some embodiments, R⁸ is selected from the group consisting of:

(a) C₃₋₁₂ cycloalkyl or C₃₋₁₂ cycloalkenyl, each of which is optionally substituted with 1-4 independently selected R⁷′; and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R⁷′.

In certain embodiments, R⁸ is selected from the group consisting of:

(a) C₃₋₁₂ cycloalkyl or C₃₋₁₂ cycloalkenyl, each of which is substituted with 1-4 independently selected R⁷′; and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R⁷′.

In certain embodiments, R⁸ is C₃₋₁₂ cycloalkyl or C₃₋₁₂ cycloalkenyl, each of which is substituted with 1-4 independently selected R⁷′.

In certain embodiments, R⁸ is C₄₋₁₀ cycloalkyl or C₄₋₁₀ cycloalkenyl, each of which is substituted with 1-4 independently selected R⁷′.

In certain of these embodiments, R⁸ is C₄₋₈ cycloalkyl or C₄₋₈ cycloalkenyl, each of which is substituted with 1-4 independently selected R⁷′

In certain of these embodiments, R⁸ is C₄₋₈ cycloalkyl which is substituted with 1-4 independently selected R⁷′.

In certain embodiments, R⁸ is C₄₋₈ cycloalkyl which is substituted with 1-3 R⁷′.

In certain of these embodiments, R⁸ is cyclohexyl which is substituted with 1-3 (e.g., 1 or 2) R⁷′.

As a non-limiting example of the foregoing embodiments, R⁸ can be

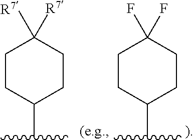

In certain embodiments, R⁸ is cyclobutyl which is substituted with 1-3 (e.g., 1 or 2) R⁷′.

As a non-limiting example of the foregoing embodiments, R⁸ can be (e.g.,

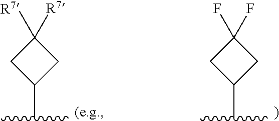

As another non-limiting example, R⁸ can be

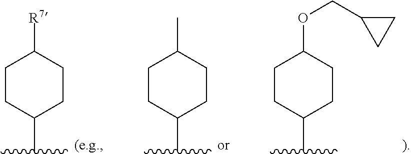

In certain embodiments, R⁸ is spirocyclic C₆₋₁₂ cycloalkyl which is substituted with 1-4 independently selected R⁷′. In certain of these embodiments, R⁸ is

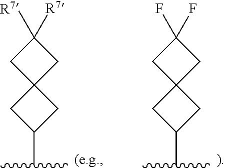

In certain embodiments, R⁸ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R⁷′.

In certain embodiments, R⁸ is heterocyclyl or heterocycloalkenyl of 4-10 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R⁷′.

In certain embodiments, R⁸ is heterocyclyl or heterocycloalkenyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R⁷′.

In certain of these embodiments, R⁸ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R⁷′.

In certain embodiments, R⁸ is heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 1-3 independently selected R⁷′.

In certain of these embodiments, R⁸ is selected from the group consisting of azetidinyl, oxetanyl, pyrrolidinyl, tetrahydrofuranyl, dioxanyl (e.g., 1,3-dioxanyl), piperidinyl, piperazinyl, morpholinyl, and tetrahydropyranyl, each of which is substituted with 1-3 (e.g., 1 or 2) independently selected R⁷′.

In certain of the foregoing embodiments, R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 1-3 (e.g., 1 or 2) independently selected R⁷′.

In certain embodiments, R⁸ is selected from the group consisting of azetidinyl, pyrrolidinyl, and piperidinyl, each of which is substituted with 1-3 (e.g., 1 or 2) independently selected R⁷′.

In certain embodiments, R⁸ is selected from the group consisting of azetidinyl, pyrrolidinyl, morpholinyl, and piperidinyl, each of which is substituted with 1-3 (e.g., 1 or 2) independently selected R⁷′.

As non-limiting examples, R⁸ can be selected from the group consisting of:

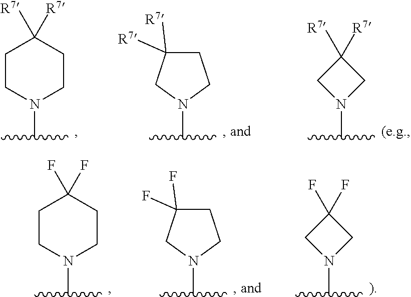

As a non-limiting example of the foregoing embodiments, R⁸ can be selected from the group consisting of:

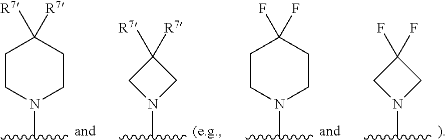

As further non-limiting examples, R⁸ can be selected from the group consisting of:

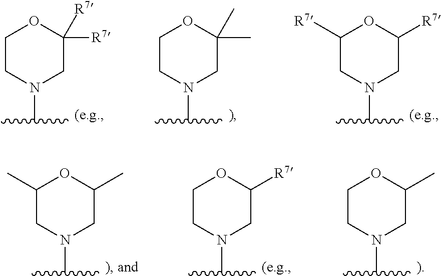

As another non-limiting example, R⁸ can be selected from the group consisting of:

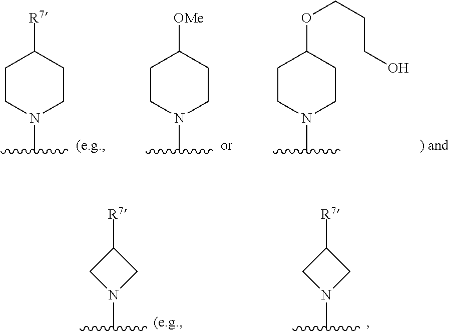

wherein R⁷′ is C₁₋₄ haloalkyl, such as —CF₃).

As another non-limiting example, R⁸ can be R⁸ is

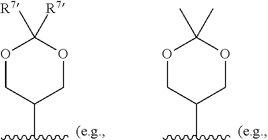

As further non-limiting examples, R⁸ can be selected from the group consisting of:

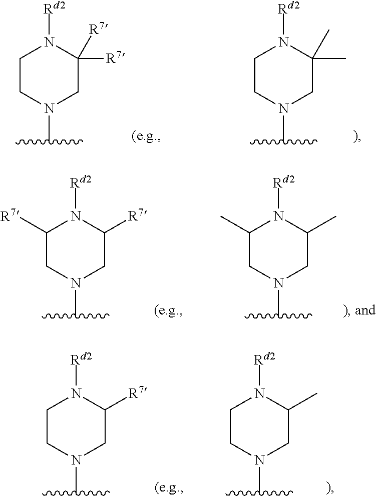

wherein R^{d2} is H or R^{d}.

In certain embodiments, R⁸ is spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′.

In certain of the foregoing embodiments, R⁸ is selected from the group consisting of: 2-azaspiro[3.3]heptanyl, 1-oxa-9-azaspiro[5.5]undecanyl, 6-azaspiro[2.5]octanyl, 1,5-dioxaspiro[5.5]undecanyl, 7-azaspiro[3.5]nonanyl, and 2,6-diazaspiro[3.3]heptanyl, each of which is optionally substituted with 1-4 independently selected R⁷′ at one or more ring carbon atoms, wherein a ring nitrogen is optionally substituted with R^{d}.

In certain of these embodiments, R⁸ is selected from the group consisting of: 2-azaspiro[3.3]heptanyl, 1-oxa-9-azaspiro[5.5]undecanyl, and 6-azaspiro[2.5]octanyl, each of which is optionally substituted with 1-4 independently selected R⁷′ at the ring carbon atoms.

As a non-limiting example of the foregoing embodiments, R⁸ can be

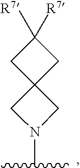

such as:

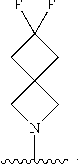 [SMILES: *N1CC2(C1)CC(F)(F)C2]

As further non-limiting examples, R⁸ can be selected from the group consisting of:

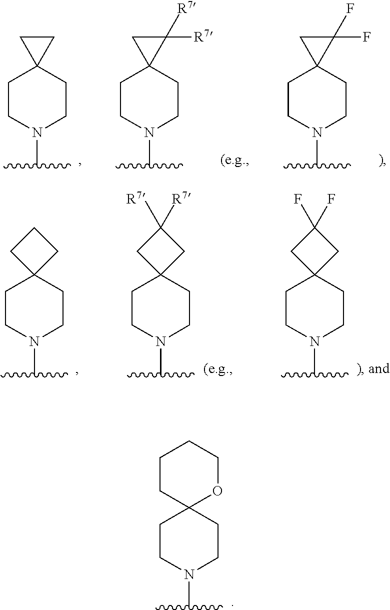

As further non-limiting examples, R⁸ can be

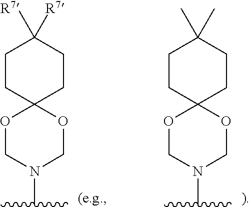

As further non-limiting examples, R⁸ can be

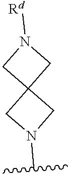

optionally wherein R^{d} is C₁₋₆ alkyl optionally substituted with 1-3 substituents each independently selected from the group consisting of halo, C₁₋₃ alkoxy, and C₁₋₃ haloalkoxy, such as wherein R^{d} is C₂₋₄ alkyl substituted with 1-3 independently selected halo

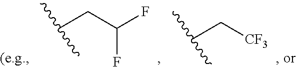 [SMILES: *CC(F)F ; *CC(F)(F)F]

In certain embodiments, R⁸ is bridged heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′. For example, R⁸ can be

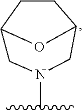 [SMILES: *N1CC2CCC(C1)O2]

which is optionally substituted with 1-2 R⁷′ at one or more ring carbon atoms.

In certain embodiments, R⁸ is C₃₋₁₂ cycloalkyl or C₃₋₁₂ cycloalkenyl which is unsubstituted.

In certain of these embodiments, R⁸ is C₃₋₈ (e.g., C₃₋₅ or C₇₋₈) monocyclic cycloalkyl which is unsubstituted. For example, R⁸ can be C₄₋₆ monocyclic cycloalkyl which is unsubstituted, such as cyclobutyl or cyclopentyl. As another non-limiting example, R⁸ can be cyclohexyl.

In certain embodiments, R⁸ is C₇₋₁₂ bicyclic cycloalkyl which is unsubstituted.

In certain of these embodiments, R⁸ is C₇₋₁₂ spirocyclic cycloalkyl which is unsubstituted. As a non-limiting example of the foregoing embodiments, R⁸ can be

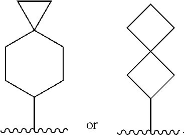

In certain embodiments, R⁸ is C₇₋₁₂ bridged bicyclic cycloalkyl which is unsubstituted. As a non-limiting example of the foregoing embodiments, R⁸ can be

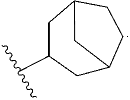 [SMILES: *C1CC2CCC(C1)C2]

In certain embodiments, R⁸ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂.

In certain embodiments, R⁸ is monocyclic heterocyclyl of 3-8 ring atoms, wherein 1-2 ring atoms are heteroatoms, each independently selected from the group consisting of N, N(H), N(R^{d}), O, and S(O)₀₋₂.

In certain of these embodiments, R⁸ is selected from the group consisting of: azetidinyl, oxetanyl, pyrrolidinyl, tetrahydrofuranyl, tetrahydropyranyl, piperidinyl, piperazinyl, morpholinyl, azepinyl, and oxepanyl, wherein a ring nitrogen atom is optionally substituted with R^{d}.

In certain of the foregoing embodiments, R⁸ is azetidinyl, pyrrolidinyl, piperidinyl, piperazinyl, morpholinyl, or oxepanyl, wherein a ring nitrogen atom is optionally substituted with R^{d}.

As a non-limiting example of the foregoing embodiments, R⁸ can be morpholinyl, piperidinyl

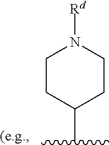

such as

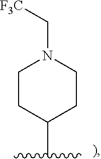 [SMILES: *C1CCN(CC(F)(F)F)CC1]

or oxepanyl, wherein a ring nitrogen atom is optionally substituted with R^{d}.

In certain embodiments, R⁸ is azetidinyl

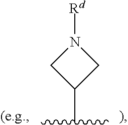

pyrrolidinyl

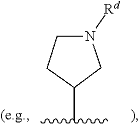

piperidinyl

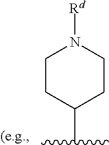

such as

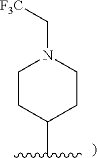 [SMILES: *C1CCN(CC(F)(F)F)CC1]

or piperazinyl

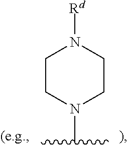

wherein a ring nitrogen atom is substituted with R^{d},

optionally wherein R^{d} is C₁₋₆ alkyl optionally substituted with 1-3 substituents each independently selected from the group consisting of halo, C₁₋₃ alkoxy, and C₁₋₃ haloalkoxy, such as wherein R^{d} is C₂₋₄ alkyl substituted with 1-3 independently selected halo

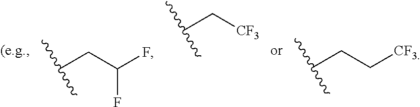

In certain embodiments, R⁸ is pyrrolidinyl, piperidinyl, or piperazinyl, wherein a ring nitrogen atom is substituted with R^{d}.

In certain of these embodiments, R⁸ is piperidinyl

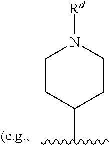

such as

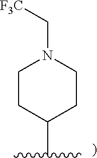 [SMILES: *C1CCN(CC(F)(F)F)CC1]

or piperazinyl

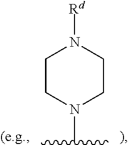

wherein a ring nitrogen atom is substituted with R^{d}, optionally wherein R^{d} is C₁₋₆ alkyl optionally substituted with 1-3 substituents each independently selected from the group consisting of halo, C₁₋₃ alkoxy, C₁₋₃ haloalkoxy, such as wherein R^{d} is C₂₋₄ alkyl substituted with 1-3 independently selected halo

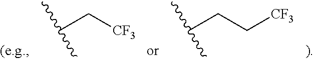 [SMILES: *CC(F)(F)F ; *CCC(F)(F)F]

In certain embodiments, R⁸ is selected from the group consisting of:

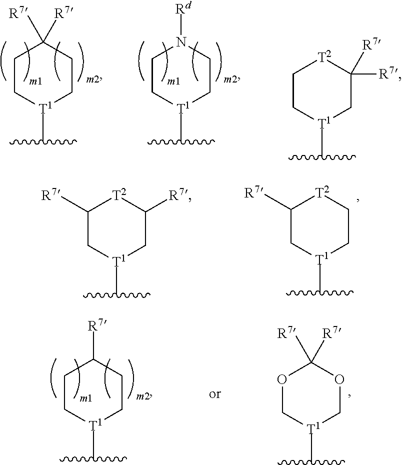

wherein m1 and m2 are independently 0, 1, or 2; T¹ is CH or N; and T² is CH₂, NH, NR^{d}, or 0;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′,

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F; and

optionally wherein R^{d} is C₁₋₆ alkyl, such as C₂₋₄ alkyl, optionally substituted with 1-3 independently selected halo, such as —F.

In certain embodiments, R⁸ is selected from the group consisting of:

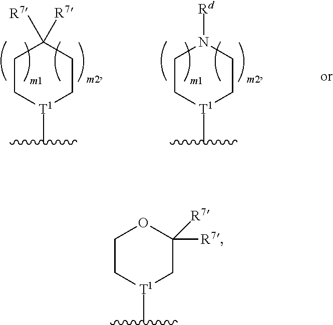

wherein m1 and m2 are independently 0, 1, or 2, and T¹ is CH or N; and
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′, such as:

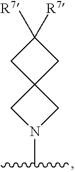

optionally wherein each R⁷′ is independently selected from the group consisting of C₁₋₃ alkyl and halo, such as methyl and —F; and optionally wherein R^{d} is C₁₋₆ alkyl, such as C₂₋₄ alkyl, optionally substituted with 1-3 independently selected halo, such as —F.

In certain of these embodiments, R⁸ is selected from the group consisting of:

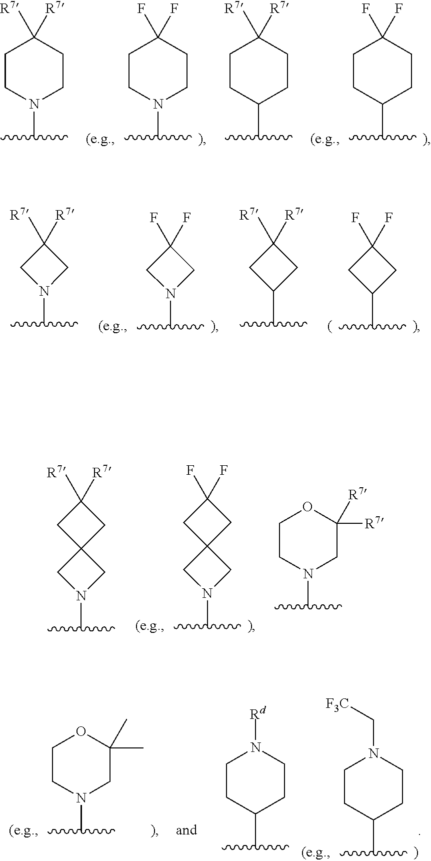

In certain embodiments, R⁸ is

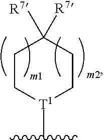

wherein m1 and m2 are independently 0, 1, or 2, and T¹ is CH or N, such as:

wherein R⁸ is selected from the group consisting of:

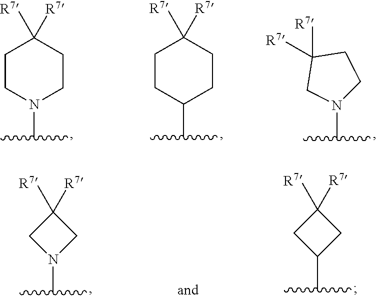

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F, such as wherein each R⁷′ is an independently selected halo, such as —F.

In certain embodiments, R⁸ is

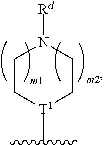

wherein m1 and m2 are independently 0, 1, or 2, and T¹ is CH or N, such as: wherein R⁸ is selected from the group consisting of:

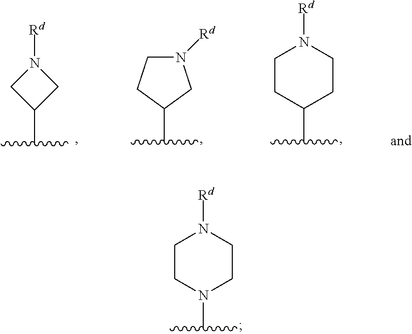

optionally wherein R^{d} is C₁₋₆ alkyl, such as C₂₋₄ alkyl, optionally substituted with 1-3 independently selected halo, such as —F.

In certain embodiments, R⁸ is selected from the group consisting of:

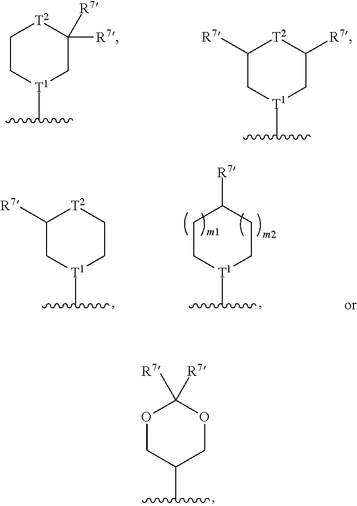

wherein m1 and m2 are independently 0, 1, or 2; T¹ is CH or N; and T² is CH₂, NH, NR^{d}, or O; such as: wherein R⁸ is selected from the group consisting of:

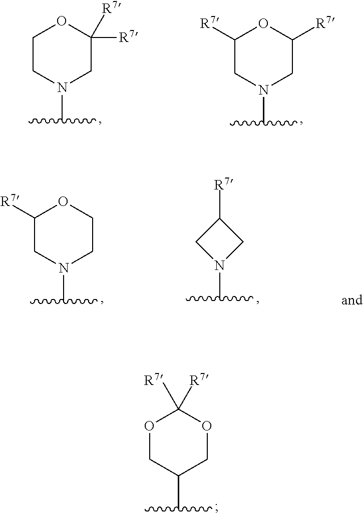

optionally wherein each R⁷′ is independently selected from the group consisting of C₁₋₃ alkyl and C₁₋₃ haloalkyl.

In certain embodiments, R⁸ is selected from the group consisting of:
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′;

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F.

In certain of these embodiments, R⁸ is

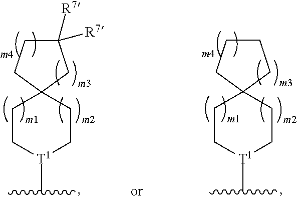

wherein m1, m2, m3, and m4 are independently 0, 1, or 2, provided that m1+m2+m3+m4≤6, and T¹ is CH or N, such as: wherein R⁸ is selected from the group consisting of:

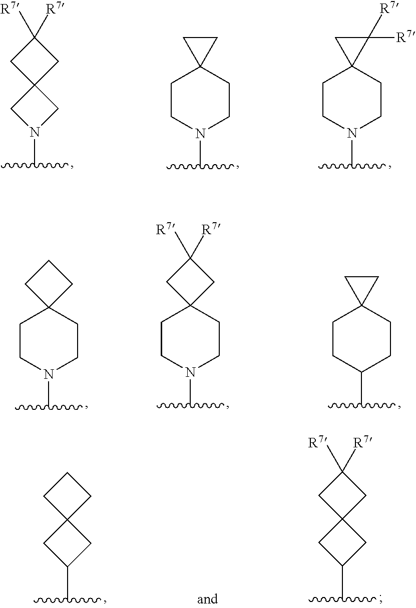

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F, such as: wherein each R⁷′ is an independently selected halo, such as —F.

In certain embodiments, R⁸ is

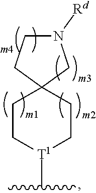

wherein m1, m2, m3, and m4 are independently 0, 1, or 2, provided that m1+m2+m3+m4≤6, and T¹ is CH or N, such as: wherein R⁸ is

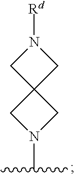

optionally wherein R^{d} is C₁₋₆ alkyl, such as C₂₋₄ alkyl, optionally substituted with 1-3 independently selected halo, such as —F.

In certain embodiments, R⁸ is

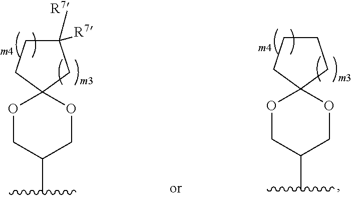

wherein m3 and m4 are independently 0, 1, or 2, provided that m3+m4≤4, such as: wherein R⁸ is

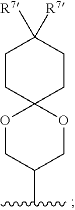

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F, such as: wherein each R⁷′ is an independently selected halo, such as —F.

In certain embodiments, R⁸ is bicyclic or polycyclic heterocyclyl or heterocycloalkenyl of 7-12 ring atoms, wherein 1-3 ring atoms are heteroatoms, each independently selected from the group consisting of N, N(H), N(R^{d}), O, and S(O)₀₋₂.

In certain of these embodiments, R⁸ is bicyclic or polycyclic heterocyclyl of 7-12 ring atoms, wherein 1-3 ring atoms are heteroatoms, each independently selected from the group consisting of N, N(H), N(R^{d}), O, and S(O)₀₋₂.

As a non-limiting example of the foregoing embodiments, R⁸ can be

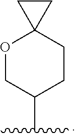 [SMILES: *C1CCC2(CC2)OC1]

In certain embodiments, R⁸ is heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R⁷′.

In certain embodiments, R⁸ is heteroaryl of 5-6 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R⁷′.

In certain of these embodiments, R⁸ is heteroaryl of 5 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R⁷′.

In certain of the foregoing embodiments, R⁸ is pyrazolyl, imidazolyl, thiazolyl, oxazolyl, triazolyl, each of which is optionally substituted with 1-2 independently selected R⁷′ at one or more ring carbon atoms and optionally substituted with one R^{d} at a ring nitrogen atom.

As a non-limiting example of the foregoing embodiments, R⁸ can be thiazolyl optionally substituted with 1-2 independently selected R⁷′

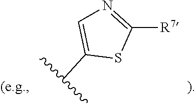

In certain embodiments, R⁸ is bicyclic heteroaryl of 7-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R⁷′.

As a non-limiting example of the foregoing embodiments, R⁸ can be

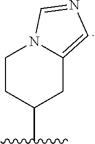 [SMILES: *C1CCn2cncc2C1]

In certain embodiments, R⁸ is C₆₋₁₀ aryl optionally substituted with 1-4 independently selected R⁷′.

In certain of these embodiments, R⁸ is phenyl optionally substituted with 1-2 independently selected R⁷′ (e.g., unsubstituted phenyl).

In some embodiments, R⁷ is -L³-R⁹.

In certain of these embodiments, -L³ is —O—. In certain embodiments, -L³ is —NH—.

In certain embodiments, -L³ is —S— or S(O)₁₋₂. In certain embodiments, -L³ is —CH₂—. In certain embodiments, -L³ is selected from the group consisting of: C(═O)NH, NHC(═O), C(═O)O, OC(═O), C(═O), NHS(O)₂, and S(O)₂NH. In certain embodiments, -L³ is C₁₋₄ alkylene, such as CH₂ or

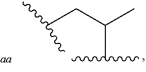

wherein aa is the point of attachment to R⁹.

In certain embodiments (when R⁷ is -L³-R⁹), R⁹ is selected from the group consisting of:

(a) C₃₋₁₂ cycloalkyl or C₃₋₁₂ cycloalkenyl, each of which is optionally substituted with 1-4 independently selected R⁷′, and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R⁷′.

In certain embodiments, R⁹ is C₃₋₁₂ cycloalkyl or C₃₋₁₂ cycloalkenyl, each of which is optionally substituted with 1-4 independently selected R⁷′.

In certain of these embodiments, R⁹ is C₄₋₈ cycloalkyl which is optionally substituted with 1-2 R⁷′.

As non-limiting examples, R⁹ can be cyclobutyl, cyclopentyl, cyclohexyl, or spiro[3.3]heptanyl, each of which is optionally substituted with 1-2 R⁷′ (e.g., unsubstituted).

In certain embodiments, R⁹ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R⁷′.

In certain of these embodiments, R⁹ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R⁷′.

As non-limiting examples of the foregoing embodiments, R⁹ is selected from the group consisting of azetidinyl, oxetanyl, pyrrolidinyl, tetrahydrofuranyl, piperidinyl, piperazinyl, morpholinyl, and azepinyl, each of which is optionally substituted with 1-2 independently selected R⁷′ (e.g., unsubstituted).

In certain embodiments, R⁷ is L³-R⁹; L³ is —O— or —NH—; and R⁹ is selected from the group consisting:

C₄₋₈ cycloalkyl which is optionally substituted with 1-2 R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R⁷′.

In certain of these embodiments, R⁷ is L³-R⁹; L³ is —O— or —NH—; and R⁹ is selected from the group consisting of cyclobutyl, cyclopentyl, cyclohexyl, and oxetanyl, each of which is optionally substituted with 1-2 independently selected R⁷′ (e.g., unsubstituted). For example, L³ can be —O—.

When R⁷ is -L³-R⁹, non-limiting examples of R⁷ can include:

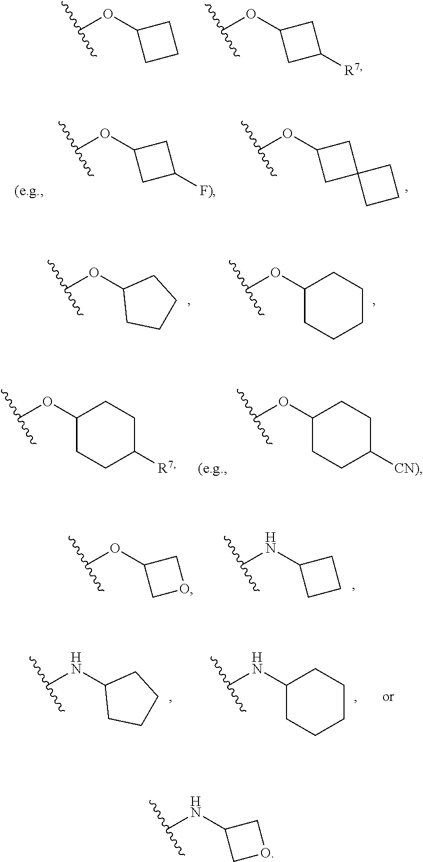

In certain embodiments, the

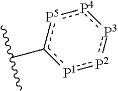

moiety has the formula:

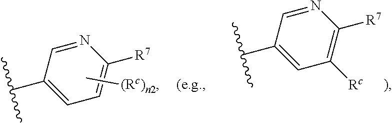

wherein n2 is 0, 1, or 2; and R⁷ is R⁸, wherein R⁸ is selected from the group consisting of:

C₄₋₈ cycloalkyl which is optionally substituted with 1-4 independently selected R⁷′; and

heterocyclyl of 4-12 (e.g., 4-8) ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′.

In certain embodiments, the

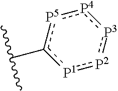

moiety has the formula:

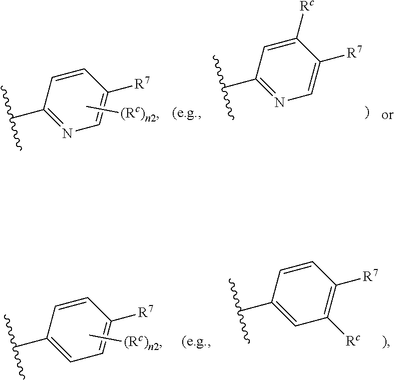

wherein n2 is 0, 1, or 2; and R⁷ is R⁸, wherein R⁸ is selected from the group consisting of:

C₄₋₈ cycloalkyl which is optionally substituted with 1-4 independently selected R⁷′; and

heterocyclyl of 4-12 (e.g., 4-8) ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′.

In certain embodiments, the

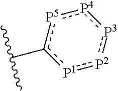

moiety has the formula:

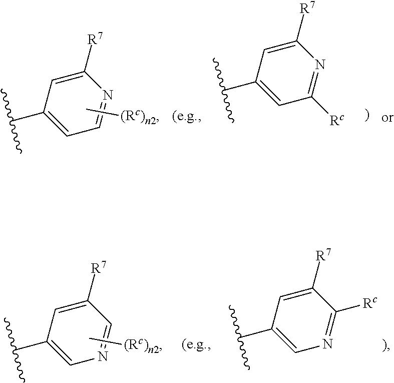

wherein n2 is 0, 1, or 2; and R⁷ is R⁸, wherein R⁸ is selected from the group consisting of:

C₄₋₈ cycloalkyl which is optionally substituted with 1-4 independently selected R^{7′}; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′.

In certain embodiments, the

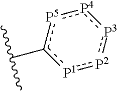

moiety has the formula:

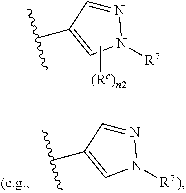

wherein n2 is 0 or 1 (e.g., 0); and R⁷ is R⁸, wherein R⁸ is selected from the group consisting of:

C₄₋₈ cycloalkyl which is optionally substituted with 1-4 independently selected R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′.

In certain embodiments (when the

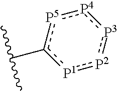

moiety has the formula:

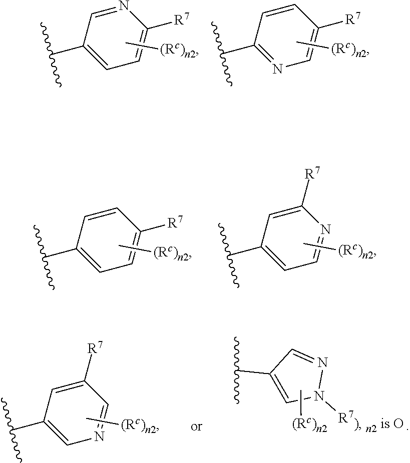

In certain embodiments (when the

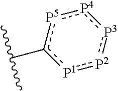

moiety has the formula:

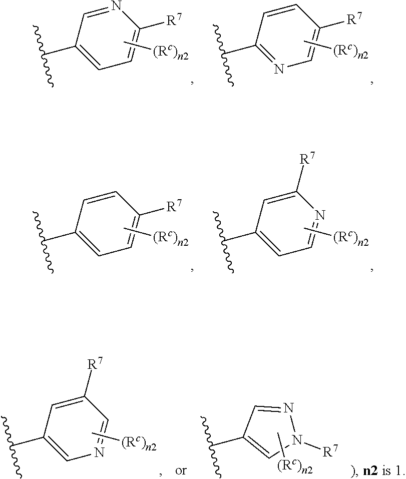

In certain of these embodiments, R^{c} is located ortho to R⁷.

In certain embodiments (when the

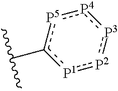

moiety has the formula:

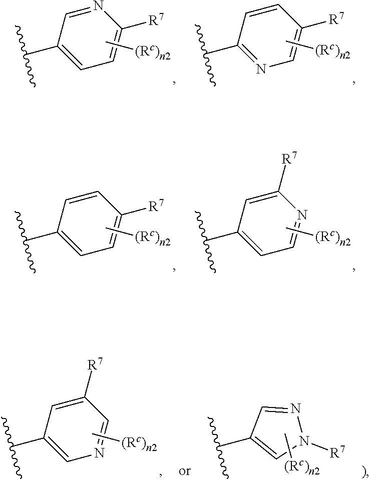

R⁷ is R⁸; and R⁸ is C₄₋₈ cycloalkyl which is substituted with 1-3 R⁷′.

In certain of these embodiments, R⁸ is cyclohexyl which is substituted with 1-3 R⁷′, such as

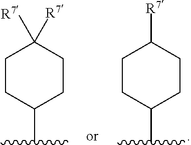

In certain embodiments, R⁸ is cyclobutyl which is substituted with 1-3 R⁷′, such as

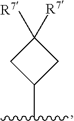

such as

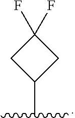 [SMILES: *C1CC(F)(F)C1]

In certain embodiments (when the

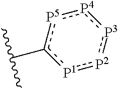

moiety has the formula:

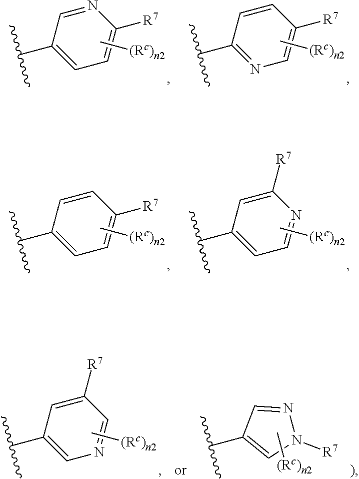

R⁷ is R⁸; and R⁸ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R⁷′.

In certain of these embodiments, R⁸ is heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 1-3 independently selected R⁷′.

In certain embodiments, R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 1-3 (e.g., 1 or 2) independently selected R⁷′.

In certain embodiments, R⁸ is selected from the group consisting of azetidinyl, pyrrolidinyl, and piperidinyl, each of which is substituted with 2-4 (e.g., 2) independently selected R⁷′.

As non-limiting examples of the foregoing embodiments, R⁸ can be selected from the group consisting of:

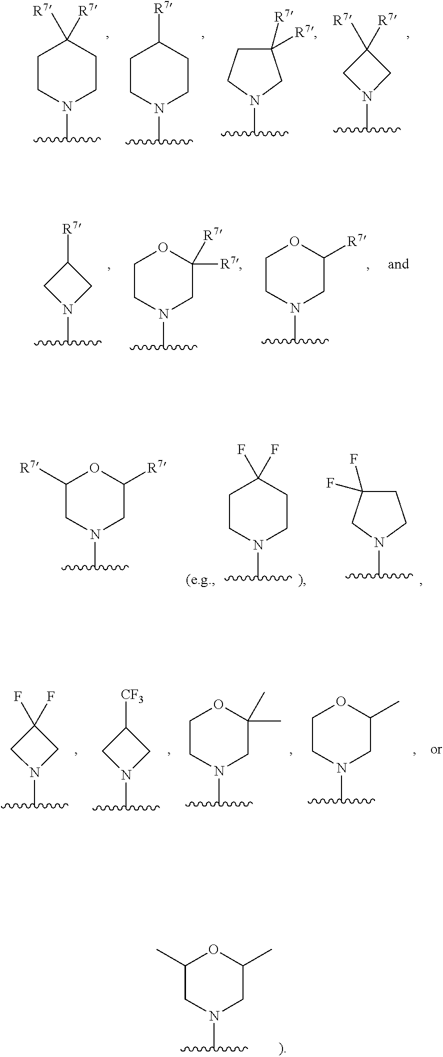

For example, R⁸ can be

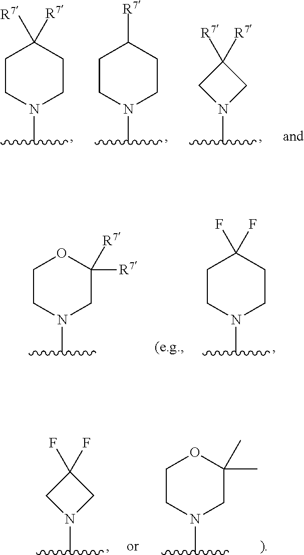

In certain embodiments (when the

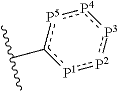

moiety has the formula:

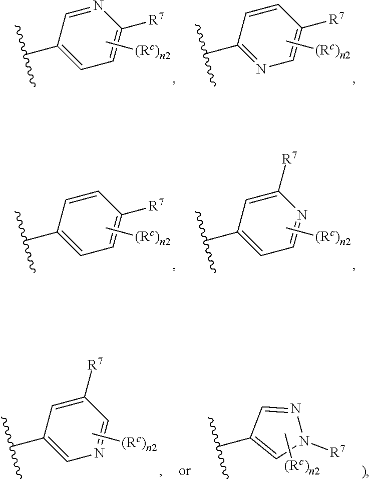

R⁷ is R⁸; and R⁸ is spirocyclic heterocyclyl of 6-12 (e.g., 6-8) ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′, such as:

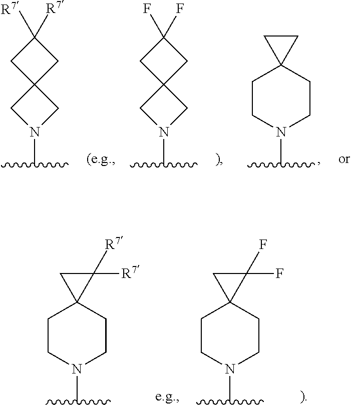

In certain embodiments (when the

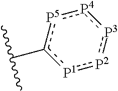

moiety has the formula:

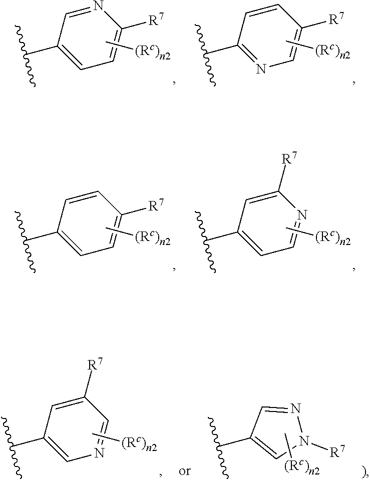

R⁷ is R⁸; and R⁸ is heterocyclyl of 4-8 ring atoms, wherein 1-3 ring atoms are heteroatoms, each independently selected from the group consisting of N, N(H), N(R^{d}), O, and S(O)₀₋₂, provided that R⁸ contains a ring N(R^{d}) group.

In certain of these embodiments, R⁸ is selected from the group consisting of: azetidinyl, pyrrolidinyl, piperidinyl, piperazinyl, and 2,6-diazaspiro[3.3]heptanyl, wherein a ring nitrogen atom is substituted with R^{d}, such as wherein R⁸ is

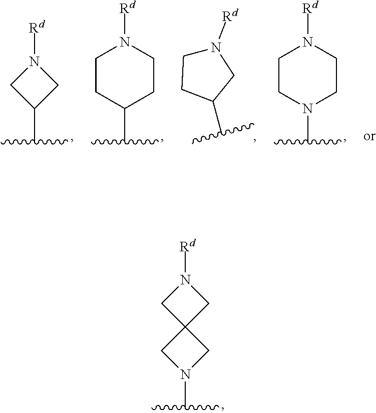

optionally wherein R^{d} is C₁₋₆ alkyl optionally substituted with 1-3 substituents each independently selected from the group consisting of halo, C₁₋₃ alkoxy, and C₁₋₃ haloalkoxy, such as wherein R^{d} is C₂₋₄ alkyl substituted with 1-3 independently selected halo

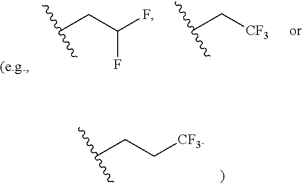 [SMILES: *CC(F)F ; *CC(F)(F)F ; *CCC(F)(F)F]

In certain embodiments (when the

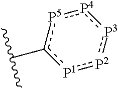

moiety has the formula:

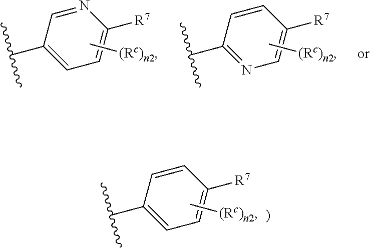

R⁷ is R⁸; and R⁸ is C₄₋₆ monocyclic cycloalkyl which is unsubstituted (e.g., cyclopentyl, cyclobutyl, or cyclohexyl); or R⁸ is C₇₋₈ bicyclic (e.g., spirocyclic) cycloalkyl which is unsubstituted

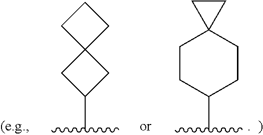 [SMILES: *C1CC2(CCC2)C1 ; *C1CCC2(CC1)CC2]

In certain embodiments, the

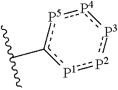

moiety has the formula:

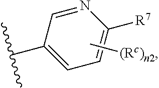

wherein n2 is 0, 1, or 2; and R⁷ is -L³-R⁹, wherein:

L³ is —NH— or —O—; and R⁹ is selected from the group consisting:

C₄₋₈ cycloalkyl which is optionally substituted with 1-2 R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R⁷′.

In certain embodiments, the

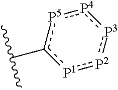

moiety has the formula:

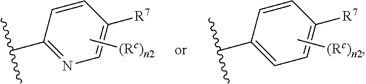

wherein n2 is 0, 1, or 2; and R⁷ is -L³-R⁹, wherein:

L³ is —NH— or —O—; and R⁹ is selected from the group consisting:

C₄₋₈ cycloalkyl which is optionally substituted with 1-2 R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R⁷′.

In certain embodiments (when the

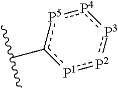

moiety has the formula:

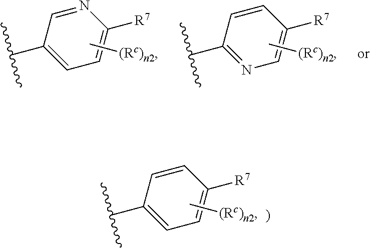

R⁷ is L³-R⁹; L³ is —O— or —NH—; and R⁹ is selected from the group consisting of cyclobutyl, cyclopentyl, cyclohexyl, and oxetanyl, each of which is optionally substituted with 1-2 independently selected R⁷′ (e.g., unsubstituted). In certain of these embodiments, L³ is —O—.

In certain embodiments (when the

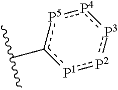

moiety has the formula:

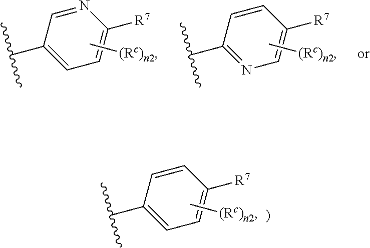

R⁷ is

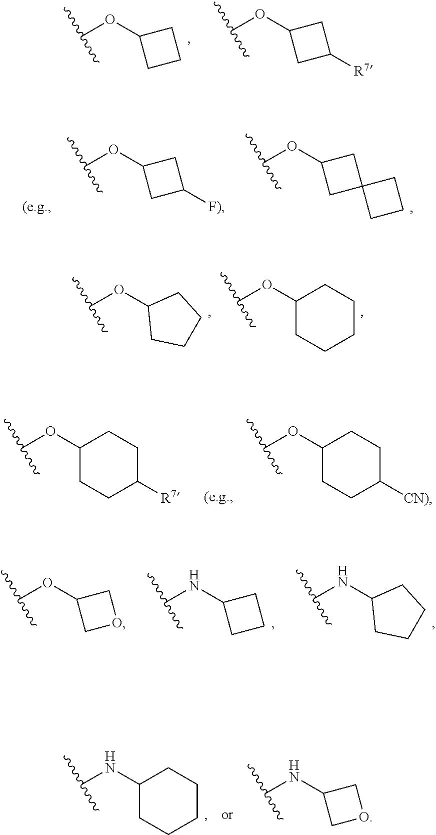

### The Variable R⁷′

In certain embodiments, each R⁷′ when present is independently selected from the group consisting of: halo, —CN, —OH, —C₁₋₄ alkyl optionally substituted with R^{a}, —C₁₋₄ haloalkyl, —C₁₋₆ alkoxy optionally substituted with R^{a}, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″).

In certain embodiments, each R⁷′ when present is independently selected from the group consisting of: halo, —CN, —C₁₋₄ alkyl optionally substituted with R^{a}, —C₁₋₄ haloalkyl, —C₁₋₆ alkoxy optionally substituted with R^{a}, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″).

In certain embodiments, each R⁷′ when present is independently halo. For example, each R⁷′ when present can be —F.

In certain embodiments, each R⁷′ when present is independently C₁₋₃ alkyl, such as methyl.

In certain embodiments, each R⁷′ when present is an independently selected C₁₋₃ haloalkyl, such as —CF₃.

In certain embodiments, one occurrence of R⁷′ is —C₁₋₄ alkyl optionally substituted with R^{a}, such as unsubstituted C₁₋₄ alkyl (e.g., methyl, ethyl, n-propyl) or R⁷′ is —C₁₋₄ alkyl substituted with R^{a} (e.g., —C₁₋₄ alkyl substituted with OH or C₃₋₆ cycloalkyl).

In certain embodiments, one occurrence of R⁷′ is —CN.

In certain embodiments, one occurrence of R⁷′ is C₁₋₆ alkoxy optionally substituted with R^{a}, such as unsubstituted C₁₋₆ alkoxy (e.g., methoxy); or C₁₋₆ alkoxy substituted with R^{a} (e.g., —C₁₋₄ alkoxy substituted with OH or C₃₋₆ cycloalkyl).

In certain of the foregoing embodiments of one occurrence of R⁷′, each remaining occurrence of R⁷′ when present is independently halo (e.g., —F).

In certain embodiments, each R^{c} when present is independently selected from the group consisting of: (a) halo; (b) cyano; (c) C₁₋₁₀ alkyl which is optionally substituted with 1-6 independently selected R^{a}; (g) C₁₋₄ alkoxy; (h) C₁₋₄ haloalkoxy; (i) —S(O)₁₋₂(C₁₋₄ alkyl); (j) —NR^{e}R^{f}; (k) —OH; (1) —S(O)₁₋₂(NR′R″); (m) —C₁₋₄ thioalkoxy; (n) —NO₂; (o) —C(═O)(C₁₋₁₀ alkyl); (p) —C(═O)O(C₁₋₄ alkyl); (q) —C(═O)OH; and (r) —C(═O)N(R′)(R″).

In certain embodiments, each R^{c} when present is independently selected from the group consisting of: (a) halo; (b) cyano; (c) C₁₋₁₀ alkyl optionally substituted with 1-6 independently selected —F or —Cl; (g) C₁₋₄ alkoxy; (h) C₁₋₄ haloalkoxy; (i) —S(O)₁₋₂(C₁₋₄ alkyl); and —C(═O)(C₁₋₁₀ alkyl).

In certain embodiments, each R^{c} is independently selected from the group consisting of: halo, cyano, C₁₋₃ alkyl, and C₁₋₃ alkoxy.

In certain embodiments, each R^{c} is an independently selected halo (e.g., —F or —Cl), C₁₋₄ alkyl (e.g., CH₃), or CF₃. For example, each R^{c} can be —F. As another non-limiting example, each R^{c} can be —Cl.

### The Variables Q and W

In some embodiments, Q is NH.

In some embodiments, Q is N(C₁₋₃ alkyl), wherein the C₁₋₃ alkyl is optionally substituted with 1-2 independently selected R^{a} (e.g., Q is NMe or NCH₂CH₂CH₂OH).

In some embodiments, Q is *—NH—(C₁₋₃ alkylene)-, wherein the asterisk represents point of attachment to W.

In some embodiments, W is C(═O).

In some embodiments, W is S(O)₂, C(═S), or C(═NR^{d}).

In some embodiments, W is C(═C—NO₂) or C(═N—CN).

In certain embodiments, Q is NH; and W is C(═O).

The Variables X¹, X²

In some embodiments, X¹ is NR². In certain embodiments, X¹ is NH.

In some embodiments, X² is CR⁵. In certain embodiments, X² is CH.

In certain embodiments, X¹ is NR²; and X² is CR⁵. In certain of these embodiments, X¹ is NH; and X² is CH.

R^{1a}, R^{1b}, R^{1c}, and R^{1d}

In some embodiments, each of R^{1a}, R^{1b}, R^{1c}, and R^{1d} is independently selected from the group consisting of H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); and —C(═O)N(R′)(R″).

In certain embodiments, each of R^{1a}, R^{1b}, R^{1c}, and R^{1d} is H.

In certain other embodiments, 1-2 of R^{1a}, R^{1b}, R^{1c}, and R^{1d} is other than H; and each remaining of R^{a}, R^{1b}, R^{1c}, and R^{1d} is H.

In certain embodiments, one of R^{1a}, R^{1b}, R^{1c}, and R^{1d} is other than H; and each remaining of R^{1a}, R^{1b}, R^{1c}, and R^{1d} is H.

In certain embodiments, two of R^{1a}, R^{1b}, R^{1c}, and R^{1d} are other than H; and each remaining of R^{1a}, R^{1b}, R^{1c}, and R^{1d} is H.

In certain embodiments, R^{1a} is H or halo. For example, R^{1a} can be H.

In certain embodiments, R^{1d} is H or halo. For example, R^{1d} can be H.

In certain embodiments, R^{1b} is other than H; each of R^{1a}, R^{1c}, and R^{1d} is H.

In certain embodiments, each of R^{1b} and R^{1c} is other than H; and each of R^{1a} and R^{1d} is H.

In certain embodiments, R^{1b} is halo, such as —F, —Cl, or —Br. For example, R^{1b} can be —F or —Cl (e.g., —F). For example, R^{1b} can be —F. As another non-limiting example, R^{1b} can be —Cl.

In certain embodiments, R^{1b} is C₁₋₆ alkyl optionally substituted with 1-2 R^{a}, such as unsubstituted C₁₋₆ alkyl.

In certain embodiments, R^{1b} is C₁₋₄ haloalkyl (e.g., —CF₃ or —CHF₂)

In certain embodiments, R^{1b} is —CN.

In certain embodiments, R^{1b} is —SF₅.

In certain embodiments, R^{1b} is C₁₋₄ thioalkoxy (e.g., SMe).

In certain embodiments, R^{1b} is S(O)₂(C₁₋₄ alkyl) (e.g., S(O)₂Me).

In certain embodiments, R^{1b} is C₁₋₄ alkoxy or C₁₋₄ haloalkoxy (e.g., OCHF₂).

In certain embodiments, R^{1c} is halo (e.g., —F).

In certain embodiments, R^{1c} is selected from the group consisting of C₁₋₆ alkyl and C₁₋₄ haloalkyl.

In certain embodiments, R^{1c} is selected from the group consisting of: C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me).

In certain embodiments, each of R^{1b} and R^{1c} is an independently selected halo; and each of R^{1a} and R^{1d} is H. For example, each of R^{1b} and R^{1c} can be —F.

In certain embodiments, R^{1c} is H; and R^{1b} is halo, such as —F or —Cl, such as —Cl; and each of R^{a} and R^{1d} is H.

In certain embodiments, R^{1c} is halo; R^{1b} is selected from the group consisting of: C₁₋₆ alkyl, C₁₋₄ haloalkyl, C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me); and each of R^{1a} and R^{1d} is H. For example, R^{1c} is —F.

In certain embodiments, R^{1c} is H; R^{1b} is selected from the group consisting of: C₁₋₆ alkyl, C₁₋₄ haloalkyl, C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me); and each of R^{1a} and R^{1d} is H.

### The Variable R²

In some embodiments, R² is H.

In some embodiments, R² is selected from the group consisting of:

(iii) —C(O)(C₁₋₆ alkyl) optionally substituted with 1-3 independently selected R^{a};

(iv) —C(O)O(C₁₋₄ alkyl) optionally substituted with 1-3 independently R^{a};

(v) —CON(R′)(R″);

(vi) —S(O)₁₋₂(NR′R″); and

(vii) —S(O)₁₋₂(C₁₋₄ alkyl) optionally substituted with 1-3 independently selected R^{a}.

In certain embodiments, R² is —C(O)(C₁₋₆ alkyl) optionally substituted with 1-3 independently selected R^{a}. In certain of these embodiments, each R^{a} substituent of R² is independently —F, —Cl, —OH, or —NR^{e}R^{f}.

As a non-limiting example of the foregoing embodiments, R² can be selected from the group consisting of: C(═O)Me,

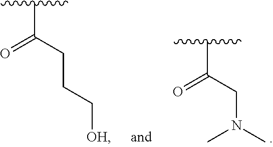

In certain embodiments, R² is —S(O)₁₋₂(C₁₋₄ alkyl) optionally substituted with 1-3 independently selected R^{a} (e.g., S(O)₂Me).

In certain embodiments, R² is -L⁴-L⁵-R. In certain of these embodiments, -L⁴ is a bond. In certain embodiments, -L⁴ is C(═O). In certain embodiments, -L⁴ is S(O)₂. In certain embodiments, -L⁵ is a bond. In certain other embodiments, -L⁵ is C₁₋₄ alkylene (e.g., C₁₋₂ alkylene).

In certain embodiments (when R² is -L⁴-L⁵-Rⁱ), Rⁱ is selected from the group consisting of: (a) C₃₋₈ cycloalkyl, optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is

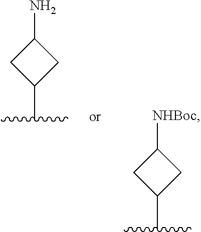 [SMILES: *C1CC(N)C1 ; *C1CC(NC(=O)OC(C)(C)C)C1]

wherein “Boc” represents tert-butoxycarbonyl); and

(b) heterocyclyl, wherein the heterocyclyl has 3-8 ring atoms, wherein 1-3 ring atoms are heteroatoms, each independently selected from the group consisting of N, N(H), N(R^{d}), O, and S(O)₀₋₂, wherein the heterocyclyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is

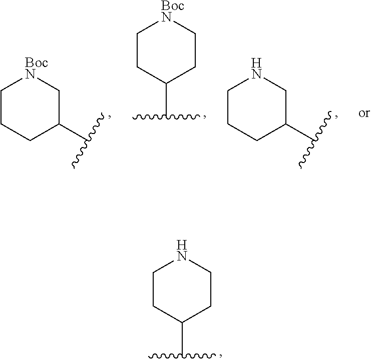

wherein “Boc” represents tert-butoxycarbonyl).

In certain embodiments (when R² is -L⁴-L⁵-Rⁱ), Rⁱ is selected from the group consisting of: (a) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(b) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

In certain embodiments, R² is -L⁴-L⁵-Rⁱ; L⁴ is a bond; L⁵ is a bond or C₁₋₄ alkylene; and Rⁱ is selected from the group consisting of:

(a) C₃₋₈ cycloalkyl, optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy

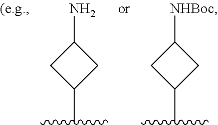 [SMILES: *C1CC(N)C1 ; *C1CC(NC(=O)OC(C)(C)C)C1]

wherein “Boc” represents tert-butoxycarbonyl);

(b) heterocyclyl, wherein the heterocyclyl has 3-8 ring atoms, wherein 1-3 ring atoms are heteroatoms, each independently selected from the group consisting of N, N(H), N(R^{d}), O, and S(O)₀₋₂, wherein the heterocyclyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

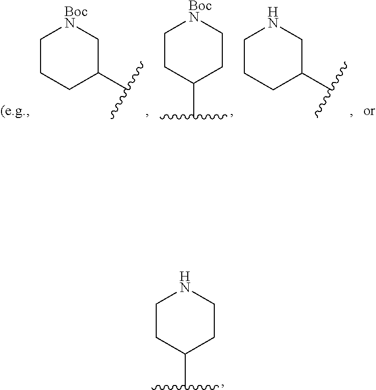

wherein “Boc” represents tert-butoxycarbonyl);

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

In certain embodiments (when R² is -L⁴-L⁵-R), R² is -L⁴-L⁵-Rⁱ; L⁴ is C(═O) or S(O)₂; L⁵ is a bond or C₁₋₄ alkylene; and Rⁱ is selected from the group consisting of:

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl optionally substituted with 1-2 substituents independently selected from halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

As non-limiting examples, R² can be selected from the group consisting of:

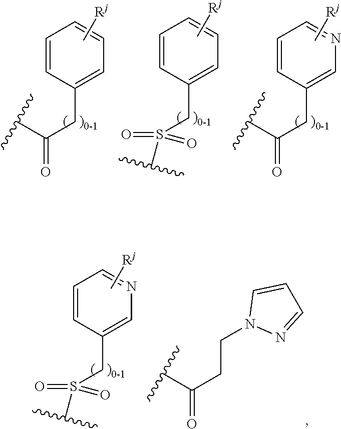

wherein R^{j} is H; halo; C₁₋₄ alkyl; C₁₋₄ haloalkyl; cyano; C₁₋₄ alkoxy; or C₁₋₄ haloalkoxy.

The Variable R⁵

In some embodiments, R⁵ is H.

The Variable R⁶

In some embodiments, R⁶ is H.

In some embodiments, R⁶ is C₁₋₃ alkyl.

Non-Limiting Combinations

In some embodiments, the compound is a compound of Formula (I-1):

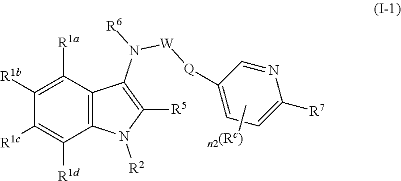

or a pharmaceutically acceptable salt thereof, wherein, n2 is 0, 1, or 2.

In certain of these embodiments, the compound has Formula (I-1-1):

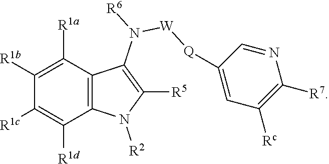

In some embodiments, the compound is a compound of Formula (I-2):

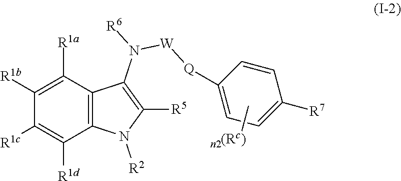

or a pharmaceutically acceptable salt thereof, wherein, n2 is 0, 1, or 2.

In certain of these embodiments, the compound has Formula (I-2-1):

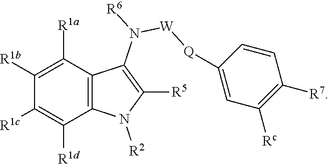

In some embodiments, the compound is a compound of Formula (I-3):

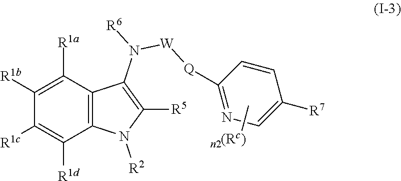

or a pharmaceutically acceptable salt thereof, wherein, n2 is 0, 1, or 2.

In certain of these embodiments, the compound has Formula (I-3-1):

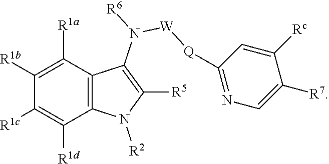

In some embodiments, the compound is a compound of Formula (I-4):

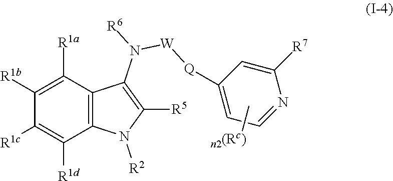

or a pharmaceutically acceptable salt thereof, wherein: n2 is 0, 1, or 2.

In certain of these embodiments, the compound has Formula (I-4-1):

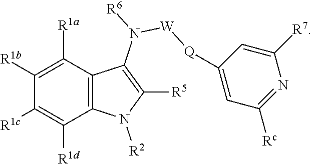

In some embodiments, the compound is a compound of Formula (I-5):

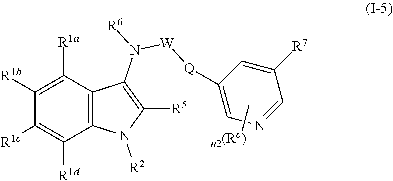

or a pharmaceutically acceptable salt thereof, wherein: n2 is 0, 1, or 2.

In certain of these embodiments, the compound has Formula (I-5-1):

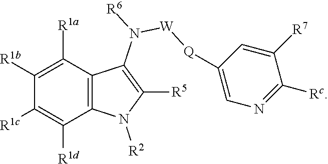

In some embodiments, the compound is a compound of Formula (I-6):

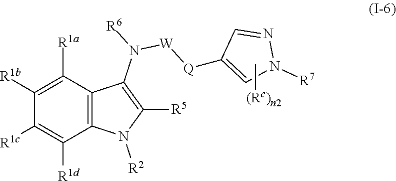

or a pharmaceutically acceptable salt thereof, wherein: n2 is 0 or 1.

In certain of these embodiments, the compound has Formula (I-6-1):

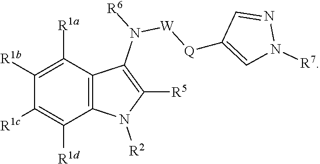

In some embodiments, the compound is a compound of Formula (I-7):

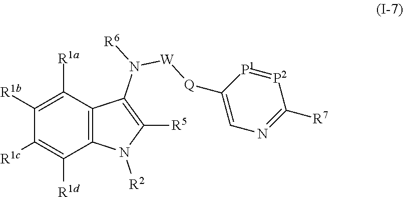

or a pharmaceutically acceptable salt thereof, wherein: one of P¹ and P² is N; and the other of P¹ and P² is CH or CR^{c} (e.g., CH).

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), R⁷ is —R⁸.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7) (when R⁷ is —R⁸), R⁸ is C₃₋₁₂ cycloalkyl or C₃₋₁₂ cycloalkenyl, each of which is optionally substituted with 1-4 independently selected R⁷′.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7) (when R⁷ is —R⁸), R⁸ is C₄₋₈ cycloalkyl which is substituted with 1-3 R⁷′.

In certain of these embodiments, R⁸ is cyclohexyl which is substituted with 1-3 R⁷′.

In certain embodiments, R⁸ is cyclobutyl which is substituted with 1-3 R⁷′.

As a non-limiting example of the foregoing embodiments, R⁸ can be

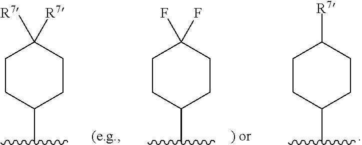

As another non-limiting example, R⁸ can be

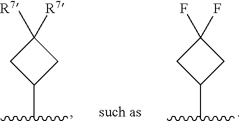

In certain embodiments, R⁸ is C₄₋₆ monocyclic cycloalkyl which is unsubstituted (e.g., cyclopentyl, cyclobutyl, or cyclohexyl); or R⁸ is C₇₋₈ bicyclic (e.g., spirocyclic) cycloalkyl which is unsubstituted

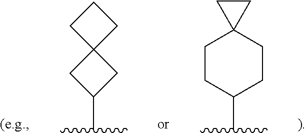 [SMILES: *C1CC2(CCC2)C1 ; *C1CCC2(CC1)CC2]

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7) (when R⁷ is —R⁸), R⁸ is heterocyclyl or heterocycloalkenyl of 4-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R⁷′.

In certain of these embodiments, R⁸ is heterocyclyl of 4-8 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 1-3 independently selected R⁷′.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7) (when R⁷ is —R⁸), R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 1-3 (e.g., 2) independently selected R⁷′ (e.g., R⁸ is selected from the group consisting of:

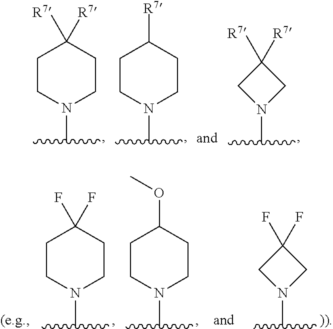

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7) (when R⁷ is —R⁸), R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 1-3 (e.g., 2) independently selected R⁷′ at one or more ring carbon atoms (e.g., R⁸ is selected from the group consisting of:

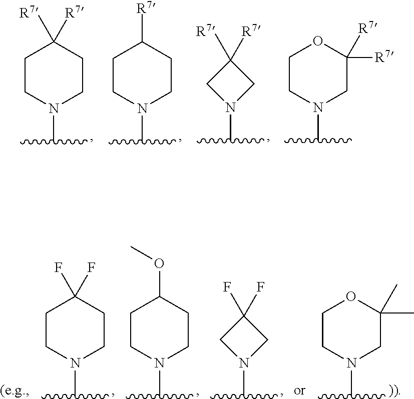

For example, R⁸ can be selected from the group consisting of: e.g., R⁸ is selected from the group consisting of:

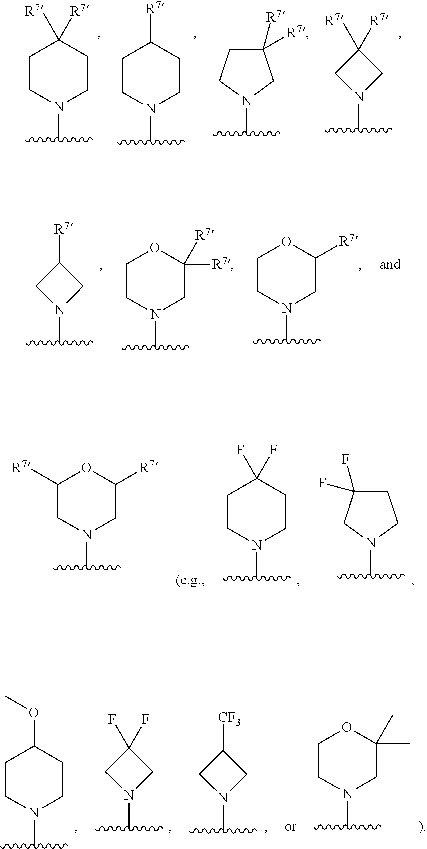

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7) when R⁷ is —R⁸, R⁸ is spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′, such as:

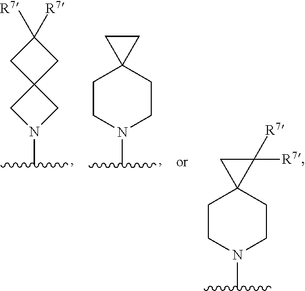

optionally wherein each R⁷′ is an independently selected halo, such as —F.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7) when R⁷ is —R⁸, R⁸ is monocyclic heterocyclyl of 3-8 ring atoms, wherein 1-2 ring atoms are heteroatoms, each independently selected from the group consisting of N, N(H), N(R^{d}), O, and S(O)₀₋₂.

In certain of these embodiments, R⁸ is azetidinyl

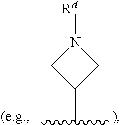

oxetanyl, pyrrolidinyl

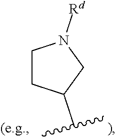

tetrahydrofuranyl, tetrahydropyranyl, piperidinyl

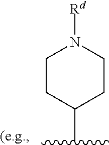

such as

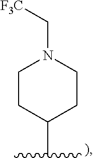 [SMILES: *C1CCN(CC(F)(F)F)CC1]

piperazinyl

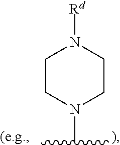

morpholinyl, and azepinyl, wherein a ring nitrogen atom is optionally substituted with R^{d}.

In certain of these embodiments, R^{d} is C₁₋₆ alkyl optionally substituted with 1-3 substituents each independently selected from the group consisting of halo, C₁₋₃ alkoxy, and C₁₋₃ haloalkoxy, such as wherein R^{d} is C₂₋₄ alkyl substituted with 1-3 independently selected halo

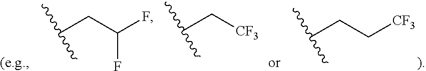

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), R⁷ is -L³-R⁹.

In certain of these embodiments, L³ is —O—.

In certain embodiments, L³ is —NH—.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), when R⁷ is -L³-R⁹, R⁹ is C₃₋₁₂ cycloalkyl or C₃₋₁₂ cycloalkenyl, each of which is optionally substituted with 1-4 independently selected R⁷′.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), when R⁷ is -L³-R⁹, R⁹ is C₄₋₈ cycloalkyl which is optionally substituted with 1-2 independently selected R⁷′.

In certain of these embodiments, R⁹ is cyclobutyl, cyclopentyl, cyclohexyl, or spiro[3.3]heptanyl, each of which is optionally substituted with 1-2 independently selected R⁷′ (e.g., unsubstituted).

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), when R⁷ is -L³-R⁹, R⁹ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R⁷′.

In certain embodiments, R⁹ is selected from the group consisting of azetidinyl, oxetanyl, pyrrolidinyl, tetrahydrofuranyl, piperidinyl, piperazinyl, morpholinyl, and azepinyl, each of which is optionally substituted with 1-2 independently selected R⁷′ (e.g., unsubstituted).

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), when R⁷ is -L³-R⁹, R⁷ is

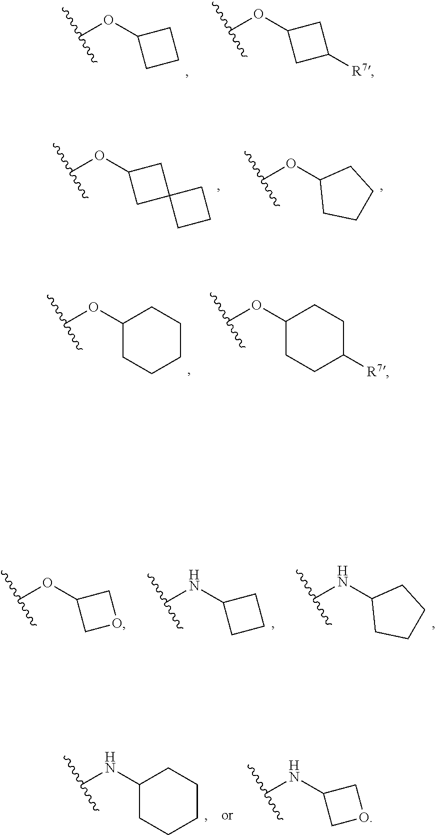

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), R⁷′ when present is independently selected from the group consisting of: halo, —CN, —OH, —C₁₋₄ alkyl optionally substituted with R^{a}, —C₁₋₄ haloalkyl, —C₁₋₆ alkoxy optionally substituted with R^{a}, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″).

In certain of these embodiments, each R⁷′ when present is independently selected from the group consisting of: halo, —CN, —C₁₋₄ alkyl optionally substituted with R^{a}, —C₁₋₄ haloalkyl, —C₁₋₆ alkoxy optionally substituted with R^{a}, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″). For example, each R⁷′ when present can be —F. As another non-limiting example, each R⁷′ when present is an independently selected C₁₋₃ alkyl such as methyl. As a further non-limiting example, each R⁷′ when present is an independently selected C₁₋₃ haloalkyl, such as —CF₃.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), one occurrence of R⁷′ is selected from the group consisting of:

—C₁₋₄ alkyl optionally substituted with R^{a}, such as unsubstituted C₁₋₄ alkyl (e.g., methyl, ethyl, n-propyl); —C₁₋₄ alkyl substituted with R^{a} (e.g., —C₁₋₄ alkyl substituted with OH or C₃₋₆ cycloalkyl); —CN; —C₁₋₆ alkoxy optionally substituted with R^{a}, such as unsubstituted C₁₋₆ alkoxy (e.g., methoxy); or C₁₋₆ alkoxy substituted with R^{a} (e.g., —C₁₋₄ alkoxy substituted with OH or C₃₋₆ cycloalkyl); and

each remaining R⁷′ when present is independently halo (e.g., —F).

In certain embodiments of Formulae (I-1), (I-2), (I-3), (I-4), (I-5), (I-6), or (I-7), n2 is 0.

In certain embodiments of Formulae (I-1), (I-2), (I-3), (I-4), (I-5), (I-6), or (I-7), n2 is 1 or 2. For example, n2 can be 1.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), each R^{c} when present is independently selected from the group consisting of: halo; cyano; C₁₋₁₀ alkyl; C₁₋₄ alkoxy; C₁₋₄ haloalkoxy; —S(O)₁₋₂(C₁₋₄ alkyl); —C(═O)(C₁₋₁₀ alkyl); and —C(═O)O(C₁₋₄ alkyl).

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), when n2 is 1 or 2, each R^{c} when present is independently selected from the group consisting of: (a) halo; (b) cyano; (c) C₁₋₁₀ alkyl; (g) C₁₋₄ alkoxy; (h) C₁₋₄ haloalkoxy; (i) —S(O)₁₋₂(C₁₋₄ alkyl); and —C(═O)(C₁₋₁₀ alkyl).

In certain of these embodiments, each R^{c} when present is halo (e.g., —F, —Br, or —Cl) or cyano. For example, R^{c} can be —F. As another non-limiting example, R^{c} can be —Cl.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), Q is NH.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), Q is N(C₁₋₃ alkyl), wherein the C₁₋₃ alkyl is optionally substituted with R^{a}.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), Q is *—NH—(C₁₋₃ alkylene), wherein the asterisk represents point of attachment to W.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), W is C(═O).

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), W is C(═C—NO₂) or C(═N—CN).

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), W is S(O)₂, C(═S), or C(═NR^{d}).

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), Q is NH; and W is C(═O).

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), each of R^{1a}, R^{1b}, R^{1c}, and R^{1d} is independently selected from the group consisting of H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); and —C(═O)N(R′)(R″).

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), each of R^{1a}, R^{1b}, R^{1c}, and R^{1d} is H.

In certain other embodiments, 1-2 of R^{1a}, R^{1b}, R^{1c}, and R^{1d} is other than H; and each remaining of R^{1a}, R^{1b}, R^{1c}, and R^{1d} is H.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), each of R^{1a} and R^{1d} is independently selected from the group consisting of H and halo. For example, each of R^{1a} and R^{1d} can be H.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), R^{1b} is other than H; each of R^{1a}, R^{1c}, and R^{1d} is H.

In certain of these embodiments, R^{1b} is halo (e.g., —F or —Cl (e.g., —F)).

In certain other embodiments, R^{1b} is selected from the group consisting of: C₁₋₆ alkyl, C₁₋₄ haloalkyl, C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me); and each of R^{1a} and R^{1d} is H.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), each of R^{1b} and R^{1c} is other than H; and each of R^{1a} and R^{1d} is H.

In certain of these embodiments, R^{1c} is halo (e.g., —F); R^{1b} is selected from the group consisting of: C₁₋₆ alkyl, C₁₋₄ haloalkyl, C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me); and each of R^{1a} and R^{1d} is H.

In certain other embodiments, each of R^{1b} and R^{1c} is an independently selected halo. For example, each of R^{1b} and R^{1c} is —F.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), R² is H.

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), R² is —C(O)(C₁₋₆ alkyl) optionally substituted with 1-3 independently selected R^{a}; or —S(O)₁₋₂(C₁₋₄ alkyl) optionally substituted with 1-3 independently selected R^{a} (e.g., S(O)₂Me).

As non-limiting examples of the foregoing embodiments, R² can be selected from the group consisting of: C(═O)Me, S(O)₂Me,

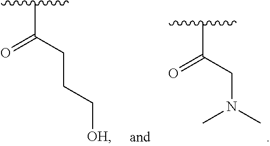

In certain embodiments of Formulae (I-1) (e.g., I-1-1), (I-2) (e.g., I-2-1), (I-3) (e.g., I-3-1), (I-4) (e.g., I-4-1), (I-5) (e.g., I-5-1), (I-6) (e.g., I-6-1), or (I-7), R⁶ is H.

In certain embodiments, the compound of Formula (I) is a compound of Formula (I-1a), (I-2a), (I-3a), (I-4a), (I-5a), or (I-6a):

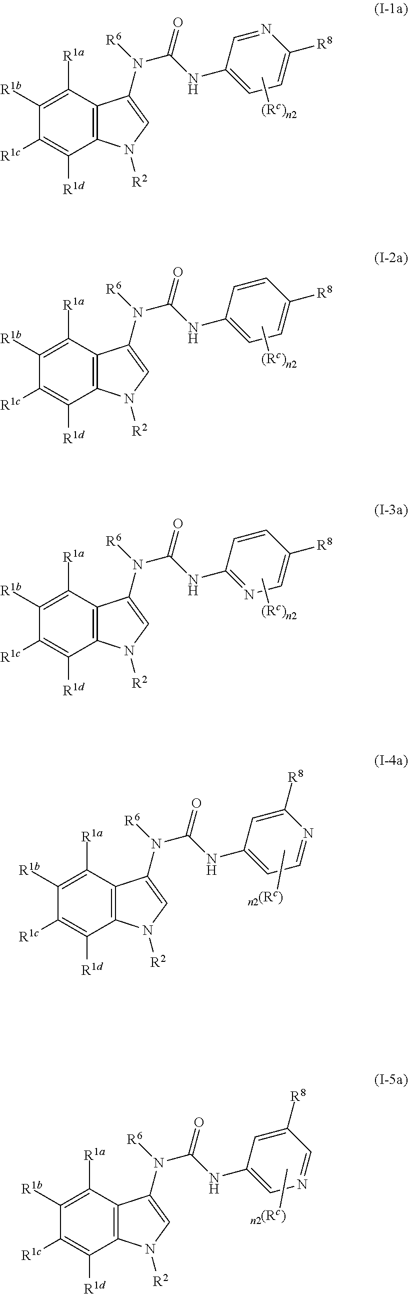

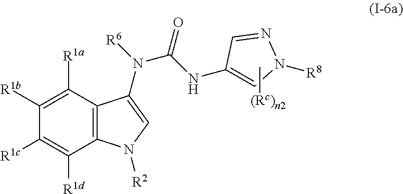

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

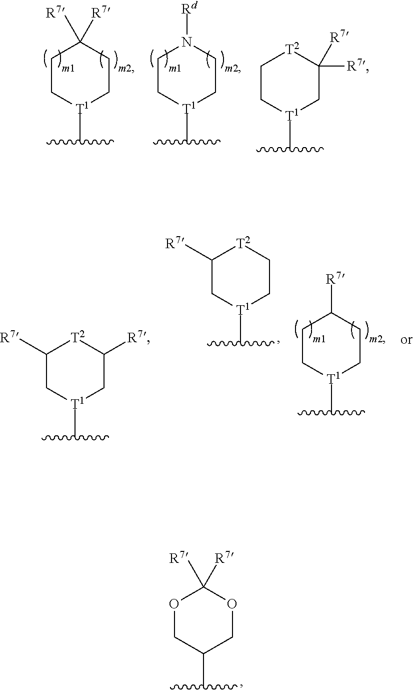

wherein m1 and m2 are independently 0, 1, or 2; T¹ is CH or N; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

In certain embodiments, the compound of Formula (I) is a compound of Formula (I-1a), (I-2a), or (I-3a):

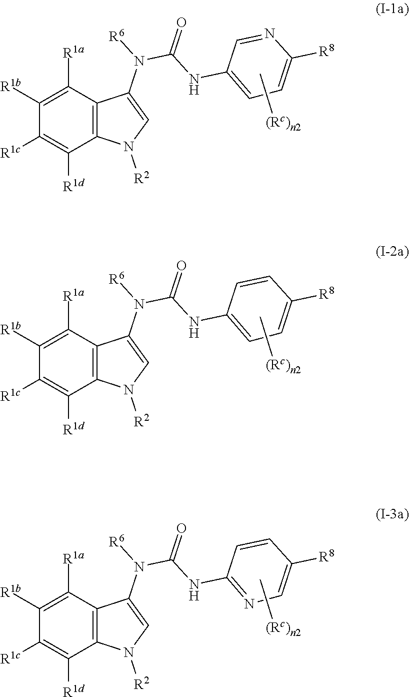

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

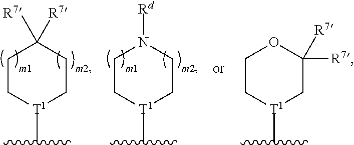

wherein m1 and m2 are independently 0, 1, or 2, and T¹ is CH or N; and
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′.

In certain embodiments, the compound has Formula (I-1a). In certain embodiments, the compound has Formula (I-2a). In certain embodiments, the compound has Formula (I-3a).

In certain embodiments of Formula (I-1a), (I-2a), (I-3a), (I-4a), (I-5a), or (I-6a), R² is H. In certain embodiments of Formula (I-1a), (I-2a), (I-3a), (I-4a), (I-5a), or (I-6a), R⁶ is H.

In certain embodiments of Formula (I-1a), (I-2a), (I-3a), (I-4a), (I-5a), or (I-6a), n2 is 1; and R^{c} is ortho to R⁸. In certain embodiments, R^{c} is halo, such as —Cl. In certain embodiments, R^{c} is C₁₋₃ alkyl, such as methyl.

In certain embodiments of Formula (I-1a), (I-2a), (I-3a), (I-4a), (I-5a), or (I-6a), R^{1a} and R^{1d} are H; and R^{1c} is H or halo.

In certain embodiments of Formula (I-1a), (I-2a), (I-3a), (I-4a), (I-5a), or (I-6a), R^{1b} is halo, such as —F or —Cl. In certain embodiments of Formulae (I-1a), (I-2a), or (I-3a), R^{1b} is C₁₋₆ alkyl or C₁₋₄ haloalkyl, such as methyl or —CHF₂.

In certain embodiments of Formula (I-1a), (I-2a), (I-3a), (I-4a), (I-5a), or (I-6a), R⁸ is

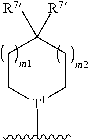

wherein m1 and m2 are independently 0, 1, or 2, and T¹ is CH or N. For example, R⁸ can be selected from the group consisting of:

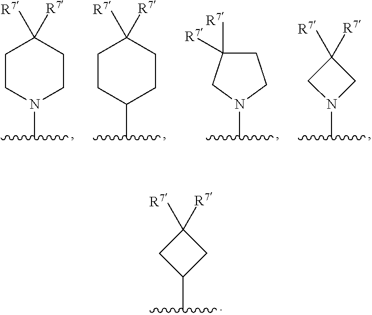

In certain embodiments of Formula (I-1a), (I-2a), (I-3a), (I-4a), (I-5a), or (I-6a), R⁸ is

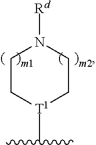

wherein m1 and m2 are independently 0, 1, or 2, and T¹ is CH or N. For example, R⁸ can be selected from the group consisting of:

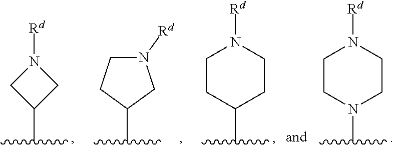

In certain embodiments of Formula (I-1a), (I-2a), (I-3a), (I-4a), (I-5a), or (I-6a), R⁸ is selected from the group consisting of:

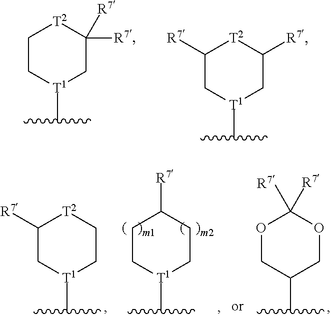

wherein m1 and m2 are independently 0, 1, or 2; T¹ is CH or N; and T² is CH₂, NH, NR^{d}, or O. For example, R⁸ can be selected from the group consisting of:

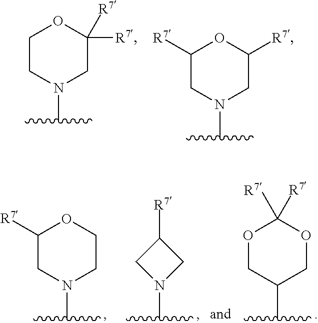

In certain embodiments of Formula (I-1a), (I-2a), (I-3a), (I-4a), (I-5a), or (I-6a), R⁸ is

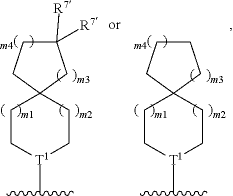

wherein m1, m2, m3, and m4 are independently 0, 1, or 2, provided that m1+m2+m3+m4≤6, and T¹ is CH or N. For example, R⁸ can be selected from the group consisting of:

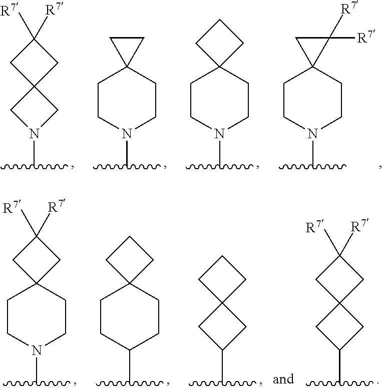

In certain embodiments of Formula (I-1a), (I-2a), (I-3a), (I-4a), (I-5a), or (I-6a), R⁸ is

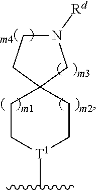

wherein m1, m2, m3, and m4 are independently 0, 1, or 2, provided that m1+m2+m3+m4≤6, and T¹ is CH or N. For example, R⁸ can be

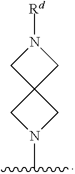

In certain embodiments of Formula (I-1a), (I-2a), (I-3a), (I-4a), (I-5a), or (I-6a), R⁸ is selected from the group consisting of:

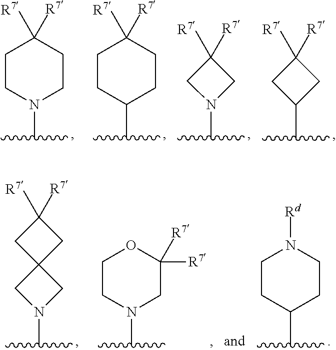

In certain embodiments of Formula (I-1a), (I-2a), (I-3a), (I-4a), (I-5a), or (I-6a), each R⁷′ is independently selected from the group consisting of C₁₋₃ alkyl; C₁₋₃ haloalkyl; and halo, such as wherein each R⁷′ is independently selected from the group consisting of methyl, CF₃, and —F; and R^{d} is C₁₋₆ alkyl, such as C₂₋₄ alkyl, optionally substituted with 1-3 independently selected halo, such as —F.

In certain embodiments of Formula (I-1a), (I-2a), (I-3a), (I-4a), (I-5a), or (I-6a), each R⁷′ is independently selected from the group consisting of C₁₋₃ alkyl and halo, such as methyl and —F.

In certain embodiments of Formula (I-1a), (I-2a), (I-3a), (I-4a), (I-5a), or (I-6a), R^{d} is C₁₋₆ alkyl, such as C₂₋₄ alkyl, optionally substituted with 1-3 independently selected halo, such as —F.

In certain embodiments, the compound of Formula (I) is a compound of Formula (I-3a):

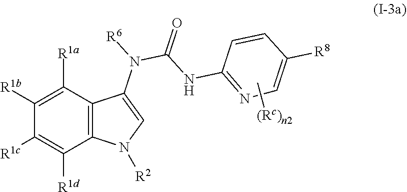

or a pharmaceutically acceptable salt thereof, wherein:

each of R^{1a}, R^{1l}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

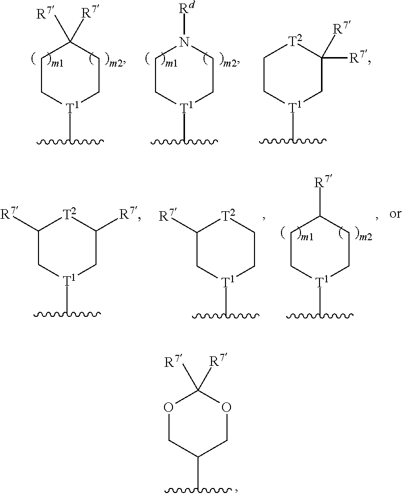

wherein m1 and m2 are independently 0, 1, or 2; T¹ is CH or N; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

In certain embodiments of Formula (I-3a), R⁸ is

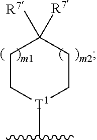

and optionally wherein each R⁷′ is an independently selected halo, such as —F. In certain of these embodiments, R⁸ is selected from the group consisting of:

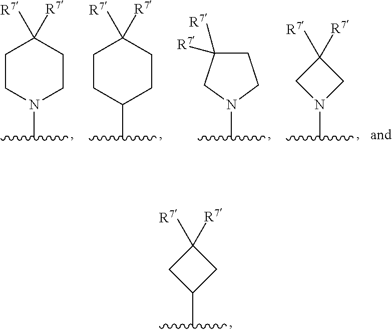

and optionally wherein each R⁷′ is —F. For example, R⁸ can be

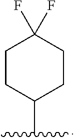 [SMILES: *C1CCC(F)(F)CC1]

In certain embodiments of Formula (I-3a), R^{1a} and R^{1d} are H; R^{1b} is halo, such as —F; R^{1c} is —H or halo, such as —H or —F; and R² is H.

In certain embodiments of Formula (I-3a), the compound has Formula (I-3a-1):

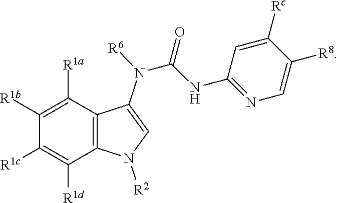

In certain embodiments of Formula (I-3a) or Formula (I-3a-1), R^{e} is halo, such as —F or —Cl.

In certain embodiments of Formula (I-3a) or Formula (I-3a-1), R⁸ is

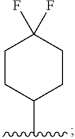 [SMILES: *C1CCC(F)(F)CC1]

and/or R^{1a} and R^{1d} are H; and/or R^{1b} is —F; and/or R^{1c} is —H or —F; and/or R² is H; and/or R^{c} is halo.

In certain embodiments, the compound of Formula (I) is a compound of Formula (I-2a):

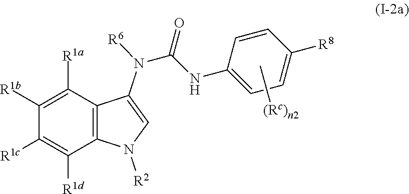

or a pharmaceutically acceptable salt thereof, wherein:

each of R^{1a}, R^{1l}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

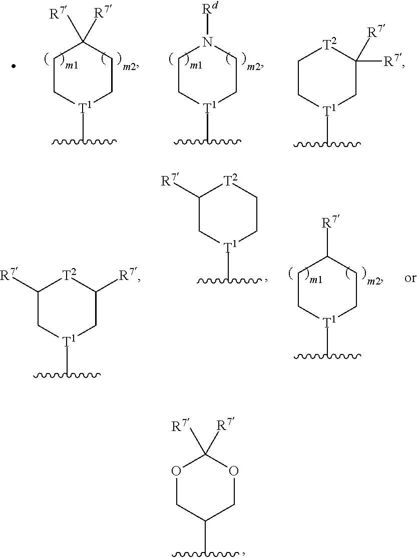

wherein m1 and m2 are independently 0, 1, or 2; T¹ is CH or N; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

In certain embodiments of Formula (I-2a), R⁸ is

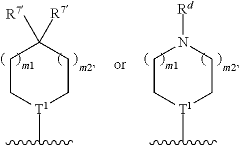

and optionally wherein each R⁷′ is an independently selected halo, such as —F; and optionally wherein R^{d} is C₂₋₄ alkyl which is substituted with 1-3 independently selected halo, such as —F. In certain of these embodiments, R⁸ is selected from the group consisting of:

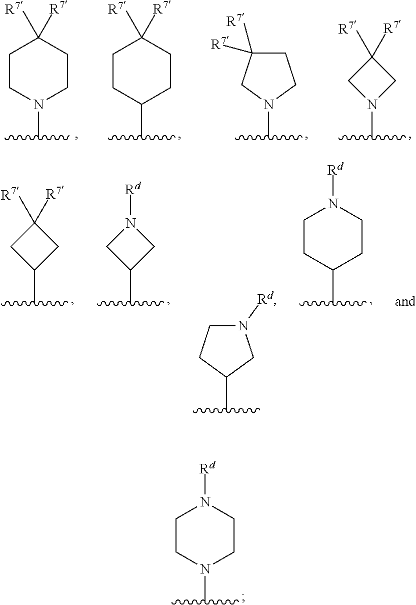

and optionally wherein each R⁷′ is —F; and optionally wherein R^{d} is C₂₋₄ alkyl which is substituted with 1-3 —F. For example, R⁸ can be

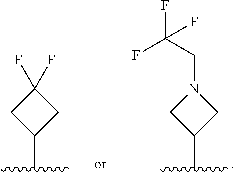 [SMILES: *C1CC(F)(F)C1 ; *C1CN(CC(F)(F)F)C1]

In certain embodiments of Formula (I-2a), R^{a}, R^{1d}, and R^{1c} are each H; R^{1b} is —H or halo, such as —H, —Cl, or —F; and R² is H.

In certain embodiments of Formula (I-2a), the compound has Formula (I-2a-1):

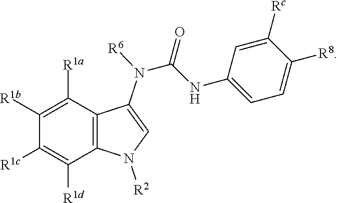

In certain embodiments of Formula (I-2a) or (I-2a-1), R^{c} is -halo.

In certain embodiments of Formula (I-2a) or (I-2a-1), R⁸ is

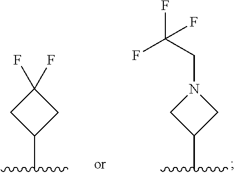 [SMILES: *C1CC(F)(F)C1 ; *C1CN(CC(F)(F)F)C1]

and/or R^{1a}, R^{1d}, and R^{1c} are H; and/or R^{1b} is —H, —Cl, or —F; and/or R² is H; and/or R^{c} is halo.

In certain embodiments, the compound of Formula (I) is a compound of Formula (I-7a):

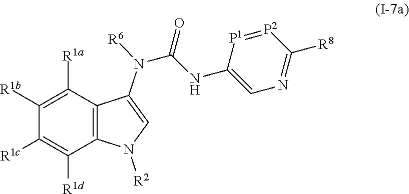

or a pharmaceutically acceptable salt thereof, wherein:

one of P¹ and P² is N; and the other of P¹ and P² is CH;

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R⁸ is selected from the group consisting of:

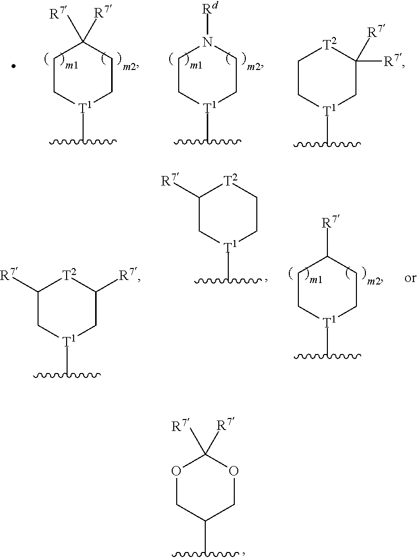

wherein m1 and m2 are independently 0, 1, or 2; T¹ is CH or N; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

In certain embodiments of Formula (I-7a), R⁸ is

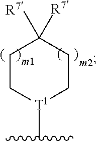

and optionally wherein each R⁷′ is an independently selected halo, such as —F. In certain of these embodiments, R⁸ is selected from the group consisting of:

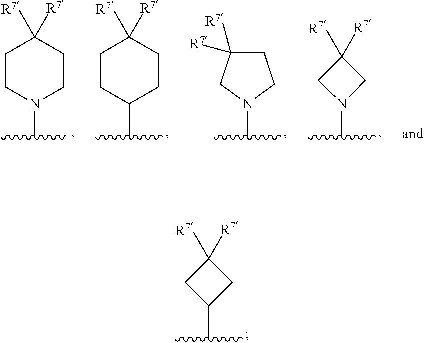

and optionally wherein each R⁷′ is —F. For example, R⁸ is

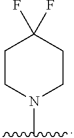 [SMILES: *N1CCC(F)(F)CC1]

In certain embodiments of Formula (I-7a), R^{1a}, R^{1d}, and R^{1c} are H; R^{1b} is halo, such as —Cl; and R² is H.

In certain embodiments of Formula (I-7a), R⁸ is

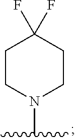 [SMILES: *N1CCC(F)(F)CC1]

and/or R^{1a}, R^{1d}, and R^{1c} are H; and/or R^{1b} is —Cl; and/or R² is H.

In certain embodiments, the compound of Formula (I) is a compound of Formula (I-1a):

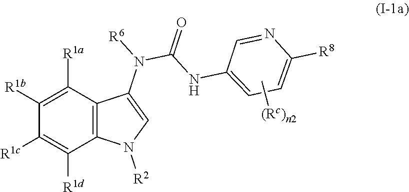

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

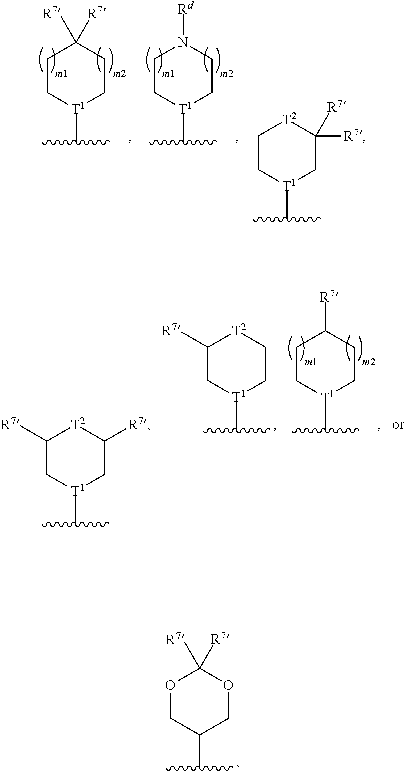

wherein m1 and m2 are independently 0, 1, or 2; T¹ is CH or N; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

In certain embodiments of Formula (I-1a), R⁸ is

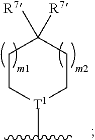

and optionally wherein each R⁷′ is an independently selected halo, such as —F. In certain of these embodiments, R⁸ is selected from the group consisting of:

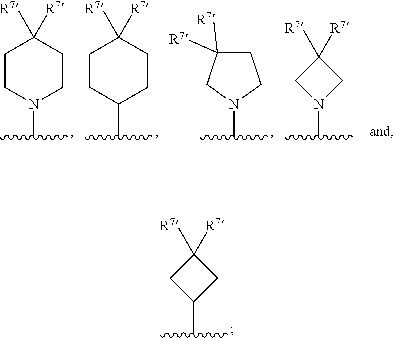

and optionally wherein each R⁷′ is —F. For example, R⁸ can be selected from the group consisting of:

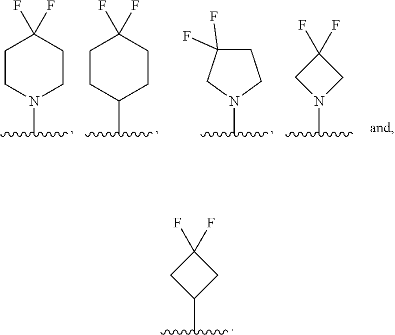

In certain embodiments of Formula (I-1a), R⁸ is

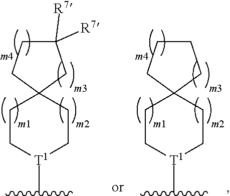

wherein: m1, m2, m3, and m4 are independently 0, 1, or 2, provided that m1+m2+m3+m4≤6; T¹ is CH or N; and each R⁷′ is independently selected from the group consisting of C₁₋₃ alkyl; C₁₋₃ haloalkyl; and halo, such as methyl, CF₃, and —F. In certain of these embodiments, R⁸ is selected from the group consisting of:

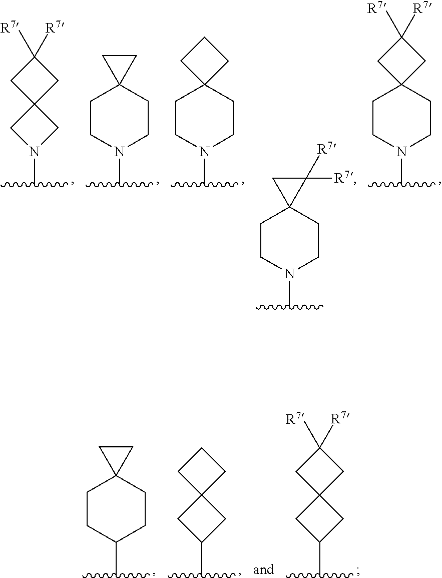

and optionally wherein each R⁷′ is —F. For example, R⁸ can be selected from the group consisting of:

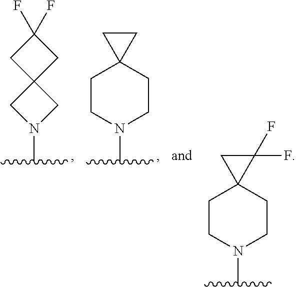

In certain embodiments of Formula (I-1a), R^{1a} and R^{1d} are H; R^{1b} is halo, such as —F or —Cl; R^{1c} is —H or halo, such as —H, —F, or —Cl; and R² is H.

In certain embodiments of Formula (I-1a), the compound has Formula (I-1a-1):

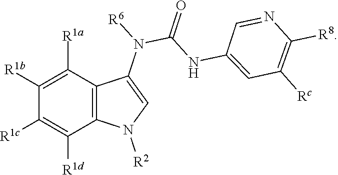

In certain embodiments of Formula (I-1a) or (I-1a-1), R^{c} is halo, such as —F or —Cl.

In certain embodiments of Formula (I-1a) or (I-1a-1), R⁸ is selected from the group consisting of:

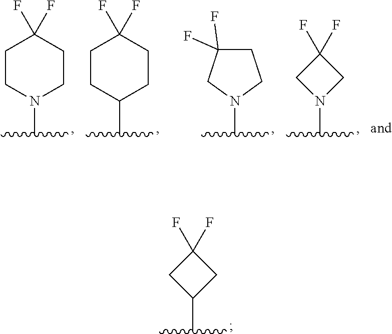

and/or R^{1a} and R^{1d} are H; and/or R^{1b} is —F or —Cl; and/or R^{1c} is —H, —F, or —Cl; and/or R² is H; and/or R^{c} is halo.

In certain embodiments of Formula (I-1a) or (I-1a-1), R⁸ is selected from the group consisting of:

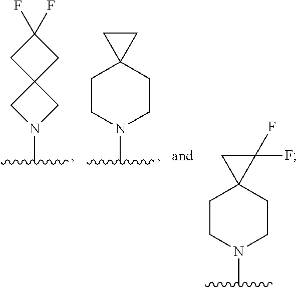

and/or R^{1a} and R^{1d} are H; and/or R^{1b} is —F or —Cl; and/or R^{1c} is —H, —F, or —Cl; and/or R² is H; and/or R^{c} is halo.

In certain embodiments, the compound of Formula (I) is a compound of Formula

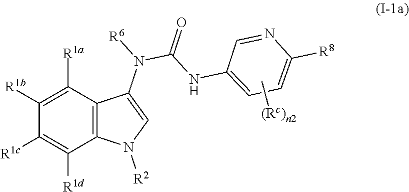

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1, or 2;

each R^{c} when present is independently selected from the group consisting of: halo, cyano, C₁₋₃ alkyl, and C₁₋₃ alkoxy;

R⁸ is

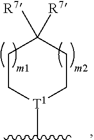

wherein:

m1 and m2 are independently 0, 1, or 2;

T¹ is CH or N; and

each R⁷′ is independently selected from the group consisting of C₁₋₃ alkyl; C₁₋₃ haloalkyl; and halo, such as methyl, CF₃, and —F.

In certain of these embodiments, the compound is a compound of Formula (I-1a):

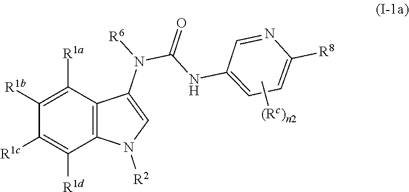

or a pharmaceutically acceptable salt thereof, wherein:

R^{1a} and R^{1d} are H;

each of R^{1b} and R^{1c} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1;

R^{c} when present is selected from the group consisting of: halo and cyano;

R⁸ is selected from the group consisting of:

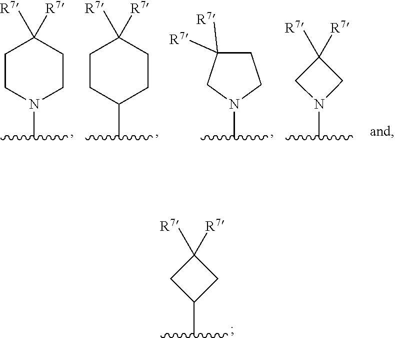

and

each R⁷′ is independently halo or C₁₋₃ alkyl, such as —F or C₁₋₃ alkyl.

In certain of the foregoing embodiments, the compound is a compound of Formula (I-1a-1):

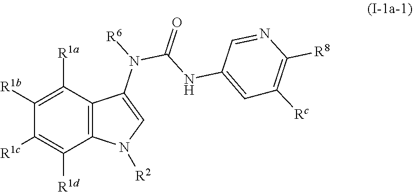

or a pharmaceutically acceptable salt thereof, wherein:

R^{1a} and R^{1d} are H;

R^{1b} is halo;

R^{1c} is H or halo;

R² is H;

R^{c} is selected from the group consisting of: —F, —Cl, —Br, and cyano; and

R⁸ is selected from the group consisting of:

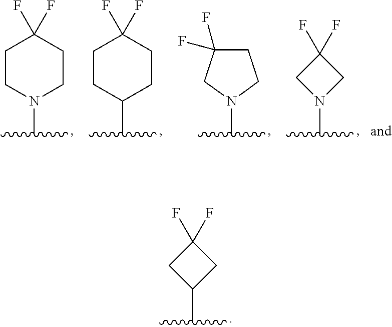

In certain embodiments, the compound of Formula (I) is a compound of Formula (I-1a):

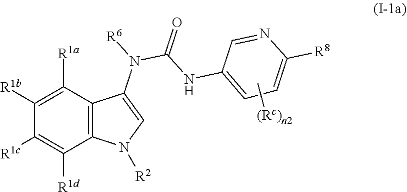

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1, or 2;

each R^{c} when present is independently selected from the group consisting of: halo, cyano, C₁₋₃ alkyl, and C₁₋₃ alkoxy;

R⁸ is

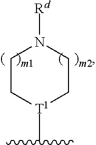

wherein:

m1 and m2 are independently 0, 1, or 2;

T¹ is CH or N; and

R^{d} is C₁₋₆ alkyl, such as C₂₋₄ alkyl, optionally substituted with 1-3 independently selected halo, such as —F.

In certain of these embodiments, the compound is a compound of Formula (I-1a):

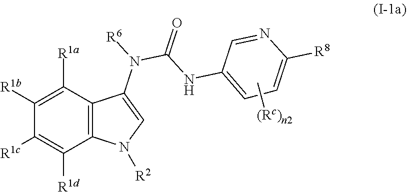

or a pharmaceutically acceptable salt thereof, wherein:

R^{1a} and R^{1d} are H;

each of R^{1b} and R^{1c} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1;

R^{c} when present is selected from the group consisting of: halo and cyano;

R⁸ is selected from the group consisting of:

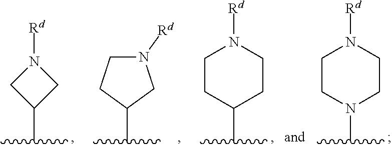

and

R^{d} is C₂₋₄ alkyl, optionally substituted with 1-3 independently selected halo, such as —F.

In certain of the foregoing embodiments, the compound is a compound of Formula (I-1a-1):

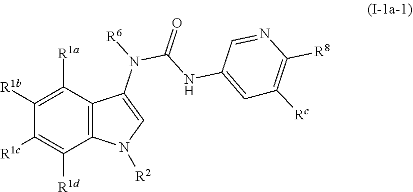

or a pharmaceutically acceptable salt thereof, wherein:

R^{1a} and R^{1d} are H;

R^{1b} is halo;

R^{1c} is H or halo;

R² is H;

R^{c} is selected from the group consisting of: —F, —Cl, —Br, and cyano;

R⁸ is selected from the group consisting of:

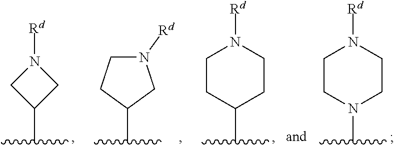

and

R^{d} is C₂₋₄ alkyl which is substituted with 1-3 independently selected halo, such as —F.

In certain embodiments, the compound of Formula (I) is a compound of Formula (I-1a):

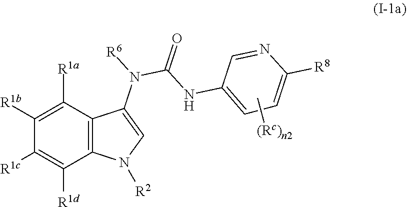

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

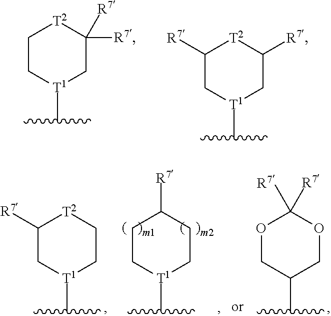

wherein:

m1 and m2 are independently 0, 1, or 2;

T¹ is CH or N;

T² is CH₂, NH, NR^{d}, or O; and

each R⁷′ is independently selected from the group consisting of C₁₋₃ alkyl and C₁₋₃ haloalkyl.

In certain of these embodiments, the compound is a compound of Formula (I-1a):

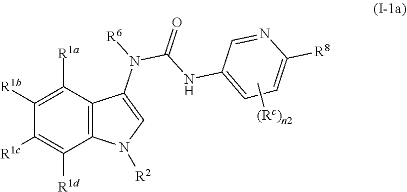

or a pharmaceutically acceptable salt thereof, wherein:

R^{1a} and R^{1d} are H;

each of R^{1b} and R^{1c} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1;

R^{c} when present is selected from the group consisting of: halo and cyano;

R⁸ is selected from the group consisting of:

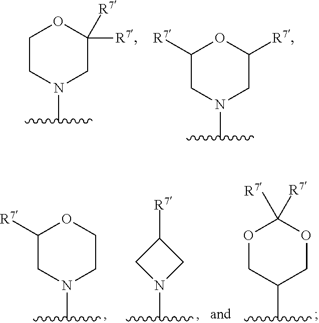

and

each R⁷′ is independently selected from the group consisting of C₁₋₃ alkyl and C₁₋₃ haloalkyl.

In certain of the foregoing embodiments, the compound is a compound of Formula (I-1a-1):

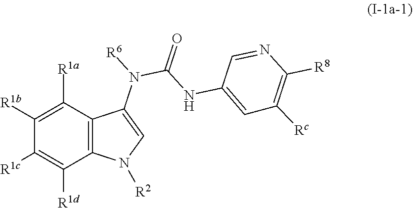

or a pharmaceutically acceptable salt thereof, wherein:

R^{1a} and R^{1d} are H;

R^{1b} is halo;

R^{1c} is H or halo;

R² is H;

R^{c} is selected from the group consisting of: —F, —Cl, —Br, and cyano;

R⁸ is selected from the group consisting of:

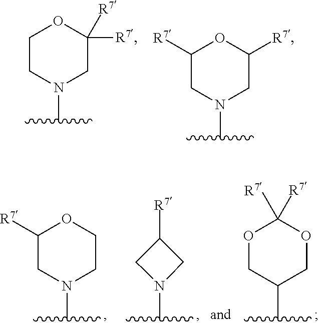

and

each R⁷′ is independently selected from the group consisting of C₁₋₃ alkyl and C₁₋₃ haloalkyl.

In certain embodiments, the compound of Formula (I) is a compound of Formula (I-1a):

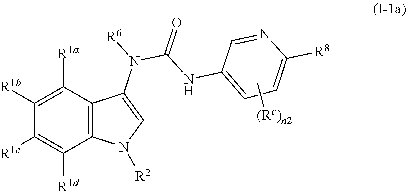

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1, or 2;

each R^{c} when present is independently selected from the group consisting of: halo, cyano, C₁₋₃ alkyl, and C₁₋₃ alkoxy;

R⁸ is

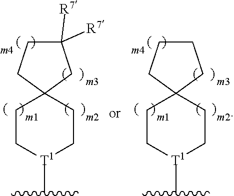

wherein:

m1, m2, m3, and m4 are independently 0, 1, or 2, provided that m1+m2+m3+m4≤6;

T¹ is CH or N; and

each R⁷′ is independently selected from the group consisting of C₁₋₃ alkyl; C₁₋₃ haloalkyl; and halo, such as methyl, CF₃, and —F.

In certain of these embodiments, the compound is a compound of Formula (I-1a):

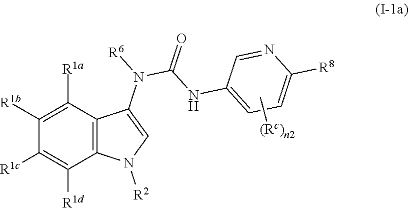

or a pharmaceutically acceptable salt thereof, wherein:

R^{1a} and R^{1d} are H;

each of R^{1b} and R^{1c} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1;

R^{c} when present is selected from the group consisting of: halo and cyano;

R⁸ is selected from the group consisting of:

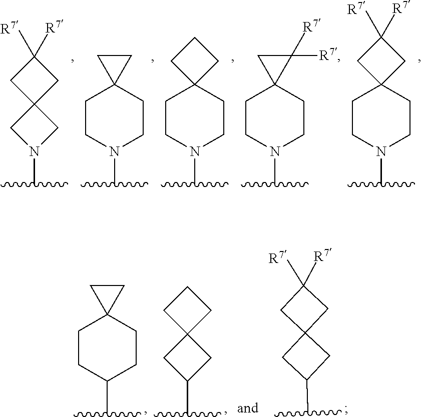

and

each R⁷′ is independently selected from the group consisting of C₁₋₃ alkyl and halo, such as methyl and —F.

In certain of the foregoing embodiments, the compound is a compound of Formula (I-1a-1):

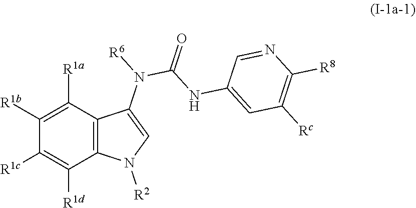

or a pharmaceutically acceptable salt thereof, wherein:

R^{1a} and R^{1d} are H;

R^{1b} is halo;

R^{1c} is H or halo;

R² is H;

R^{c} is selected from the group consisting of: —F, —Cl, —Br, and cyano; and

R⁸ is selected from the group consisting of:

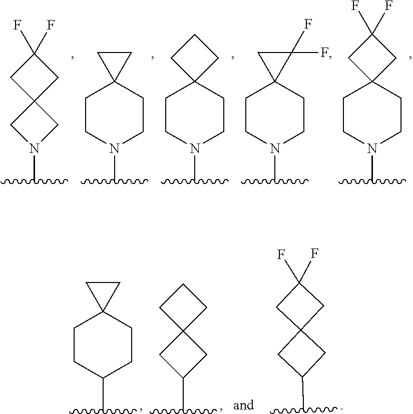

In certain embodiments, the compound of Formula (I) is a compound of Formula (I-1a):

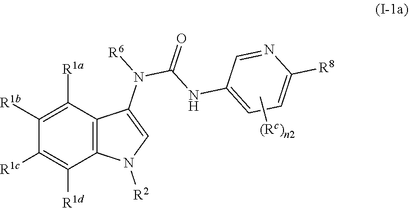

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1, or 2;

each R^{c} when present is independently selected from the group consisting of: halo, cyano, C₁₋₃ alkyl, and C₁₋₃ alkoxy;

R⁸ is

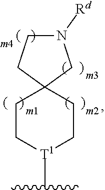

wherein:

m1, m2, m3, and m4 are independently 0, 1, or 2, provided that m1+m2+m3+m4≤6;

T¹ is CH or N; and

R^{d} is C₁₋₆ alkyl, such as C₂₋₄ alkyl, optionally substituted with 1-3 independently selected halo, such as —F.

In certain of these embodiments, the compound is a compound of Formula (I-1a):

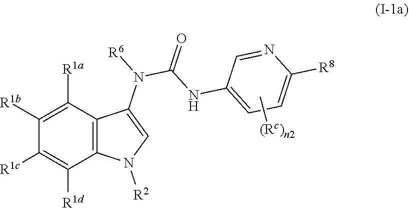

or a pharmaceutically acceptable salt thereof, wherein:

R^{1a} and R^{1d} are H;

each of R^{1b} and R^{1c} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R² is H;

n2 is 0, 1;

R^{c} when present is selected from the group consisting of: halo and cyano;

R⁸ is

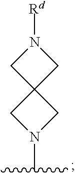

and

R^{d} is C₂₋₄ alkyl, optionally substituted with 1-3 independently selected halo, such as —F.

In certain of the foregoing embodiments, the compound is a compound of Formula (I-1a-1):

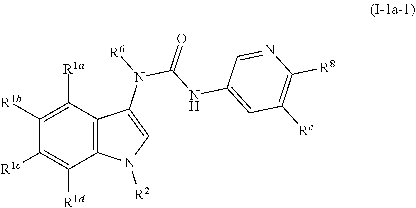

or a pharmaceutically acceptable salt thereof, wherein:

R^{1a} and R^{1d} are H;

R^{1b} is halo;

R^{1c} is H or halo;

R² is H;

R^{c} is selected from the group consisting of: —F, —Cl, —Br, and cyano; and

R⁸ is

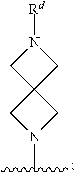

and

R^{d} is C₂₋₄ alkyl which is substituted with 1-3 independently selected halo, such as —F.

In certain embodiments, the compound of Formula (I) is a compound of Formula (I-6a):

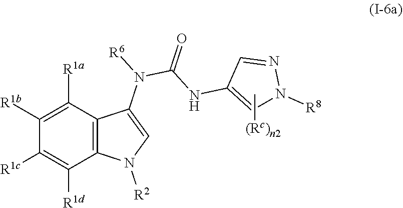

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

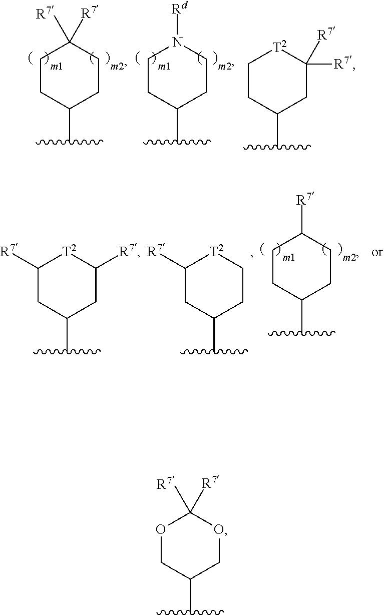

wherein m1 and m2 are independently 0, 1, or 2; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

In certain embodiments of Formula (I-6a), R⁸ is

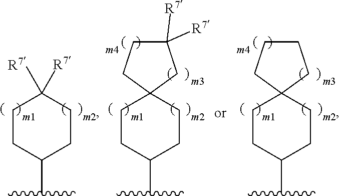

wherein: m1, m2, m3, and m4 are independently 0, 1, or 2, provided that m1+m2+m3+m4≤6; and

each R⁷′ is independently selected from the group consisting of C₁₋₃ alkyl; C₁₋₃ haloalkyl; and halo, such as methyl, CF₃, and —F.

In certain of these embodiments, R⁸ is

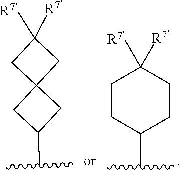

For example, R⁸ can be

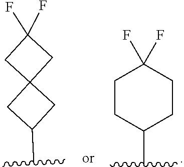 [SMILES: *C1CC2(C1)CC(F)(F)C2 ; *C1CCC(F)(F)CC1]

In certain embodiments of Formula (I-6a), R^{1a}, R^{1d}, and R^{1c} are H; R^{1b} is halo, such as —Cl; and R² is H.

In certain embodiments of Formula (I-6a), n2 is 0.

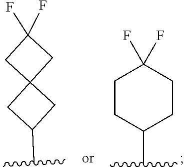 [SMILES: *C1CC2(C1)CC(F)(F)C2 ; *C1CCC(F)(F)CC1]

In certain embodiments of Formula (I-6a), n2 is 0; and/or R⁸ is or; and/or R^{1a}, R^{1d}, and R^{1c} are H; and/or R^{1b} is —Cl; and/or R² is H.

In certain embodiments, the compound of Formula (I) is a compound of Formula (I-4a):

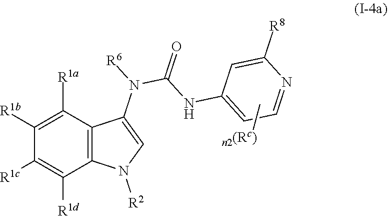

or a pharmaceutically acceptable salt thereof, wherein:

each of R^{1a}, R^{1l}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

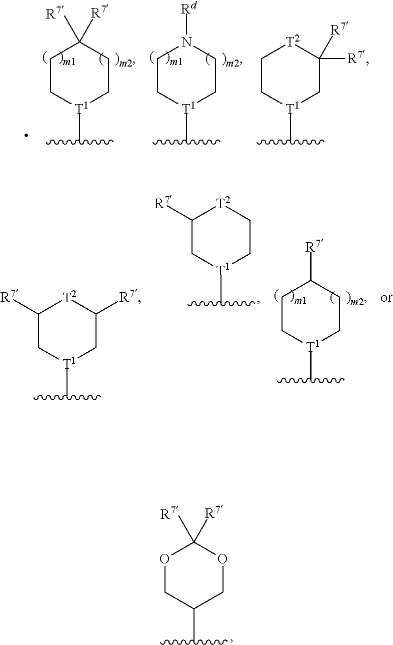

wherein m1 and m2 are independently 0, 1, or 2; T¹ is CH or N; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

In certain embodiments of Formula (I-4a), R⁸ is

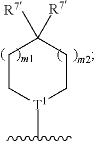

and optionally wherein each R⁷′ is an independently selected halo, such as —F. In certain of these embodiments, R⁸ is selected from the group consisting of:

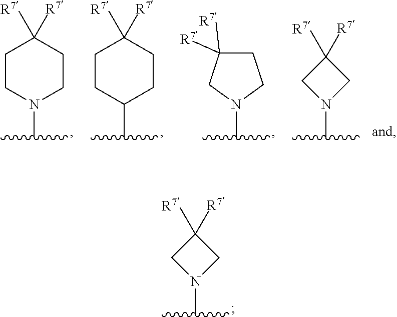

and optionally wherein each R⁷′ is —F. For example, R⁸ can be

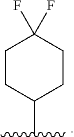 [SMILES: *C1CCC(F)(F)CC1]

In certain embodiments of Formula (I-4a), R^{1a} and R^{1d} are H; R^{1b} is halo, such as —F or —Cl; R^{1c} is H or halo, such as —H or —F; and R² is H.

In certain embodiments of Formula (I-4a), n2 is 1; and the compound has Formula (I-4a-1):

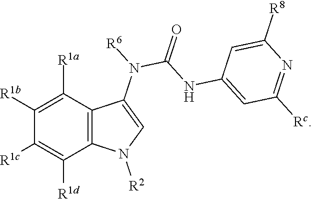

In certain embodiments of Formula (I-4a) or Formula (I-4a-1), R^{e} is halo.

In certain embodiments of Formula (I-4a), n2 is 0.

In certain embodiments of Formula (I-4a) or Formula (I-4a-1), R⁸ is

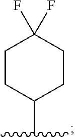 [SMILES: *C1CCC(F)(F)CC1]

and/or R^{1a} and R^{1d} are H; and/or R^{1b} is —F or —Cl; and/or R^{1c} is —H or —F; and/or R² is H.

In certain embodiments of Formula (I-1a) (e.g., I-1a-1), (I-2a) (e.g., I-2a-1), (I-3a) (e.g., I-3a-1), (I-4a) (e.g., I-4a-1), (I-5a), (I-6a), or (I-7a), R⁶ is H.

### Non-Limiting Exemplary Formula I Compounds

In some embodiments, the compound is selected from the group consisting of the compounds delineated in Table C1, or a pharmaceutically acceptable salt thereof.

TABLE C1

| Compound | |
|---|---|
| # | Structure |
| 101 | 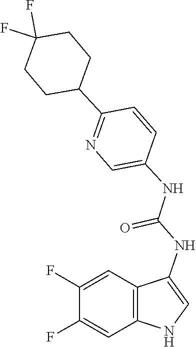 [SMILES: O=C(Nc1ccc(C2CCC(F)(F)CC2)nc1)Nc1c[nH]c2cc(F)c(F)cc12] |
| 102 | 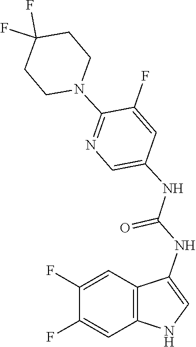 [SMILES: O=C(Nc1cnc(N2CCC(F)(F)CC2)c(F)c1)Nc1c[nH]c2cc(F)c(F)cc12] |
| 103 | 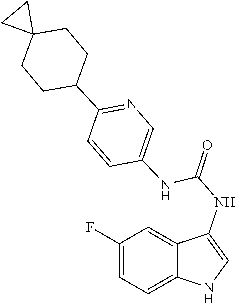 [SMILES: O=C(Nc1ccc(C2CCC3(CC2)CC3)nc1)Nc1c[nH]c2ccc(F)cc12] |
| 104 | 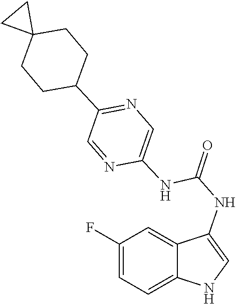 [SMILES: O=C(Nc1cnc(C2CCC3(CC2)CC3)cn1)Nc1c[nH]c2ccc(F)cc12] |
| 105 | 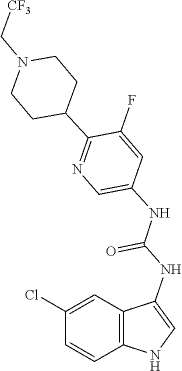 [SMILES: O=C(Nc1cnc(C2CCN(CC(F)(F)F)CC2)c(F)c1)Nc1c[nH]c2ccc(Cl)cc12] |
| 106 | 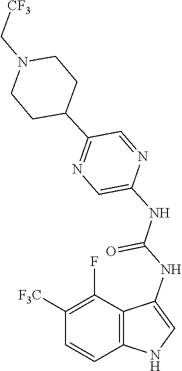 [SMILES: O=C(Nc1cnc(C2CCN(CC(F)(F)F)CC2)cn1)Nc1c[nH]c2ccc(C(F)(F)F)c(F)c12] |
| 107 | 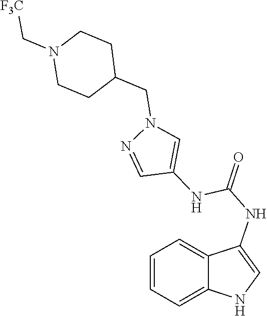 [SMILES: O=C(Nc1cnn(CC2CCN(CC(F)(F)F)CC2)c1)Nc1c[nH]c2ccccc12] |
| 108 | 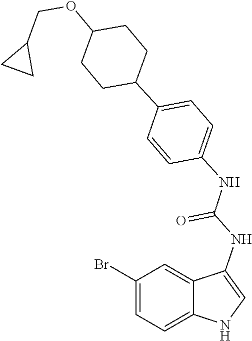 [SMILES: O=C(Nc1ccc(C2CCC(OCC3CC3)CC2)cc1)Nc1c[nH]c2ccc(Br)cc12] |
| 109 | 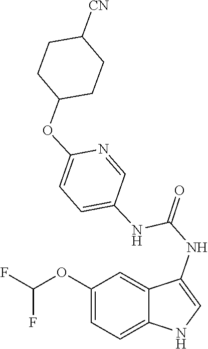 [SMILES: N#CC1CCC(Oc2ccc(NC(=O)Nc3c[nH]c4ccc(OC(F)F)cc34)cn2)CC1] |
| 110 | 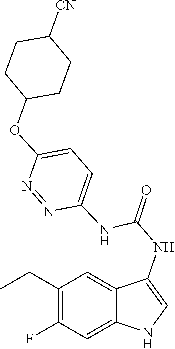 [SMILES: CCc1cc2c(NC(=O)Nc3ccc(OC4CCC(C#N)CC4)nn3)c[nH]c2cc1F] |
| 111 | 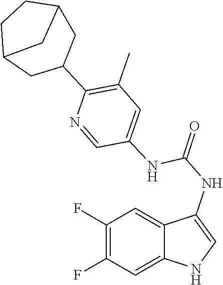 [SMILES: Cc1cc(NC(=O)Nc2c[nH]c3cc(F)c(F)cc23)cnc1C1CC2CCC(C2)C1] |
| 112 | 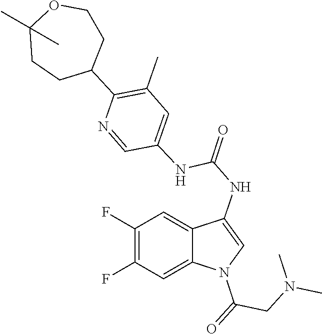 |
| 113 | 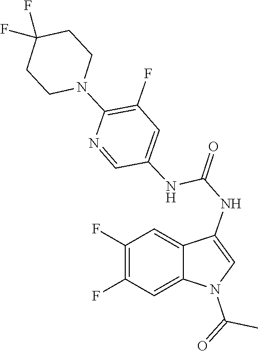 [SMILES: CC(=O)n1cc(NC(=O)Nc2cnc(N3CCC(F)(F)CC3)c(F)c2)c2cc(F)c(F)cc21] |
| 114 | 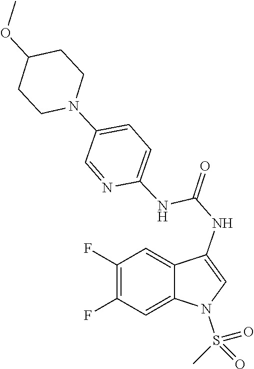 [SMILES: COC1CCN(c2ccc(NC(=O)Nc3cn(S(C)(=O)=O)c4cc(F)c(F)cc34)nc2)CC1] |
| 115 | 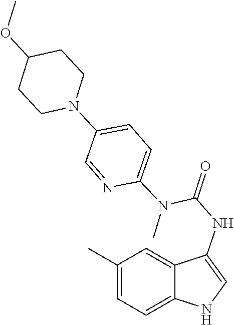 [SMILES: COC1CCN(c2ccc(N(C)C(=O)Nc3c[nH]c4ccc(C)cc34)nc2)CC1] |
| 116 | 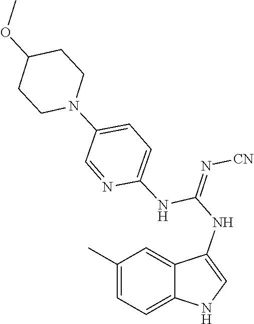 [SMILES: COC1CCN(c2ccc(N/C(=N/C#N)Nc3c[nH]c4ccc(C)cc34)nc2)CC1] |
| 117 | 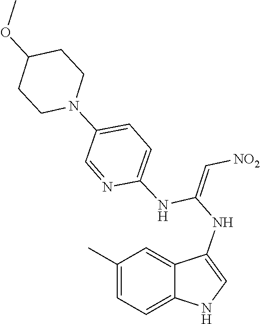 [SMILES: COC1CCN(c2ccc(N/C(=C/[N+](=O)[O-])Nc3c[nH]c4ccc(C)cc34)nc2)CC1] |
| 118 | 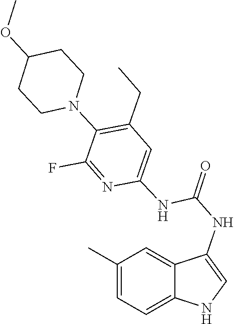 [SMILES: CCc1cc(NC(=O)Nc2c[nH]c3ccc(C)cc23)nc(F)c1N1CCC(OC)CC1] |
| 119 | 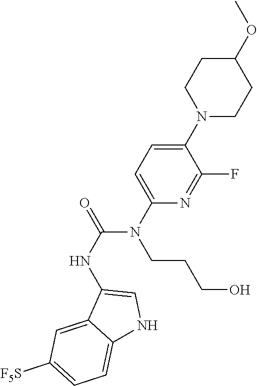 |
| 120 | 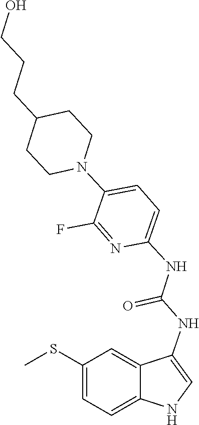 [SMILES: CSc1ccc2[nH]cc(NC(=O)Nc3ccc(N4CCC(CCCO)CC4)c(F)n3)c2c1] |
| 121 | 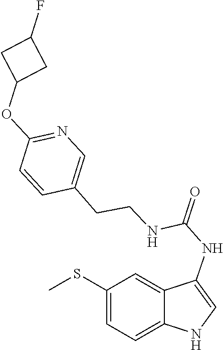 [SMILES: CSc1ccc2[nH]cc(NC(=O)NCCc3ccc(OC4CC(F)C4)nc3)c2c1] |
| 122 | 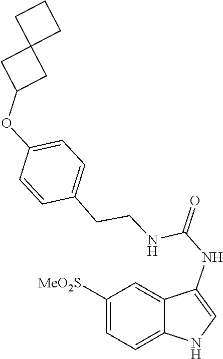 [SMILES: CS(=O)(=O)c1ccc2[nH]cc(NC(=O)NCCc3ccc(OC4CC5(CCC5)C4)cc3)c2c1] |
| 123 | 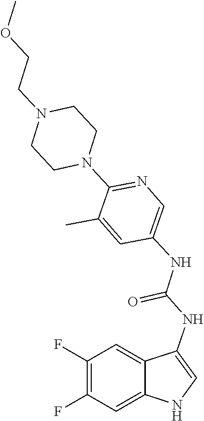 [SMILES: COCCN1CCN(c2ncc(NC(=O)Nc3c[nH]c4cc(F)c(F)cc34)cc2C)CC1] |
| 124 | 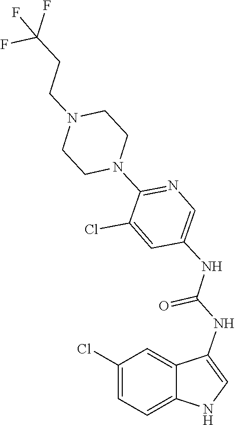 [SMILES: O=C(Nc1cnc(N2CCN(CCC(F)(F)F)CC2)c(Cl)c1)Nc1c[nH]c2ccc(Cl)cc12] |
| 125 | 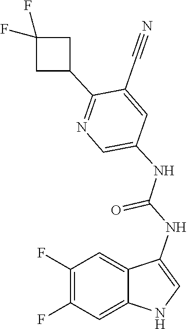 [SMILES: N#Cc1cc(NC(=O)Nc2c[nH]c3cc(F)c(F)cc23)cnc1C1CC(F)(F)C1] |
| 126 | 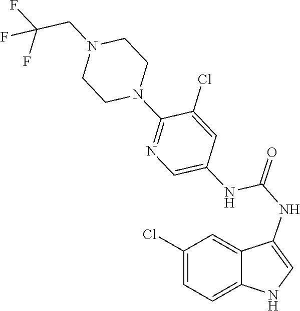 [SMILES: O=C(Nc1cnc(N2CCN(CC(F)(F)F)CC2)c(Cl)c1)Nc1c[nH]c2ccc(Cl)cc12] |
| 127 | 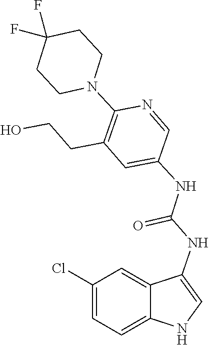 [SMILES: O=C(Nc1cnc(N2CCC(F)(F)CC2)c(CCO)c1)Nc1c[nH]c2ccc(Cl)cc12] |
| 128 | 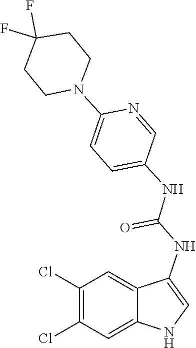 [SMILES: O=C(Nc1ccc(N2CCC(F)(F)CC2)nc1)Nc1c[nH]c2cc(Cl)c(Cl)cc12] |
| 129 | 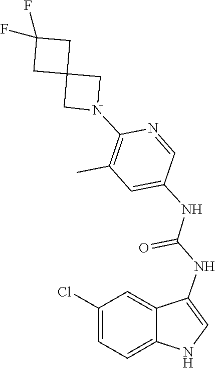 [SMILES: Cc1cc(NC(=O)Nc2c[nH]c3ccc(Cl)cc23)cnc1N1CC2(C1)CC(F)(F)C2] |
| 130 | 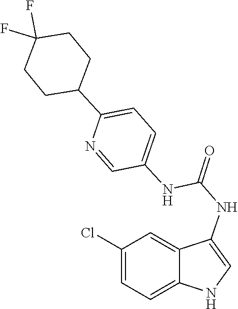 [SMILES: O=C(Nc1ccc(C2CCC(F)(F)CC2)nc1)Nc1c[nH]c2ccc(Cl)cc12] |
| 131 | 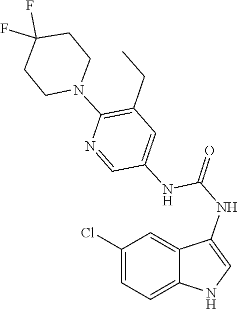 [SMILES: CCc1cc(NC(=O)Nc2c[nH]c3ccc(Cl)cc23)cnc1N1CCC(F)(F)CC1] |
| 132 | 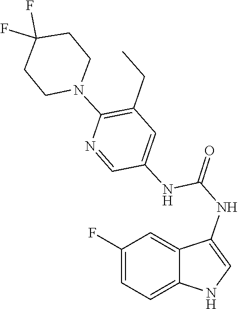 [SMILES: CCc1cc(NC(=O)Nc2c[nH]c3ccc(F)cc23)cnc1N1CCC(F)(F)CC1] |
| 133 | 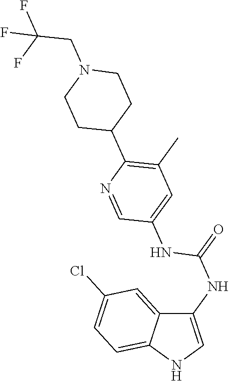 [SMILES: Cc1cc(NC(=O)Nc2c[nH]c3ccc(Cl)cc23)cnc1C1CCN(CC(F)(F)F)CC1] |
| 134 | 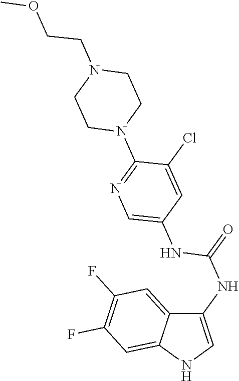 [SMILES: COCCN1CCN(c2ncc(NC(=O)Nc3c[nH]c4cc(F)c(F)cc34)cc2Cl)CC1] |
| 135 | 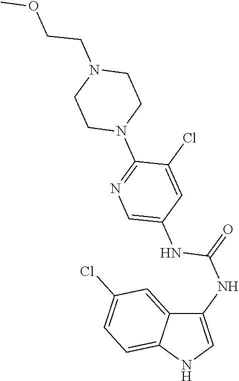 [SMILES: COCCN1CCN(c2ncc(NC(=O)Nc3c[nH]c4ccc(Cl)cc34)cc2Cl)CC1] |
| 136 | 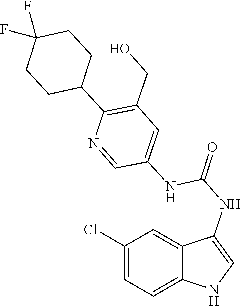 [SMILES: O=C(Nc1cnc(C2CCC(F)(F)CC2)c(CO)c1)Nc1c[nH]c2ccc(Cl)cc12] |
| 137 | 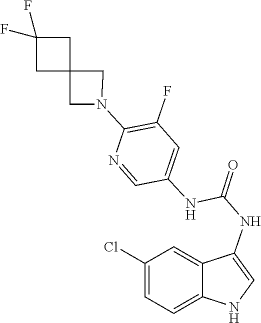 [SMILES: O=C(Nc1cnc(N2CC3(C2)CC(F)(F)C3)c(F)c1)Nc1c[nH]c2ccc(Cl)cc12] |
| 138 | 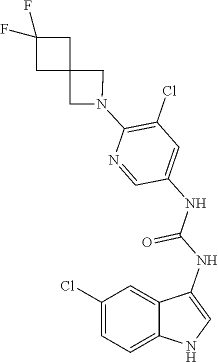 [SMILES: O=C(Nc1cnc(N2CC3(C2)CC(F)(F)C3)c(Cl)c1)Nc1c[nH]c2ccc(Cl)cc12] |
| 139 | 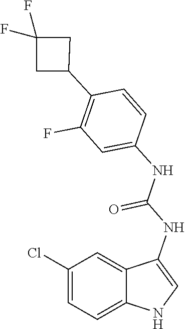 [SMILES: O=C(Nc1ccc(C2CC(F)(F)C2)c(F)c1)Nc1c[nH]c2ccc(Cl)cc12] |
| 140 | 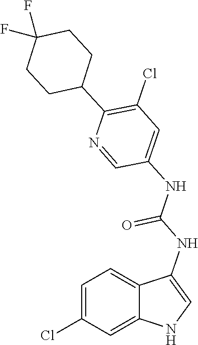 [SMILES: O=C(Nc1cnc(C2CCC(F)(F)CC2)c(Cl)c1)Nc1c[nH]c2cc(Cl)ccc12] |
| 141 | 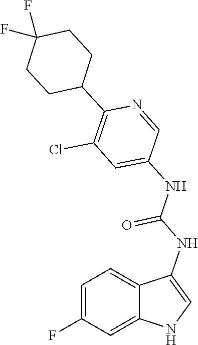 [SMILES: O=C(Nc1cnc(C2CCC(F)(F)CC2)c(Cl)c1)Nc1c[nH]c2cc(F)ccc12] |
| 142 | 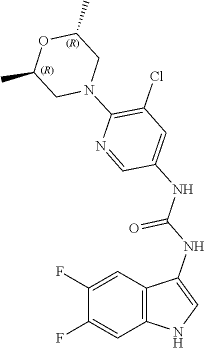 |
| 143 | 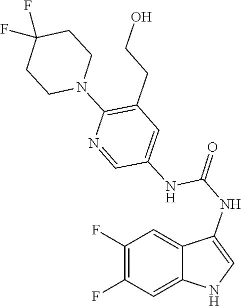 [SMILES: O=C(Nc1cnc(N2CCC(F)(F)CC2)c(CCO)c1)Nc1c[nH]c2cc(F)c(F)cc12] |
| 144 | 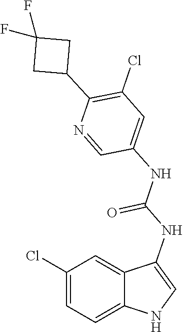 [SMILES: O=C(Nc1cnc(C2CC(F)(F)C2)c(Cl)c1)Nc1c[nH]c2ccc(Cl)cc12] |
| 145 | 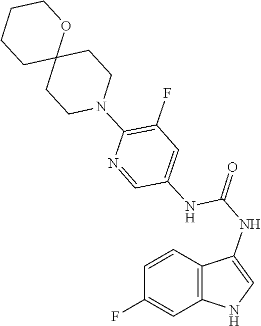 [SMILES: O=C(Nc1cnc(N2CCC3(CCCCO3)CC2)c(F)c1)Nc1c[nH]c2cc(F)ccc12] |
| 146 | 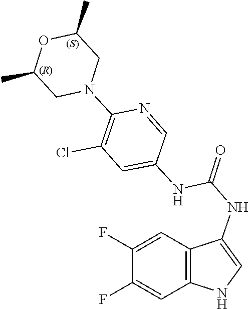 |
| 147 | 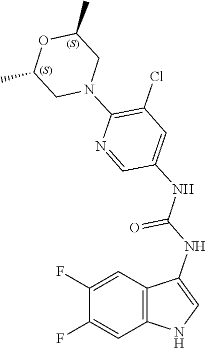 |
| 148 | 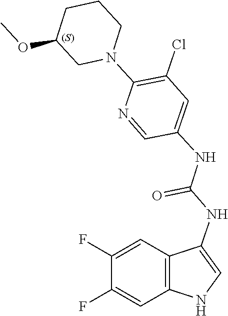 |
| 149 | 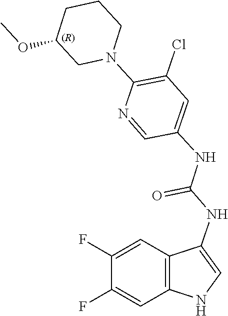 |
| 150 | 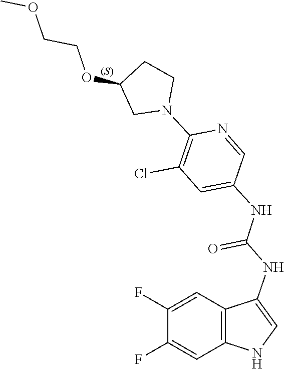 |
| 151 | 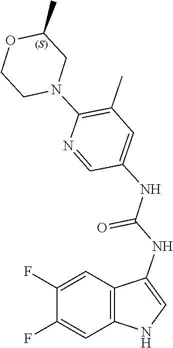 |
| 152 | 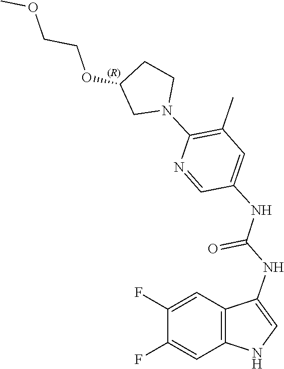 |
| 153 | 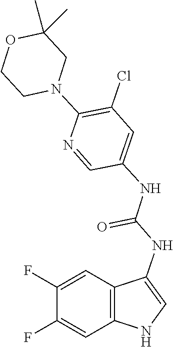 [SMILES: CC1(C)CN(c2ncc(NC(=O)Nc3c[nH]c4cc(F)c(F)cc34)cc2Cl)CCO1] |
| 154 | 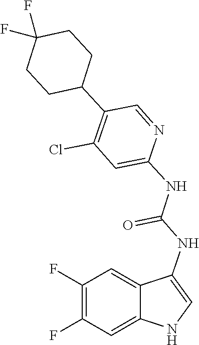 [SMILES: O=C(Nc1cc(Cl)c(C2CCC(F)(F)CC2)cn1)Nc1c[nH]c2cc(F)c(F)cc12] |
| 155 | 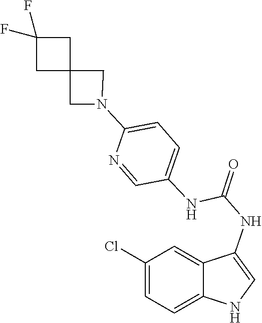 [SMILES: O=C(Nc1ccc(N2CC3(C2)CC(F)(F)C3)nc1)Nc1c[nH]c2ccc(Cl)cc12] |
| 156 | 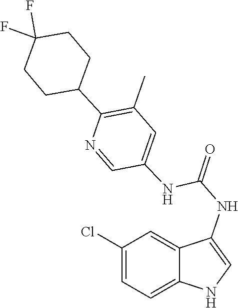 [SMILES: Cc1cc(NC(=O)Nc2c[nH]c3ccc(Cl)cc23)cnc1C1CCC(F)(F)CC1] |
| 157 | 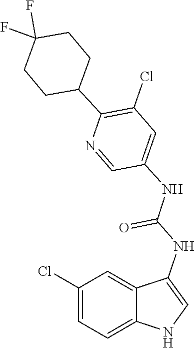 [SMILES: O=C(Nc1cnc(C2CCC(F)(F)CC2)c(Cl)c1)Nc1c[nH]c2ccc(Cl)cc12] |
| 158 | 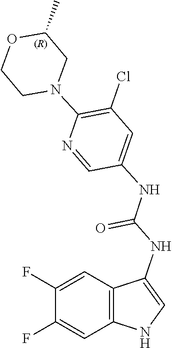 |
| 159 | 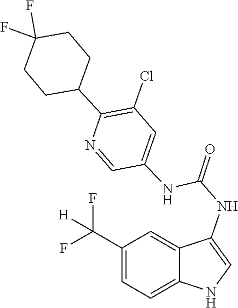 [SMILES: O=C(Nc1cnc(C2CCC(F)(F)CC2)c(Cl)c1)Nc1c[nH]c2ccc(C(F)F)cc12] |
| 160 | 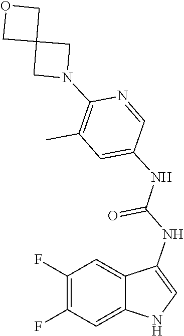 [SMILES: Cc1cc(NC(=O)Nc2c[nH]c3cc(F)c(F)cc23)cnc1N1CC2(COC2)C1] |
| 161 | 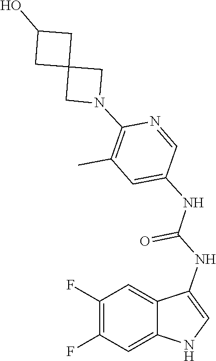 [SMILES: Cc1cc(NC(=O)Nc2c[nH]c3cc(F)c(F)cc23)cnc1N1CC2(CC(O)C2)C1] |
| 162 | 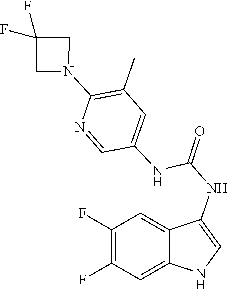 [SMILES: Cc1cc(NC(=O)Nc2c[nH]c3cc(F)c(F)cc23)cnc1N1CC(F)(F)C1] |
| 163 | 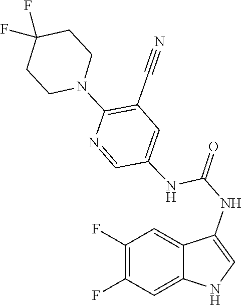 [SMILES: N#Cc1cc(NC(=O)Nc2c[nH]c3cc(F)c(F)cc23)cnc1N1CCC(F)(F)CC1] |
| 164 | 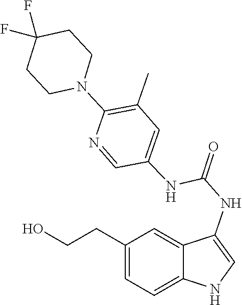 [SMILES: Cc1cc(NC(=O)Nc2c[nH]c3ccc(CCO)cc23)cnc1N1CCC(F)(F)CC1] |
| 165 | 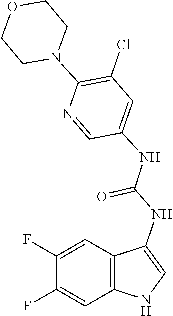 [SMILES: O=C(Nc1cnc(N2CCOCC2)c(Cl)c1)Nc1c[nH]c2cc(F)c(F)cc12] |
| 166 | 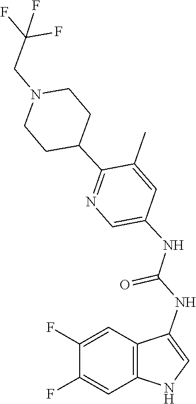 [SMILES: Cc1cc(NC(=O)Nc2c[nH]c3cc(F)c(F)cc23)cnc1C1CCN(CC(F)(F)F)CC1] |
| 167 | 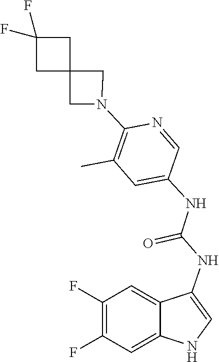 [SMILES: Cc1cc(NC(=O)Nc2c[nH]c3cc(F)c(F)cc23)cnc1N1CC2(C1)CC(F)(F)C2] |
| 168 | 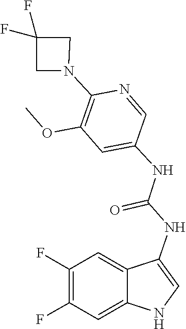 [SMILES: COc1cc(NC(=O)Nc2c[nH]c3cc(F)c(F)cc23)cnc1N1CC(F)(F)C1] |
| 169 | 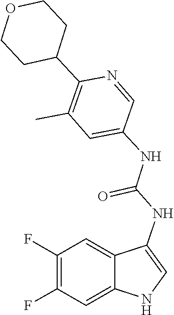 [SMILES: Cc1cc(NC(=O)Nc2c[nH]c3cc(F)c(F)cc23)cnc1C1CCOCC1] |
| 170 | 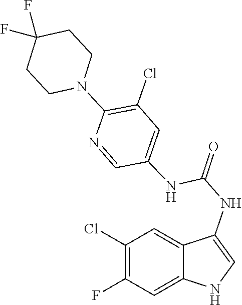 [SMILES: O=C(Nc1cnc(N2CCC(F)(F)CC2)c(Cl)c1)Nc1c[nH]c2cc(F)c(Cl)cc12] |
| 171 | 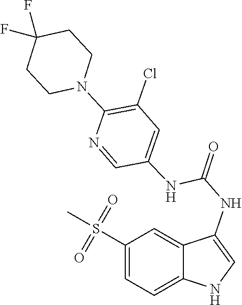 [SMILES: CS(=O)(=O)c1ccc2[nH]cc(NC(=O)Nc3cnc(N4CCC(F)(F)CC4)c(Cl)c3)c2c1] |
| 172 | 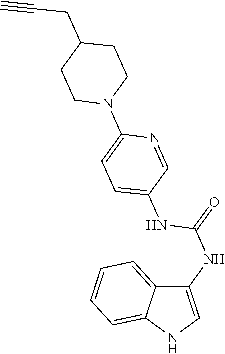 [SMILES: C#CCC1CCN(c2ccc(NC(=O)Nc3c[nH]c4ccccc34)cn2)CC1] |
| 173 | 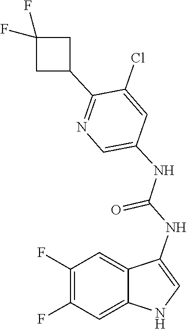 [SMILES: O=C(Nc1cnc(C2CC(F)(F)C2)c(Cl)c1)Nc1c[nH]c2cc(F)c(F)cc12] |
| 174 | 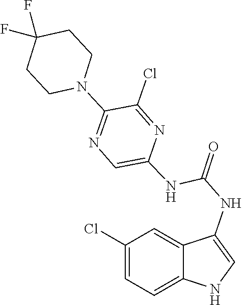 [SMILES: O=C(Nc1cnc(N2CCC(F)(F)CC2)c(Cl)n1)Nc1c[nH]c2ccc(Cl)cc12] |
| 175 | 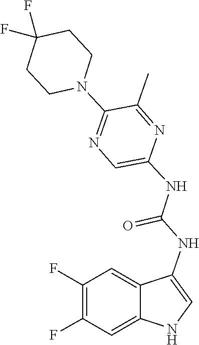 [SMILES: Cc1nc(NC(=O)Nc2c[nH]c3cc(F)c(F)cc23)cnc1N1CCC(F)(F)CC1] |
| 176 | 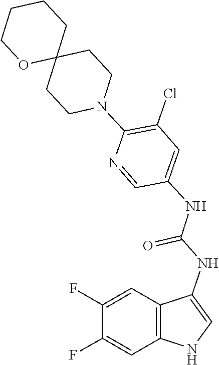 [SMILES: O=C(Nc1cnc(N2CCC3(CCCCO3)CC2)c(Cl)c1)Nc1c[nH]c2cc(F)c(F)cc12] |
| 177 | 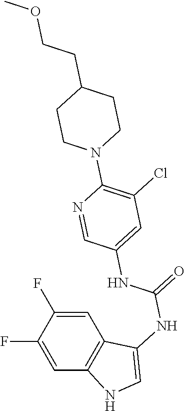 [SMILES: COCCC1CCN(c2ncc(NC(=O)Nc3c[nH]c4cc(F)c(F)cc34)cc2Cl)CC1] |
| 178 | 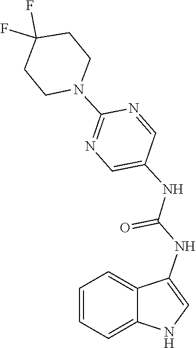 [SMILES: O=C(Nc1cnc(N2CCC(F)(F)CC2)nc1)Nc1c[nH]c2ccccc12] |
| 179 | 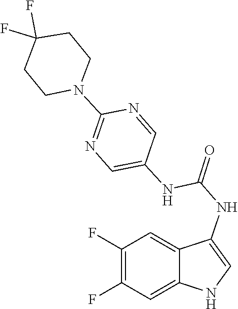 [SMILES: O=C(Nc1cnc(N2CCC(F)(F)CC2)nc1)Nc1c[nH]c2cc(F)c(F)cc12] |
| 180 | 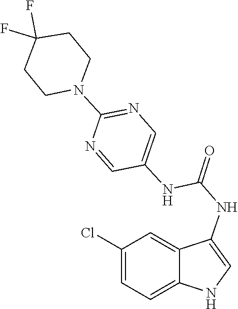 [SMILES: O=C(Nc1cnc(N2CCC(F)(F)CC2)nc1)Nc1c[nH]c2ccc(Cl)cc12] |
| 181 | 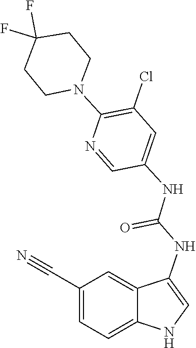 [SMILES: N#Cc1ccc2[nH]cc(NC(=O)Nc3cnc(N4CCC(F)(F)CC4)c(Cl)c3)c2c1] |
| 182 | 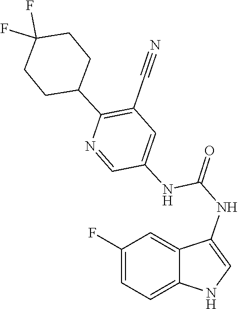 [SMILES: N#Cc1cc(NC(=O)Nc2c[nH]c3ccc(F)cc23)cnc1C1CCC(F)(F)CC1] |
| 183 | 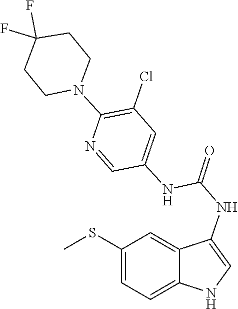 [SMILES: CSc1ccc2[nH]cc(NC(=O)Nc3cnc(N4CCC(F)(F)CC4)c(Cl)c3)c2c1] |
| 184 | 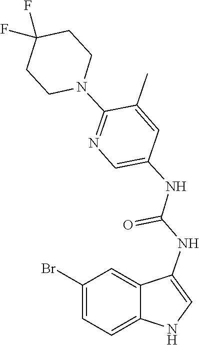 [SMILES: Cc1cc(NC(=O)Nc2c[nH]c3ccc(Br)cc23)cnc1N1CCC(F)(F)CC1] |
| 185 | 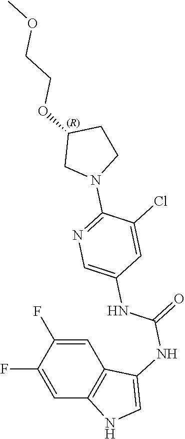 |
| 186 | 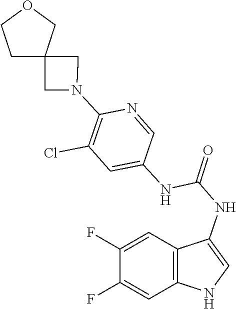 [SMILES: O=C(Nc1cnc(N2CC3(CCOC3)C2)c(Cl)c1)Nc1c[nH]c2cc(F)c(F)cc12] |
| 187 | 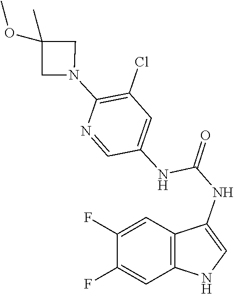 [SMILES: COC1(C)CN(c2ncc(NC(=O)Nc3c[nH]c4cc(F)c(F)cc34)cc2Cl)C1] |
| 188 | 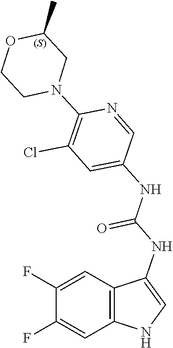 |
| 189 | 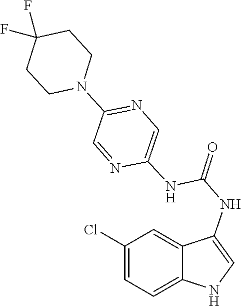 [SMILES: O=C(Nc1cnc(N2CCC(F)(F)CC2)cn1)Nc1c[nH]c2ccc(Cl)cc12] |
| 190 | 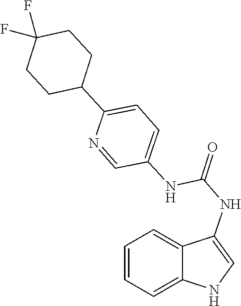 [SMILES: O=C(Nc1ccc(C2CCC(F)(F)CC2)nc1)Nc1c[nH]c2ccccc12] |
| 191 | 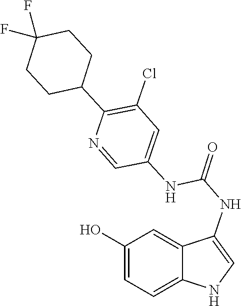 [SMILES: O=C(Nc1cnc(C2CCC(F)(F)CC2)c(Cl)c1)Nc1c[nH]c2ccc(O)cc12] |
| 192 | 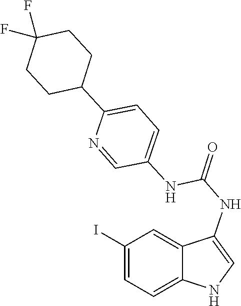 [SMILES: O=C(Nc1ccc(C2CCC(F)(F)CC2)nc1)Nc1c[nH]c2ccc(I)cc12] |
| 193 | 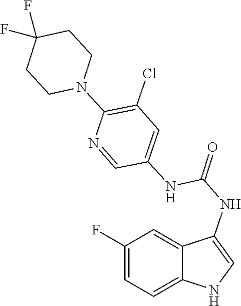 [SMILES: O=C(Nc1cnc(N2CCC(F)(F)CC2)c(Cl)c1)Nc1c[nH]c2ccc(F)cc12] |
| 194 | 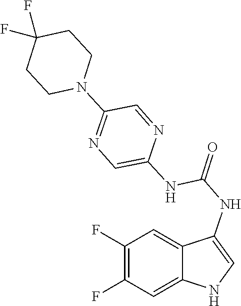 [SMILES: O=C(Nc1cnc(N2CCC(F)(F)CC2)cn1)Nc1c[nH]c2cc(F)c(F)cc12] |
| 195 | 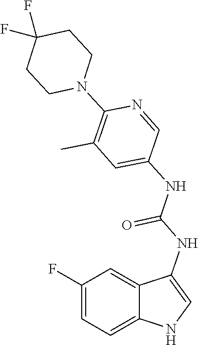 [SMILES: Cc1cc(NC(=O)Nc2c[nH]c3ccc(F)cc23)cnc1N1CCC(F)(F)CC1] |
| 196 | 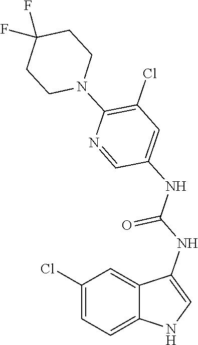 [SMILES: O=C(Nc1cnc(N2CCC(F)(F)CC2)c(Cl)c1)Nc1c[nH]c2ccc(Cl)cc12] |
| 197 | 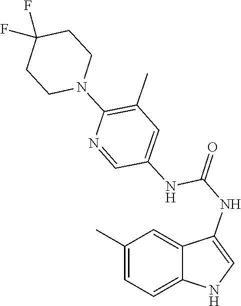 [SMILES: Cc1ccc2[nH]cc(NC(=O)Nc3cnc(N4CCC(F)(F)CC4)c(C)c3)c2c1] |
| 198 | 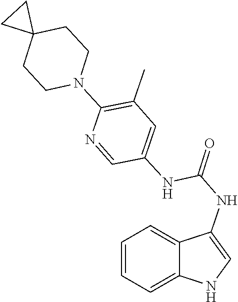 [SMILES: Cc1cc(NC(=O)Nc2c[nH]c3ccccc23)cnc1N1CCC2(CC1)CC2] |
| 199 | 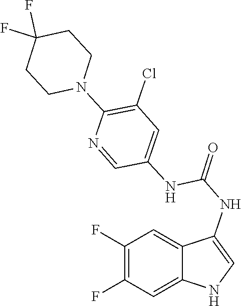 [SMILES: O=C(Nc1cnc(N2CCC(F)(F)CC2)c(Cl)c1)Nc1c[nH]c2cc(F)c(F)cc12] |
| 200 | 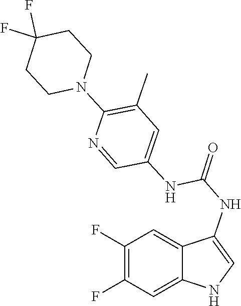 [SMILES: Cc1cc(NC(=O)Nc2c[nH]c3cc(F)c(F)cc23)cnc1N1CCC(F)(F)CC1] |
| 201 | 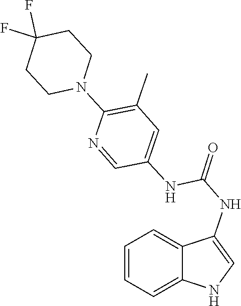 [SMILES: Cc1cc(NC(=O)Nc2c[nH]c3ccccc23)cnc1N1CCC(F)(F)CC1] |
| 202 | 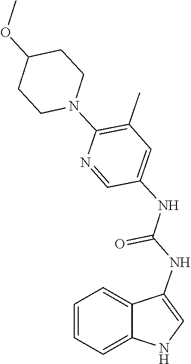 [SMILES: COC1CCN(c2ncc(NC(=O)Nc3c[nH]c4ccccc34)cc2C)CC1] |
| 203 | 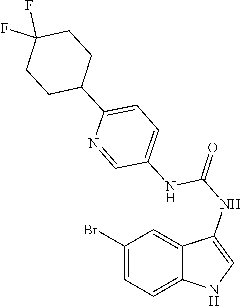 [SMILES: O=C(Nc1ccc(C2CCC(F)(F)CC2)nc1)Nc1c[nH]c2ccc(Br)cc12] |
| 204 | 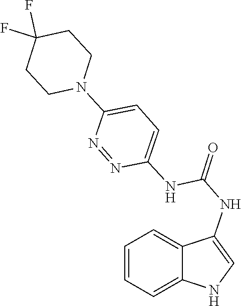 [SMILES: O=C(Nc1ccc(N2CCC(F)(F)CC2)nn1)Nc1c[nH]c2ccccc12] |
| 205 | 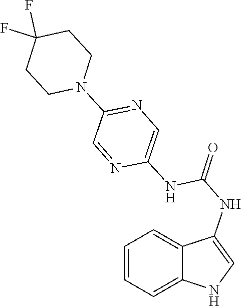 [SMILES: O=C(Nc1cnc(N2CCC(F)(F)CC2)cn1)Nc1c[nH]c2ccccc12] |
| 206 | 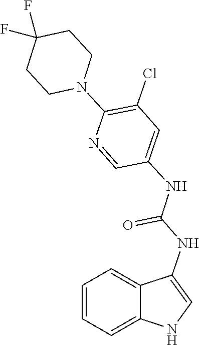 [SMILES: O=C(Nc1cnc(N2CCC(F)(F)CC2)c(Cl)c1)Nc1c[nH]c2ccccc12] |
| 207 | 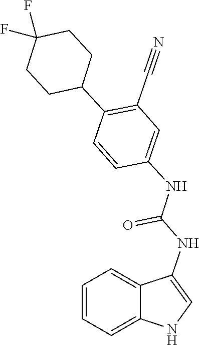 [SMILES: N#Cc1cc(NC(=O)Nc2c[nH]c3ccccc23)ccc1C1CCC(F)(F)CC1] |
| 208 | 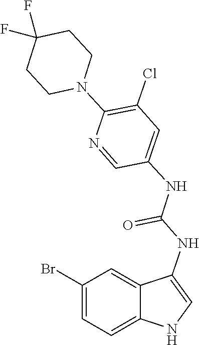 [SMILES: O=C(Nc1cnc(N2CCC(F)(F)CC2)c(Cl)c1)Nc1c[nH]c2ccc(Br)cc12] |
| 209 | 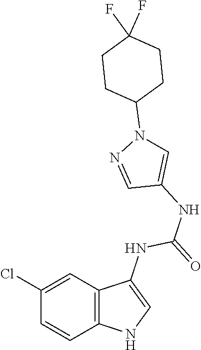 [SMILES: O=C(Nc1cnn(C2CCC(F)(F)CC2)c1)Nc1c[nH]c2ccc(Cl)cc12] |
| 210 | 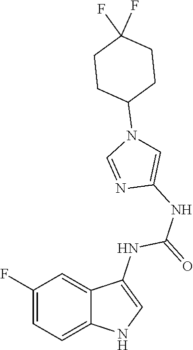 [SMILES: O=C(Nc1cn(C2CCC(F)(F)CC2)cn1)Nc1c[nH]c2ccc(F)cc12] |
| 211 | 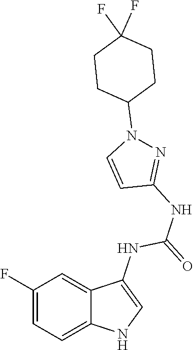 [SMILES: O=C(Nc1ccn(C2CCC(F)(F)CC2)n1)Nc1c[nH]c2ccc(F)cc12] |
| 212 | 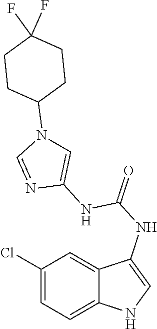 [SMILES: O=C(Nc1cn(C2CCC(F)(F)CC2)cn1)Nc1c[nH]c2ccc(Cl)cc12] |
| 213 | 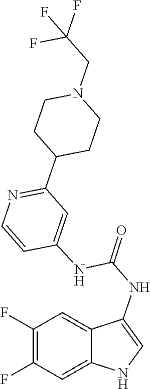 [SMILES: O=C(Nc1ccnc(C2CCN(CC(F)(F)F)CC2)c1)Nc1c[nH]c2cc(F)c(F)cc12] |
| 214 | 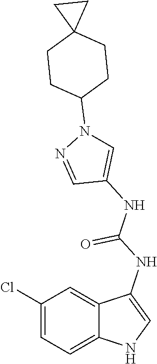 [SMILES: O=C(Nc1cnn(C2CCC3(CC2)CC3)c1)Nc1c[nH]c2ccc(Cl)cc12] |
| 215 | 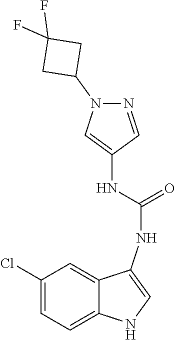 [SMILES: O=C(Nc1cnn(C2CC(F)(F)C2)c1)Nc1c[nH]c2ccc(Cl)cc12] |
| 216 | 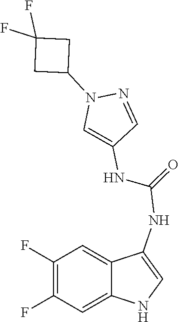 [SMILES: O=C(Nc1cnn(C2CC(F)(F)C2)c1)Nc1c[nH]c2cc(F)c(F)cc12] |
| 217 | 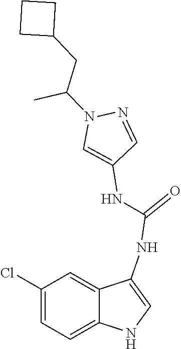 [SMILES: CC(CC1CCC1)n1cc(NC(=O)Nc2c[nH]c3ccc(Cl)cc23)cn1] |
| 218 | 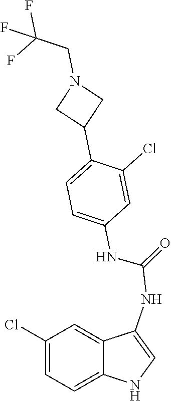 [SMILES: O=C(Nc1ccc(C2CN(CC(F)(F)F)C2)c(Cl)c1)Nc1c[nH]c2ccc(Cl)cc12] |
| 219 | 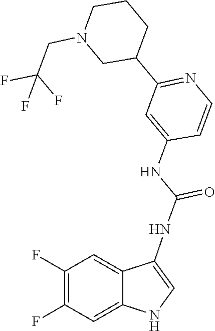 [SMILES: O=C(Nc1ccnc(C2CCCN(CC(F)(F)F)C2)c1)Nc1c[nH]c2cc(F)c(F)cc12] |
| 220 | 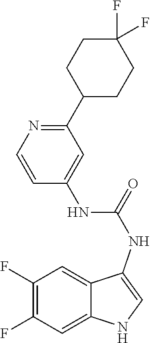 [SMILES: O=C(Nc1ccnc(C2CCC(F)(F)CC2)c1)Nc1c[nH]c2cc(F)c(F)cc12] |
| 221 | 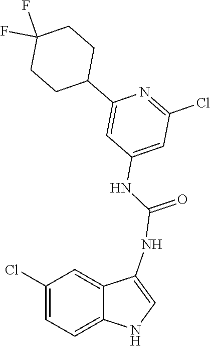 [SMILES: O=C(Nc1cc(Cl)nc(C2CCC(F)(F)CC2)c1)Nc1c[nH]c2ccc(Cl)cc12] |
| 222 | 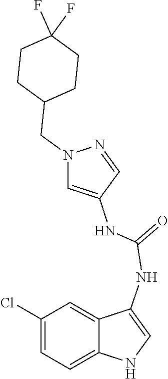 [SMILES: O=C(Nc1cnn(CC2CCC(F)(F)CC2)c1)Nc1c[nH]c2ccc(Cl)cc12] |
| 223 | 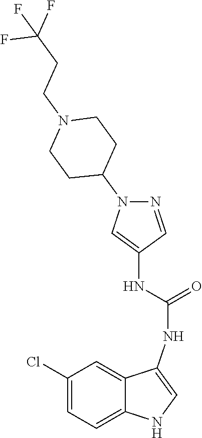 [SMILES: O=C(Nc1cnn(C2CCN(CCC(F)(F)F)CC2)c1)Nc1c[nH]c2ccc(Cl)cc12] |
| 224 | 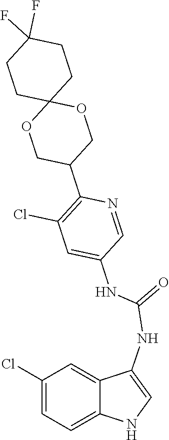 [SMILES: O=C(Nc1cnc(C2COC3(CCC(F)(F)CC3)OC2)c(Cl)c1)Nc1c[nH]c2ccc(Cl)cc12] |
| 225 | 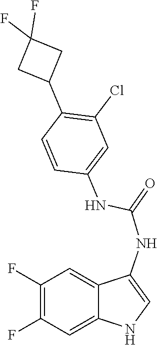 [SMILES: O=C(Nc1ccc(C2CC(F)(F)C2)c(Cl)c1)Nc1c[nH]c2cc(F)c(F)cc12] |
| 226 | 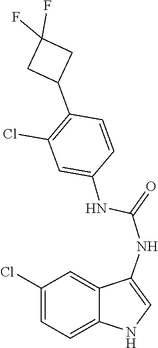 [SMILES: O=C(Nc1ccc(C2CC(F)(F)C2)c(Cl)c1)Nc1c[nH]c2ccc(Cl)cc12] |
| 227 | 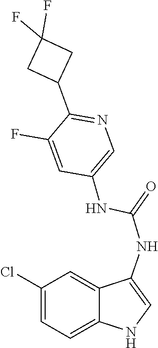 [SMILES: O=C(Nc1cnc(C2CC(F)(F)C2)c(F)c1)Nc1c[nH]c2ccc(Cl)cc12] |
| 228 | 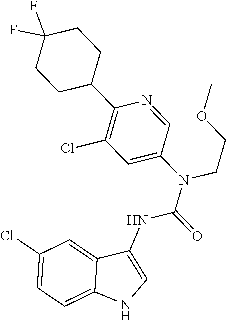 [SMILES: COCCN(C(=O)Nc1c[nH]c2ccc(Cl)cc12)c1cnc(C2CCC(F)(F)CC2)c(Cl)c1] |
| 229 | 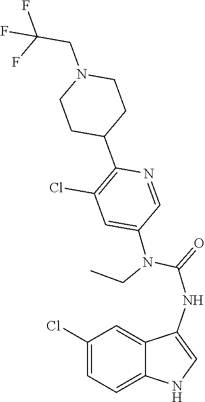 [SMILES: CCN(C(=O)Nc1c[nH]c2ccc(Cl)cc12)c1cnc(C2CCN(CC(F)(F)F)CC2)c(Cl)c1] |
| 230 | 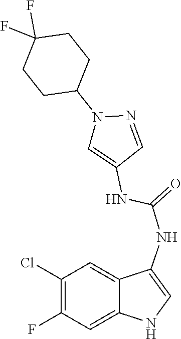 [SMILES: O=C(Nc1cnn(C2CCC(F)(F)CC2)c1)Nc1c[nH]c2cc(F)c(Cl)cc12] |
| 231 | 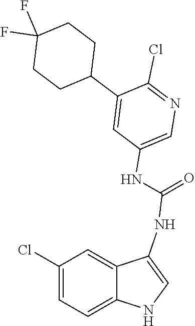 [SMILES: O=C(Nc1cnc(Cl)c(C2CCC(F)(F)CC2)c1)Nc1c[nH]c2ccc(Cl)cc12] |
| 232 | 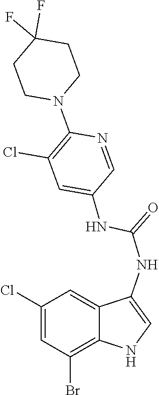 [SMILES: O=C(Nc1cnc(N2CCC(F)(F)CC2)c(Cl)c1)Nc1c[nH]c2c(Br)cc(Cl)cc12] |
| 233 | 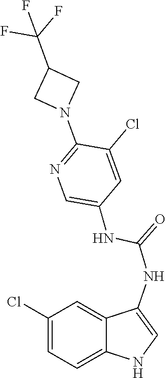 [SMILES: O=C(Nc1cnc(N2CC(C(F)(F)F)C2)c(Cl)c1)Nc1c[nH]c2ccc(Cl)cc12] |
| 234 | 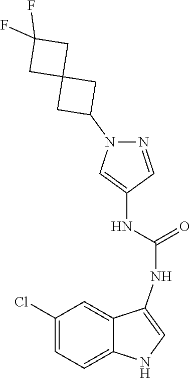 [SMILES: O=C(Nc1cnn(C2CC3(C2)CC(F)(F)C3)c1)Nc1c[nH]c2ccc(Cl)cc12] |
| 235 | 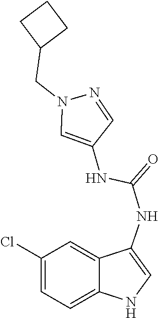 [SMILES: O=C(Nc1cnn(CC2CCC2)c1)Nc1c[nH]c2ccc(Cl)cc12] |
| 236 | 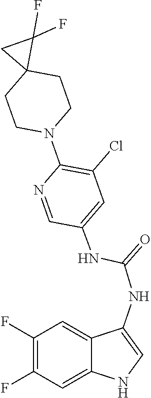 [SMILES: O=C(Nc1cnc(N2CCC3(CC2)CC3(F)F)c(Cl)c1)Nc1c[nH]c2cc(F)c(F)cc12] |
| 237 | 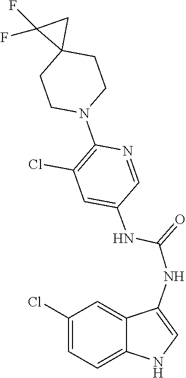 [SMILES: O=C(Nc1cnc(N2CCC3(CC2)CC3(F)F)c(Cl)c1)Nc1c[nH]c2ccc(Cl)cc12] |
| 238 | 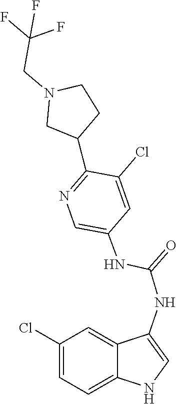 [SMILES: O=C(Nc1cnc(C2CCN(CC(F)(F)F)C2)c(Cl)c1)Nc1c[nH]c2ccc(Cl)cc12] |
| 239 | 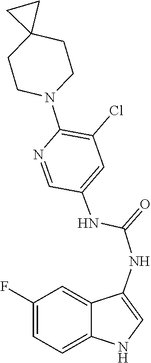 [SMILES: O=C(Nc1cnc(N2CCC3(CC2)CC3)c(Cl)c1)Nc1c[nH]c2ccc(F)cc12] |
| 240 | 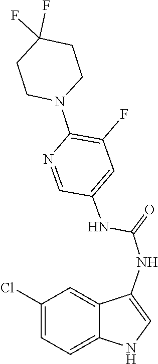 [SMILES: O=C(Nc1cnc(N2CCC(F)(F)CC2)c(F)c1)Nc1c[nH]c2ccc(Cl)cc12] |
| 241 | 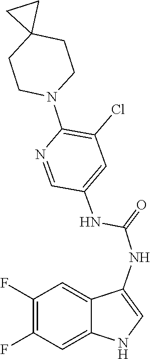 [SMILES: O=C(Nc1cnc(N2CCC3(CC2)CC3)c(Cl)c1)Nc1c[nH]c2cc(F)c(F)cc12] |
| 242 | 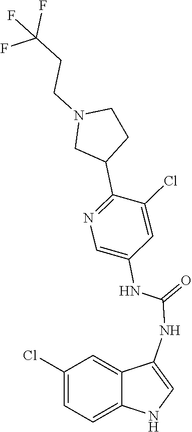 [SMILES: O=C(Nc1cnc(C2CCN(CCC(F)(F)F)C2)c(Cl)c1)Nc1c[nH]c2ccc(Cl)cc12] |
| 243 | 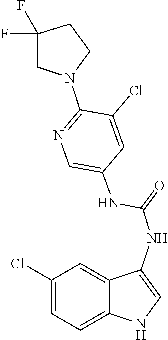 [SMILES: O=C(Nc1cnc(N2CCC(F)(F)C2)c(Cl)c1)Nc1c[nH]c2ccc(Cl)cc12] |
| 244 | 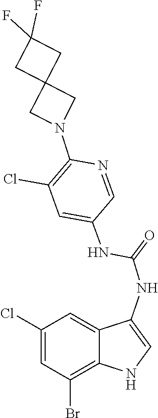 [SMILES: O=C(Nc1cnc(N2CC3(C2)CC(F)(F)C3)c(Cl)c1)Nc1c[nH]c2c(Br)cc(Cl)cc12] |
| 245 | 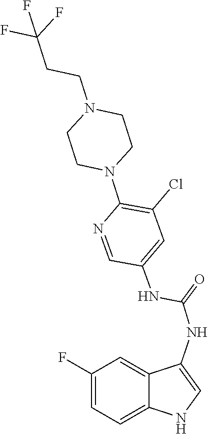 [SMILES: O=C(Nc1cnc(N2CCN(CCC(F)(F)F)CC2)c(Cl)c1)Nc1c[nH]c2ccc(F)cc12] |
| 246 | 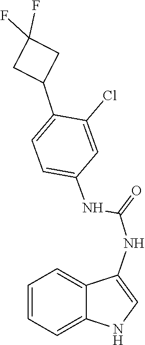 [SMILES: O=C(Nc1ccc(C2CC(F)(F)C2)c(Cl)c1)Nc1c[nH]c2ccccc12] |
| 247 | 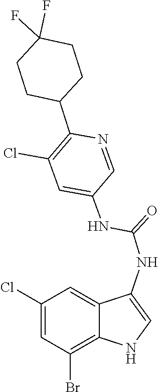 [SMILES: O=C(Nc1cnc(C2CCC(F)(F)CC2)c(Cl)c1)Nc1c[nH]c2c(Br)cc(Cl)cc12] |
| 248 | 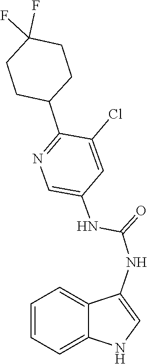 [SMILES: O=C(Nc1cnc(C2CCC(F)(F)CC2)c(Cl)c1)Nc1c[nH]c2ccccc12] |
| 249 | 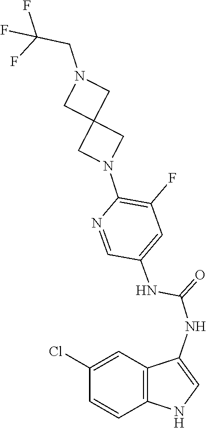 [SMILES: O=C(Nc1cnc(N2CC3(CN(CC(F)(F)F)C3)C2)c(F)c1)Nc1c[nH]c2ccc(Cl)cc12] |
| 250 | 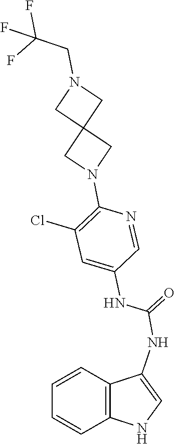 [SMILES: O=C(Nc1cnc(N2CC3(CN(CC(F)(F)F)C3)C2)c(Cl)c1)Nc1c[nH]c2ccccc12] |
| 251 | 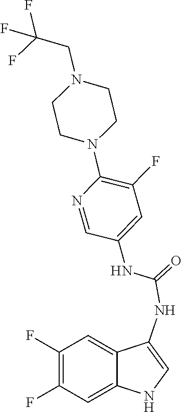 [SMILES: O=C(Nc1cnc(N2CCN(CC(F)(F)F)CC2)c(F)c1)Nc1c[nH]c2cc(F)c(F)cc12] |
| 252 | 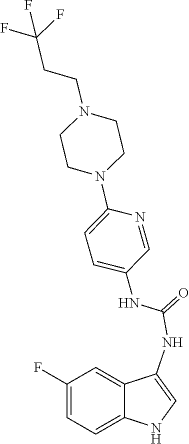 [SMILES: O=C(Nc1ccc(N2CCN(CCC(F)(F)F)CC2)nc1)Nc1c[nH]c2ccc(F)cc12] |
| 253 | 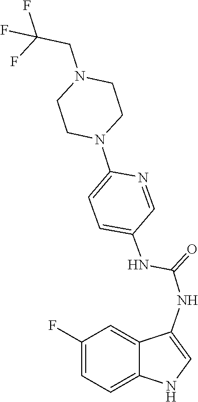 [SMILES: O=C(Nc1ccc(N2CCN(CC(F)(F)F)CC2)nc1)Nc1c[nH]c2ccc(F)cc12] |
| 254 | 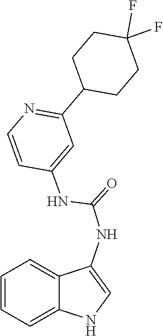 [SMILES: O=C(Nc1ccnc(C2CCC(F)(F)CC2)c1)Nc1c[nH]c2ccccc12] |
| 255 | 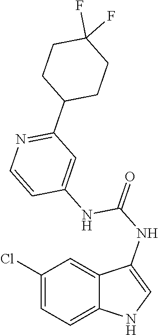 [SMILES: O=C(Nc1ccnc(C2CCC(F)(F)CC2)c1)Nc1c[nH]c2ccc(Cl)cc12] |
| 256 | 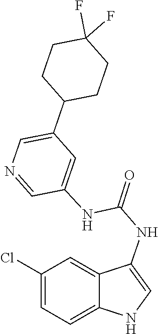 [SMILES: O=C(Nc1cncc(C2CCC(F)(F)CC2)c1)Nc1c[nH]c2ccc(Cl)cc12] |
| 257 | 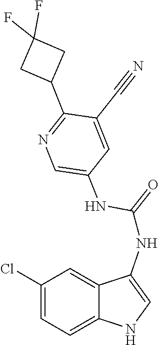 [SMILES: N#Cc1cc(NC(=O)Nc2c[nH]c3ccc(Cl)cc23)cnc1C1CC(F)(F)C1] |
| 258 | 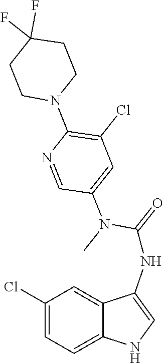 [SMILES: CN(C(=O)Nc1c[nH]c2ccc(Cl)cc12)c1cnc(N2CCC(F)(F)CC2)c(Cl)c1] |
| 260 | 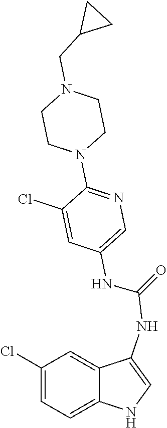 [SMILES: O=C(Nc1cnc(N2CCN(CC3CC3)CC2)c(Cl)c1)Nc1c[nH]c2ccc(Cl)cc12] |
| 261 | 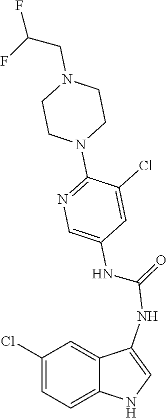 [SMILES: O=C(Nc1cnc(N2CCN(CC(F)F)CC2)c(Cl)c1)Nc1c[nH]c2ccc(Cl)cc12] |
| 262 | 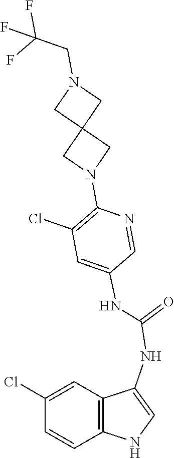 [SMILES: O=C(Nc1cnc(N2CC3(CN(CC(F)(F)F)C3)C2)c(Cl)c1)Nc1c[nH]c2ccc(Cl)cc12] |
| 263 | 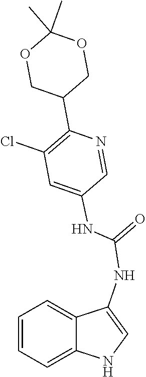 [SMILES: CC1(C)OCC(c2ncc(NC(=O)Nc3c[nH]c4ccccc34)cc2Cl)CO1] |
| 264 | 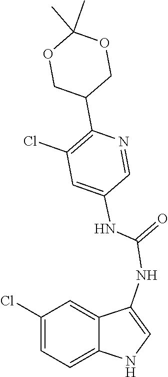 [SMILES: CC1(C)OCC(c2ncc(NC(=O)Nc3c[nH]c4ccc(Cl)cc34)cc2Cl)CO1] |
| 265 | 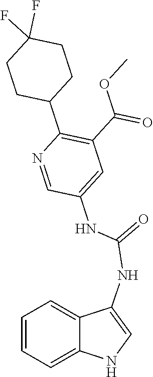 [SMILES: COC(=O)c1cc(NC(=O)Nc2c[nH]c3ccccc23)cnc1C1CCC(F)(F)CC1] |
| 266 | 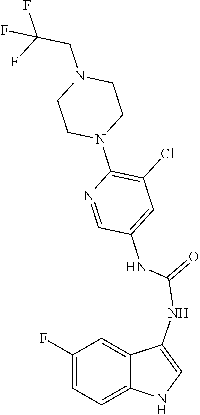 [SMILES: O=C(Nc1cnc(N2CCN(CC(F)(F)F)CC2)c(Cl)c1)Nc1c[nH]c2ccc(F)cc12] |
| 267 | 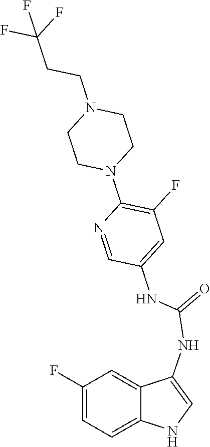 [SMILES: O=C(Nc1cnc(N2CCN(CCC(F)(F)F)CC2)c(F)c1)Nc1c[nH]c2ccc(F)cc12] |
| 268 | 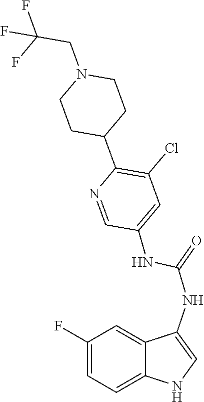 [SMILES: O=C(Nc1cnc(C2CCN(CC(F)(F)F)CC2)c(Cl)c1)Nc1c[nH]c2ccc(F)cc12] |
| 269 | 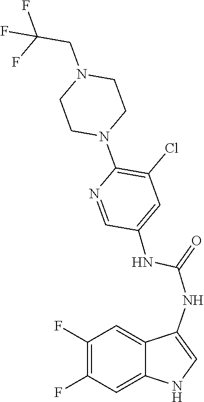 [SMILES: O=C(Nc1cnc(N2CCN(CC(F)(F)F)CC2)c(Cl)c1)Nc1c[nH]c2cc(F)c(F)cc12] |
| 270 | 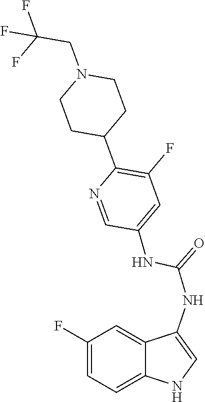 [SMILES: O=C(Nc1cnc(C2CCN(CC(F)(F)F)CC2)c(F)c1)Nc1c[nH]c2ccc(F)cc12] |
| 271 | 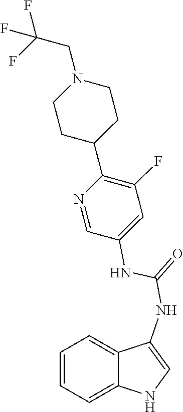 [SMILES: O=C(Nc1cnc(C2CCN(CC(F)(F)F)CC2)c(F)c1)Nc1c[nH]c2ccccc12] |
| 272 | 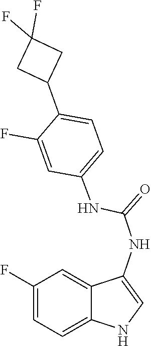 [SMILES: O=C(Nc1ccc(C2CC(F)(F)C2)c(F)c1)Nc1c[nH]c2ccc(F)cc12] |
| 273 | 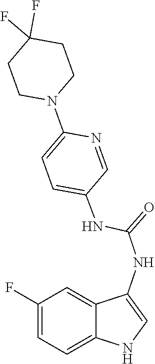 [SMILES: O=C(Nc1ccc(N2CCC(F)(F)CC2)nc1)Nc1c[nH]c2ccc(F)cc12] |
| 274 | 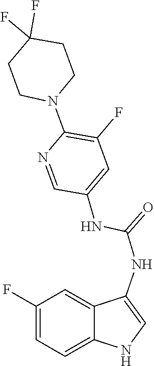 [SMILES: O=C(Nc1cnc(N2CCC(F)(F)CC2)c(F)c1)Nc1c[nH]c2ccc(F)cc12] |
| 275 | 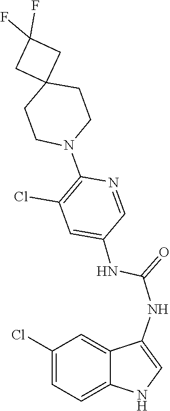 [SMILES: O=C(Nc1cnc(N2CCC3(CC2)CC(F)(F)C3)c(Cl)c1)Nc1c[nH]c2ccc(Cl)cc12] |
| 276 | 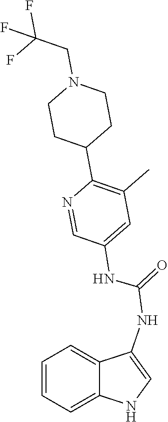 [SMILES: Cc1cc(NC(=O)Nc2c[nH]c3ccccc23)cnc1C1CCN(CC(F)(F)F)CC1] |
| 277 | 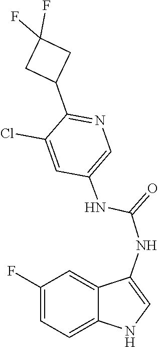 [SMILES: O=C(Nc1cnc(C2CC(F)(F)C2)c(Cl)c1)Nc1c[nH]c2ccc(F)cc12] |
| 278 | 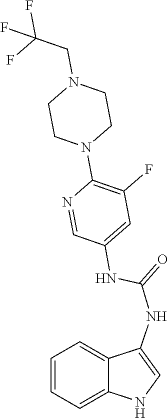 [SMILES: O=C(Nc1cnc(N2CCN(CC(F)(F)F)CC2)c(F)c1)Nc1c[nH]c2ccccc12] |
| 279 | 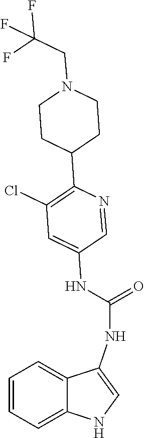 [SMILES: O=C(Nc1cnc(C2CCN(CC(F)(F)F)CC2)c(Cl)c1)Nc1c[nH]c2ccccc12] |
| 280 | 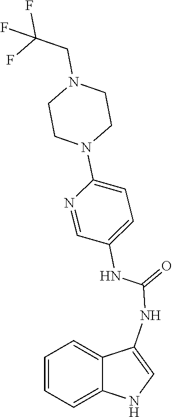 [SMILES: O=C(Nc1ccc(N2CCN(CC(F)(F)F)CC2)nc1)Nc1c[nH]c2ccccc12] |
| 281 | 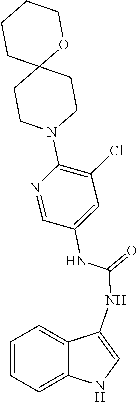 [SMILES: O=C(Nc1cnc(N2CCC3(CCCCO3)CC2)c(Cl)c1)Nc1c[nH]c2ccccc12] |
| 282 | 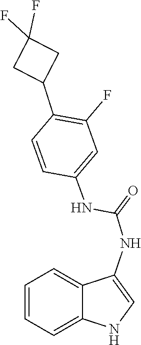 [SMILES: O=C(Nc1ccc(C2CC(F)(F)C2)c(F)c1)Nc1c[nH]c2ccccc12] |
| 283 | 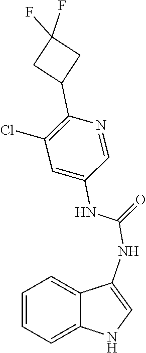 [SMILES: O=C(Nc1cnc(C2CC(F)(F)C2)c(Cl)c1)Nc1c[nH]c2ccccc12] |
| 284 | 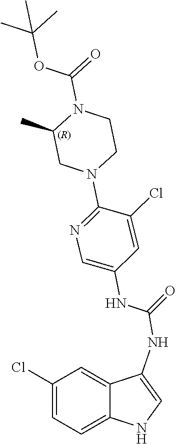 |
| 285 | 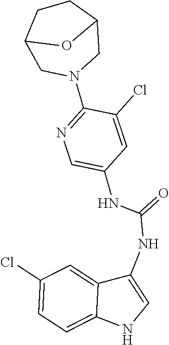 [SMILES: O=C(Nc1cnc(N2CC3CCC(C2)O3)c(Cl)c1)Nc1c[nH]c2ccc(Cl)cc12] |
| 286 | 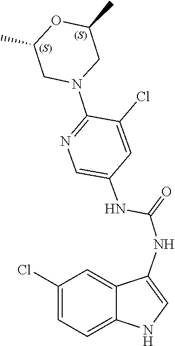 |
| 287 | 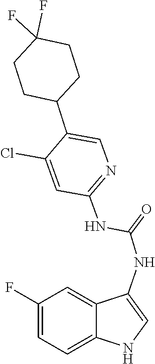 [SMILES: O=C(Nc1cc(Cl)c(C2CCC(F)(F)CC2)cn1)Nc1c[nH]c2ccc(F)cc12] |
| 288 | 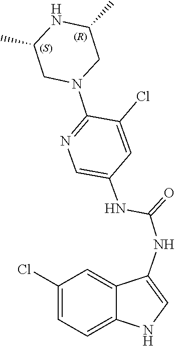 |
| 289 | 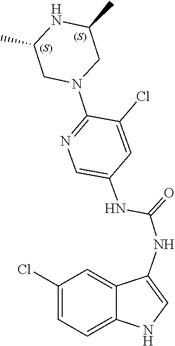 |
| 290 | 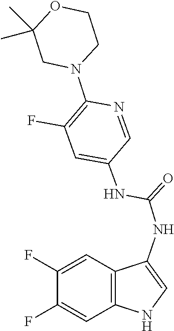 [SMILES: CC1(C)CN(c2ncc(NC(=O)Nc3c[nH]c4cc(F)c(F)cc34)cc2F)CCO1] |
| 291 | 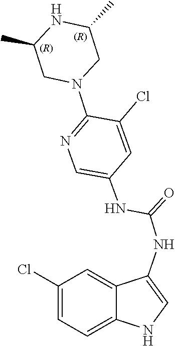 |
| 292 | 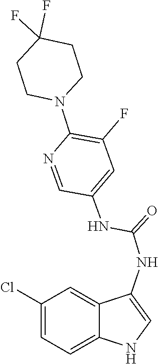 [SMILES: O=C(Nc1cnc(N2CCC(F)(F)CC2)c(F)c1)Nc1c[nH]c2ccc(Cl)cc12] |
| 293 | 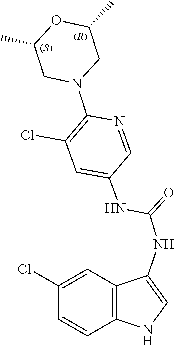 |
| 294 | 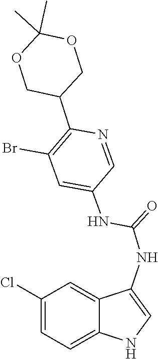 [SMILES: CC1(C)OCC(c2ncc(NC(=O)Nc3c[nH]c4ccc(Cl)cc34)cc2Br)CO1] |
| 295 | 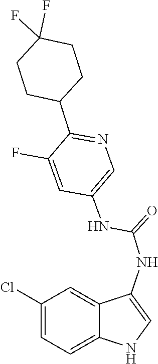 [SMILES: O=C(Nc1cnc(C2CCC(F)(F)CC2)c(F)c1)Nc1c[nH]c2ccc(Cl)cc12] |
| 296 | 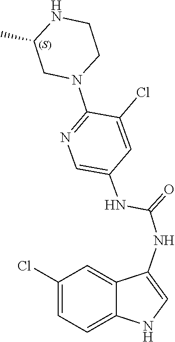 |
| 297 | 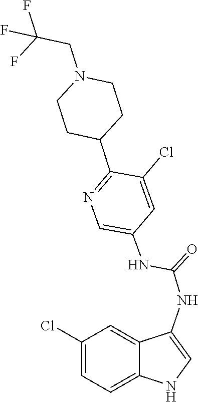 [SMILES: O=C(Nc1cnc(C2CCN(CC(F)(F)F)CC2)c(Cl)c1)Nc1c[nH]c2ccc(Cl)cc12] |
| 298 | 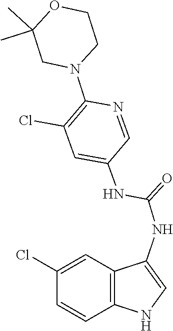 [SMILES: CC1(C)CN(c2ncc(NC(=O)Nc3c[nH]c4ccc(Cl)cc34)cc2Cl)CCO1] |
| 299 | 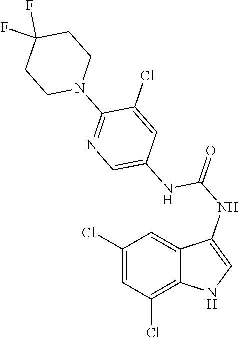 [SMILES: O=C(Nc1cnc(N2CCC(F)(F)CC2)c(Cl)c1)Nc1c[nH]c2c(Cl)cc(Cl)cc12] |
| 300 | 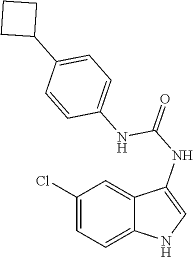 [SMILES: O=C(Nc1ccc(C2CCC2)cc1)Nc1c[nH]c2ccc(Cl)cc12] |
| 301 | 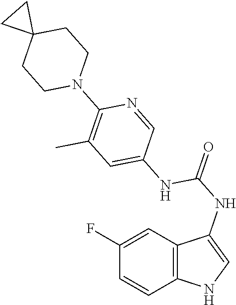 [SMILES: Cc1cc(NC(=O)Nc2c[nH]c3ccc(F)cc23)cnc1N1CCC2(CC1)CC2] |

Pharmaceutical Compositions and Administration

General

In some embodiments, a chemical entity (e.g., a compound that inhibits (e.g., antagonizes) STING, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, ρ, and γ-cyclodextrin, or chemically modified derivatives such as hydroxyalkylcyclodextrins, including 2- and 3-hydroxypropyl-o-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which increased (e.g., excessive)STING activity (e.g., e.g., STING signaling) contributes to the pathology and/or symptoms and/or progression of the condition, disease or disorder (e.g., immune disorders, cancer) are provided.

Indications

In some embodiments, the condition, disease or disorder is cancer. Non-limiting examples of cancer include melanoma, carcinoma, lymphoma, blastoma, sarcoma, and leukemia or lymphoid malignancies. More particular examples of such cancers include breast cancer, colon cancer, rectal cancer, colorectal cancer, kidney or renal cancer, clear cell cancer lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, prostatic neoplasms, liver cancer, bladder cancer, cancer of the peritoneum, hepatocellular cancer, gastric or stomach cancer including gastrointestinal cancer, gastrointestinal stromal tumor, pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma, myelodysplasia disorders, myeloproliferative disorders, chronic myelogenous leukemia, and acute hematologic malignancies, endometrial or uterine carcinoma, endometriosis, endometrial stromal sarcoma, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, mast cell sarcoma, ovarian sarcoma, uterine sarcoma, melanoma, malignant mesothelioma, skin carcinomas, Schwannoma, oligodendroglioma, neuroblastomas, neuroectodermal tumor, rhabdomyosarcoma, osteogenic sarcoma, leiomyosarcomas, Ewing Sarcoma, peripheral primitive neuroectodermal tumor, urinary tract carcinomas, thyroid carcinomas, Wilm's tumor, as well as abnormal vascular proliferation associated with phakomatoses, edema (such as that associated with brain tumors), and Meigs' syndrome. In some cases, the cancer is melanoma.

In some embodiments, the condition, disease or disorder is a neurological disorder, which includes disorders that involve the central nervous system (brain, brainstem and cerebellum), the peripheral nervous system (including cranial nerves), and the autonomic nervous system (parts of which are located in both central and peripheral nervous system). Non-limiting examples of such neurological disorders include acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other “tauopathies”; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease—neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjögren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffman disease; West syndrome; whiplash; Williams syndrome; Wildon's disease; amyotrophe lateral sclerosis and Zellweger syndrome.

In some embodiments, the condition, disease or disorder is STING-associated conditions, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis. In certain embodiments, the condition, disease or disorder is an autoimmune disease (e.g., a cytosolic DNA-triggered autoinflammatory disease). Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, modulation of the immune system by STING provides for the treatment of diseases, including diseases caused by foreign agents. Exemplary infections by foreign agents which may be treated and/or prevented by the method of the present invention include an infection by a bacterium (e.g., a Gram-positive or Gram-negative bacterium), an infection by a fungus, an infection by a parasite, and an infection by a virus. In one embodiment of the present invention, the infection is a bacterial infection (e.g., infection by E. coli, Klebsiella pneumoniae, Pseudomonas aeruginosa, Salmonella spp., Staphylococcus aureus, Streptococcus spp., or vancomycin-resistant enterococcus), or sepsis. In another embodiment, the infection is a fungal infection (e.g. infection by a mould, a yeast, or a higher fungus). In still another embodiment, the infection is a parasitic infection (e.g., infection by a single-celled or multicellular parasite, including Giardia duodenalis, Cryptosporidium parvum, Cyclospora cayetanensis, and Toxoplasma gondiz). In yet another embodiment, the infection is a viral infection (e.g., infection by a virus associated with AIDS, avian flu, chickenpox, cold sores, common cold, gastroenteritis, glandular fever, influenza, measles, mumps, pharyngitis, pneumonia, rubella, SARS, and lower or upper respiratory tract infection (e.g., respiratory syncytial virus)).

In some embodiments, the condition, disease or disorder is hepatits B (see, e.g., WO 2015/061294).

In some embodiments, the condition, disease or disorder is selected from cardiovascular diseases (including e.g., myocardial infarction).

In some embodiments, the condition, disease or disorder is age-related macular degeneration.

In some embodiments, the condition, disease or disorder is mucositis, also known as stomatitits, which can occur as a result of chemotherapy or radiation therapy, either alone or in combination as well as damage caused by exposure to radiation outside of the context of radiation therapy.

In some embodiments, the condition, disease or disorder is uveitis, which is inflammation of the uvea (e.g., anterior uveitis, e.g., iridocyclitis or iritis; intermediate uveitis (also known as pars planitis); posterior uveitis; or chorioretinitis, e.g., pan-uveitis).

In some embodiments, the condition, disease or disorder is selected from the group consisting of a cancer, a neurological disorder, an autoimmune disease, hepatitis B, uvetitis, a cardiovascular disease, age-related macular degeneration, and mucositis.

Still other examples can include those indications discussed herein and below in contemplated combination therapy regimens.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the methods described herein can further include administering one or more additional cancer therapies.

The one or more additional cancer therapies can include, without limitation, surgery, radiotherapy, chemotherapy, toxin therapy, immunotherapy, cryotherapy, cancer vaccines (e.g., HPV vaccine, hepatitis B vaccine, Oncophage, Provenge) and gene therapy, as well as combinations thereof. Immunotherapy, including, without limitation, adoptive cell therapy, the derivation of stem cells and/or dendritic cells, blood transfusions, lavages, and/or other treatments, including, without limitation, freezing a tumor.

In some embodiments, the one or more additional cancer therapies is chemotherapy, which can include administering one or more additional chemotherapeutic agents.

In certain embodiments, the additional chemotherapeutic agent is an immunomodulatory moiety, e.g., an immune checkpoint inhibitor. In certain of these embodiments, the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155; e.g., CTLA-4 or PD1 or PD-L1). See, e.g., Postow, M. J. Clin. Oncol. 2015, 33, 1.

In certain of these embodiments, the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

In certain embodiments, the additional chemotherapeutic agent is an alkylating agent. Alkylating agents are so named because of their ability to alkylate many nucleophilic functional groups under conditions present in cells, including, but not limited to cancer cells. In a further embodiment, an alkylating agent includes, but is not limited to, Cisplatin, carboplatin, mechlorethamine, cyclophosphamide, chlorambucil, ifosfamide and/or oxaliplatin. In an embodiment, alkylating agents can function by impairing cell function by forming covalent bonds with the amino, carboxyl, sulfhydryl, and phosphate groups in biologically important molecules or they can work by modifying a cell's DNA. In a further embodiment an alkylating agent is a synthetic, semisynthetic or derivative.

In certain embodiments, the additional chemotherapeutic agent is an anti-metabolite. Anti-metabolites masquerade as purines or pyrimidines, the building-blocks of DNA and in general, prevent these substances from becoming incorporated in to DNA during the “S” phase (of the cell cycle), stopping normal development and division. Anti-metabolites can also affect RNA synthesis. In an embodiment, an antimetabolite includes, but is not limited to azathioprine and/or mercaptopurine. In a further embodiment an anti-metabolite is a synthetic, semisynthetic or derivative.

In certain embodiments, the additional chemotherapeutic agent is a plant alkaloid and/or terpenoid. These alkaloids are derived from plants and block cell division by, in general, preventing microtubule function. In an embodiment, a plant alkaloid and/or terpenoid is a vinca alkaloid, a podophyllotoxin and/or a taxane. Vinca alkaloids, in general, bind to specific sites on tubulin, inhibiting the assembly of tubulin into microtubules, generally during the M phase of the cell cycle. In an embodiment, a vinca alkaloid is derived, without limitation, from the Madagascar periwinkle, Catharanthus roseus (formerly known as Vinca rosea). In an embodiment, a vinca alkaloid includes, without limitation, Vincristine, Vinblastine, Vinorelbine and/or Vindesine. In an embodiment, a taxane includes, but is not limited, to Taxol, Paclitaxel and/or Docetaxel.

In a further embodiment a plant alkaloid or terpernoid is a synthetic, semisynthetic or derivative. In a further embodiment, a podophyllotoxin is, without limitation, an etoposide and/or teniposide. In an embodiment, a taxane is, without limitation, docetaxel and/or ortataxel. [021] In an embodiment, a cancer therapeutic is a topoisomerase. Topoisomerases are essential enzymes that maintain the topology of DNA. Inhibition of type I or type II topoisomerases interferes with both transcription and replication of DNA by upsetting proper DNA supercoiling. In a further embodiment, a topoisomerase is, without limitation, a type I topoisomerase inhibitor or a type II topoisomerase inhibitor. In an embodiment a type I topoisomerase inhibitor is, without limitation, a camptothecin. In another embodiment, a camptothecin is, without limitation, exatecan, irinotecan, lurtotecan, topotecan, BNP 1350, CKD 602, DB 67 (AR67) and/or ST 1481. In an embodiment, a type II topoisomerase inhibitor is, without limitation, epipodophyllotoxin. In a further embodiment an epipodophyllotoxin is, without limitation, an amsacrine, etoposid, etoposide phosphate and/or teniposide. In a further embodiment a topoisomerase is a synthetic, semisynthetic or derivative, including those found in nature such as, without limitation, epipodophyllotoxins, substances naturally occurring in the root of American Mayapple (Podophyllum peltatum).

In certain embodiments, the additional chemotherapeutic agent is a stilbenoid. In a further embodiment, a stilbenoid includes, but is not limited to, Resveratrol, Piceatannol, Pinosylvin, Pterostilbene, Alpha-Viniferin, Ampelopsin A, Ampelopsin E, Diptoindonesin C, Diptoindonesin F, Epsilon-Vinferin, Flexuosol A, Gnetin H, Hemsleyanol D, Hopeaphenol, Trans-Diptoindonesin B, Astringin, Piceid and Diptoindonesin A. In a further embodiment a stilbenoid is a synthetic, semisynthetic or derivative.

In certain embodiments, the additional chemotherapeutic agent is a cytotoxic antibiotic. In an embodiment, a cytotoxic antibiotic is, without limitation, an actinomycin, an anthracenedione, an anthracycline, thalidomide, dichloroacetic acid, nicotinic acid, 2-deoxyglucose and/or chlofazimine. In an embodiment, an actinomycin is, without limitation, actinomycin D, bacitracin, colistin (polymyxin E) and/or polymyxin B. In another embodiment, an antracenedione is, without limitation, mitoxantrone and/or pixantrone. In a further embodiment, an anthracycline is, without limitation, bleomycin, doxorubicin (Adriamycin), daunorubicin (daunomycin), epirubicin, idarubicin, mitomycin, plicamycin and/or valrubicin. In a further embodiment a cytotoxic antibiotic is a synthetic, semisynthetic or derivative.

In certain embodiments, the additional chemotherapeutic agent is selected from endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, signal transduction inhibitors, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment) and the like.

In certain embodiments, the additional chemotherapeutic agent is selected from abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

In certain embodiments, the additional chemotherapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In still other embodiments, the additional chemotherapeutic agent can be selected from those delineated in U.S. Pat. No. 7,927,613, which is incorporated herein by reference in its entirety.

In some embodiments, the additional therapeutic agent and/or regimen are those that can be used for treating other STING-associated conditions, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis and the like.

Non-limiting examples of additional therapeutic agents and/or regimens for treating rheumatoid arthritis include non-steroidal anti-inflammatory drugs (NSAIDs; e.g., ibuprofen and naproxen), corticosteroids (e.g, prednisone), disease-modifying antirheumatic drugs (DMARDs; e.g., methotrexate (Trexall®, Otrexup®, Rasuvo®, Rheumatrex®), leflunomide (Arava®), hydroxychloroquine (Plaquenil), PF-06650833, iguratimod, tofacitinib (Xeljanz®), ABBV-599, evobrutinib, and sulfasalazine (Azulfidine®)), and biologics (e.g., abatacept (Orencia®), adalimumab (Humira®), anakinra (Kineret®), certolizumab (Cimzia®), etanercept (Enbrel®), golimumab (Simponi®), infliximab (Remicade®), rituximab (Rituxan®), tocilizumab (Actemra®), vobarilizumab, sarilumab (Kevzara®), secukinumab, ABP 501, CHS-0214, ABC-3373, and tocilizumab (ACTEMRA®)).

Non-limiting examples of additional therapeutic agents and/or regimens for treating lupus include steroids, topical immunomodulators (e.g., tacrolimus ointment (Protopic®) and pimecrolimus cream (Elidel®)), thalidomide (Thalomid®), non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., evobrutinib, iberdomide, voclosporin, cenerimod, azathioprine (Imuran®), cyclophosphamide (Cytoxan®, Neosar®, Endoxan®), and cyclosporine (Neoral, Sandimmune®, Gengraf®), and mycophenolate mofetil) baricitinb, iguratimod, filogotinib, GS-9876, rapamycin, and PF-06650833), and biologics (e.g., belimumab (Benlysta®), anifrolumab, prezalumab, MEDIO700, obinutuzumab, vobarilizumab, lulizumab, atacicept, PF-06823859, and lupizor, rituximab, BT063, BI655064, BIIB059, aldesleukin (Proleukin®), dapirolizumab, edratide, IFN-α-kinoid, OMS721, RC18, RSLV-132, theralizumab, XmAb5871, and ustekinumab (Stelara®)). For example, non-limiting treatments for systemic lupus erythematosus include non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., iberdomide, voclosporin, azathioprine (Imuran®), cyclophosphamide (Cytoxan®, Neosar®, Endoxan®), and cyclosporine (Neoral, Sandimmune®, Gengraf®), and mycophenolate mofetil, baricitinb, filogotinib, and PF-06650833), and biologics (e.g., belimumab (Benlysta®), anifrolumab, prezalumab, MEDIO700, vobarilizumab, lulizumab, atacicept, PF-06823859, lupizor, rituximab, BT063, BI655064, BIIB059, aldesleukin (Proleukin®), dapirolizumab, edratide, IFN-α-kinoid, RC18, RSLV-132, theralizumab, XmAb5871, and ustekinumab (Stelara®)). As another example, non-limiting examples of treatments for cutaneous lupus include steroids, immunomodulators (e.g., tacrolimus ointment (Protopic®) and pimecrolimus cream (Elidel®)), GS-9876, filogotinib, and thalidomide (Thalomid®). Agents and regimens for treating drug-induced and/or neonatal lupus can also be administered.

Non-limiting examples of additional therapeutic agents and/or regimens for treating STING-associated vasculopathy with onset in infancy (SAVI) include JAK inhibitors (e.g., tofacitinib, ruxolitinib, filgotinib, and baricitinib).

Non-limiting examples of additional therapeutic agents and/or regimens for treating Aicardi-Goutières Syndrome (AGS) include physiotherapy, treatment for respiratory complications, anticonvulsant therapies for seizures, tube-feeding, nucleoside reverse transcriptase inhibitors (e.g., emtricitabine (e.g., Emtriva®), tenofovir (e.g., Viread®), emtricitabine/tenofovir (e.g., Truvada®), zidovudine, lamivudine, and abacavir), and JAK inhibitors (e.g., tofacitinib, ruxolitinib, filgotinib, and baricitinib).

Non-limiting examples of additional therapeutic agents and/or regimens for treating IBDs include 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, corticosteroids (e.g., prednisone, Methylprednisolone, prednisone), CP-690,550, CT-P13, cyclosporine, DIMS0150, E6007, E6011, etrasimod, etrolizumab, fecal microbial transplantation, figlotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, infliximab, Interleukin 2 (IL-2), Janus kinase (JAK) inhibitors, laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

Non-limiting examples of additional therapeutic agents and/or regimens for treating irritable bowel syndrome include alosetron, bile acid sequesterants (e.g., cholestyramine, colestipol, colesevelam), chloride channel activators (e.g., lubiprostone), coated peppermint oil capsules, desipramine, dicyclomine, ebastine, eluxadoline, farnesoid X receptor agonist (e.g., obeticholic acid), fecal microbiota transplantation, fluoxetine, gabapentin, guanylate cyclase-C agonists (e.g., linaclotide, plecanatide), ibodutant, imipramine, JCM-16021, loperamide, lubiprostone, nortriptyline, ondansetron, opioids, paroxetine, pinaverium, polyethylene glycol, pregabalin, probiotics, ramosetron, rifaximin, and tanpanor.

Non-limiting examples of additional therapeutic agents and/or regimens for treating scleroderma include non-steroidal anti-inflammatory drugs (NSAIDs; e.g., ibuprofen and naproxen), corticosteroids (e.g, prednisone), immunomodulators (e.g., azathioprine, methotrexate (Trexall®, Otrexup®, Rasuvo®, Rheumatrex®), cyclophosphamide (Cytoxan®, Neosar®, Endoxan®), and cyclosporine (Neoral®, Sandimmune®, Gengraf®), antithymocyte globulin, mycophenolate mofetil, intravenous immunoglobulin, rituximab, sirolimus, and alefacept), calcium channel blockers (e.g., nifedipine), alpha blockers, serotonin receptor antagonists, angiotensin II receptor inhibitors, statins, local nitrates, iloprost, phosphodiesterase 5 inhibitors (e.g., sildenafil), bosentan, tetracycline antibiotics, endothelin receptor antagonists, prostanoids, and tyrosine kinase inhibitors (e.g., imatinib, nilotinib and dasatinib).

Non-limiting examples of additional therapeutic agents and/or regimens for treating Crohn's Disease (CD) include adalimumab, autologous CD34-selected peripheral blood stem cells transplant, 6-mercaptopurine, azathioprine, certolizumab pegol (Cimzia®), corticosteroids (e.g., prednisone), etrolizumab, E6011, fecal microbial transplantation, figlotinib, guselkumab, infliximab, IL-2, JAK inhibitors, matrix metalloproteinase 9 (MMP 9) inhibitors (e.g., GS-5745), MEDI2070, mesalamine, methotrexate, natalizumab, ozanimod, RHB-104, rifaximin, risankizumab, SHP647, sulfasalazine, thalidomide, upadacitinib, V565, and vedolizumab.

Non-limiting examples of additional therapeutic agents and/or regimens for treating UC include AbGn-168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol (Cimzia®), CP-690,550, corticosteroids (e.g., multimax budesonide, Methylprednisolone), cyclosporine, E6007, etrasimod, etrolizumab, fecal microbial transplantation, figlotinib, guselkumab, golimumab, IL-2, IMU-838, infliximab, matrix metalloproteinase 9 (MMP9) inhibitors (e.g., GS-5745), mesalamine, mesalamine, mirikizumab (LY3074828), RPC1063, risankizumab (BI 6555066), SHP647, sulfasalazine, TD-1473, TJ301, tildrakizumab (MK 3222), tofacitinib, tofacitinib, ustekinumab, UTTR1147A, and vedolizumab.

Non-limiting examples of additional therapeutic agents and/or regimens for treating autoimmune colitis include corticosteroids (e.g., budesonide, prednisone, prednisolone, Beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating iatrogenic autoimmune colitis include corticosteroids (e.g., budesonide, prednisone, prednisolone, Beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis induced by one or more chemotherapeutics agents include corticosteroids (e.g., budesonide, prednisone, prednisolone, beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating colitis induced by treatment with adoptive cell therapy include corticosteroids (e.g., budesonide, prednisone, prednisolone, beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis associated with one or more alloimmune diseases include corticosteroids (e.g., budesonide, prednisone, prednisolone, beclometasone dipropionate), sulfasalazine, and eicopentaenoic acid.

Non-limiting examples of additional therapeutic agents and/or regimens for treating radiation enteritis include teduglutide, amifostine, angiotensin-converting enzyme (ACE) inhibitors (e.g., benazepril, captopril, enalapril, fosinopril, lisinopril, moexipril, perindopril, quinapril, ramipril, and trandolapril), probiotics, selenium supplementation, statins (e.g., atorvastatin, fluvastatin, lovastatin, pravastatin, rosuvastatin, simvastatin, and pitavastatin), sucralfate, and vitamin E.

Non-limiting examples of additional therapeutic agents and/or regimens for treating collagenous colitis include 6-mercaptopurine, azathaioprine, bismuth subsalicate, Boswellia serrata extract, cholestyramine, colestipol, corticosteroids (e.g., budesonide, prednisone, prednisolone, beclometasone dipropionate), loperamide, mesalamine, methotrexate, probiotics, and sulfasalazine.

Non-limiting examples of additional therapeutic agents and/or regimens for treating lyphocytic colitis include 6-mercaptopurine, azathioprine, bismuth subsalicylate, cholestyramine, colestipol, corticosteroids (e.g., budesonide, prednisone, prednisolone, beclometasone dipropionate), loperamide, mesalamine, methotrexate, and sulfasalazine.

Non-limiting examples of additional therapeutic agents and/or regimens for treating microscopic colitis include 6-mercaptopurine, azathioprine, bismuth subsalicylate, Boswellia serrata extract, cholestyramine, colestipol, corticosteroids (e.g., budesonide, prednisone, prednisolone, beclometasone dipropionate), fecal microbial transplantation, loperamide, mesalamine, methotrexate, probiotics, and sulfasalazine.

Non-limiting examples of additional therapeutic agents and/or regimens for treating alloimmune disease include intrauterine platelet transfusions, intravenous immunoglobin, maternal steroids, abatacept, alemtuzumab, alphal-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating multiple sclerosis (MS) include alemtuzumab (Lemtrada®), ALKS 8700, amiloride, ATX-MS-1467, azathioprine, baclofen (Lioresal®), beta interferons (e.g., IFN-β-1a, IFN-β-1b), cladribine, corticosteroids (e.g., methylprednisolone), daclizumab, dimethyl fumarate (Tecfidera®), fingolimod (Gilenya®), fluoxetine, glatiramer acetate (Copaxone®), hydroxychloroquine, ibudilast, idebenone, laquinimod, lipoic acid, losartan, masitinib, MD1003 (biotin), mitoxantrone, montelukast, natalizumab (Tysabri®), NeuroVax™, ocrelizumab, ofatumumab, pioglitazone, and RPC1063.

Non-limiting examples of additional therapeutic agents and/or regimens for treating graft-vs-host disease include abatacept, alemtuzumab, alphal-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, imatinib, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating acute graft-vs-host disease include alemtuzumab, alpha-1 antitrypsin, antithymocyte globulin, basiliximab, brentuximab, corticosteroids (e.g., methylprednisone, prednisone), cyclosporine, dacilzumab, defribrotide, denileukin diftitox, ibrutinib, infliximab, itacitinib, LBH589, mycophenolate mofetil, natalizumab, neihulizumab, pentostatin, photopheresis, ruxolitinib, sirolimus, tacrolimus, and tocilizumab.

Non-limiting examples of additional therapeutic agents and/or regimens for treating chronic graft vs. host disease include abatacept, alemtuzumab, AMG592, antithymocyte globulin, basiliximab, bortezomib, corticosteroids (e.g., methylprednisone, prednisone), cyclosporine, dacilzumab, denileukin diftitox, glasdegib, ibrutinib, IL-2, imatinib, infliximab, mycophenolate mofetil, pentostatin, photobiomodulation, photopheresis, ruxolitinib, sirolimus, sonidegib, tacrolimus, tocilizumab, and vismodegib.

Non-limiting examples of additional therapeutic agents and/or regimens for treating celiac disease include AMG 714, AMY01, Aspergillus niger prolyl endoprotease, BL-7010, CALY-002, GBR 830, Hu-Mik-Beta-1, IMGX003, KumaMax, Larazotide Acetate, Nexvan2®, pancrelipase, TIMP-GLIA, vedolizumab, and ZED1227.

Non-limiting examples of additional therapeutic agents and/or regimens for treating psoriasis include topical corticosteroids, topical crisaborole/AN2728, topical SNA-120, topical SAN021, topical tapinarof, topical tocafinib, topical IDP-118, topical M518101, topical calcipotriene and betamethasone dipropionate (e.g., MC2-01 cream and Taclonex®), topical P-3073, topical LEO 90100 (Enstilar®), topical betamethasone dipropriate (Sernivo®), halobetasol propionate (Ultravate®), vitamin D analogues (e.g., calcipotriene (Dovonex®) and calcitriol (Vectical®)), anthralin (e.g., Dritho-scalp® and Dritho-creme®), topical retinoids (e.g., tazarotene (e.g., Tazorac® and Avage®)), calcineurin inhibitors (e.g., tacrolimus (Prograf®) and pimecrolimus (Elidel®)), salicylic acid, coal tar, moisturizers, phototherapy (e.g., exposure to sunlight, UVB phototherapy, narrow band UVB phototherapy, Goeckerman therapy, psoralen plus ultraviolet A (PUVA) therapy, and excimer laser), retinoids (e.g., acitretin (Soriatane®)), methotrexate (Trexall®, Otrexup®, Rasuvo®, Rheumatrex®), Apo805K1, baricitinib, FP187, KD025, prurisol, VTP-43742, XP23829, ZPL-389, CF101 (piclidenoson), LAS41008, VPD-737 (serlopitant), upadacitinib (ABT-494), aprmilast, tofacitibin, cyclosporine (Neoral®, Sandimmune®, Gengraf®), biologics (e.g., etanercept (Enbrel@), entanercept-szzs (Elrezi®), infliximab (Remicade®), adalimumab (Humira®), adalimumab-adbm (Cyltezo®), ustekinumab (Stelara®), golimumab (Simponi®), apremilast (Otezla®), secukinumab (Cosentyx®), certolixumab pegol, secukinumab, tildrakizumab-asmn, infliximab-dyyb, abatacept, ixekizumab (Taltz®), ABP 710, BCD-057, BI695501, bimekizumab (UCB4940), CHS-1420, GP2017, guselkumab (CNTO 1959), HD203, M923, MSB11022, Mirikizumab (LY3074828), PF-06410293, PF-06438179, risankizumab (BI655066), SB2, SB4, SB5, siliq (brodalumab), namilumab (MT203, tildrakizumab (MK-3222), and ixekizumab (Taltz®)), thioguanine, and hydroxyurea (e.g., Droxia® and Hydrea®).

Non-limiting examples of additional therapeutic agents and/or regimens for treating cutaneous T-cell lymphoma include phototherapy (e.g., exposure to sunlight, UVB phototherapy, narrow band UVB phototherapy, Goeckerman therapy, psoralen plus ultraviolet A (PUVA) therapy, and excimer laser), extracorporeal photopheresis, radiation therapy (e.g., spot radiation and total skin body electron beam therapy), stem cell transplant, corticosteroids, imiquimod, bexarotene gel, topical bis-chloroethyl-nitrourea, mechlorethamine gel, vorinostat (Zolinza®), romidepsin (Istodax®), pralatrexate (Folotyn®) biologics (e.g., alemtuzumab (Campath®), brentuximab vedotin (SGN-35), mogamulizumab, and IPH4102).

Non-limiting examples of additional therapeutic agents and/or regimens for treating uveitis include corticosteroids (e.g., intravitreal triamcinolone acetonide injectable suspensions), antibiotics, antivirals (e.g., acyclovir), dexamethasone, immunomodulators (e.g., tacrolimus, leflunomide, cyclophosphamide (Cytoxan®, Neosar®, Endoxan®), and cyclosporine (Neoral®, Sandimmune®, Gengraf®), chlorambucil, azathioprine, methotrexate, and mycophenolate mofetil), biologics (e.g., infliximab (Remicade®), adalimumab (Humira®), etanercept (Enbrel®), golimumab (Simponi®), certolizumab (Cimzia®), rituximab (Rituxan®), abatacept (Orencia®), basiliximab (Simulect®), anakinra (Kineret®), canakinumab (Ilaris®), gevokixumab (XOMA052), tocilizumab (Actemra®), alemtuzumab (Campath®), efalizumab (Raptiva®), LFG316, sirolimus (Santen®), abatacept, sarilumab (Kevzara®), and daclizumab (Zenapax®)), cytotoxic drugs, surgical implant (e.g., fluocinolone insert), and vitrectomy.

Non-limiting examples of additional therapeutic agents and/or regimens for treating mucositis include AG013, SGX942 (dusquetide), amifostine (Ethyol®), cryotherapy, cepacol lonzenges, capsaicin lozenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, granules comprising Vaccinium myrtillus extract, Macleaya cordata alkaloids and Echinacea angustifolia extract (e.g., SAMITAL®), and gastrointestinal cocktail (an acid reducer such aluminum hydroxide and magnesium hydroxide (e.g., Maalox), an antifungal (e.g., nystatin), and an analgesic (e.g., hurricane liquid)). For example, non-limiting examples of treatments for oral mucositis include AG013, amifostine (Ethyol®), cryotherapy, cepacol lonzenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble p-1,3/1,6 glucan, P276, LP-0004-09, CR-3294, ALD-518, IZN-6N4, quercetin, and gastrointestinal cocktail (an acid reducer such aluminum hydroxide and magnesium hydroxide (e.g., Maalox), an antifungal (e.g., nystatin), and an analgesic (e.g., hurricane liquid)). As another example, non-limiting examples of treatments for esophageal mucositis include xylocaine (e.g., gel viscous Xylocaine 2%). As another example, treatments for intestinal mucositis, treatments to modify intestinal mucositis, and treatments for intestinal mucositis signs and symptoms include gastrointestinal cocktail (an acid reducer such aluminum hydroxide and magnesium hydroxide (e.g., Maalox), an antifungal (e.g., nystatin), and an analgesic (e.g., hurricane liquid)).

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of such treatment (e.g., by way of biopsy, endoscopy, or other conventional method known in the art). In certain embodiments, the STING protein can serve as a biomarker for certain types of cancer, e.g., colon cancer and prostate cancer. In other embodiments, identifying a subject can include assaying the patient's tumor microenvironment for the absence of T-cells and/or presence of exhausted T-cells, e.g., patients having one or more cold tumors. Such patients can include those that are resistant to treatment with checkpoint inhibitors. In certain embodiments, such patients can be treated with a chemical entity herein, e.g., to recruit T-cells into the tumor, and in some cases, further treated with one or more checkpoint inhibitors, e.g., once the T-cells become exhausted.

In some embodiments, the chemical entities, methods, and compositions described herein can be administered to certain treatment-resistant patient populations (e.g., patients resistant to checkpoint inhibitors; e.g., patients having one or more cold tumors, e.g., tumors lacking T-cells or exhausted T-cells).

Compound Preparation

As can be appreciated by the skilled artisan, methods of synthesizing the compounds of the formulae herein will be evident to those of ordinary skill in the art.

Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and R G M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds of the invention are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g. diisopropylamine, 1,8-diazabicycloundec-7-ene, 2,6-di-tert-butylpyridine, or tetrabutylphosphazene).

The skilled artisan will recognize a variety of analytical methods that can be used to characterize the compounds described herein, including, for example, ¹H NMR, heteronuclear NMR, mass spectrometry, liquid chromatography, and infrared spectroscopy. The foregoing list is a subset of characterization methods available to a skilled artisan and is not intended to be limiting.

To further illustrate the foregoing, the following non-limiting, exemplary synthetic schemes are included. Variations of these examples within the scope of the claims are within the purview of one skilled in the art and are considered to fall within the scope of the invention as described, and claimed herein. The reader will recognize that the skilled artisan, provided with the present disclosure, and skill in the art is able to prepare and use the invention without exhaustive examples.

The following abbreviations have the indicated meanings:

Ac = acetyl
ACN = acetonitrile
BINAP = 2,2′-bis(diphenylphosphino)-1,1′-binaphthalene
Boc₂O = di-tert-butyl dicarbonate
Bu = butyl
DAST = diethylaminosulphur trifluoride
DCM = dichloromethane
DMF = N,N-dimethylformamide
DMSO = dimethyl sulfoxide
DPPA = diphenyl azidophosphate
Dppf = bis(diphenylphosphino)ferrocene
DEAD = diethyl azodicarboxylate
DIEA = Ethyldiisopropylamine
PTSA = P-toluenesulfonic acid
Et = ethyl
HPLC = high performance liquid chromatography
HMDS = hexamethyldisilazane
LC-MS = liquid chromatography-mass spectrometry
Me = methyl
NMR = nuclear magnetic resonance
RT = retention time
TEA = trimethylamine
TFA = trifluoroacetic acid
THF = tetrahydrofuran
Xphos = 2-9dicyclohexylphosphino)-2,4,6-triisopropylbiphenyl
T₃P = 2,4,6itripropyl-1,3,5,2,4,6-trioxatriphosphorinane-
2,4,6-trioxide

### Examples

### Materials and Methods

The progress of reactions was often monitored by TLC or LC-MS. The identity of the products was often confirmed by LC-MS. The LC-MS was recorded using one of the following methods.

Method AB: Poroshell HPH-C18, 50*3.0 mm, 2.7 μm, 4 μL injection, 1.2 mL/min flowrate, 90-900 amu scan range, 254 nm UV detection. Mobile phase A: Water/0.04% NH₃.H₂O and Mobile Phase B (MPB): ACN. 10% MPB to 95% in 1.99 min, hold at 95% MPB for 0.6 min, 95% MPB to 10% in 0.2 min, then equilibration to 10% MPB for 0.5 min.

Method AH: EVO C18, 50*3 mm, 2.0 μL injection, 1.2 mL/min flowrate, 90-900 amu scan range, 254 nm UV detection. Mobile Phase A (MPA): Water/5 mM NH4HCO3 and Mobile Phase B (MPB): Acetonitrile. Elution 10% MPB to 95% in 2.00 min, hold at 95% MPB for 0.6 min, 95% MPB to 10% in 0.05 min, then equilibration to 10% MPB for 0.25 min.

LCMS Method A: Kinetex EVO C18 100A, 30 *3 mm, 0.5 μL injection, 1.2 mL/min flowrate, 90-900 amu scan range, 254 nm UV detection. Mobile Phase A (MPA): Water/5 mM NH₄HCO₃ and Mobile Phase B (MPB): Acetonitrile. Elution 10% MPB to 95% in 2.0 min, hold at 95% MPB for 0.30 min, 95% MPB to 10% in 0.10 min.

LCMS Method B: Xselect CSH C18, 50*3 mm, 1.0 μL injection, 1.2 mL/min flowrate, 90-900 amu scan range, 254 nm UV detection. Mobile Phase A (MPA): Water/0.1% FA and Mobile Phase B (MPB): Acetonitrile/0.1% FA. Elution 5% MPB to 100% in 2.00 min, hold at 100% MPB for 0.70 min, 100% MPB to 5% in 0.05 min, then equilibration to 5% MPB for 0.15 min.

LCMS Method C: XBridge Shield RP18, 50 *4.6 mm, 0.5 μL injection, 1.2 mL/min flowrate, 90-900 amu scan range, 254 nm UV detection. Mobile Phase A (MPA): Water/0.04% NH3.H2O and Mobile Phase B (MPB): Acetonitrile. Elution 10% MPB to 95% in 2.00 min, hold at 95% MPB for 0.79 min, 95% MPB to 10% in 0.06 min, then equilibration to 10% MPB for 0.15 min.

LCMS Method D: Shim-pack XR-ODS, 50*3 mm, 0.3 μL injection, 1.2 mL/min flowrate, 30-2000 amu scan range, 254 nm UV detection. Mobile Phase A (MPA): Water/0.05 TFA and Mobile Phase B (MPB): Acetonitrile/0.05% TFA. Elution 5% MPB to 100% in 1.10 min, hold at 100% MPB for 0.60 min, 100% MPB to 5% in 0.05 min, then equilibration to 5% MPB for 0.25 min.

LCMS Method E: Kinetex 2.6 um EVO C18 100A, 50*3 mm, 0.6 μL injection, 1.2 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 1.20 min, hold at 95% MPB for 0.50 min, 95% MPB to 10% in 0.05 min, then equilibration to 10% MPB for 0.10 min.

LCMS Method F: EVO C18, 50*3 mm, 0.1 μL injection, 1.2 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 2.00 min, hold at 95% MPB for 0.60 min, 95% MPB to 10% in 0.15 min, then equilibration to 10% MPB for 0.25 min.

LCMS Method G: Titank C18, 50*3 mm, 0.5 μL injection, 1.5 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 1.80 min, hold at 95% MPB for 0.80 min, 95% MPB to 10% in 0.15 min, then equilibration to 10% MPB for 0.25 min.

LCMS Method H: Poroshell HPH C18, 50*3 mm, 0.5 μL injection, 1.2 mL/min flowrate, 30-2000 amu scan range, 254 nm UV detection. Mobile Phase A (MPA): Water/5 mM NH₄HCO₃+5 mM NH₄OH and Mobile Phase B (MPB): Acetonitrile. Elution 10% MPB to 95% in 2.00 min, hold at 95% MPB for 0.70 min, 95% MPB to 5% in 0.05 min, then equilibration to 5% MPB for 0.25 min.

LCMS Method I: HALOC18, 30*3 mm, 0.5 μL injection, 1.5 mL/min flowrate, 30-2000 amu scan range, 254 nm UV detection. Mobile Phase A (MPA): Water/0.05% TFA and Mobile Phase B (MPB): Acetonitrile/0.05% TFA. Elution 5% MPB to 100% in 1.20 min, hold at 100% MPB for 0.60 min, 100% MPB to 5% in 0.02 min, then equilibration to 5% MPB for 0.18 min.

LCMS Method J: HALOC18, 30*3 mm, 0.5 μL injection, 1.5 mL/min flowrate, 30-2000 amu scan range, 254 nm UV detection. Mobile Phase A (MPA): Water/0.1% FA and Mobile Phase B (MPB): Acetonitrile/0.1% FA. Elution 5% MPB to 100% in 1.20 min, hold at 100% MPB for 0.60 min, 100% MPB to 5% in 0.02 min, then equilibration to 5% MPB for 0.18 min.

NMR was recorded on BRUKER NMR 300.03 Mz, DUL-C-H, ULTRASHIELD™ 300, AVANCE II 300 B-ACS™ 120 or BRUKER NMR 400.13 Mz, BBFO, ULTRASHIELD™ 400, AVANCE III 400, B-ACS™ 120.

### SYNTHESIS OF EXEMPLARY INTERMEDIATES

### Intermediate 1: 5,6-difluoro-1H-indol-3-amine

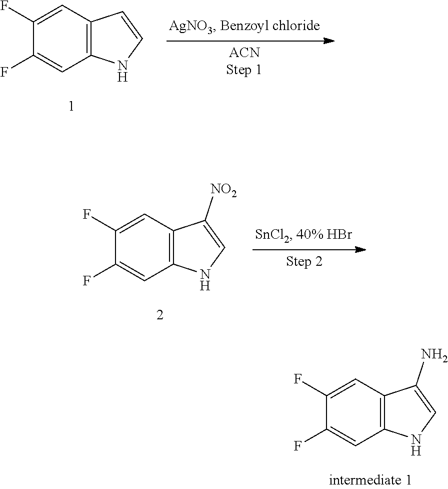 [SMILES: Fc1cc2cc[nH]c2cc1F ; O=[N+]([O-])c1c[nH]c2cc(F)c(F)cc12 ; Nc1c[nH]c2cc(F)c(F)cc12]

### Step 1—Synthesis of 5,6-difluoro-3-nitrol-1H-indole

5,6-Difluoro-1H-indole (5.0 g, 32.7 mmol, 1.0 equiv) was dissolved in CH₃CN (50.0 mL), and AgNO₃ (6.1 g, 36.0 mmol, 1.1 equiv) was added in portions. The resulting solution was then cooled to 0° C., and after 5 minutes, benzoyl chloride (4.1 mL, 36.0 mmol, 1.1 equiv) was added. The resulting solution was allowed to warm to RT for 2 h, and then the pH of the reaction mixture was adjusted to pH 8 by dropwise addition of 1 M aqueous Na₂CO₃ solution. The mixture was extracted with EtOAc (150 mL×3) and the organic layers were combined and concentrated in vacuo. The residue was purified by flash column chromatography on silica gel, eluting with ethyl acetate/petroleum ether (5/95) to give 5,6-difluoro-3-nitro-1H-indole (3.5 g, 17.7 mmol) as a yellow solid. LC-MS Method B, MS-ESI: 199.1 [M+H⁺]. Alternatively, the residue can be purified by flash silica gel chromatography (ISCO®; 24 g SepaFlash® Silica Flash Column, Eluent of 0-100% EtOAc/Petroleum ether gradient @ 30 mL/min) to give 5,6-difluoro-3-nitro-1H-indole (2.9 g, 13.5 mmol) as a yellow solid. MS-ESI, 199.1 [M+H⁺].

### Step 2—Synthesis of 5,6-difluoro-1H-indol-3-amine (Intermediate 1)

5,6-Difluoro-3-nitro-1H-indole (3.5 g, 17.7 mmol, 1.0 equiv) was dissolved in 40% HBr/H₂O (40 mL), then SnCl₂ (16.8 g, 88.5 mmol, 5.0 equiv) was added and the reaction mixture was heated to 70° C. for 30 minutes. The reaction mixture was cooled to RT, and the pH was adjusted to pH 8 by dropwise addition of 1 M aqueous NaOH. The mixture was extracted with DCM (150 mL×5) and the combined organic layers were concentrated in vacuo. The residue was used in the next step directly without further purification. LCMS Method B, MS-ESI: 169.1 [M+H⁺].

### Intermediate 2: Synthesis of (6-(4,4-difluorocyclohexyl)pyridin-3-amine)

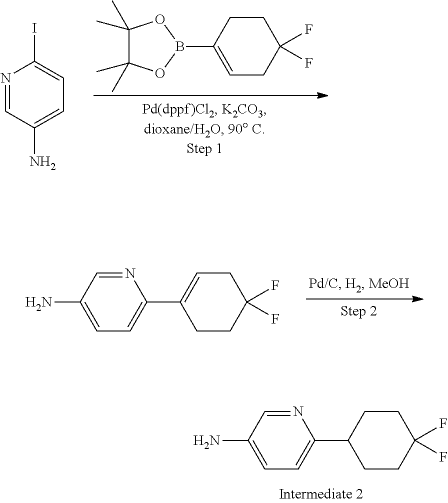

### Step 1: 6-(4,4-difluorocyclohex-1-en-1-yl)pyridin-3-amine

6-Iodopyridin-3-amine (5.0 g, 22.7 mmol, 1.0 eq.) was dissolved dioxane (80 mL) and H₂O (8 mL), then K₂CO₃ (9.4 g, 68.2 mmol, 3.0 eq.), 2-(4,4-difluorocyclohex-1-en-1-yl)-4,4,5,5-tetramethyl-1,3,2-dioxaborolane (9.5 g, 27.3 mmol, 1.2 eq.) and Pd(dppf)Cl₂ CH₂Cl₂ (185.6 mg, 0.2 mmol, 0.1 eq.) were added under nitrogen. The resulting solution was stirred for 12 hour at 90° C. and then concentrated under vacuum. The residue was purified by flash column chromatography on silica gel, eluting with ethyl acetate/petroleum ether (1:5) to give 6-(4,4-difluorocyclohex-1-en-1-yl)pyridin-3-amine (5.2 g) as a light yellow solid. LCMS Method H: [M+H]⁺=211.

### Step 2: 6-(4,4-difluorocyclohexyl)pyridin-3-amine

6-(4,4-difluorocyclohex-1-en-1-yl)pyridin-3-amine (5.2 g, 14.3 mmol, 1.0 eq.) was dissolved in MeOH (50 mL), then Pd/C (10% wt, 1.5 g, 1.4 mmol, 0.1 eq.) was added. The reaction vessel was evacuated then back filled with hydrogen three times, then stirred for 16 hour under an atmosphere of hydrogen. Filtration and concentration give 6-(4,4-difluorocyclohexyl)pyridin-3-amine (4.4 g) as a off-white solid. LCMS Method H: [M+H]⁺=213.

### Intermediate 3: 2-(4,4-difluorocyclohexyl)-5-isocyanatopyridine

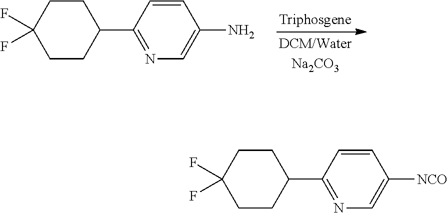

6-(4,4-difluorocyclohexyl)pyridin-3-amine (1 mmol) was dissolved in 5 mL of DCM/water (1:1 mixture) and cooled to ° C. Triphosgene (0.5 mmol) was dissolved in 2 mL of DCM and added slowly to DCM layer. The solution was stirred for 30 minutes and the two layers were separated. The organic layer was washed with brine and dried over anhydrous Mg₂SO₄. The organic layer was rotavaped and used as is for next step.

### Synthesis of Intermediate 5 (5-chloro-6-(4,4-difluoropiperidin-1-yl)pyridin-3-amine)

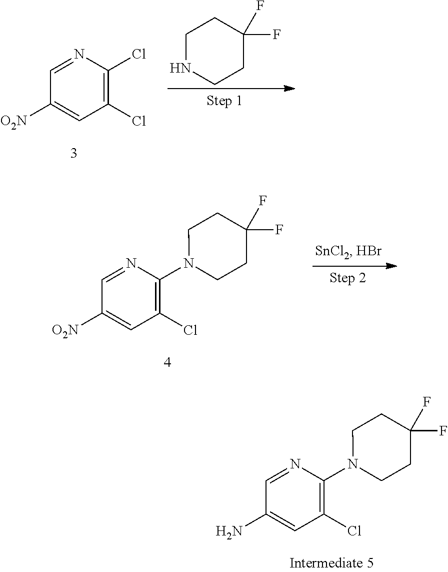 [SMILES: O=[N+]([O-])c1cnc(Cl)c(Cl)c1 ; FC1(F)CCNCC1 ; O=[N+]([O-])c1cnc(N2CCC(F)(F)CC2)c(Cl)c1 ; Nc1cnc(N2CCC(F)(F)CC2)c(Cl)c1]

### Step 1: 3-chloro-2-(4,4-difluoropiperidin-1-yl)-5-nitropyridine

2,3-Dichloro-5-nitropyridine (600.0 mg, 3.1 mmol, 1.0 equiv.) was dissolved in DMF (30 mL), Cs₂CO₃ (4.1 g, 12.4 mmol, 4.0 equiv.) and 4,4-difluoropiperidine (375.1 mg, 3.1 mmol, 1.0 equiv.) were added. The reaction mixture was stirred for 6 hours at 60° C. and then quenched by the addition of water. The resulting solution was extracted with ethyl acetate, washed with brine, dried over anhydrous sodium sulfate and concentrated under vacuum to give 3-chloro-2-(4,4-difluoropiperidin-1-yl)-5-nitropyridine (420 mg) as a yellow solid. LCMS Method C: [M+H]⁺=278.

### Step 2: 5-chloro-6-(4,4-difluoropiperidin-1-yl)pyridin-3-amine

3-Chloro-2-(4,4-difluoropiperidin-1-yl)-5-nitropyridine (3.4 g, 12.2 mmol, 1.0 equiv.) was dissolved in 40% HBr (10.0 mL), then SnCl₂ (5.5 g, 29.0 mmol, 2.4 equiv.).

The resulting solution was stirred for 2 hours at ambient temperature and adjusted to pH 8 with aqueous NaOH (1 mol/L). The mixture was extracted with ethyl acetate, dried over anhydrous Na₂SO₄ and concentrated under vacuum. The residue was purified by flash column chromatography on silica gel, eluting with DCM/MeOH (10:1) to give 5-chloro-6-(4,4-difluoropiperidin-1-yl)pyridin-3-amine (2.8 g) as a brown solid. LCMS Method C: [M+H]⁺=248.

The following intermediates were prepared using the same method described for Intermediate 5.

| Intermediate | Starting material A | Starting material B | Structure | LCMS data |
|---|---|---|---|---|
| Intermediate 6 | 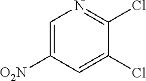 [SMILES: O=[N+]([O-])c1cnc(Cl)c(Cl)c1] | 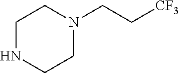 [SMILES: FC(F)(F)CCN1CCNCC1] | 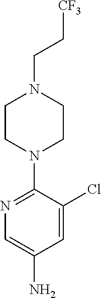 [SMILES: Nc1cnc(N2CCN(CCC(F)(F)F)CC2)c(Cl)c1] | Method A: MS-ESI: 309 [M + H]⁺ |
| Intermediate 7 | 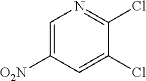 [SMILES: O=[N+]([O-])c1cnc(Cl)c(Cl)c1] | 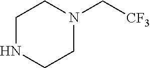 [SMILES: FC(F)(F)CN1CCNCC1] | 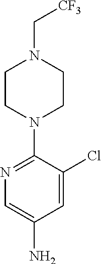 [SMILES: Nc1cnc(N2CCN(CC(F)(F)F)CC2)c(Cl)c1] | Method A: MS-ESI: 295 [M + H]⁺ |
| Intermediate 8 | 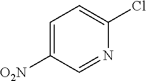 | 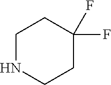 | 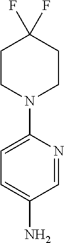 [SMILES: Nc1ccc(N2CCC(F)(F)CC2)nc1] | Method A: MS-ESI: 214 [M + H]⁺ |
| Intermediate 9 | 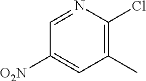 | 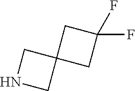 | 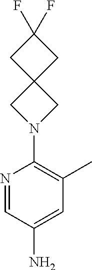 [SMILES: Cc1cc(N)cnc1N1CC2(C1)CC(F)(F)C2] | Method A: MS-ESI: 240 [M + H]⁺ |
| Intermediate 10 | 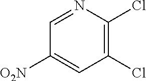 [SMILES: O=[N+]([O-])c1cnc(Cl)c(Cl)c1] | 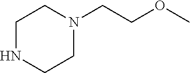 | 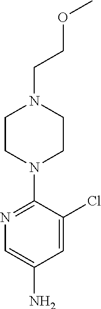 [SMILES: COCCN1CCN(c2ncc(N)cc2Cl)CC1] | Method A: MS-ESI: 271 [M + H]⁺ |
| Intermediate 11 | 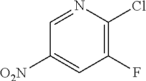 [SMILES: O=[N+]([O-])c1cnc(Cl)c(F)c1] | 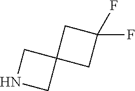 | 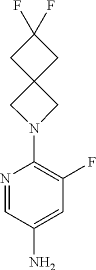 [SMILES: Nc1cnc(N2CC3(C2)CC(F)(F)C3)c(F)c1] | Method A: MS-ESI: 244 [M + H]⁺ |
| Intermediate 12 | 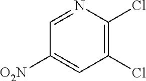 [SMILES: O=[N+]([O-])c1cnc(Cl)c(Cl)c1] | 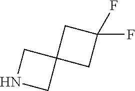 | 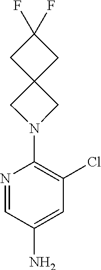 [SMILES: Nc1cnc(N2CC3(C2)CC(F)(F)C3)c(Cl)c1] | Method A: MS-ESI: 260 [M + H]⁺ |
| Intermediate 13 | 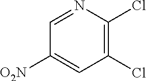 [SMILES: O=[N+]([O-])c1cnc(Cl)c(Cl)c1] | 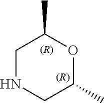 | 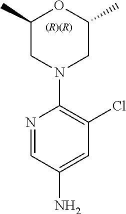 | Method A: MS-ESI: 242 [M + H]⁺ |
| Intermediate 14 | 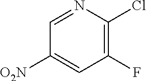 [SMILES: O=[N+]([O-])c1cnc(Cl)c(F)c1] | 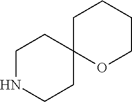 [SMILES: C1CCC2(CCNCC2)OC1] | 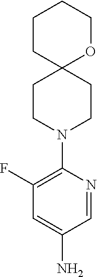 [SMILES: Nc1cnc(N2CCC3(CCCCO3)CC2)c(F)c1] | Method A: MS-ESI: 266 [M + H]⁺ |
| Intermediate 15 | 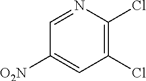 [SMILES: O=[N+]([O-])c1cnc(Cl)c(Cl)c1] | 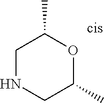 | 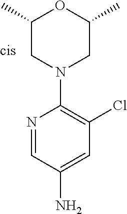 | Method A: MS-ESI: 242 [M + H]⁺ |
| Intermediate 16 | 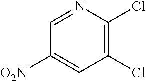 [SMILES: O=[N+]([O-])c1cnc(Cl)c(Cl)c1] | 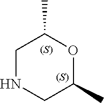 | 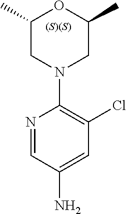 | Method A: MS-ESI: 242 [M + H]⁺ |
| Intermediate 17 | 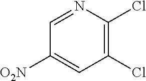 [SMILES: O=[N+]([O-])c1cnc(Cl)c(Cl)c1] | 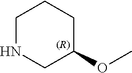 | 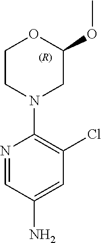 | Method A: MS-ESI: 242 [M + H]⁺ |
| Intermediate 18 | 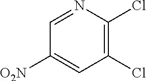 [SMILES: O=[N+]([O-])c1cnc(Cl)c(Cl)c1] | 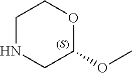 | 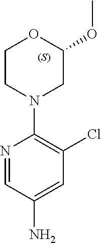 | Method A: MS-ESI: 242 [M + H]⁺ |
| Intermediate 19 | 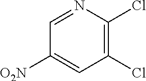 [SMILES: O=[N+]([O-])c1cnc(Cl)c(Cl)c1] | 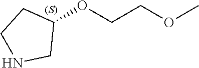 | 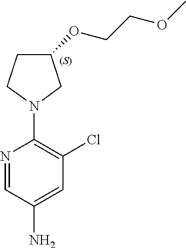 | Method A: MS-ESI: 272 [M + H]⁺ |
| Intermediate 20 | 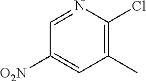 | 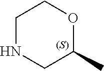 | 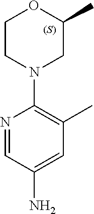 | Method A: MS-ESI: 208 [M + H]⁺ |
| Intermediate 21 | 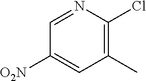 | 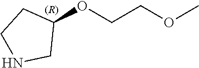 | 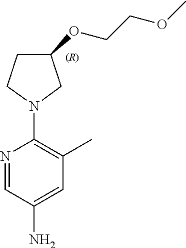 | Method A: MS-ESI: 252 [M + H]⁺ |
| Intermediate 22 | 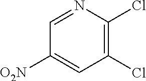 [SMILES: O=[N+]([O-])c1cnc(Cl)c(Cl)c1] | 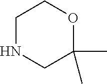 | 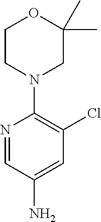 [SMILES: CC1(C)CN(c2ncc(N)cc2Cl)CCO1] | Method A: MS-ESI: 242 [M + H]⁺ |
| Intermediate 23 | 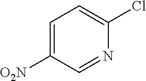 | 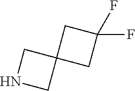 | 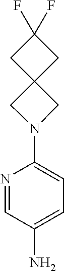 [SMILES: Nc1ccc(N2CC3(C2)CC(F)(F)C3)nc1] | Method A: MS-ESI: 226 [M + H]⁺ |
| Intermediate 24 | 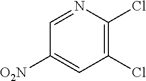 [SMILES: O=[N+]([O-])c1cnc(Cl)c(Cl)c1] | 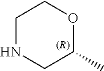 | 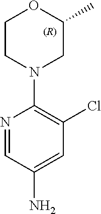 | Method A: MS-ESI: 228 [M + H]⁺ |
| Intermediate 25 | 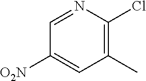 | 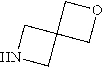 | 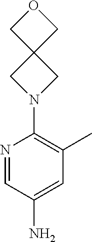 | Method A: MS-ESI: 206 [M + H]⁺ |
| Intermediate 26 | 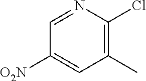 | 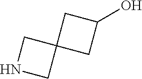 | 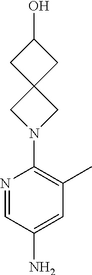 [SMILES: Cc1cc(N)cnc1N1CC2(CC(O)C2)C1] | Method A: MS-ESI: 220 [M + H]⁺ |
| Intermediate 27 | 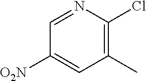 | 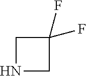 | 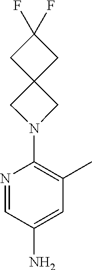 | Method A: MS-ESI: 200 [M + H]⁺ |
| Intermediate 28 | 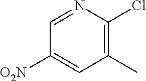 | 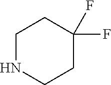 | 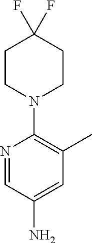 [SMILES: Cc1cc(N)cnc1N1CCC(F)(F)CC1] | Method A: MS-ESI: 228 [M + H]⁺ |
| Intermediate 29 | 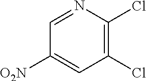 [SMILES: O=[N+]([O-])c1cnc(Cl)c(Cl)c1] | 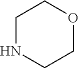 | 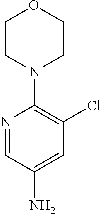 [SMILES: Nc1cnc(N2CCOCC2)c(Cl)c1] | Method A: MS-ESI: 214 [M + H]⁺ |
| Intermediate 30 | 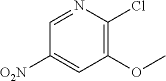 | 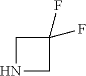 | 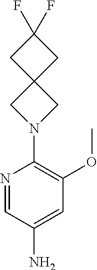 | Method A: MS-ESI: 216 [M + H]⁺ |
| Intermediate 31 | 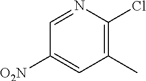 | 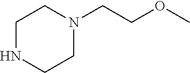 | 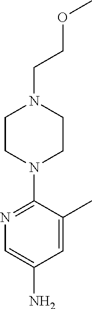 [SMILES: COCCN1CCN(c2ncc(N)cc2C)CC1] | Method A: MS-ESI: 251 [M + H]⁺ |
| Intermediate 32 | 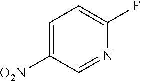 | 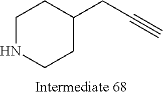 | 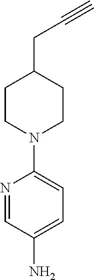 [SMILES: C#CCC1CCN(c2ccc(N)cn2)CC1] | Method A: MS-ESI: 216 [M + H]⁺ |
| Intermediate 33 | 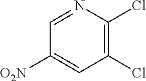 [SMILES: O=[N+]([O-])c1cnc(Cl)c(Cl)c1] | 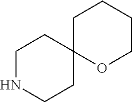 [SMILES: C1CCC2(CCNCC2)OC1] | 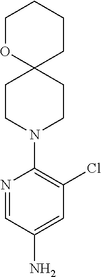 [SMILES: Nc1cnc(N2CCC3(CCCCO3)CC2)c(Cl)c1] | Method A: MS-ESI: 282 [M + H]⁺ |
| Intermediate 34 | 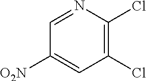 [SMILES: O=[N+]([O-])c1cnc(Cl)c(Cl)c1] | 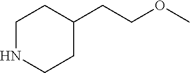 | 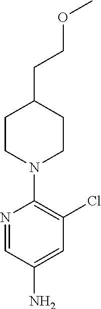 [SMILES: COCCC1CCN(c2ncc(N)cc2Cl)CC1] | Method A: MS-ESI: 270 [M + H]⁺ |
| Intermediate 35 | 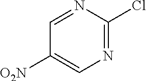 | 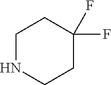 | 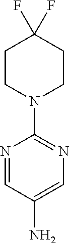 [SMILES: Nc1cnc(N2CCC(F)(F)CC2)nc1] | Method A: MS-ESI: 215 [M + H]⁺ |
| Intermediate 36 | 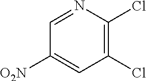 [SMILES: O=[N+]([O-])c1cnc(Cl)c(Cl)c1] | 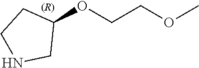 | 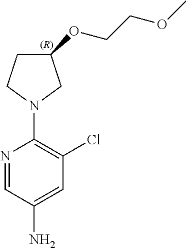 | Method A: MS-ESI: 272 [M + H]⁺ |
| Intermediate 37 | 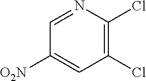 [SMILES: O=[N+]([O-])c1cnc(Cl)c(Cl)c1] | 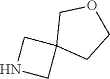 | 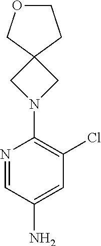 [SMILES: Nc1cnc(N2CC3(CCOC3)C2)c(Cl)c1] | Method A: MS-ESI: 240 [M + H]⁺ |
| Intermediate 38 | 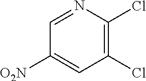 [SMILES: O=[N+]([O-])c1cnc(Cl)c(Cl)c1] | 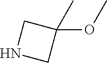 | 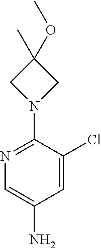 | Method A: MS-ESI: 228 [M + H]⁺ |
| Intermediate 39 | 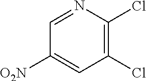 [SMILES: O=[N+]([O-])c1cnc(Cl)c(Cl)c1] | 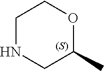 | 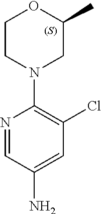 | Method A: MS-ESI: 228 [M + H]⁺ |
| Intermediate 40 | 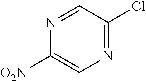 | 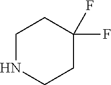 | 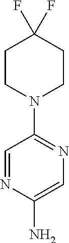 [SMILES: Nc1cnc(N2CCC(F)(F)CC2)cn1] | Method A: MS-ESI: 215 [M + H]⁺ |
| Intermediate 41 | 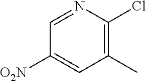 | 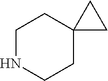 | 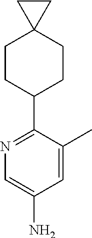 [SMILES: Cc1cc(N)cnc1C1CCC2(CC1)CC2] | Method A: MS-ESI: 218 [M + H]⁺ |
| Intermediate 42 | 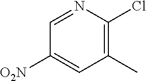 | 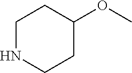 | 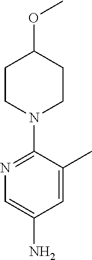 [SMILES: COC1CCN(c2ncc(N)cc2C)CC1] | Method A: MS-ESI: 222 [M + H]⁺ |
| Intermediate 79 | 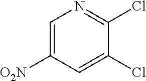 [SMILES: O=[N+]([O-])c1cnc(Cl)c(Cl)c1] | 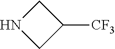 | 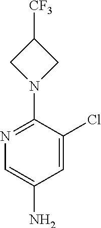 [SMILES: Nc1cnc(N2CC(C(F)(F)F)C2)c(Cl)c1] | Method A: MS-ESI: 252 [M + H]+ |
| Intermediate 80 | 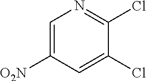 [SMILES: O=[N+]([O-])c1cnc(Cl)c(Cl)c1] | 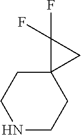 | 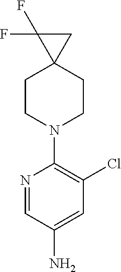 [SMILES: Nc1cnc(N2CCC3(CC2)CC3(F)F)c(Cl)c1] | Method A: MS-ESI: 274 [M + H]+ |
| Intermediate 81 | 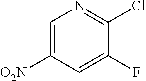 [SMILES: O=[N+]([O-])c1cnc(Cl)c(F)c1] | 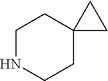 | 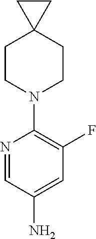 [SMILES: Nc1cnc(N2CCC3(CC2)CC3)c(F)c1] | Method A: MS-ESI: 222 [M + H]+ |
| Intermediate 82 | 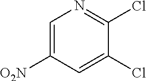 [SMILES: O=[N+]([O-])c1cnc(Cl)c(Cl)c1] | 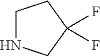 | 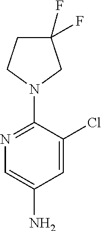 [SMILES: Nc1cnc(N2CCC(F)(F)C2)c(Cl)c1] | Method A: MS-ESI: 234 [M + H]+ |
| Intermediate 83 | 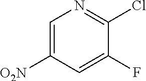 [SMILES: O=[N+]([O-])c1cnc(Cl)c(F)c1] | 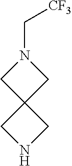 [SMILES: FC(F)(F)CN1CC2(CNC2)C1] | 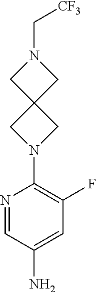 [SMILES: Nc1cnc(N2CC3(CN(CC(F)(F)F)C3)C2)c(F)c1] | Method A: MS-ESI: 291 [M + H]+ |
| Intermediate 84 | 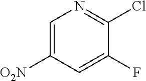 [SMILES: O=[N+]([O-])c1cnc(Cl)c(F)c1] | 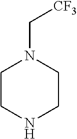 [SMILES: FC(F)(F)CN1CCNCC1] | 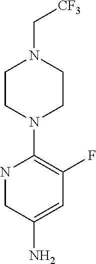 [SMILES: NC1=CC(F)=C(N2CCN(CC(F)(F)F)CC2)NC1] | Method A: MS-ESI: 279 [M + H]+ |
| Intermediate 85 | 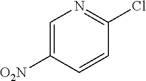 | 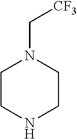 [SMILES: FC(F)(F)CN1CCNCC1] | 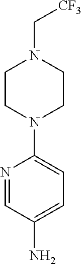 [SMILES: Nc1ccc(N2CCN(CC(F)(F)F)CC2)nc1] | Method A: MS-ESI: 261 [M + H]+ |
| Intermediate 86 | 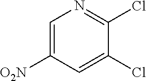 [SMILES: O=[N+]([O-])c1cnc(Cl)c(Cl)c1] | 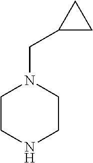 | 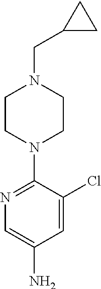 [SMILES: Nc1cnc(N2CCN(CC3CC3)CC2)c(Cl)c1] | Method A: MS-ESI: 267 [M + H]+ |
| Intermediate 87 | 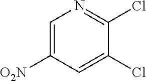 [SMILES: O=[N+]([O-])c1cnc(Cl)c(Cl)c1] | 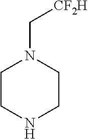 | 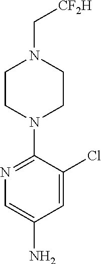 [SMILES: Nc1cnc(N2CCN(CC(F)F)CC2)c(Cl)c1] | Method A: MS-ESI: 277 [M + H]+ |
| Intermediate 88 | 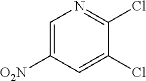 [SMILES: O=[N+]([O-])c1cnc(Cl)c(Cl)c1] | 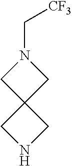 [SMILES: FC(F)(F)CN1CC2(CNC2)C1] | 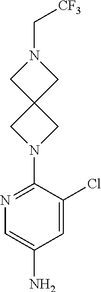 [SMILES: Nc1cnc(N2CC3(CN(CC(F)(F)F)C3)C2)c(Cl)c1] | Method A: MS-ESI: 307 [M + H]+ |
| Intermediate 89 | 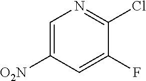 [SMILES: O=[N+]([O-])c1cnc(Cl)c(F)c1] | 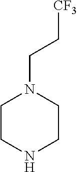 [SMILES: FC(F)(F)CCN1CCNCC1] | 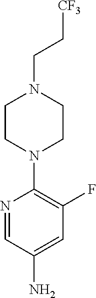 [SMILES: Nc1cnc(N2CCN(CCC(F)(F)F)CC2)c(F)c1] | Method A: MS-ESI: 293 [M + H]+ |
| Intermediate 90 | 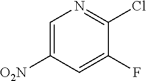 [SMILES: O=[N+]([O-])c1cnc(Cl)c(F)c1] | 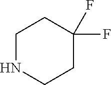 | 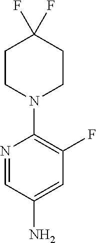 [SMILES: Nc1cnc(N2CCC(F)(F)CC2)c(F)c1] | Method A: MS-ESI: 232 [M + H]+ |
| Intermediate 91 | 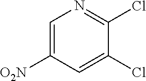 [SMILES: O=[N+]([O-])c1cnc(Cl)c(Cl)c1] | 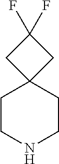 [SMILES: FC1(F)CC2(CCNCC2)C1] | 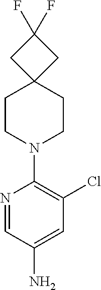 [SMILES: Nc1cnc(N2CCC3(CC2)CC(F)(F)C3)c(Cl)c1] | Method A: MS-ESI: 288 [M + H]+ |
| Intermediate 92 | 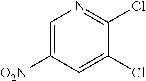 [SMILES: O=[N+]([O-])c1cnc(Cl)c(Cl)c1] | 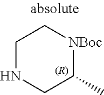 | 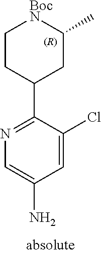 | Method A: MS-ESI: 327 [M + H]+ |
| Intermediate 93 | 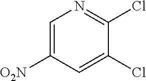 [SMILES: O=[N+]([O-])c1cnc(Cl)c(Cl)c1] | 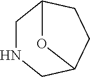 | 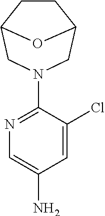 [SMILES: Nc1cnc(N2CC3CCC(C2)O3)c(Cl)c1] | Method A: MS-ESI: 240 [M + H]+ |
| Intermediate 94 | 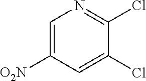 [SMILES: O=[N+]([O-])c1cnc(Cl)c(Cl)c1] | 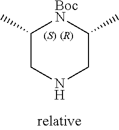 | 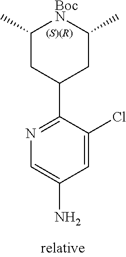 | Method A: MS-ESI: 341 [M + H]+ |
| Intermediate 95 | 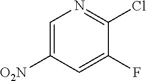 [SMILES: O=[N+]([O-])c1cnc(Cl)c(F)c1] | 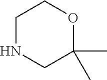 | 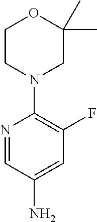 [SMILES: CC1(C)CN(c2ncc(N)cc2F)CCO1] | Method A: MS-ESI: 226 [M + H]+ |
| Intermediate 96 | 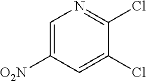 [SMILES: O=[N+]([O-])c1cnc(Cl)c(Cl)c1] | 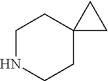 | 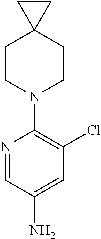 [SMILES: Nc1cnc(N2CCC3(CC2)CC3)c(Cl)c1] | Method A: MS-ESI: 238 [M + H]+ |
| Intermediate 97 | 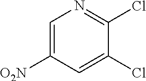 [SMILES: O=[N+]([O-])c1cnc(Cl)c(Cl)c1] | 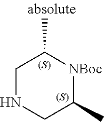 | 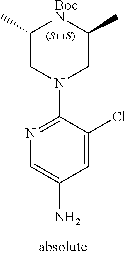 | Method A: MS-ESI: 341 [M + H]+ |
| Intermediate 98 | 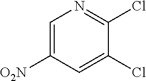 [SMILES: O=[N+]([O-])c1cnc(Cl)c(Cl)c1] | 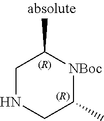 | 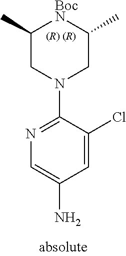 | Method A: MS-ESI: 341 [M + H]+ |
| Intermediate 99 | 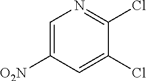 [SMILES: O=[N+]([O-])c1cnc(Cl)c(Cl)c1] | 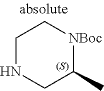 | 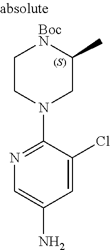 | Method A: MS-ESI: 327 [M + H]+ |

### Synthesis of Intermediate 43 (6-(4,4-difluorocyclohexyl)pyridin-3-amine)

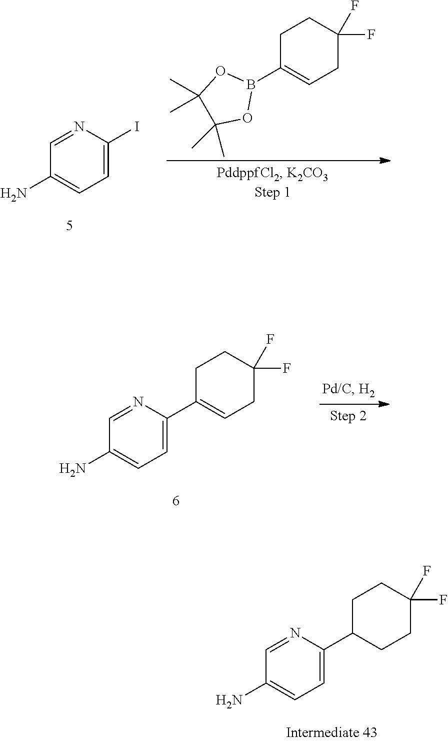

### Step 1: 6-(4,4-difluorocyclohex-1-en-1-yl)pyridin-3-amine

6-Iodopyridin-3-amine (4.0 g, 18.2 mmol, 1.0 equiv.) and 2-(4,4-difluorocyclohex-1-en-1-yl)-4,4,5,5-tetramethyl-1,3,2-dioxaborolane (5.3 g, 21.8 mmol, 1.2 equiv.) were dissolved in 1,4-dixoane (40 mL) and water (8 mL), then K₂CO₃ (7.5 g, 54.5 mmol, 3.0 equiv.) and Pd(dppf)Cl₂ (1.5 g, 1.8 mmol, 0.1 equiv.) were added under an atmosphere of nitrogen. The reaction mixture was heated 90° C. for 12 hours, then concentrated under vacuum. The residue was purified by flash column chromatography on silica gel, eluting with ethyl acetate/petroleum ether (1:5) to give 6-(4,4-difluorocyclohex-1-en-1-yl)pyridin-3-amine (2.7 g) as a light yellow solid. LCMS Method D: [M+H]⁺=211.

### Step 2: 6-(4,4-difluorocyclohexyl)pyridin-3-amine

6-(4,4-difluorocyclohex-1-en-1-yl)pyridin-3-amine (10.0 g, 47.6 mmol, 1.0 equiv.) was dissolved in MeOH (40 mL), Pd/C (1.0 g, 9.5 mmol, 0.2 equiv.) was added. The mixture was sparged with nitrogen, placed under an atmosphere of hydrogen gas (balloon), then stirred for 2 hours at ambient temperature. The solids were removed by filtration and the filtrate was concentrated under vacuum to give 6-(4,4-difluorocyclohexyl)pyridin-3-amine (9.1 g) as an off-white solid. LCMS Method C: [M+H]⁺=213.

The following intermediates were prepared using the same method described for Intermediate 43.

| | | | | LCMS |
|---|---|---|---|---|
| Intermediate | Starting material A | Starting material B | Structure | data |
| Intermediate 44 | 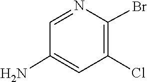 | 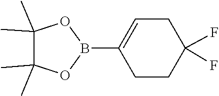 [SMILES: CC1(C)OB(C2=CCC(F)(F)CC2)OC1(C)C] | 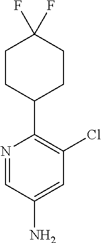 [SMILES: Nc1cnc(C2CCC(F)(F)CC2)c(Cl)c1] | Method C: MS-ESI: 247 [M + H]⁺ |
| Intermediate 45 | 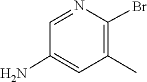 | 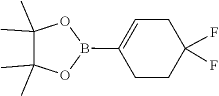 [SMILES: CC1(C)OB(C2=CCC(F)(F)CC2)OC1(C)C] | 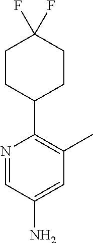 [SMILES: Cc1cc(N)cnc1C1CCC(F)(F)CC1] | Method A: MS-ESI: 227 [M + H]⁺ |
| Intermediate 46 | 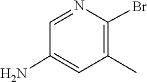 | 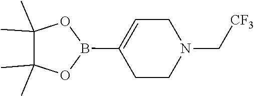 [SMILES: CC1(C)OB(C2=CCN(CC(F)(F)F)CC2)OC1(C)C] | 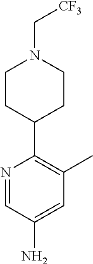 [SMILES: Cc1cc(N)cnc1C1CCN(CC(F)(F)F)CC1] | Method C: MS-ESI: 274 [M + H]⁺ |
| Intermediate 47 | 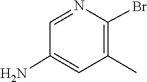 | 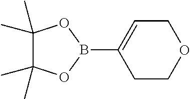 | 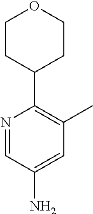 | Method C: MS-ESI: 193 [M + H]⁺ |
| Intermediate 48 | 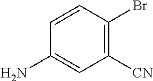 | 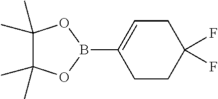 | 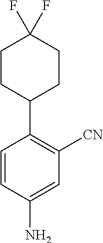 [SMILES: N#Cc1cc(N)ccc1C1CCC(F)(F)CC1] | Method C: MS-ESI: 237 [M + H]⁺ |
| Intermediate 71 | 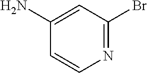 | 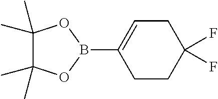 | 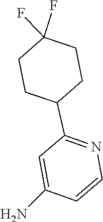 [SMILES: Nc1ccnc(C2CCC(F)(F)CC2)c1] | Method C: MS-ESI: 213 [M + H]+ |
| Intermediate 72 | 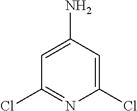 | 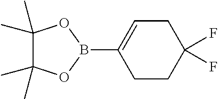 [SMILES: CC1(C)OB(C2=CCC(F)(F)CC2)OC1(C)C] | 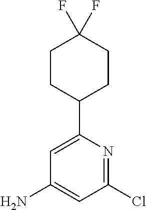 [SMILES: Nc1cc(Cl)nc(C2CCC(F)(F)CC2)c1] | Method C: MS-ESI: 247 [M + H]+ |
| Intermediate 73 | 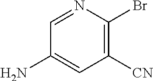 | 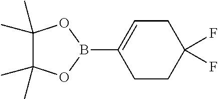 [SMILES: CC1(C)OB(C2=CCC(F)(F)CC2)OC1(C)C] | 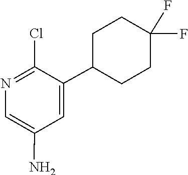 [SMILES: Nc1cnc(Cl)c(C2CCC(F)(F)CC2)c1] | Method C: MS-ESI: 247 [M + H]+ |
| Intermediate 74 | 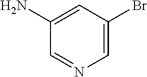 | 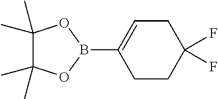 | 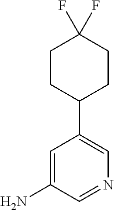 [SMILES: Nc1cncc(C2CCC(F)(F)CC2)c1] | Method C: MS-ESI: 213 [M + H]+ |
| Intermediate 75 | 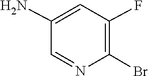 | 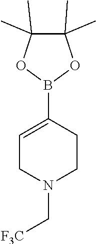 | 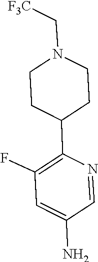 [SMILES: Nc1cnc(C2CCN(CC(F)(F)F)CC2)c(F)c1] | Method C: MS-ESI: 278 [M + H]+ |
| Intermediate 76 | 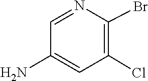 | 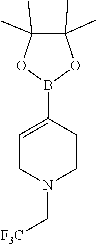 | 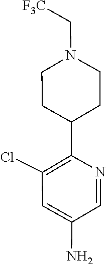 [SMILES: Nc1cnc(C2CCN(CC(F)(F)F)CC2)c(Cl)c1] | Method C: MS-ESI: 294 [M + H]+ |
| Intermediate 77 | 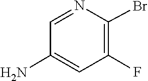 | 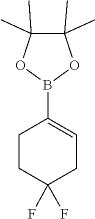 | 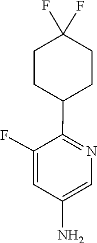 [SMILES: Nc1cnc(C2CCC(F)(F)CC2)c(F)c1] | Method C: MS-ESI: 231 [M + H]+ |
| Intermediate 78 | 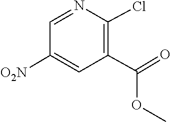 | 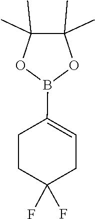 | 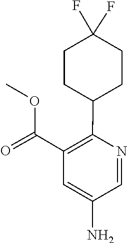 [SMILES: COC(=O)c1cc(N)cnc1C1CCC(F)(F)CC1] | Method C: MS-ESI: 271 [M + H]+ |
| Intermediate 105 | 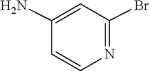 | 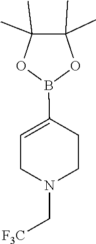 | 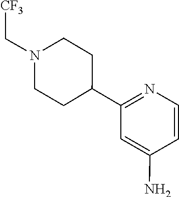 [SMILES: Nc1ccnc(C2CCN(CC(F)(F)F)CC2)c1] | Method C: MS-ESI: 260 [M + H]+ |

### Synthesis of Intermediate 49 (6-(4,4-difluoropiperidin-1-yl)pyridazin-3-amine)

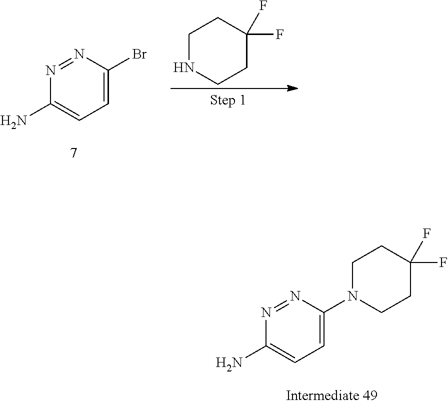 [SMILES: Nc1ccc(Br)nn1 ; FC1(F)CCNCC1 ; Nc1ccc(N2CCC(F)(F)CC2)nn1]

4,4-difluoropiperidine (1.0 g, 8.3 mmol, 1.0 equiv.) was dissolved in EtOH (10 mL), then 6-bromopyridazin-3-amine (1.4 g, 8.3 mmol, 1.0 equiv.) was added. The reaction mixture was heated to 80° C. overnight and concentrated under vacuum. The residue was purified by reverse flash chromatography with following conditions: column, C18 silica gel; mobile phase, ACN/water, 0% ACN increasing to 100% within 30 min; detector, UV 254 nm. This resulted in 6-(4,4-difluoropiperidin-1-yl)pyridazin-3-amine (410 mg) as a brown solid. LCMS Method D: [M+H]⁺=215.

### Synthesis of Intermediate 50 (4-(3,3-difluorocyclobutyl)-3-fluoroaniline)

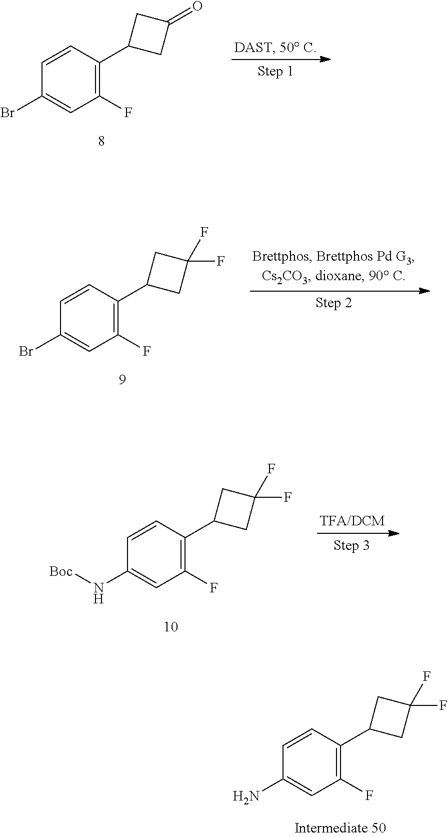 [SMILES: O=C1CC(c2ccc(Br)cc2F)C1 ; Fc1cc(Br)ccc1C1CC(F)(F)C1 ; CC(C)(C)OC(=O)Nc1ccc(C2CC(F)(F)C2)c(F)c1 ; Nc1ccc(C2CC(F)(F)C2)c(F)c1]

### Step 1: 4-bromo-1-(3,3-difluorocyclobutyl)-2-fluorobenzene

3-(4-Bromo-2-fluorophenyl)cyclobutan-1-one (1.3 g, 5.3 mmol, 1.0 equiv.) was dissolved in DAST (30.0 mL) at 0° C. under atmosphere of nitrogen. The resulting mixture was stirred for overnight at room temperature and then quenched by the addition of aqueous NaHCO₃ at 0° C. The resulting mixture was extracted with DCM, dried over anhydrous Na₂SO₄ and concentrated under vacuum. The residue was purified by flash column chromatography on silica gel, eluting with ethyl acetate/petroleum ether (1:2) to give 4-bromo-1-(3,3-difluorocyclobutyl)-2-fluorobenzene (1.1 g) as a yellow oil. ¹H NMR (300 MHz, DMSO-d₄): δ 7.53-7.49 (m, 1H), 7.43-7.34 (m, 2H), 3.52-3.46 (m, 1H), 3.07-2.94 (m, 2H), 2.84-2.66 (m, 2H).

### Step 2: tert-butyl (4-(3,3-difluorocyclobutyl)-3-fluorophenyl)carbamate

4-Bromo-1-(3,3-difluorocyclobutyl)-2-fluorobenzene (1.1 g, 4.2 mmol, 1.0 equiv.) and BocNH₂ (2.4 g, 20.7 mmol, 5.0 equiv.) were dissolved in toluene (11.0 mL). Pd₂(dba)₃ (0.4 g, 0.4 mmol, 0.1 equiv.), XPhos (0.4 g, 0.8 mmol, 0.2 equiv.) and t-BuOK (2.3 g, 20.7 mmol, 5.0 equiv.) were added at room temperature under atmosphere of nitrogen. The resulting mixture was stirred for overnight at 100° C. and then quenched by the addition of water. The resulting solution was extracted with ethyl acetate, dried over anhydrous sodium sulfate and concentrated under vacuum. The residue was purified by flash column chromatography on silica gel, eluting with ethyl acetate/petroleum ether (1:8) to give tert-butyl [4-(3,3-difluorocyclobutyl)-3-fluorophenyl]carbamate (1.0 g, 80.0%) as a white solid. LCMS Method A: [M+H]⁺=302.

### Step 3: 4-(3,3-difluorocyclobutyl)-3-fluoroaniline

tert-Butyl [4-(3,3-difluorocyclobutyl)-3-fluorophenyl]carbamate (1.2 g, 4.0 mmol, 1.0 equiv.) was dissolved in DCM (12.0 mL), TFA (3.0 mL) was added dropwise at 0° C. The resulting mixture was stirred for 2 hours at room temperature and then concentrated under vacuum. The residue was dissolved in DCM, and the solution was washed with sat. NaHCO₃ aqueous and brine, dried over anhydrous sodium sulfate and concentrated under vacuum to give crude 4-(3,3-difluorocyclobutyl)-3-fluoroaniline (800 mg) as a red oil. LCMS Method A: [M+H]⁺=202.

### Synthesis of Intermediate 51 (5-chloro-6-(3,3-difluorocyclobutyl)pyridin-3-amine)

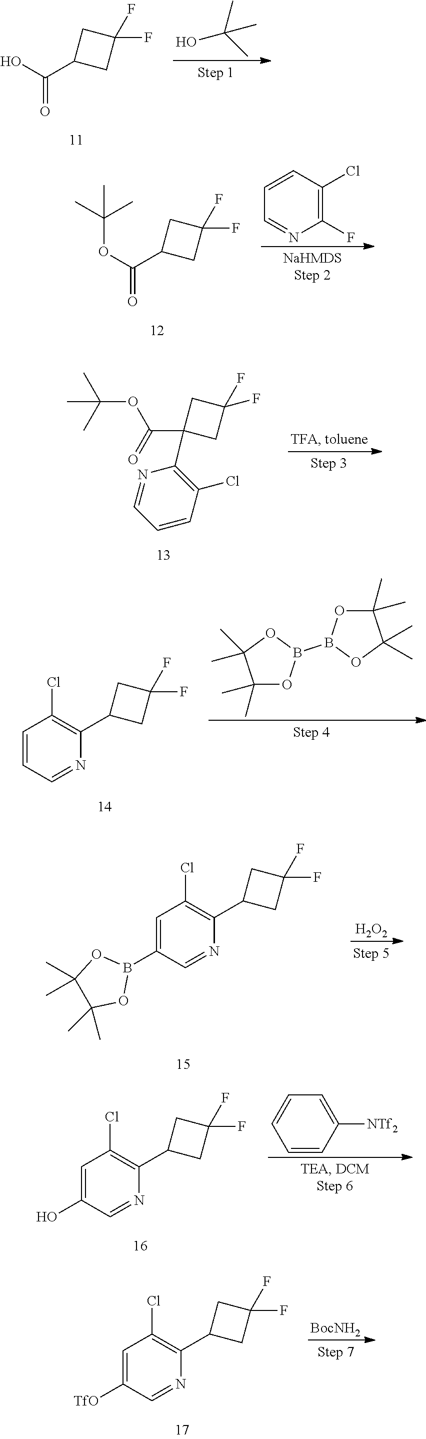

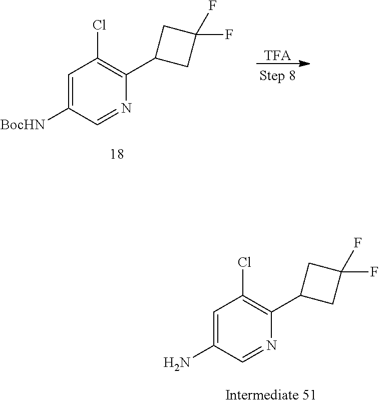 [SMILES: CC(C)(C)OC(=O)Nc1cnc(C2CC(F)(F)C2)c(Cl)c1 ; Nc1cnc(C2CC(F)(F)C2)c(Cl)c1 ; "TFA"]

### Step 1: tert-butyl 3,3-difluorocyclobutane-1-carboxylate

3,3-Difluorocyclobutanecarboxylic acid (1.0 g, 7.3 mmol, 1.0 equiv.) was dissolved in DCM (10 mL), N,N-dimethylpyridin-4-amine (92.0 mg, 0.7 mmol, 0.1 equiv.), 2-methylpropan-2-ol (1.1 g, 14.7 mmol, 2.0 equiv.) and N,N′-dicyclohexylcarbodiimide (1.7 g, 8.1 mmol, 1.1 equiv.) were added at 10° C. The reaction mixture was warmed up to room temperature and stirred for 18 hours. The solid was removed by filtration and the filtrate was washed with aqueous HCl (2N), saturated aqueous NaHCO₃, brine, dried over anhydrous Na₂SO₄, and concentrated under vacuum to give crude tert-butyl 3,3-difluorocyclobutane-1-carboxylate (896.1 mg) as colorless oil. ¹H NMR (400 MHz, CDCl₃): δ 2.83-2.78 (m, 5H), 1.47 (s, 9H).

### Step 2: tert-butyl 1-(3-chloropyridin-2-yl)-3,3-difluorocyclobutane-1-carboxylate

3-Chloro-2-fluoropyridine (1.2 g, 10.4 mmol, 1.0 equiv.) and tert-butyl 3,3-difluorocyclobutane-1-carboxylate (2.0 g, 10.4 mmol, 1.0 equiv.) were dissolved in toluene (60 mL). This was followed by the addition of NaHMDS (2 M in THF, 6.2 m1, 12.4 mmol, 1.2 equiv.) dropwise with stirring at 0° C. in 10 min. The resulting solution was stirred for 2 hours at 0° C. and then quenched by the addition of saturated aqueous NH₄Cl. The resulting solution was extracted with ethyl acetate, dried over anhydrous sodium sulfate and concentrated under vacuum. The residue was purified by flash column chromatography on silica gel, eluting with ethyl acetate/petroleum ether (1:5) to give tert-butyl 1-(3-chloropyridin-2-yl)-3,3-difluorocyclobutane-1-carboxylate (1.6 g) as colorless oil. LCMS Method D: [M+H]⁺=304.

### Step 3: 3-chloro-2-(3,3-difluorocyclobutyl)pyridine

tert-Butyl 1-(3-chloropyridin-2-yl)-3,3-difluorocyclobutane-1-carboxylate (1.5 g, 5.2 mmol, 1.0 equiv.) was dissolved in DCM (30 mL) and TFA (3 m1). The resulting solution was stirred for 10 hours at ambient temperature and then concentrated under vacuum. The residue was dissolved in toluene (30 mL) and stirred for 18 hours at 90° C. After cooling down to ambient temperature and quenching by addition of water, the pH value of the solution was adjusted to 7.5 with saturated aqueous Na₂CO₃. The solution was extracted with ethyl acetate, dried over anhydrous sodium sulfate and concentrated under vacuum.

The residue was purified by flash column chromatography on silica gel, eluting with ethyl acetate/petroleum ether (1:7) to give 3-chloro-2-(3,3-difluorocyclobutyl)pyridine (700 mg) as colorless oil. LCMS Method D: [M+H]⁺=204. ¹H NMR (400 MHz, DMSO-d₆): δ 8.45-8.43 (m, 1H), 7.69-7.67 (m, 1H), 7.40-7.38 (m, 1H), 3.72-3.70 (m, 1H), 3.02-2.85 (m, 4H).

### Step 4: 3-chloro-2-(3,3-difluorocyclobutyl)-5-(4,4,5,5-tetramethyl-1,3,2-dioxaborolan-2-yl)pyridine

3-chloro-2-(3,3-difluorocyclobutyl)pyridine (700.0 mg, 3.7 mmol, 1.0 equiv.) was dissolved in heptane (30 mL), bis(pinacolato)diboron (1.1 g, 4.4 mmol, 1.2 equiv.), 4,4-di-tert-butyl-2,2-dipyridyl (1.0 g, 3.7 mmol, 1.0 equiv.) and di-methanolatodiiridium(Ir—Ir)-cycloocta-1,5-diene (1:2) (495.8 mg, 0.7 mmol, 0.2 equiv.) were added under an atmosphere of nitrogen. The resulting solution was stirred for 18 hours at ambient temperature and then quenched by the addition of water. The resulting solution was extracted with ethyl acetate, dried over anhydrous sodium sulfate and concentrated under vacuum. The residue was purified by flash column chromatography on silica gel, eluting with ethyl acetate/petroleum ether (1:5) to give 3-chloro-2-(3,3-difluorocyclobutyl)-5-(4,4,5,5-tetramethyl-1,3,2-dioxaborolan-2-yl)pyridine (300 mg) as a white solid. LCMS Method D: [M+H]⁺=330.

### Step 5: 5-chloro-6-(3,3-difluorocyclobutyl)pyridin-3-ol

3-chloro-2-(3,3-difluorocyclobutyl)-5-(4,4,5,5-tetramethyl-1,3,2-dioxaborolan-2-yl)pyridine (300.0 mg, 0.9 mmol, 1.0 equiv.) was dissolved in MeOH (10 mL) and H₂O (3 mL). Then H₂O₂ (30%, 0.14 ml, 1.4 mmol, 1.5 equiv.) was added. The resulting solution was stirred for 30 min at ambient temperature and then quenched by the addition of saturated aqueous Na₂S₂O₃. The resulting solution was extracted with ethyl acetate, dried over anhydrous sodium sulfate and concentrated under vacuum. The residue was purified by flash column chromatography on silica gel, eluting with ethyl acetate/petroleum ether (1:2) to give 5-chloro-6-(3,3-difluorocyclobutyl)pyridin-3-ol (160 mg) as a white solid. LCMS Method D: [M+H]⁺=220. ¹H NMR (400 MHz, CD₃OD-d₄): δ 8.0 (s, 1H), 6.97-6.93 (m, 1H), 3.69-3.58 (m, 1H), 3.01-2.78 (m, 4H).

### Step 6: 5-chloro-6-(3,3-difluorocyclobutyl)pyridin-3-yl trifluoromethanesulfonate

5-chloro-6-(3,3-difluorocyclobutyl)pyridin-3-ol (160.0 mg, 0.7 mmol, 1.0 equiv.), was dissolved in DCM (20 mL), TEA (0.1 ml, 0.9 mmol, 1.2 equiv.) and 1,1,1-trifluoro-N-phenyl-N-trifluoromethanesulfonylmethanesulfonamide (309.4 mg, 0.8 mmol, 1.1 equiv.) were added. The resulting solution was stirred for 30 min at ambient temperature and then quenched by the addition of water. The solution was extracted with ethyl acetate, dried over anhydrous sodium sulfate and concentrated under vacuum. The residue was purified by flash column chromatography on silica gel, eluting with ethyl acetate/petroleum ether (1:8) to give 5-chloro-6-(3,3-difluorocyclobutyl)pyridin-3-yl trifluoromethanesulfonate (220 mg) as a white solid. LCMS Method D: [M+H]⁺=352.

### Step 7: tert-butyl (5-chloro-6-(3,3-difluorocyclobutyl)pyridin-3-yl)carbamate

5-chloro-6-(3,3-difluorocyclobutyl)pyridin-3-yl trifluoromethanesulfonate (220.0 mg, 0.6 mmol, 1.0 equiv.) was dissolved in 1,4-dioxane (30 mL). Then NH₂Boc (230.3 mg, 1.9 mmol, 3.0 equiv.), 4,5-Bis(diphenylphosphino)-9,9-dimethylxanthene (75.8 mg, 0.1 mmol, 0.2 equiv.) and Pd₂(dba)₃ (120.1 mg, 0.1 mmol, 0.2 equiv.) were added under an atmosphere of nitrogen. The resulting solution was stirred for 3 hours at 90° C. under atmosphere of nitrogen and then concentrated under vacuum. The residue was purified by flash column chromatography on silica gel, eluting with ethyl acetate/petroleum ether (1:9) to give tert-butyl (5-chloro-6-(3,3-difluorocyclobutyl)pyridin-3-yl)carbamate (120 mg) as a white solid. LCMS Method D: [M+H]⁺=319.

### Step 8: 5-chloro-6-(3,3-difluorocyclobutyl)pyridin-3-amine

tert-Butyl (5-chloro-6-(3,3-difluorocyclobutyl)pyridin-3-yl)carbamate (120.0 mg, 0.3 mmol, 1.0 equiv.) was dissolved in DCM (10 mL) and TFA (2 ml). The resulting solution was stirred for 30 min at ambient temperature and then diluted with water. The pH value of the solution was adjusted to 7.5 with saturated aqueous Na₂CO₃ and extracted with ethyl acetate. The organic layer was dried over anhydrous sodium sulfate and concentrated under vacuum. The residue was purified by flash column chromatography on silica gel, eluting with ethyl acetate/petroleum ether (1:3) to give 5-chloro-6-(3,3-difluorocyclobutyl)pyridin-3-amine (60 mg) as a white solid. LCMS Method D: [M+H]+=219.

The following intermediate was synthesized using the method described for Intermediate 51.

| Intermediate | Starting material | Structure | LCMS data |
|---|---|---|---|
| Intermediate 125 | 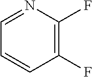 | 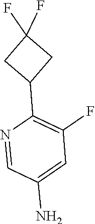 [SMILES: Nc1cnc(C2CC(F)(F)C2)c(F)c1] | Method D: MS-ESI: 203 [M + H]⁺ |

### Synthesis of Intermediate 52 (6-(4,4-difluoropiperidin-1-yl)-5-ethylpyridin-3-amine)

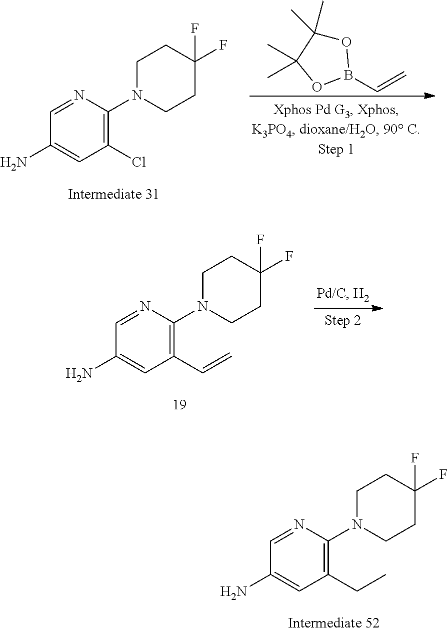

### Step 1: 6-(4,4-difluoropiperidin-1-yl)-5-ethenylpyridin-3-amine

5-Chloro-6-(4,4-difluoropiperidin-1-yl)pyridin-3-amine (3.0 g, 12.1 mmol, 1.0 equiv.) and K₃PO₄ (5.1 g, 24.2 mmol, 2.0 equiv.) were dissolved in 1,4-dioxane (60 mL) and water (6 mL), then Xphos Pd G3 (1.0 g, 1.2 mmol, 0.1 equiv.) and XPhos (577.4 mg, 1.2 mmol, 0.1 equiv.) were added under an atmosphere of nitrogen. The resulting mixture was heated to 90° C. overnight and then cooled to ambient temperature and quenched by the addition of water. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:1) to give 6-(4,4-difluoropiperidin-1-yl)-5-ethenylpyridin-3-amine (5.1 g) as a yellow solid. LCMS Method D: [M+H]⁺=240. ¹H NMR (300 MHz, DMSO-d₆): δ 7.62 (d, 1H), 7.13 (d, 1H), 6.85-6.81 (m, 1H), 5.70-5.65 (m, 1H), 5.32-5.28 (m, 1H), 3.04-2.97 (m, 4H), 2.15-2.00 (m, 4H).

### Step 2: 6-(4,4-difluoropiperidin-1-yl)-5-ethylpyridin-3-amine

6-(4,4-difluoropiperidin-1-yl)-5-ethenylpyridin-3-amine (1.2 g, 2.5 mmol, 1.0 equiv.) was dissolved in THE (12 mL), then Pd/C (0.2 g, 2.5 mmol, 1.0 equiv.) was added. The mixture was sparged with nitrogen, placed under an atmosphere of hydrogen gas (balloon), then stirred overnight at ambient temperature. The solids were removed by filtration and the filtrate was concentrated under vacuum. The residue was purified by flash column chromatography on silica gel, eluting with ethyl acetate/petroleum ether (1:1) to give 6-(4,4-difluoropiperidin-1-yl)-5-ethylpyridin-3-amine (860 mg) as a dark yellow solid. LCMS Method D: [M+H]⁺=242. ¹H NMR (300 MHz, DMSO-d₆): δ 7.52 (d, 1H), 6.84 (d, 1H), 2.96-2.91 (m, 5H), 2.56-2.54 (m, 2H), 2.07-2.01 (m, 4H), 1.14 (t, 3H).

### Synthesis of intermediate 53 (2-(5-amino-2-(4,4-difluoropiperidin-1-yl)pyridin-3-yl)ethan-1-ol)

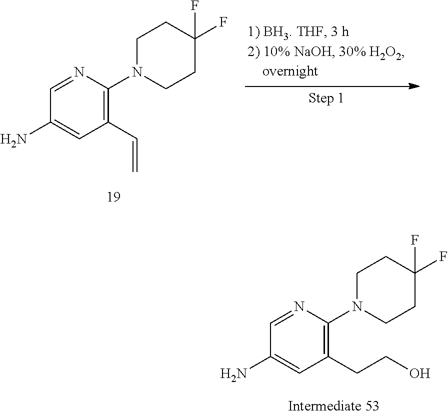 [SMILES: C=Cc1cc(N)cnc1N1CCC(F)(F)CC1 ; Nc1cnc(N2CCC(F)(F)CC2)c(CCO)c1]

6-(4,4-difluoropiperidin-1-yl)-5-ethenylpyridin-3-amine (2.0 g, 8.4 mmol, 1.0 equiv.) was dissolved in THE (40 mL) and cooled to 0° C., then BH₃.THF (1M, 16.7 mL, 16.7 mmol, 2.0 equiv.) was added dropwise, maintaining the solution at 0° C. The resulting mixture was stirred for 3 hours at ambient temperature. To the above mixture was added NaOH (5.0 g, 12.5 mmol, 1.5 equiv.) and H₂O₂ (30%, 1.3 mL, 16.7 mmol, 2.0 equiv.). The resulting mixture was stirred for additional 4 hours at ambient temperature and quenched by the addition of water. The resulting mixture was extracted with ethyl acetate, washed with brine, dried over anhydrous Na₂SO₄ and concentrated under vacuum. The residue was purified by reverse flash chromatography with the following conditions: Column: C18; Mobile Phase A: Water/0.1% NH₃HCO₃, Mobile Phase B: ACN; Flow rate: 100 mL/min; Gradient: 5% B to 35% B in 30 min; 254 nm. This resulted in 2-[5-amino-2-(4,4-difluoropiperidin-1-yl)pyridin-3-yl]ethanol (front peak, 740 mg) as a yellow solid and 1-[5-amino-2-(4,4-difluoropiperidin-1-yl)pyridin-3-yl]ethanol (second peak, 540 mg) as a yellow solid. LCMS Method A: [M+H]⁺=258. ¹H NMR (400 MHz, DMSO-d₆): δ 7.53 (d, 1H), 6.85 (d, 1H), 4.92 (s, 2H), 4.67 (t, 1H), 3.65-3.60 (m, 2H), 2.94 (t, 4H), 2.66 (t, 2H), 2.08-2.03 (m, 4H).

### Synthesis of Intermediate 54 ((5-amino-2-(4,4-difluorocyclohexyl)pyridin-3-yl)methanol)

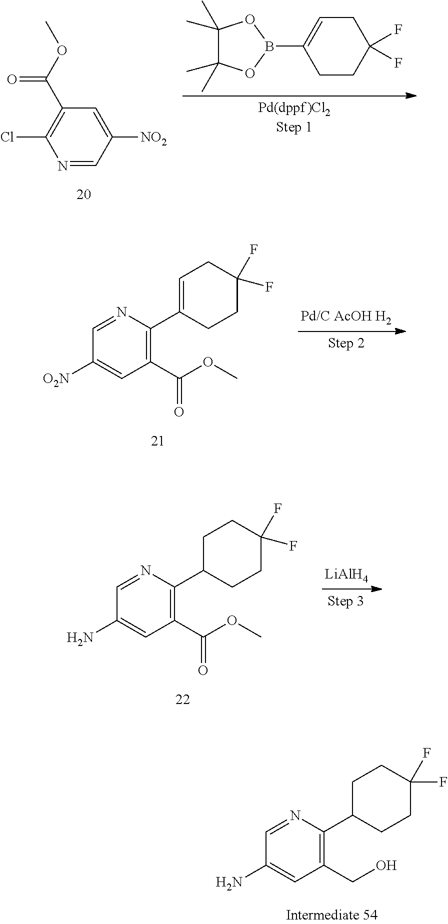

### Step 1: methyl 2-(4,4-difluorocyclohex-1-en-1-yl)-5-nitronicotinate

2-Chloro-5-nitropyridine-3-carboxylate (1.0 g, 4.6 mmol, 1.0 equiv.) was dissolved in 1,4-dioxane (30 mL) and water (5 mL), then K₂CO₃ (1.0 g, 7.2 mmol, 1.5 equiv.), 2-(4,4-difluorocyclohex-1-en-1-yl)-4,4,5,5-tetramethyl-1,3,2-dioxaborolane (1.4 g, 5.7 mmol, 1.2 equiv.) and Pd(dppf)Cl₂ (0.7 g, 1.0 mmol, 0.2 equiv.) were added under an atmosphere of nitrogen. The resulting solution was heated to 90° C. for 2 hours and concentrated under vacuum. The residue was purified by flash column chromatography on silica gel, eluting with ethyl acetate/petroleum ether (1:6) to give methyl 2-(4,4-difluorocyclohex-1-en-1-yl)-5-nitropyridine-3-carboxylate (700 mg) as a white solid. LCMS Method A: [M+H]⁺=299.

### Step 2: methyl 5-amino-2-(4,4-difluorocyclohexyl)nicotinate

2-(4,4-difluorocyclohex-1-en-1-yl)-5-nitropyridine-3-carboxylate (700.0 mg, 2.3 mmol, 1.0 equiv.) was dissolved in MeOH (20 mL), then Pd/C (70.0 mg, 0.7 mmol, 0.3 equiv.) and AcOH (28.2 mg, 0.5 mmol, 0.2 equiv.) were added. The mixture was sparged with nitrogen, placed under an atmosphere of hydrogen gas (balloon), then stirred for 3 days at ambient temperature. The solids were removed by filtration and the filtrate was concentrated under vacuum. The residue was purified by flash column chromatography on silica gel, eluting with ethyl acetate/petroleum ether (1:2) to give methyl 5-amino-2-(4,4-difluorocyclohexyl) pyridine-3-carboxylate (350 mg) as a white solid. LCMS Method C: [M+H]⁺=271.

### Step 3: (5-amino-2-(4,4-difluorocyclohexyl)pyridin-3-yl)methanol

5-amino-2-(4,4-difluorocyclohexyl) pyridine-3-carboxylate (300.0 mg, 1.1 mmol, 1.0 equiv.) was dissolved in THF (20 mL) and cooled to 0° C., then LiAlH₄ (189.6 mg, 5.0 mmol, 4.5 equiv.) was added, maintaining the solution at 0° C. The resulting solution was stirred for 10 min at 0° C. and then quenched by the addition of aqueous HCl (1M). The solution was adjusted to pH 7 with aqueous Na₂CO₃. The resulting solution was extracted with dichloromethane and concentrated under vacuum. The residue was purified by flash column chromatography on silica gel, eluting with ethyl acetate/petroleum ether (1:1) to give [5-amino-2-(4,4-difluorocyclohexyl) pyridin-3-yl] methanol (200 mg) as a white solid. LCMS Method C: [M+H]⁺=243.

### Synthesis of Intermediate 57 (5-amino-2-(4,4-difluoropiperidin-1-yl)nicotinonitrile)

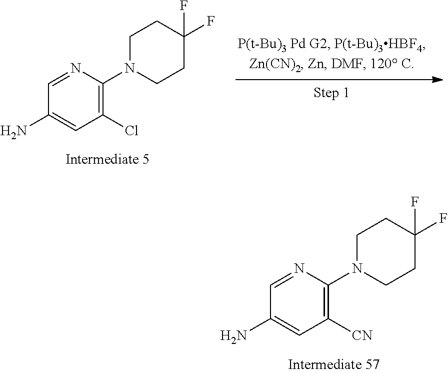 [SMILES: Nc1cnc(N2CCC(F)(F)CC2)c(Cl)c1 ; N#Cc1cc(N)cnc1N1CCC(F)(F)CC1]

5-chloro-6-(4,4-difluorocyclohexyl)pyridin-3-amine (300.0 mg, 1.2 mmol, 1.0 equiv.) was dissolved in DMF (20 mL), then P(t-Bu)₃ Palladacycle Gen. 3 (69.5 mg, 0.1 mmol, 0.1 equiv.), P(t-Bu)₃.HBF₄ (35.2 mg, 0.1 mmol, 0.1 equiv.), Zn(CN)₂ (285.6 mg, 2.4 mmol, 2.0 equiv.) and Zn (11.9 mg, 0.2 mmol, 0.2 equiv.) were added under an atmosphere of nitrogen. The resulting mixture was heated to 120° C. overnight and then quenched with NH₄OH. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:2) to give 5-amino-2-(4,4-difluorocyclohexyl)pyridine-3-carbonitrile (160 mg) as a colorless oil. LCMS Method D: [M+H]⁺=239. ¹H NMR (300 MHz, Methanol-d₄): δ 8.15 (d, 1H), 7.25 (d, 1H), 3.21-3.05 (m, 1H), 2.26-1.80 (m, 8H).

The following intermediates were prepared using the method described for Intermediate 57.

| Intermediate | Starting material | Structure | LCMS data |
|---|---|---|---|
| Intermediate 55 | 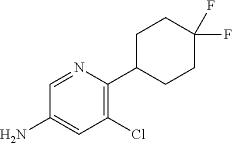 [SMILES: Nc1cnc(C2CCC(F)(F)CC2)c(Cl)c1] Intermediate 44 | 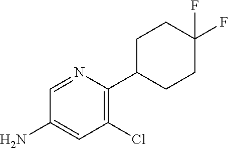 [SMILES: Nc1cnc(C2CCC(F)(F)CC2)c(Cl)c1] | Method A: MS- ESI: 238 [M + H]⁺ |
| Intermediate 56 | 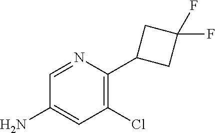 [SMILES: Nc1cnc(C2CC(F)(F)C2)c(Cl)c1] Intermediate 51 | 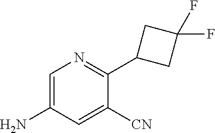 [SMILES: N#Cc1cc(N)cnc1C1CC(F)(F)C1] | Method A: MS- ESI: 210 [M + H]⁺ |

### Synthesis of Intermediate 58 (6-chloro-5-(4,4-difluoropiperidin-1-yl)pyrazin-2-amine)

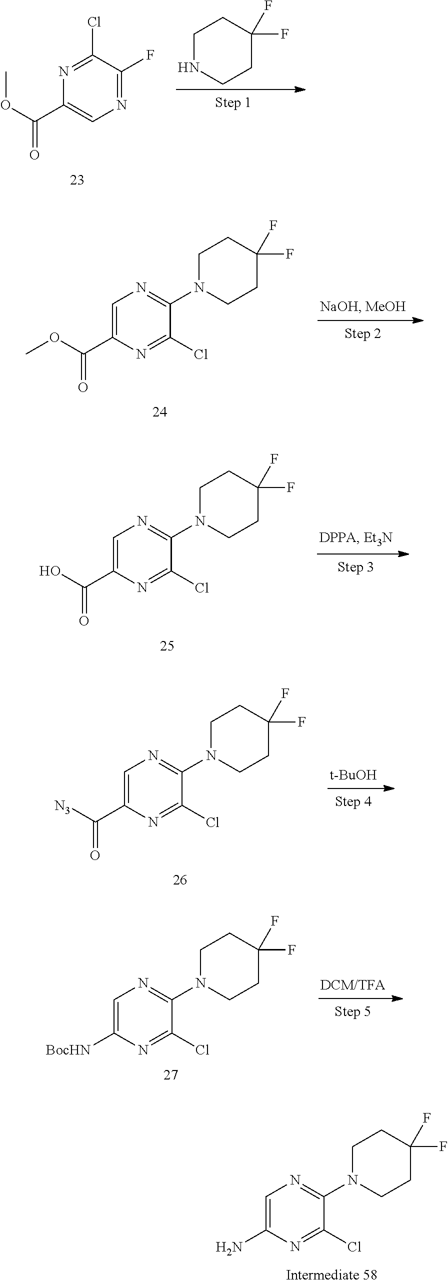

### Step 1: methyl 6-chloro-5-(4,4-difluoropiperidin-1-yl)pyrazine-2-carboxylate

6-Chloro-5-fluoropyrazine-2-carboxylate (1.0 g, 5.2 mmol, 1.0 equiv.) and 4,4-difluoropiperidine (0.8 g, 6.3 mmol, 1.2 equiv.) were dissolved in DMF (20 mL), then Cs₂CO₃ (5.1 g, 15.7 mmol, 3.0 equiv.) was added. The reaction mixture was heated to 50° C. for 3 hours, then cooled to ambient temperature and quenched by the addition of water. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:1) to give methyl 6-chloro-5-(4,4-difluoropiperidin-1-yl)pyrazine-2-carboxylate (1.5 g) as a yellow solid. LCMS Method D: [M+H]⁺=292.

### Step 2: 6-chloro-5-(4,4-difluoropiperidin-1-yl)pyrazine-2-carboxylic acid

Methyl 6-chloro-5-(4,4-difluoropiperidin-1-yl)pyrazine-2-carboxylate (1.0 g, 3.4 mmol, 1.0 equiv.) was dissolved in MeOH (10 mL) and water (10 mL), then NaOH (548.5 mg, 13.7 mmol, 4.0 equiv.) was added. The resulting mixture was stirred for 2 hours at ambient temperature and concentrated under vacuum. The residue was diluted with water and the solution was adjusted to pH 2 with concentrated aqueous HCl. The solid was collected by filtration and dried to give 6-chloro-5-(4,4-difluoropiperidin-1-yl)pyrazine-2-carboxylic acid (950 mg) as a yellow solid. LCMS Method B: [M−H]⁻=276.

### Step 3: 6-chloro-5-(4,4-difluoropiperidin-1-yl)pyrazine-2-carbonyl azide

6-Chloro-5-(4,4-difluoropiperidin-1-yl)pyrazine-2-carboxylic acid (450.0 mg, 1.6 mmol, 1.0 equiv.) was dissolved in THE (15 mL), then DPPA (669.0 mg, 2.4 mmol, 1.5 equiv.) and TEA (0.45 mL, 3.2 mmol, 2.0 equiv.) were added. The resulting mixture was stirred for 3 hours at ambient temperature and concentrated under vacuum to give 6-chloro-5-(4,4-difluoropiperidin-1-yl)pyrazine-2-carbonyl azide (100 mg) as an off-white solid. LCMS Method C: [M+H]⁺=303.

### Step 4: tert-butyl (6-chloro-5-(4,4-difluoropiperidin-1-yl)pyrazin-2-yl)carbamate

6-Chloro-5-(4,4-difluoropiperidin-1-yl)pyrazine-2-carbonyl azide (90.0 mg, 0.3 mmol, 1.0 equiv.) was dissolved in t-BuOH (5 mL). The reaction mixture was heated to 90° C. for 3 hours and concentrated under vacuum. The residue was purified by flash column chromatography on silica gel, eluting with ethyl acetate/petroleum ether (1:1) to give tert-butyl (6-chloro-5-(4,4-difluoropiperidin-1-yl)pyrazin-2-yl)carbamate (95.2 mg) as colorless oil. LCMS Method C: [M+H]⁺=349.

### Step 5: 6-chloro-5-(4,4-difluoropiperidin-1-yl)pyrazin-2-amine

tert-Butyl (6-chloro-5-(4,4-difluoropiperidin-1-yl)pyrazin-2-yl)carbamate (80.0 mg, 0.02 mmol, 1.0 equiv.) was dissolved in DCM (4 mL) and TFA (1 mL). The reaction mixture was stirred for 2 hours at ambient temperature and concentrated under vacuum. The residue was purified by flash column chromatography on silica gel, eluting with ethyl acetate/petroleum ether (1:1) to give 6-chloro-5-(4,4-difluoropiperidin-1-yl)pyrazin-2-amine (51.2 mg) as a yellow solid. LCMS Method C: [M+H]⁺=249.

The following intermediates were prepared using the method described for Intermediate 58.

| Intermediate | Starting material | Structure | LCMS data |
|---|---|---|---|
| Intermediate 59 | 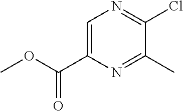 | 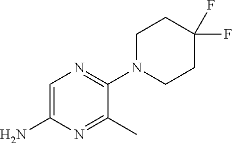 [SMILES: Cc1nc(N)cnc1N1CCC(F)(F)CC1] | Method A: MS-ESI: 229 [M + H]⁺ |

### Synthesis of Intermediate 60 (4-chloro-5-(4,4-difluorocyclohexyl)pyridin-2-amine)

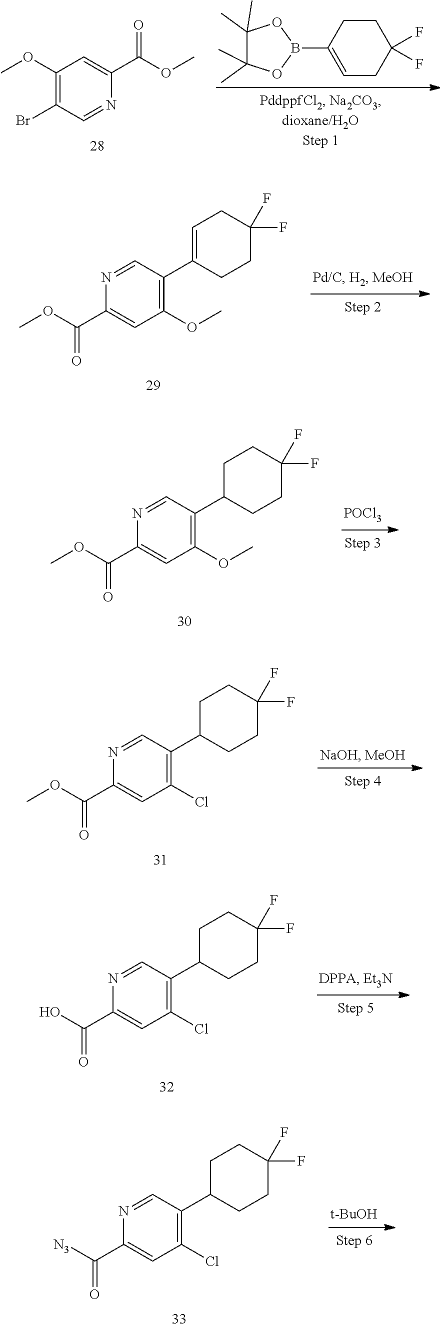

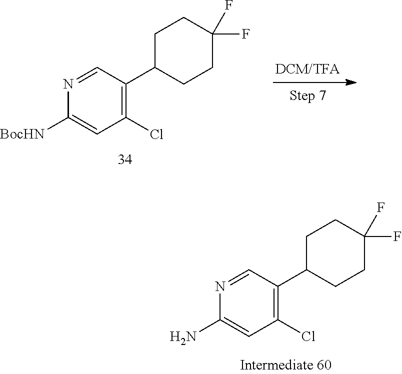 [SMILES: CC(C)(C)OC(=O)Nc1cc(Cl)c(C2CCC(F)(F)CC2)cn1 ; "DCM/TFA" ; Nc1cc(Cl)c(C2CCC(F)(F)CC2)cn1]

### Step 1: methyl 5-(4,4-difluorocyclohex-1-en-1-yl)-4-methoxypicolinate

Methyl 5-bromo-4-hydroxypyridine-2-carboxylate (1.5 g, 6.5 mmol, 1.0 equiv.) and 2-(4,4-difluorocyclohex-1-en-1-yl)-4,4,5,5-tetramethyl-1,3,2-dioxaborolane (4.7 g, 19.4 mmol, 3.0 equiv.) were dissolved in 1.4-dioxane (15 mL) and water (1.5 mL), then Pd(dppf)Cl₂ (0.5 g, 0.6 mmol, 0.1 equiv.) and Na₂CO₃ (2.1 g, 19.4 mmol, 3.0 equiv.) were added. The reaction mixture was heated 70° C. overnight, then quenched by the addition of water. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:1) to give methyl 5-(4,4-difluorocyclohex-1-en-1-yl)-4-hydroxypyridine-2-carboxylate (1.1 g) as a white solid. LCMS Method D: [M+H]⁺=284.

### Step 2: methyl 5-(4,4-difluorocyclohexyl)-4-methoxypicolinate

Methyl 5-(4,4-difluorocyclohex-1-en-1-yl)-4-methoxypyridine-2-carboxylate (6.0 g, 21.2 mmol, 1.0 equiv.) was dissolved in ethyl acetate (60 mL), then Pd/C (10% wt., 1.2 g) was added. The reaction mixture was sparged with nitrogen, placed under an atmosphere of hydrogen gas (balloon), then stirred overnight at ambient temperature. The solids were removed by filtration and the filtrate was concentrated under vacuum to give methyl 5-(4,4-difluorocyclohexyl)-4-methoxypyridine-2-carboxylate (5.3 g) as an off-white solid. LCMS Method D: [M+H]⁺=286.

### Step 3: methyl 4-chloro-5-(4,4-difluorocyclohexyl)picolinate

Methyl 5-(4,4-difluorocyclohexyl)-4-methoxypyridine-2-carboxylate (0.8 g, 2.6 mmol, 1.0 equiv.) was dissolved in toluene (30 mL) and DMF (1 mL) and cooled to 0° C., then POCl₃ (1.1 mL, 13.1 mmol, 5.0 equiv.) was added dropwise, maintaining the temperature at 0° C. The reaction mixture was heated to 90° C. overnight, then cooled to 0° C. and quenched by the addition of ice-water. The mixture was adjusted to pH 8 with saturated aqueous NaHCO₃, then extracted with ethyl acetate, washed with brine, dried over anhydrous Na₂SO₄ and concentrated under vacuum. The residue was purified by flash column chromatography on silica gel, eluting with ethyl acetate/petroleum ether (1:3) to give methyl 4-chloro-5-(4,4-difluorocyclohexyl)pyridine-2-carboxylate (355.0 mg) as a white solid. LCMS Method D: [M+H]⁺=290.

### Step 4: 4-chloro-5-(4,4-difluorocyclohexyl)picolinic acid

Methyl 4-chloro-5-(4,4-difluorocyclohexyl)pyridine-2-carboxylate (2.0 g, 6.9 mmol, 1.0 equiv.) was dissolved in MeOH (20 mL) and water (20 mL), then NaOH (1.1 g, 27.6 mmol, 4.0 equiv.) was added. The reaction mixture was stirred overnight at ambient temperature and concentrated under vacuum. The residue was diluted with water, then adjusted to pH 5 with aqueous HCl (6 M). The resulting solution was extracted with ethyl acetate, washed with brine, dried over anhydrous Na₂SO₄ and concentrated under vacuum to give 4-chloro-5-(4,4-difluorocyclohexyl)pyridine-2-carboxylic acid (705.1 mg) as a white solid. LCMS Method D: [M−H]⁻=274.

### Step 5: 4-chloro-5-(4,4-difluorocyclohexyl)picolinoyl azide

4-Chloro-5-(4,4-difluorocyclohexyl)pyridine-2-carboxylic acid (430.0 mg, 1.6 mmol, 1.0 equiv.) and TEA (189 mg, 1.9 mmol, 1.2 equiv.) were dissolved in toluene (6 mL), then DPPA (515.0 mg, 1.9 mmol, 1.2 equiv.) was added. The reaction mixture was stirred overnight at ambient temperature and quenched by the addition of water. The resulting solution was extracted with ethyl acetate, washed with saturated aqueous NaHCO₃, dried over anhydrous Na₂SO₄ and concentrated under vacuum to give 4-chloro-5-(4,4-difluorocyclohexyl)pyridine-2-carbonyl azide (400.0 mg) as a light brown solid. LCMS Method D: [M+H]⁺=301.

### Step 6: tert-butyl (4-chloro-5-(4,4-difluorocyclohexyl)pyridin-2-yl)carbamate

4-Chloro-5-(4,4-difluorocyclohexyl)pyridine-2-carbonyl azide (400.0 mg, 1.3 mmol, 1.0 equiv.) was dissolved in t-BuOH (4 mL). The solution was heated to 90° C. overnight. The precipitated solids were collected by filtration and washed with ethyl acetate to five tert-butyl N-[4-chloro-5-(4,4-difluorocyclohexyl)pyridin-2-yl]carbamate (380 mg) as a white solid. LCMS Method D: [M+H]⁺=347.

### Step 7: 4-chloro-5-(4,4-difluorocyclohexyl)pyridin-2-amine

tert-Butyl N-[4-chloro-5-(4,4-difluorocyclohexyl)pyridin-2-yl]carbamate (190.0 mg, 0.5 mmol, 1.0 equiv.) was dissolved in DCM (2 mL) and TFA (0.5 mL). The reaction mixture was stirred for 2 hours at ambient temperature, and then concentrated under vacuum. The residue was dissolved in water and adjusted to pH=7 with saturated aqueous NaHCO₃. The resulting mixture was extracted with ethyl acetate, washed with brine, dried over anhydrous Na₂SO₄ and concentrated under vacuum. The residue was purified by flash column chromatography on silica gel, eluting with DCM/MeOH (20:1) to give 4-chloro-5-(4,4-difluorocyclohexyl)pyridin-2-amine (130 mg) as a light yellow solid. LCMS Method D: [M+H]⁺=247.

### Synthesis of Intermediate 61 (5,6-dichloro-1H-indole-3-carboxylic acid)

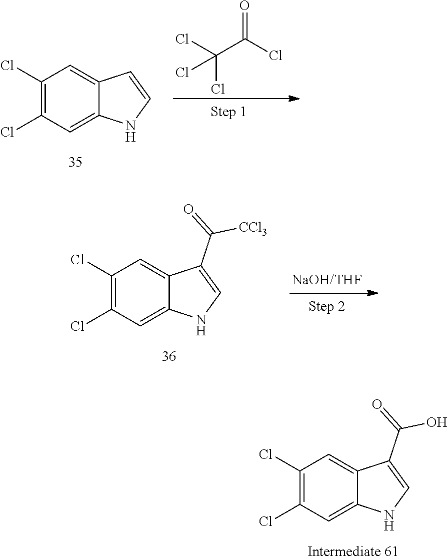

### Step 1: 2,2,2-trichloro-1-(5,6-dichloro-1H-indol-3-yl)ethan-1-one

5,6-dichloro-1H-indole (500.0 mg, 2.7 mmol, 1.0 equiv.) and pyridine (0.4 mL, 5.0 mmol, 2.0 equiv.) were dissolved in DCM (20 mL), then trichloroacetyl chloride (736.3 mg, 4.0 mmol, 1.5 equiv.) was added at ambient temperature. The reaction mixture was heated to 65° for 2 hours, then concentrated under vacuum. The residue was purified by flash column chromatography on silica gel, eluting with ethyl acetate/petroleum ether (1:1) to give 2,2,2-trichloro-1-(5,6-dichloro-1H-indol-3-yl)ethanone (667.3 mg) as a yellow solid. LCMS Method A: [M+H]⁺=330.

### Step 2: 5,6-dichloro-1H-indole-3-carboxylic acid

2,2,2-trichloro-1-(5,6-dichloro-1H-indol-3-yl)ethanone (1.0 g, 3.0 mmol, 1.0 equiv.) was dissolved in THF (10 mL), then NaOH (120.7 mg, 3.0 mmol, 1.0 equiv.) was added. The reaction mixture was stirred for 24 hours at ambient temperature and then concentrated under vacuum. The residue was diluted with water, then adjusted to pH 4 with aqueous HCl (6M). The resulting mixture was extracted with Et₂O, washed with brine, dried over anhydrous Na₂SO₄ and concentrated under vacuum to give 5,6-dichloro-1H-indole-3-carboxylic acid (650 mg) as a pink solid. LCMS Method B: [M−H⁻]=228.

The following intermediates were prepared using the method described for Intermediate 61.

| Intermediate | Starting material | Structure | LCMS data |
|---|---|---|---|
| Intermediate 62 | 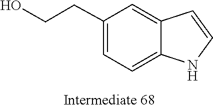 [SMILES: OCCc1ccc2[nH]ccc2c1] | 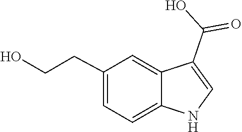 [SMILES: O=C(O)c1c[nH]c2ccc(CCO)cc12] | Method B: MS-ESI: 204 [M − H]⁻ |
| Intermediate 63 | 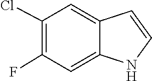 [SMILES: Fc1cc2[nH]ccc2cc1Cl] | 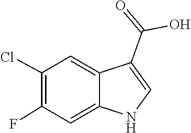 [SMILES: O=C(O)c1c[nH]c2cc(F)c(Cl)cc12] | Method B: MS-ESI: 212 [M − H]⁻ |
| Intermediate 64 | 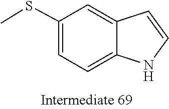 | 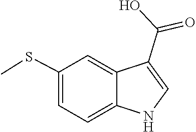 | Method B: MS-ESI: 206 [M − H]⁻ |
| Intermediate 100 | 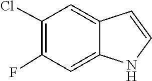 [SMILES: Fc1cc2[nH]ccc2cc1Cl] | 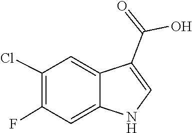 [SMILES: O=C(O)c1c[nH]c2cc(F)c(Cl)cc12] | Method B: MS-ESI: 212 [M − H]− |
| Intermediate 101 | 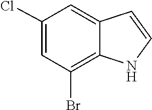 [SMILES: Clc1cc(Br)c2[nH]ccc2c1] | 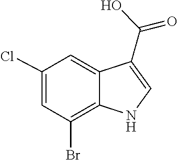 [SMILES: O=C(O)c1c[nH]c2c(Br)cc(Cl)cc12] | Method B: MS-ESI: 272 [M − H]− |
| Intermediate 101a | 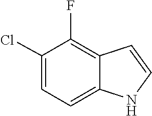 [SMILES: Fc1c(Cl)ccc2[nH]ccc12] | 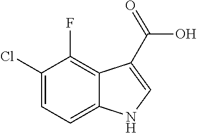 [SMILES: O=C(O)c1c[nH]c2ccc(Cl)c(F)c12] | Method B: MS-ESI: 212.1 [M − H]− |
| Intermediate 101b | 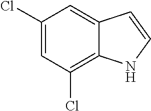 [SMILES: Clc1cc(Cl)c2[nH]ccc2c1] | 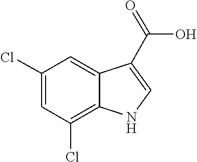 [SMILES: O=C(O)c1c[nH]c2c(Cl)cc(Cl)cc12] | Method B: MS-ESI: 227.1 [M − H]− |

### Synthesis of Intermediate 65 (3-amino-1H-indol-5-ol)

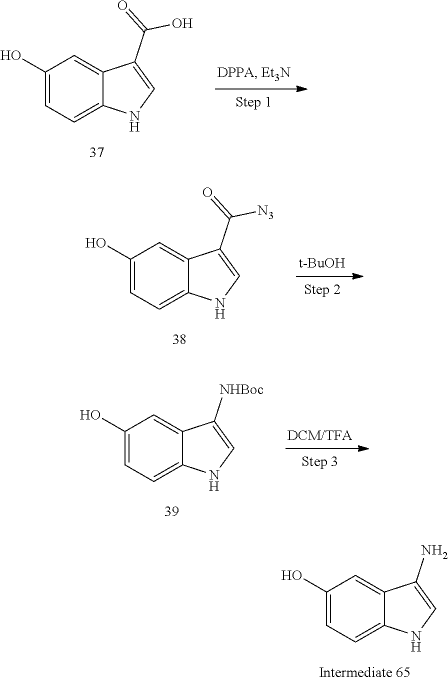 [SMILES: "t-BuOH" ; O=C(O)c1c[nH]c2ccc(O)cc12 ; [N-]=[N+]=NC(=O)c1c[nH]c2ccc(O)cc12 ; CC(C)(C)OC(=O)Nc1c[nH]c2ccc(O)cc12 ; "DCM/TFA" ; Nc1c[nH]c2ccc(O)cc12]

### Step 1: 5-hydroxy-1H-indole-3-carbonyl azide

5-hydroxy-1H-indole-3-carboxylic acid (1.0 g, 5.6 mmol, 1.0 equiv.) was dissolved in THE (40 mL), then TEA (1.2 mL, 8.5 mmol, 1.5 equiv.) and DPPA (2.0 g, 7.3 mmol, 1.3 equiv.) were added. The reaction mixture was stirred for 8 hours at ambient temperature and then concentrated under vacuum to give crude 5-hydroxy-1H-indole-3-carbonyl azide (1.2 g) as a white solid. LCMS Method C: [M+H]⁺=203.

### Step 2: tert-butyl (5-hydroxy-1H-indol-3-yl)carbamate

5-hydroxy-1H-indole-3-carbonyl azide (1.2 g, 5.9 mmol, 1.0 equiv.) was dissolved in t-BuOH (40 mL). The resulting solution was heated to 90° C. for 4 hours, then cooled to ambient temperature and concentrated under vacuum. The residue was purified by flash column chromatography on silica gel, eluting with ethyl acetate/petroleum ether (1:6) to give tert-butyl (5-hydroxy-1H-indol-3-yl)carbamate (1.0 g) as a white solid. LCMS Method C: [M+H]⁺=249.

### Step 3: 3-amino-1H-indol-5-ol

tert-Butyl (5-hydroxy-1H-indol-3-yl)carbamate (300.0 mg, 1.2 mmol, 1.0 equiv.) was dissolved in DCM (6 mL) and TFA (2 ml). The resulting solution was stirred for 30 min at ambient temperature and then concentrated under vacuum to give crude 3-amino-1H-indol-5-ol (420 mg) as a yellow solid. LCMS Method C: [M+H]⁺=149.

### Synthesis of Intermediate 66 (5-(difluoromethyl)-1H-indol-3-amine)

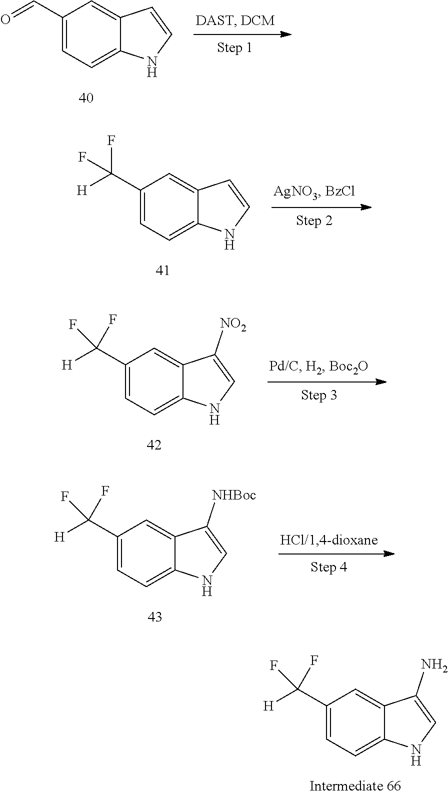

### Step 1: 5-(difluoromethyl)-1H-indole

1H-indole-5-carbaldehyde (15.0 g, 103.3 mmol, 1.0 equiv.) was dissolved in DCM (150 mL) and cooled to 0° C., then DAST (83.3 g, 516.7 mmol, 5.0 equiv.) was added dropwise, maintaining the solution at 0° C. under nitrogen atmosphere. The resulting mixture was stirred overnight at ambient temperature, then cooled to 0° C. and quenched by the addition of ice-water. The resulting solution was adjusted to pH 7 with saturated aqueous NaHCO₃. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:5) to give 5-(difluoromethyl)-1H-indole (0.6 g) as a yellow solid. LCMS Method C: [M+H]⁺=168.

### Step 2: 5-(difluoromethyl)-3-nitro-1H-indole

5-(Difluoromethyl)-1H-indole (5.8 g, 6.0 mmol, 1.0 equiv.) and AgNO₃ (1.5 g, 9.0 mmol, 1.5 equiv.) were dissolved in MeCN (15 mL) and cooled to 0° C. After 10 min at 0° C., benzoyl chloride (1.1 mL, 9.2 mmol, 1.5 equiv.) was added dropwise, maintaining the solution at 0° C. The reaction mixture was stirred for additional 2 hours at 0° C. and then quenched by the addition of ice-water. The resulting solution was extracted with ethyl acetate, dried over anhydrous Na₂SO₄ and concentrated under vacuum. The residue was purified by flash column chromatography on silica gel, eluting with ethyl acetate/petroleum ether (1:8) to give 5-(difluoromethyl)-3-nitro-1H-indole (490 mg) as a yellow oil. LCMS Method D: [M+H]⁺=213.

### Step 3: tert-butyl (5-(difluoromethyl)-1H-indol-3-yl)carbamate

5-(Difluoromethyl)-3-nitro-1H-indole (480.0 mg, 0.9 mmol, 1.0 equiv.) was dissolved MeOH (10 mL), then Pd/C (10% wt., 100.3 mg) and Boc₂O (411.5 mg, 1.9 mmol, 2.0 equiv.) were added. The mixture was sparged with nitrogen, placed under an atmosphere of hydrogen gas (balloon), then stirred overnight at ambient temperature. The solids were removed by filtration and the filtrate was concentrated under vacuum. The residue was purified by flash column chromatography on silica gel, eluting with ethyl acetate/petroleum ether (1:10) to give tert-butyl N-[5-(difluoromethyl)-1H-indol-3-yl]carbamate (320 mg) as an off-white solid. LCMS Method C: [M+H]⁺=283.

### Step 4: 5-(difluoromethyl)-1H-indol-3-amine

tert-Butyl N-[5-(difluoromethyl)-1H-indol-3-yl]carbamate (320.0 mg, 0.5 mmol, 1.0 equiv.) was dissolved HCl (4M in 1,4-dioxane, 5 mL). The resulting solution was stirred for 1 hour at ambient temperature and then concentrated under vacuum to give 5-(difluoromethyl)-1H-indol-3-amine hydrogen chloride (210 mg) as a yellow solid, that was used to next step directly without further purification. LCMS Method A: [M+H]⁺=183.

### Synthesis of Intermediate 67 (5-(methylsulfonyl)-1H-indole-3-carboxylic acid)

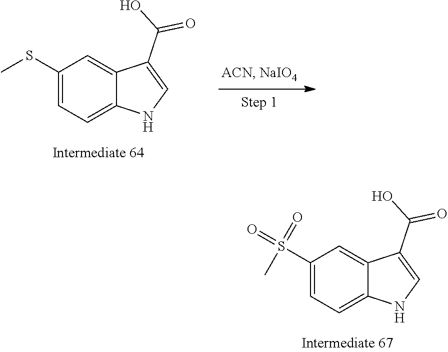 [SMILES: CSc1ccc2[nH]cc(C(=O)O)c2c1 ; CS(=O)(=O)c1ccc2[nH]cc(C(=O)O)c2c1]

5-(Methylsulfanyl)-1H-indole-3-carboxylic acid (400.0 mg, 1.9 mmol, 1.0 equiv.) was dissolved ACN (400 mL), NaIO₄ (1.6 g, 7.7 mmol, 4.0 equiv.) was added. The resulting solution was heated to 80° C. for 2 hours and then concentrated under vacuum. The residue was purified by flash column chromatography on silica gel, eluting with ethyl acetate/petroleum ether (1:1) to give 5-methanesulfonyl-1H-indole-3-carboxylic acid (300 mg) as an off-white solid. LCMS Method B: [M−H]⁻=238.

### Synthesis of Intermediate 68 (2-(1H-indol-5-yl)ethan-1-ol)

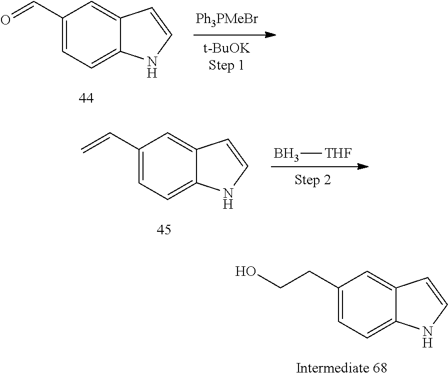

### Step 1: 5-ethenyl-1H-indole

Methyltriphenylphosphanium bromide (14.8 g, 41.4 mmol, 2.0 equiv.) and t-BuOK (4.6 g, 42.1 mmol, 2.0 equiv.) were dissolved in THF (50 mL) and cooled to 0° C., then a solution of 1H-indole-5-carbaldehyde (3.0 g, 20.7 mmol, 1.0 equiv.) in THF (5 mL) was added dropwise. The reaction mixture was stirred for 2 hours at ambient temperature and concentrated under vacuum. The residue was purified by flash column chromatography on silica gel, eluting with ethyl acetate/petroleum ether (1:10) to give 5-ethenyl-1H-indole (1.8 g) as an off-white solid. LCMS Method A: [M+H]⁺=144.

### Step 2: 2-(1H-indol-5-yl)ethan-1-ol

5-Ethenyl-1H-indole (1.0 g, 7.0 mmol, 1.0 equiv.) was dissolved in THF (40 mL) and cooled to 0° C., then BH₃-THF (1M, 8.4 mL, 8.4 mmol, 1.2 equiv.) was added dropwise. The reaction mixture was stirred for 20 min at 0° C., and then NaOH (1.1 g, 27.5 mmol, 4.0 equiv.) was added. The resulting mixture was stirred for 1 hour at ambient temperature and then quenched by the addition of sodium hydrosulfite. The resulting mixture was extracted with ethyl acetate, washed with brine, dried over anhydrous Na₂SO₄ and concentrated under vacuum to give 2-(1H-indol-5-yl)ethan-1-ol (650.1 mg) as a yellow solid. LCMS Method A: [M+H]⁺=162.

### Synthesis of Intermediate 69 (5-(methylthio)-1H-indole)

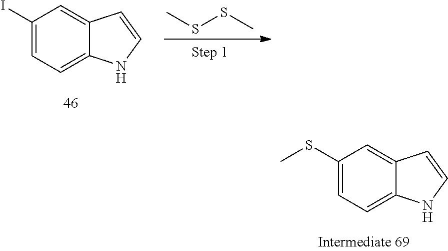

5-iodo-1H-indole (15.0 g, 61.7 mmol, 1.0 equiv.) was dissolved in THF (200 mL) under an atmosphere of nitrogen, cooled to −78° C., then a solution of n-BuLi in hexanes (2.5 M, 49.4 mL, 123.5 mmol, 2.0 equiv.) was added dropwise, maintaining the temperature at −78° C. After 30 min at −78° C., dimethyl disulfide (11.6 g, 123.5 mmol, 2.0 equiv.) was added dropwise at −78° C. The reaction mixture was stirred for additional 1 hour at ambient temperature and then quenched by the addition of aqueous NH₄Cl. The resulting mixture was extracted with ethyl acetate, washed with brine, dried over anhydrous Na₂SO₄ and concentrated under vacuum. The residue was purified by reverse flash chromatography with following conditions: column, C18 silica gel; mobile phase, MeCN and water (0.5% TFA), 35% MeCN increasing to 70% in 30 min; detector, UV 254 nm. This resulted in 5-(methylsulfanyl)-1H-indole (1.7 g) as a yellow solid. LCMS Method C: [M+H]⁺=164.

### Synthesis of Intermediate 70 (1-(4-ethylphenyl)-4-(4,4,5,5-tetramethyl-1,3,2-dioxaborolan-2-yl)-1H-pyrazole)

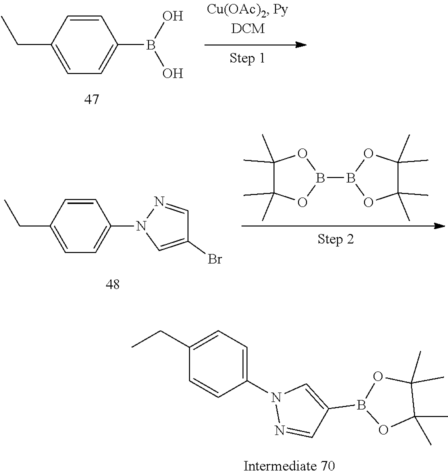

### Step 1: 4-bromo-1-(4-ethylphenyl)-1H-pyrazole

4-Ethylphenylboronic acid (10.0 g, 66.7 mmol, 1.0 equiv.) and 4-bromopyrazole (9.8 g, 66.7 mmol, 1.0 equiv.) were dissolved in DCM (300.0 mL), then Cu(OAc)₂ (24.2 g, 133.4 mmol, 2.0 equiv.) and pyridine (2.1 mL, 26.7 mmol, 2.0 equiv.) were added under nitrogen. The reaction mixture was stirred overnight at ambient temperature and concentrated under vacuum. The residue was purified by flash column chromatography on silica gel, eluting with ethyl acetate/petroleum ether (1:10) to give 4-bromo-1-(4-ethylphenyl)pyrazole (9.5 g) as a white solid. LCMS Method F: [M+H]⁺=251.

### Step 2: 1-(4-ethylphenyl)-4-(4,4,5,5-tetramethyl-1,3,2-dioxaborolan-2-yl)-1H-pyrazole

4-Bromo-1-(4-ethylphenyl)pyrazole (9.5 g, 37.8 mmol, 1.0 equiv.) was dissolved in dioxane (200.0 ml), then bis(pinacolato)diboron (9.6 g, 37.8 mmol, 1.0 equiv.), AcOK (7.4 g, 75.7 mmol, 2.0 equiv.) and Pd(dppf)Cl₂ (5.5 g, 7.6 mmol, 0.2 equiv.) were added under nitrogen. The reaction mixture was heated to 80° C. overnight, then cooled to ambient temperature and concentrated under vacuum. The residue was purified by flash column chromatography on silica gel, eluting with ethyl acetate/petroleum ether (1:4) to give 1-(4-ethylphenyl)-4-(4,4,5,5-tetramethyl-1,3,2-dioxaborolan-2-yl)-1H-pyrazole (4.0 g) as a yellow solid. LCMS Method D: [M+H]⁺=299.

### Synthesis of Intermediate 106 (2-(1-(2,2,2-trifluoroethyl)piperidin-3-yl)pyridin-4-amine)

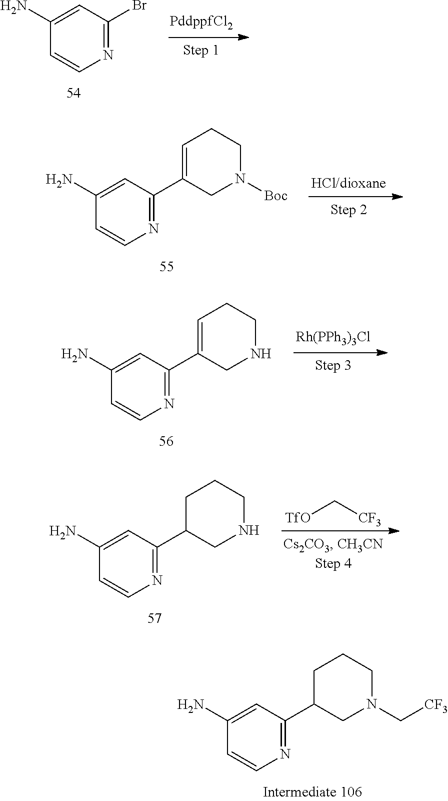

### Step 1: tert-butyl 4-amino-5,6-dihydro-2H-[2,3-bipyridine]-1-carboxylate

2-Bromopyridin-4-amine (500.0 mg, 2.9 mmol, 1.0 equiv.) and tert-butyl 3-(4,4,5,5-tetramethyl-1,3,2-dioxaborolan-2-yl)-5,6-dihydro-2H-pyridine-1-carboxylate (1072.3 mg, 3.5 mmol, 1.2 equiv.) were dissolved in 1,4-dioxane/water (25/5 mL), Cs₂CO₃ (1883.2 mg, 5.8 mmol, 2.0 equiv.) and Pd(dppf)Cl₂ (211.5 mg, 0.3 mmol, 0.1 equiv.) were added under an atmosphere of nitrogen. The reaction mixture was heated to 90° C. overnight under nitrogen, the cooled to ambient temperature and concentrated under vacuum. The residue was purified by flash column chromatography on silica gel, eluting with ethyl acetate/petroleum ether (1:1) to give tert-butyl 4-amino-5,6-dihydro-2H-[2,3-bipyridine]-1-carboxylate (580.0 mg) as a brown solid. LCMS Method A: [M+H]⁺=276.

### Step 2: 1,2,5,6-tetrahydro-[2,3-bipyridin]-4-amine

tert-Butyl 4-amino-5,6-dihydro-2H-[2,3-bipyridine]-1-carboxylate (605.0 mg, 2.2 mmol, 1.0 equiv.) was dissolved in HCl (4M in 1,4-dioxane, 10 mL). The reaction mixture was stirred for 2 hours at ambient temperature and concentrated under vacuum. The residue was diluted with water, then adjusted to pH 8 with saturated NaHCO₃ aqueous. The resulting solution was extracted with ethyl acetate and concentrated under vacuum. The residue was purified by flash column chromatography on silica gel, eluting with DCM/MeOH (10:1) to give 1,2,5,6-tetrahydro-[2,3-bipyridin]-4-amine (332.2 mg) as a brown yellow solid. LCMS Method D: [M+H]⁺=176.

### Step 3: 2-(piperidin-3-yl)pyridin-4-amine

1,2,5,6-Tetrahydro-[2,3-bipyridin]-4-amine (332.0 mg, 1.9 mmol, 1.0 equiv.) was dissolved in MeOH (10 mL), R^{h}(PPh₃)₃Cl (175.3 mg, 0.2 mmol, 0.1 equiv.) was added.

The mixture was sparged with nitrogen, placed under an atmosphere of hydrogen gas (balloon), then stirred overnight at 50° C. The solids were removed by filtration and the filtrate was concentrated under vacuum. The residue was purified by flash column chromatography on silica gel, eluting with ethyl acetate/petroleum ether (1:10) to give 2-(piperidin-3-yl)pyridin-4-amine (215.2 mg) as a brown solid. LCMS Method D: [M+H]⁺=178.

### Step 4: 2-[1-(2,2,2-trifluoroethyl)piperidin-3-yl]pyridin-4-amine

2-(Piperidin-3-yl)pyridin-4-amine (200.0 mg, 1.1 mmol, 1.0 equiv.) and 2,2,2-trifluoroethyl trifluoromethanesulfonate (314.3 mg, 1.4 mmol, 1.2 equiv.) were dissolved in ACN (10 mL), Cs₂CO₃ (1102.9 mg, 3.4 mmol, 3.0 equiv.) was added. The reaction mixture was stirred overnight at ambient temperature. After removing the sloid by filtration, the solution was concentrated under vacuum. The residue was purified by flash column chromatography on silica gel, eluting with ethyl acetate/petroleum ether (1:1) to give 2-[1-(2,2,2-trifluoroethyl)piperidin-3-yl]pyridin-4-amine (180.0 mg) as a brown solid. LCMS Method A: [M+H]⁺=260.

The following intermediates were prepared using the method described for Intermediate 106.

| Intermediate | Starting material | Structure | LCMS data |
|---|---|---|---|
| Intermediate 107 | 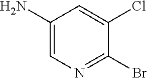 | 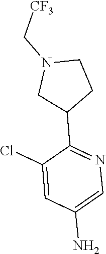 [SMILES: Nc1cnc(C2CCN(CC(F)(F)F)C2)c(Cl)c1] | Method C: MS-ESI: 280 [M + H]⁺ |
| Intermediate 108 | 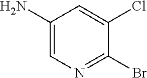 | 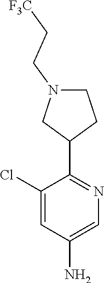 [SMILES: Nc1cnc(C2CCN(CCC(F)(F)F)C2)c(Cl)c1] | Method C: MS-ESI: 294 [M + H]⁺ |

### Synthesis of Intermediate 109 (3-chloro-4-(1-(2,2,2-trifluoroethyl)azetidin-3-yl)aniline hydrochloride)

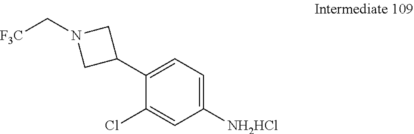

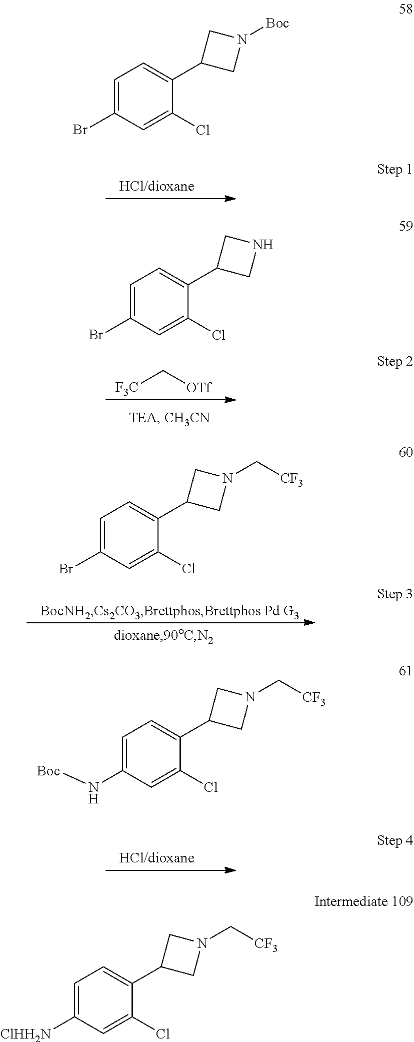

### Step 1: 3-(4-bromo-2-chlorophenyl)azetidine

tert-Butyl 3-(4-bromo-2-chlorophenyl)azetidine-1-carboxylate (2.0 g, 5.8 mmol, 1.0 equiv.) was dissolved in HCl (4M in 1,4-dioxane, 10 mL). The resulting solution was stirred for 2 hours at ambient temperature and then concentrated under vacuum to give 3-(4-bromo-2-chlorophenyl)azetidine hydrochloride (1.4 g) as a white solid. LCMS Method F: [M+H]⁺=246.

### Step 2: 3-(4-bromo-2-chlorophenyl)-1-(2,2,2-trifluoroethyl)azetidine

3-(4-Bromo-2-chlorophenyl)azetidine hydrochloride (800.0 mg, 2.8 mmol, 1.0 equiv.) and TEA (2.2 mL, 16.2 mmol, 5.0 equiv.) were dissolved in ACN (15 mL), 2,2,2-trifluoroethyl trifluoromethanesulfonate (1129.8 mg, 4.9 mmol, 1.5 equiv.) was added. The reaction mixture was heated to 50° C. for 4 hours, then cooled to ambient temperature and concentrated under vacuum. The residue was purified by flash column chromatography on silica gel, eluting with ethyl acetate/petroleum ether (1:1) to give 3-(4-bromo-2-chlorophenyl)-1-(2,2,2-trifluoroethyl)azetidine (789.2 mg) as a brown oil. LCMS Method D: [M+H]⁺=328.

### Step 3: tert-butyl (3-chloro-4-(1-(2,2,2-trifluoroethyl)azetidin-3-yl)phenyl)carbamate

3-(4-Bromo-2-chlorophenyl)-1-(2,2,2-trifluoroethyl)azetidine (400.0 mg, 1.2 mmol, 1.0 equiv.) was dissolved in dioxane (10 mL), BocNH₂ (213.9 mg, 1.8 mmol, 1.5 equiv.), Cs₂CO₃ (793.3 mg, 2.4 mmol, 2.0 equiv.), Brettphos (65.4 mg, 0.1 mmol, 0.1 equiv.) and Brettphos Pd G3 (110.4 mg, 0.1 mmol, 0.1 equiv.) were added under an atmosphere of nitrogen. The reaction mixture was heated to 50° C. for 4 hours, then cooled to ambient temperature and concentrated under vacuum. The residue was purified by flash column chromatography on silica gel, eluting with ethyl acetate/petroleum ether (1:1) to give tert-butyl (3-chloro-4-(1-(2,2,2-trifluoroethyl)azetidin-3-yl)phenyl)carbamate (280.5 mg) of as a brown oil. LCMS Method D: [M+H]⁺=365.

### Step 4: 3-chloro-4-(1-(2,2,2-trifluoroethyl)azetidin-3-yl)aniline hydrochloride

tert-Butyl (3-chloro-4-(1-(2,2,2-trifluoroethyl)azetidin-3-yl)phenyl)carbamate (200.0 mg, 0.5 mmol, 1.0 equiv.) was dissolved in HCl (4M in 1,4-dioxane, 5 mL). The resulting solution was stirred for 2 hours at ambient temperature and then concentrated under vacuum to give 3-chloro-4-(1-(2,2,2-trifluoroethyl)azetidin-3-yl)aniline hydrochloride (131.5 mg) as an off-white solid. LCMS Method A: [M+H]⁺=265.

### Synthesis of Intermediate 113 (1-(4,4-difluorocyclohexyl)pyrazol-4-amine)

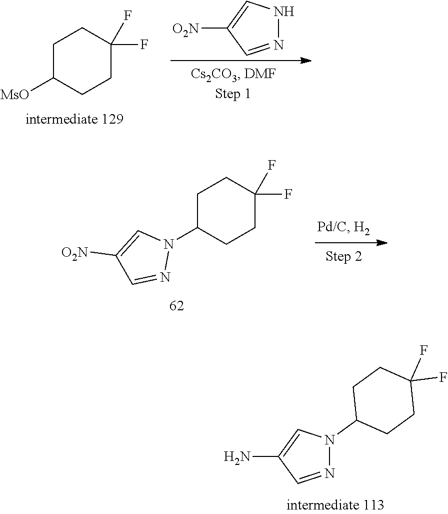 [SMILES: CS(=O)(=O)OC1CCC(F)(F)CC1 ; O=[N+]([O-])c1cn[nH]c1 ; O=[N+]([O-])c1cnn(C2CCC(F)(F)CC2)c1 ; Nc1cnn(C2CCC(F)(F)CC2)c1]

### Step 1: 1-(4,4-difluorocyclohexyl)-4-nitropyrazole

4,4-Difluorocyclohexyl methanesulfonate (500.0 mg, 2.3 mmol, 1.0 equiv.) was dissolved in DMF (10 mL), then 4-nitropyrazole (316.7 mg, 2.8 mmol, 1.2 equiv.), Cs₂CO₃ (1.5 g, 4.7 mmol, 2.0 equiv.) were added. The reaction mixture was heated to 90° C. for 12 hours, then cooled to ambient temperature and quenched by the addition of water. The resulting solution was extracted with ethyl acetate, washed with brine, dried over anhydrous sodium sulfate and concentrated under vacuum. The residue was purified by flash column chromatography on silica gel column, eluting with ethyl acetate/petroleum ether (1:10) to give 1-(4,4-difluorocyclohexyl)-4-nitropyrazole (420.0 mg) as an off-white solid. LCMS Method C: [M+H]⁺=232.

### Step 2: 1-(4,4-difluorocyclohexyl)pyrazol-4-amine

1-(4,4-difluorocyclohexyl)-4-nitropyrazole (400.0 mg, 1.7 mmol, 1.0 equiv.) was dissolved in MeOH (10 mL), then Pd/C (184.1 mg, 10% wt.) was added. The reaction mixture was sparged with nitrogen, placed under an atmosphere of hydrogen gas (balloon), then stirred overnight at ambient temperature. The solids were removed by filtration and the filtrate was concentrated under vacuum to give 1-(4,4-difluorocyclohexyl)pyrazol-4-amine (243.1 mg) as a yellow solid. LCMS Method C: [M+H]⁺=202.

The following intermediates were prepared using the method described for Intermediate 113.

| Intermediate | Starting material | Structure | LCMS data |
|---|---|---|---|
| Intermediate 110 | 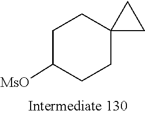 [SMILES: CS(=O)(=O)OC1CCC2(CC1)CC2] | 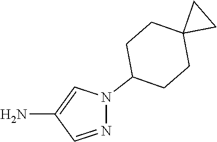 [SMILES: Nc1cnn(C2CCC3(CC2)CC3)c1] | Method A: MS- ESI: 192 [M + H]⁺ |
| Intermediate 111 | 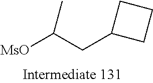 | 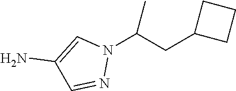 | Method A: MS- ESI: 180 [M + H]⁺ |
| Intermediate 112 | 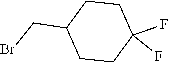 | 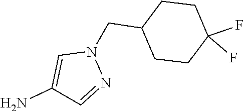 [SMILES: Nc1cnn(CC2CCC(F)(F)CC2)c1] | Method A: MS- ESI: 216 [M + H]⁺ |
| Intermediate 114 | 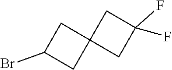 | 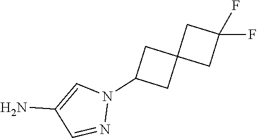 [SMILES: Nc1cnn(C2CC3(C2)CC(F)(F)C3)c1] | Method A: MS- ESI: 214 [M + H]⁺ |
| Intermediate 115 | 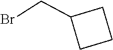 | 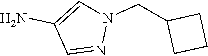 [SMILES: Nc1cnn(CC2CCC2)c1] | Method A: MS- ESI: 152 [M + H]⁺ |

### Synthesis of Intermediate 116 (1-(3,3-difluorocyclobutyl)-1H-pyrazol-4-amine)

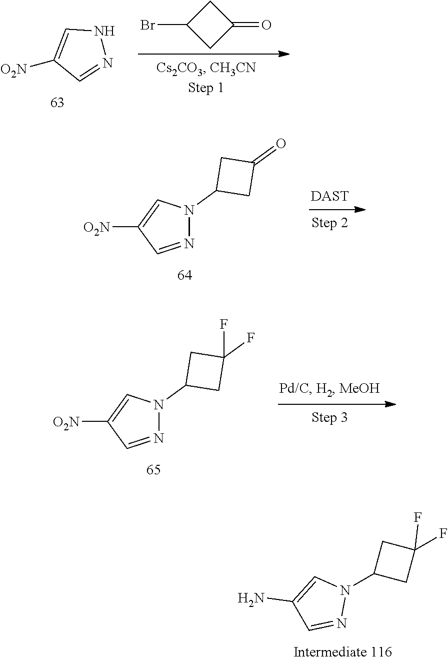

### Step 1: 3-(4-nitropyrazol-1-yl)cyclobutan-1-one

4-Nitropyrazole (1.0 g, 8.8 mmol, 1.0 equiv.) and K₂CO₃ (2.4 g, 17.7 mmol, 2.0 equiv.) were dissolved in ACN (20 mL), 3-bromocyclobutan-1-one (5.3 g, 35.4 mmol, 4.0 equiv.) was added. The reaction mixture was stirred overnight at ambient temperature, then removed the solid by filtration and the filtrate was concentrated under vacuum. The residue was purified by flash column chromatography on silica gel column, eluting with ethyl acetate/petroleum ether (1:1) to give 3-(4-nitropyrazol-1-yl)cyclobutan-1-one (530.0 mg) as an off-white solid. LCMS Method D: [M+H]⁺=182.

### Step 2: 1-(3,3-difluorocyclobutyl)-4-nitropyrazole

3-(4-Nitropyrazol-1-yl)cyclobutan-1-one (470.0 mg, 2.6 mmol, 1.0 equiv.) was dissolved DCM (20 mL) and cooled to 0° C., DAST (836.4 mg, 5.2 mmol, 2.0 equiv.) was added. The reaction mixture was stirred overnight at ambient temperature and quenched by the addition of ice-water. The resulting solution was concentrated under vacuum and the residue was purified by flash column chromatography on silica gel column, eluting with ethyl acetate/petroleum ether (1:1) to give 1-(3,3-difluorocyclobutyl)-4-nitropyrazole (420.0 mg) as a brown solid. LCMS Method A: [M+H]⁺=204.

### Step 3: 1-(3,3-difluorocyclobutyl)pyrazol-4-amine

1-(3,3-Difluorocyclobutyl)-4-nitropyrazole (400.0 mg, 2.0 mmol, 1.0 equiv.) was dissolved in MeOH (10 mL), Pd/C (41.9 mg, 10% wt.) was added. The reaction mixture was sparged with nitrogen, placed under an atmosphere of hydrogen gas (balloon), then stirred overnight at ambient temperature. The solids were removed by filtration and the filtrate was concentrated under vacuum. The residue was purified by flash column chromatography on silica gel column, eluting with DCM/MeOH (12:1) to give 1-(3,3-difluorocyclobutyl)pyrazol-4-amine (300.0 mg) as an off-white solid. LCMS Method E: [M+H]⁺=174.

### Synthesis of Intermediate 117 (1-(1-(3,3,3-trifluoropropyl)piperidin-4-yl)-1H-pyrazol-4-amine)

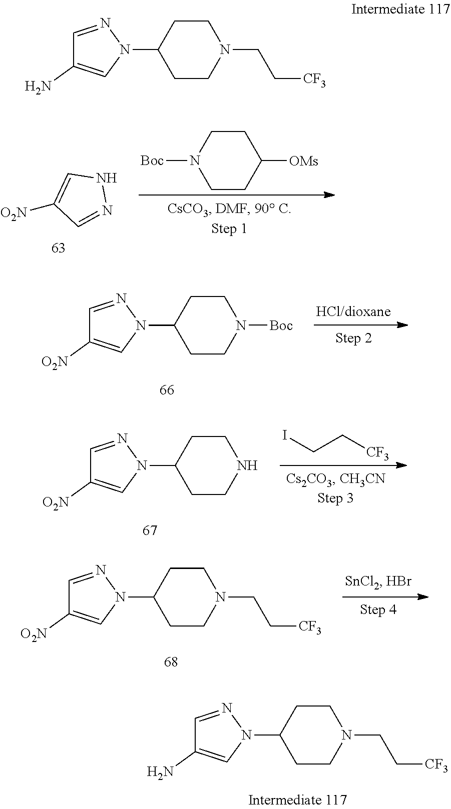

### Step 1: tert-butyl 4-(4-nitro-1H-pyrazol-1-yl)piperidine-1-carboxylate

4-Nitropyrazole (1.0 g, 8.8 mmol, 1.0 equiv.) and Cs₂CO₃ (5.8 g, 17.7 mmol, 2.0 equiv.) were dissolved in DMF (20 mL), tert-butyl 4-(methanesulfonyloxy)piperidine-1-carboxylate (3.7 g, 13.3 mmol, 1.5 equiv.) was added. The reaction mixture was heated to 90° C. for 4 hours, then cooled to ambient temperature and quenched by the addition of water. The resulting solution was extracted with ethyl acetate, washed with brine, dried over anhydrous sodium sulfate and concentrated under vacuum. The residue was purified by flash column chromatography on silica gel column, eluting with ethyl acetate/petroleum ether (1:1) to give tert-butyl 4-(4-nitro-1H-pyrazol-1-yl)piperidine-1-carboxylate (1.5 g) as a white solid. LCMS Method D: [M+H]⁺=297.

### Step 2: 4-(4-nitro-1H-pyrazol-1-yl)piperidine

tert-Butyl 4-(4-nitropyrazol-1-yl)piperidine-1-carboxylate (1.5 g, 5.1 mmol, 1.0 equiv.) was dissolved in HCl (4M in 1,4-dioxane, 15 mL). The resulting solution was stirred for 1 hour at ambient temperature and concentrated under vacuum to give 4-(4-nitro-1H-pyrazol-1-yl)piperidine hydrochloride (1.5 g) as a brown solid. LCMS Method D: [M+H]⁺=197.

### Step 3: 4-(4-nitropyrazol-1-yl)-1-(3,3,3-trifluoropropyl)piperidine

4-(4-nitropyrazol-1-yl)piperidine hydrochloride (1.5 g, 7.6 mmol, 1.0 equiv.) and 1,1,1-trifluoro-3-iodopropane (5.1 g, 22.9 mmol, 3.0 equiv.) were dissolved in ACN (40 mL), Cs₂CO₃ (12.5 g, 38.2 mmol, 5.0 equiv.) was added. The reaction mixture was heated to 50° C., then cooled to ambient temperature, filtrated out the solid and the solution was concentrated under vacuum. The residue was purified by flash column chromatography on silica gel column, eluting with ethyl acetate/petroleum ether (1:1) to give 4-(4-nitropyrazol-1-yl)-1-(3,3,3-trifluoropropyl)piperidine (1.2 g) as a colorless oil. LCMS Method A: [M+H]⁺=293.

### Step 4: 1-(1-(3,3,3-trifluoropropyl)piperidin-4-yl)-1H-pyrazol-4-amine

4-(4-Nitropyrazol-1-yl)-1-(3,3,3-trifluoropropyl)piperidine (500.0 mg, 1.7 mmol, 1.0 equiv.) was dissolved in HBr (40%, 15 mL) and cooled to 0° C., then SnCl₂.2H₂O (772.1 mg, 3.4 mmol, 2.0 equiv.) was added, maintaining the solution at 0° C. The resulting solution was stirred for 2 hours at ambient temperature and concentrated under vacuum.

The residue was diluted with water and adjusted to pH 9 with aqueous NaOH (4 M). The resulting mixture was extracted with ethyl acetate, washed with brine and concentrated under vacuum. The residue was purified by flash column chromatography on silica gel, eluting with ethyl acetate/petroleum ether (1:3) to give. 1-(1-(3,3,3-trifluoropropyl)piperidin-4-yl)-1H-pyrazol-4-amine (230.0 mg) as a white solid. LCMS Method A: [M+H]⁺=263.

### Synthesis of Intermediate 118 (1-(1-(3,3,3-trifluoropropyl)piperidin-4-yl)-1H-pyrazol-4-amine)

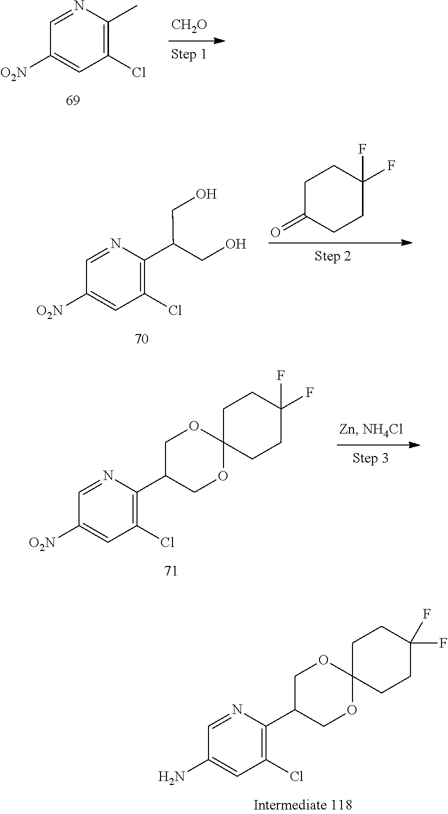

### Step 1: 2-(3-chloro-5-nitropyridin-2-yl)propane-1,3-diol

3-chloro-2-methyl-5-nitropyridine (14.0 g, 81.1 mmol, 1.0 equiv.) was dissolved in formaldehyde aqueous (37-40% wt., 50 mL). The reaction mixture was heated to 130° C. for 3 days. After filtration to remove the solid, the filtrate was concentrated under vacuum. The residue was purified by flash column chromatography on silica gel column, eluting with ethyl acetate/petroleum ether (1:1) to give the crude product, which was further purified by reverse flash chromatography with the following conditions: column, C18 silica gel; mobile phase, MeOH in water, 10% MeOH to 50% gradient in 20 min; detector, UV 254 nm. This resulted in 2-(3-chloro-5-nitropyridin-2-yl)propane-1,3-diol (1.1 g) as an off-white solid. LCMS Method C: [M+H]⁺=233.

### Step 2: 3-chloro-2-[9,9-difluoro-1,5-dioxaspiro[5.5]undecan-3-yl]-5-nitropyridine

2-(3-Chloro-5-nitropyridin-2-yl)propane-1,3-diol (200.0 mg, 0.9 mmol, 1.0 equiv.) and 4,4-difluorocyclohexan-1-one (115.3 mg, 0.9 mmol, 1.0 equiv.) were dissolved in DCM (30 mL), PTSA (29.6 mg, 0.2 mmol, 0.2 equiv.) was added. The reaction mixture was stirred for 1.5 hours at ambient temperature and then concentrated under vacuum. The residue was purified by flash column chromatography on silica gel column, eluting with ethyl acetate/petroleum ether (1:1) to give 3-chloro-2-[9,9-difluoro-1,5-dioxaspiro[5.5]undecan-3-yl]-5-nitropyridine (150.0 mg) as an off-white solid. LCMS Method A: [M+H]⁺=349.

### Step 3: 5-chloro-6-[9,9-difluoro-1,5-dioxaspiro[5.5]undecan-3-yl]pyridin-3-amine

Zn (131.3 mg, 2.0 mmol, 7.0 equiv.) and NH₄Cl (153.4 mg, 2.9 mmol, 10.0 equiv.) were dissolved in water (20 mL), after stirred for 10 min, a solution of 3-chloro-2-[9,9-difluoro-1,5-dioxaspiro[5.5]undecan-3-yl]-5-nitropyridine (100.0 mg, 0.3 mmol, 1.0 equiv.) in MeOH (3 mL) was added dropwise. The reaction mixture was stirred for 2.5 hours at ambient temperature, then filtrated out the solid and the solution was concentrated under vacuum. The residue was purified by flash column chromatography on silica gel column, eluting with ethyl acetate/petroleum ether (1:1) to give 5-chloro-6-[9,9-difluoro-1,5-dioxaspiro[5.5]undecan-3-yl]pyridin-3-amine (60.0 mg) as a white solid. LCMS Method E: [M+H]⁺=319.

The following intermediates were prepared using the method described for Intermediate 118.

| Intermediate | Starting material | Structure | LCMS data |
|---|---|---|---|
| Intermediate 119 | 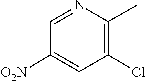 | 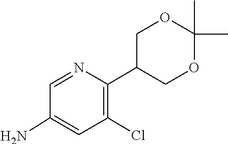 [SMILES: CC1(C)OCC(c2ncc(N)cc2Cl)CO1] | Method C: MS-ESI: 243 [M + H]⁺ |
| Intermediate 120 | 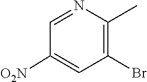 | 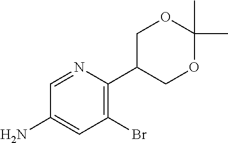 [SMILES: CC1(C)OCC(c2ncc(N)cc2Br)CO1] | Method C: MS-ESI: 287 [M + H]⁺ |

### Synthesis of Intermediate 121 (5-chloro-6-(4,4-difluorocyclohexyl)-N-(2-methoxyethyl)pyridin-3-amine)

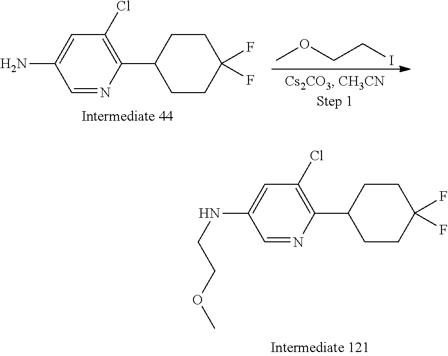

5-Chloro-6-(4,4-difluorocyclohexyl)pyridin-3-amine (300.0 mg, 1.2 mmol, 1.0 equiv.) and Cs₂CO₃ (792.5 mg, 2.4 mmol, 2.0 equiv.) were dissolved in DMF (10 mL), 1-iodo-2-methoxyethane (1583.3 mg, 8.5 mmol, 7.0 equiv.) was added. The reaction mixture was heated to 100° C. overnight, then cooled to ambient temperature and quenched by the addition of water. The resulting solution was extracted with ethyl acetate, dried over anhydrous Na₂SO₄ and concentrated under vacuum. The residue was purified by flash column chromatography on silica gel, eluting with DCM/MeOH (10:1) to give 5-chloro-6-(4,4-difluorocyclohexyl)-N-(2-methoxyethyl)pyridin-3-amine (195.2 mg) as a brown solid. LCMS Method E: [M+H]⁺=305.1.

The following intermediates were prepared using the same method described for Intermediate 121.

| Intermediate | Starting material | Structure | LCMS data |
|---|---|---|---|
| Intermediate 122 | 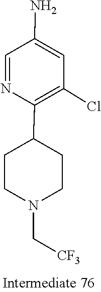 [SMILES: Nc1cnc(C2CCN(CC(F)(F)F)CC2)c(Cl)c1] | 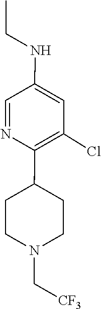 [SMILES: CCNc1cnc(C2CCN(CC(F)(F)F)CC2)c(Cl)c1] | Method A: MS-ESI: 322 [M + H]⁺ |
| Intermediate 123 | 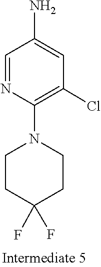 [SMILES: Nc1cnc(N2CCC(F)(F)CC2)c(Cl)c1] | 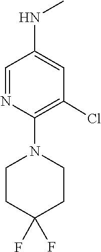 [SMILES: CNc1cnc(N2CCC(F)(F)CC2)c(Cl)c1] | Method A: MS-ESI: 262 [M + H]⁺ |
| Intermediate 123a | 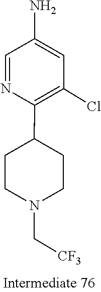 [SMILES: Nc1cnc(C2CCN(CC(F)(F)F)CC2)c(Cl)c1] | 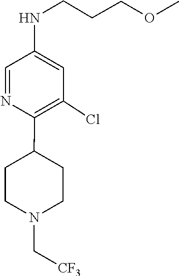 [SMILES: COCCCNc1cnc(C2CCN(CC(F)(F)F)CC2)c(Cl)c1] | Method A: MS-ESI: 366 [M + H]⁺ |

### Synthesis of Intermediate 124 (3-chloro-4-(3,3-difluorocyclobutyl)aniline)

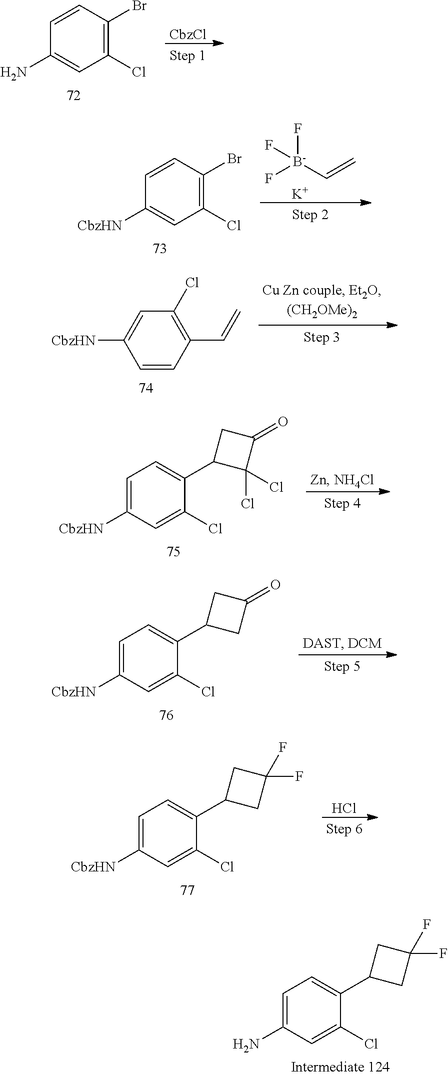

### Step 1: Benzyl N-(4-bromo-3-chlorophenyl)carbamate

4-Bromo-3-chloroaniline (10.0 g, 48.4 mmol, 1.0 equiv.) was dissolved in THE (100 mL) and water (20 mL), then K₂CO₃ (13.4 g, 96.9 mmol, 2.0 equiv.) and CbzCl (12.4 g, 72.7 mmol, 1.5 equiv.) were added. The resulting solution was stirred for 12 hours at ambient temperature and quenched by the addition of water. The resulting solution was extracted with ethyl acetate, dried over anhydrous Na₂SO₄ and concentrated under vacuum to give benzyl N-(4-bromo-3-chlorophenyl)carbamate (15.2 g) as a white solid. LCMS Method A: [M+H]⁺=340.

### Step 2: benzyl N-(3-chloro-4-ethenylphenyl)carbamate

Benzyl N-(4-bromo-3-chlorophenyl)carbamate (1.0 g, 2.9 mmol, 1.0 equiv.) were dissolved in 1,4-dioxane/water (20/4 mL), then Cs₂CO₃ (1.9 g, 5.9 mmol, 2.0 equiv.), potassium trifluoro(vinyl)borate (0.59 g, 4.4 mmol, 1.5 equiv.) and Pd(PPh₃)₄ (0.3 g, 0.3 mmol, 0.1 equiv.) were added under an atmosphere of nitrogen. The reaction mixture was heated to 90° C. for 12 hours, then cooled to ambient temperature and concentrated under vacuum. The residue was purified by flash column chromatography on silica gel column, eluting with ethyl acetate/petroleum ether (1:12) to give benzyl N-(3-chloro-4-ethenylphenyl)carbamate (0.6 g) as an off-white solid. LCMS Method D: [M+H]⁺=288.

### Step 3: Benzyl N-[3-chloro-4-(2,2-dichloro-3-oxocyclobutyl)phenyl]carbamate

Benzyl N-(3-chloro-4-ethenylphenyl)carbamate (35.0 g, 121.6 mmol, 1.0 equiv.) was dissolved in Et₂O (100 mL) and DME (20 mL), then trichloroacetyl chloride (33.2 g, 182.4 mmol, 1.5 equiv.) and, Zn—Cu (35.0 g, 271.3 mmol, 2.2 equiv.). The reaction was heated to 50° C. for 12 hours, then cooled to ambient temperature and quenched by the addition of water. After removing the solid by filtration, the filtrate was adjusted to pH 7 with NaOH aqueous (2N). The resulting solution was extracted with ethyl acetate, washed with brine and concentrated under vacuum. The residue was purified by flash column chromatography on silica gel column, eluting with ethyl acetate/petroleum ether (1:15) to give benzyl N-[3-chloro-4-(2,2-dichloro-3-oxocyclobutyl)phenyl]carbamate (10.3 g) as a yellow solid. LCMS Method A: [M+H]⁺=398.

### Step 4: Benzyl N-[3-chloro-4-(3-oxocyclobutyl)phenyl]carbamate

Benzyl N-[3-chloro-4-(2,2-dichloro-3-oxocyclobutyl)phenyl]carbamate (10.0 g, 25.1 mmol, 1.0 equiv.) was dissolved in THE (100 mL) and water (20 mL), then NH4C1 (2.7 g, 50.2 mmol, 2.0 equiv.) and Zn (3.3 g, 50.5 mmol, 2.0 equiv.) were added. The reaction mixture was heated to 70° C. for 12 hours. After cooled to ambient temperature and filtration, the resulting solution was extracted with ethyl acetate, dried over anhydrous sodium sulfate and concentrated under vacuum to give benzyl N-[3-chloro-4-(3-oxocyclobutyl)phenyl]carbamate (6.1 g) as a white solid. LCMS Method C: [M+H]⁺=330.

### Step 5: Benzyl N-[3-chloro-4-(3,3-difluorocyclobutyl)phenyl]carbamate

Benzyl N-[3-chloro-4-(3-oxocyclobutyl)phenyl]carbamate (10.0 g, 30.3 mmol, 1.0 equiv.) was dissolved in DCM (100 mL) and cooled to 0° C., then DAST (9.8 g, 60.7 mmol, 2.0 equiv.) was added dropwise. The reaction mixture was stirred for 12 hours at 0° C. and then quenched by the addition of ice-water. The resulting solution was extracted with DCM, dried over anhydrous sodium sulfate and concentrated under vacuum. The residue was purified by flash column chromatography on silica gel column, eluting with ethyl acetate/petroleum ether (1:10) to give benzyl N-[3-chloro-4-(3,3-difluorocyclobutyl)phenyl]carbamate (4.2 g) as a yellow oil. LCMS Method C: [M+H]⁺=352.

### Step 6: 3-chloro-4-(3,3-difluorocyclobutyl)aniline

Benzyl N-[3-chloro-4-(3,3-difluorocyclobutyl)phenyl]carbamate (1.0 g, 2.8 mmol, 1.0 equiv.) was dissolved in conc. HCl (10 mL). The resulting solution was heated to 70° C. for 12 hours, then cooled to ambient temperature and diluted with water. The solution was adjusted to pH 8 with NaOH aqueous (20%), extracted with ethyl acetate, washed with brine, dried over anhydrous Na₂SO₄ and concentrated under vacuum. The residue was purified by flash column chromatography on silica gel column, eluting with ethyl acetate/petroleum ether (1:10) to give 3-chloro-4-(3,3-difluorocyclobutyl)aniline (0.3 g) as a yellow solid. LCMS Method A: [M+H]⁺=218.

### Synthesis of Intermediate 127 (6-(4-(3,3,3-trifluoropropyl)piperazin-1-yl)pyridin-3-amine)

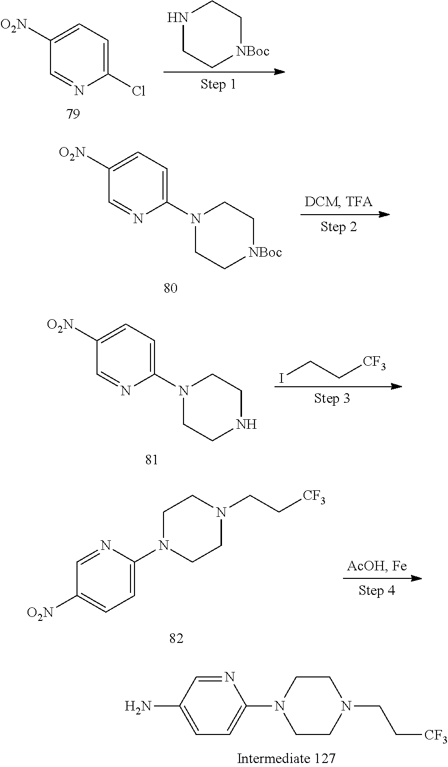

### Step 1: tert-butyl 4-(5-nitropyridin-2-yl)piperazine-1-carboxylate

2-Chloro-5-nitropyridine (2.0 g, 12.6 mmol, 1.0 equiv.) was dissolved in DMF (20 mL), Cs₂CO₃ (8.2 g, 25.2 mmol, 2.0 equiv.) and tert-butyl piperazine-1-carboxylate (2.4 g, 12.6 mmol, 1.0 equiv.) were added. The reaction mixture was heated to 90° C. for 5 hours, the cooled to ambient temperature and quenched by the addition of water. The resulting solution was extracted with ethyl acetate, washed with brine, dried over anhydrous Na₂SO₄ and concentrated under vacuum. The residue was purified by flash column chromatography on silica gel column, eluting with ethyl acetate/petroleum ether (1:1) to give tert-butyl 4-(5-nitropyridin-2-yl)piperazine-1-carboxylate (1.8 g) as an off-white solid. LCMS Method F: [M+H]⁺=309.

### Step 2: 1-(5-nitropyridin-2-yl)piperazine

tert-Butyl 4-(5-nitropyridin-2-yl)piperazine-1-carboxylate (1.7 g, 5.5 mmol, 1.0 equiv.) was dissolved in DCM (20 mL), TFA (3.1 g, 27.5 mmol, 5.0 equiv.) was added. The reaction mixture was stirred for 3 hours at ambient temperature and concentrated under vacuum. The residue was dissolved in water and adjusted to pH 7 with NaOH aqueous (3 mol/L). The resulting solution was extracted with ethyl acetate, washed with brine, dried over anhydrous Na₂SO₄ and concentrated under vacuum to give 1-(5-nitropyridin-2-yl)piperazine (910.0 mg) as an off-white solid. LCMS Method C: [M+H]⁺=209.

### Step 3: 1-(5-nitropyridin-2-yl)-4-(3,3,3-trifluoropropyl)piperazine

1-(5-Nitropyridin-2-yl)piperazine (1.7 g, 8.2 mmol, 1.0 equiv.) was dissolved in ACN (20 mL), Cs₂CO₃ (5320.3 mg, 16.3 mmol, 2.0 equiv.) and 1,1,1-trifluoro-3-iodopropane (1.8 g, 8.2 mmol, 1.0 equiv.) were added. The reaction mixture was heated to 50° C. for 3 hours, then cooled to ambient temperature, filtrated and concentrated under vacuum. The residue was purified by flash column chromatography on silica gel column, eluting with ethyl acetate/petroleum ether (1:1) to give 1-(5-nitropyridin-2-yl)-4-(3,3,3-trifluoropropyl)piperazine (1.1 g) as an off-white solid. LCMS Method A: [M+H]⁺=305.

### Step 4: 6-[4-(3,3,3-trifluoropropyl)piperazin-1-yl]pyridin-3-amine

1-(5-Nitropyridin-2-yl)-4-(3,3,3-trifluoropropyl)piperazine (800.0 mg, 2.6 mmol, 1.0 equiv.) was dissolved in AcOH (8 mL), Fe (293.7 mg, 5.3 mmol, 2.0 equiv.) was added. The reaction mixture was heated to 90° C. for 5 hours, then cooled to ambient temperature, filtrated our the solid, and the filtrate was concentrated under vacuum to give 6-[4-(3,3,3-trifluoropropyl)piperazin-1-yl]pyridin-3-amine (685.5 mg) as an off-white solid. LCMS Method C: [M+H]⁺=275.

### Synthesis of Intermediate 129 (4,4-difluorocyclohexyl methanesulfonate)

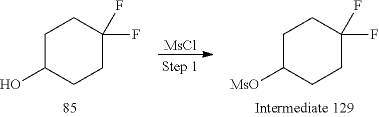 [SMILES: "MsCl" ; OC1CCC(F)(F)CC1 ; CS(=O)(=O)OC1CCC(F)(F)CC1]

4,4-Difluorocyclohexan-1-ol (2.5 g, 18.4 mmol, 1.0 equiv.) and TEA (7.6 mL, 55.1 mmol, 3.0 equiv.) were dissolved in DCM (80 mL) and cooled to 0° C., MsCl (2.8 mL, 36.7 mmol, 2.0 equiv.) was added dropwise under an atmosphere of nitrogen, maintaining the solution at 0° C. The reaction mixture was stirred for 1 hour at 0° C. and quenched by the addition of water. The organic layer was separated, washed with brine, dried over anhydrous Na₂SO₄ and concentrated under vacuum to give 4,4-difluorocyclohexyl methanesulfonate (3.8 g) as light yellow oil.

The following intermediates were prepared using the same method described for Intermediate 129.

| Intermediate | Starting material | Structure |
|---|---|---|
| Intermediate 130 | 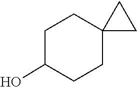 | 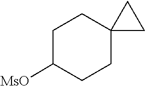 [SMILES: CS(=O)(=O)OC1CCC2(CC1)CC2] |
| Intermediate 131 | 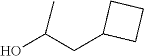 | 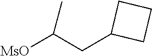 |

### Synthesis of Intermediate 132 (4-chloro-5-(4,4-difluoropiperidin-1-yl)pyridin-2-amine)

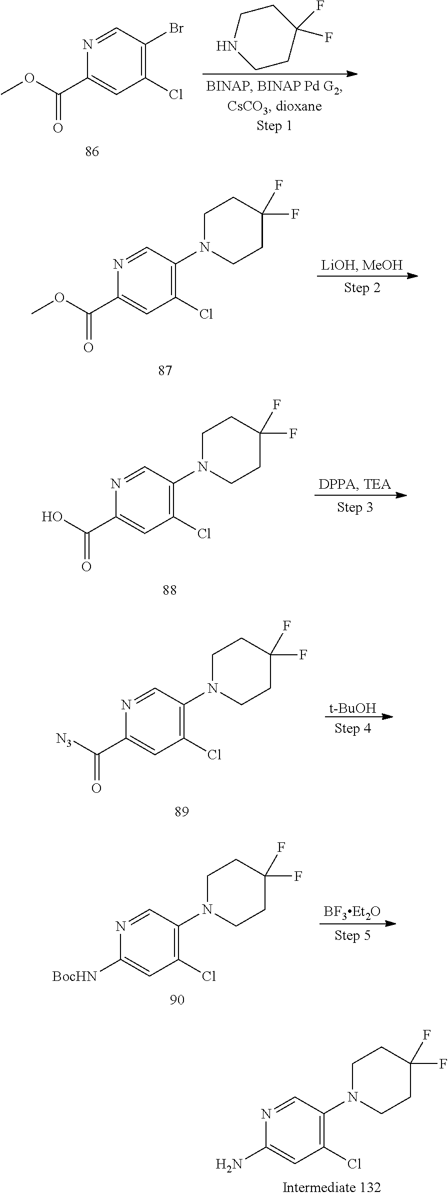

### Step 1: methyl 4-chloro-5-(4,4-difluoropiperidin-1-yl)picolinate

Methyl 5-bromo-4-chloropyridine-2-carboxylate (1.0 g, 3.9 mmol, 1.0 equiv.) was dissolved dioxane (10 mL), then Cs₂CO₃ (2.6 g, 7.9 mmol, 2.0 equiv.), BINAP (248.5 mg, 0.4 mmol, 0.1 equiv.), Binap Palladacycle Gen. 2 (0.3 mg, 0.1 equiv.) and 4,4-difluoropiperidine (967.2 mg, 7.9 mmol, 2.0 equiv.) were added under an atmosphere of nitrogen. The resulting solution was heated to 100° C. for 7 hours, then cooled to ambient temperature and concentrated under vacuum. The residue was purified by flash column chromatography on silica gel, eluting with ethyl acetate/petroleum ether (1:3) to give methyl 4-chloro-5-(4,4-difluoropiperidin-1-yl)pyridine-2-carboxylate (711.2 mg) as a yellow solid. LCMS Method A: [M+H]⁺=291.

### Step 2: 4-chloro-5-(4,4-difluoropiperidin-1-yl)pyridine-2-carboxylic acid

Methyl 4-chloro-5-(4,4-difluoropiperidin-1-yl)pyridine-2-carboxylate (700.0 mg, 2.4 mmol, 1.0 equiv.) was dissolved MeOH (5 mL) and water (2 mL), then LiGH (288.3 mg, 12.0 mmol, 5.0 equiv.) was added. The reaction mixture was stirred for 3 hours at ambient temperature and then concentrated under vacuum. The residue was diluted with water, then the solution was adjusted to pH 5 with aqueous HCl (3 M). The solids were collected by filtration and dried to give 4-chloro-5-(4,4-difluoropiperidin-1-yl)pyridine-2-carboxylic acid (500.0 mg) as an off-white solid. LCMS Method A: [M−H]⁻=275.

### Step 3: 4-chloro-5-(4,4-difluoropiperidin-1-yl)picolinoyl azide

4-Chloro-5-(4,4-difluoropiperidin-1-yl)pyridine-2-carboxylic acid (450.0 mg, 1.6 mmol, 1.0 equiv.) was dissolved THE (5 mL), then TEA (0.5 mL, 3.5 mmol, 2.2 equiv.), DPPA (671.4 mg, 2.4 mmol, 1.5 equiv.) were added. The resulting mixture was stirred for 6 hours at ambient temperature and then concentrated under vacuum. This resulted in 4-chloro-5-(4,4-difluoropiperidin-1-yl)picolinoyl azide (350.0 mg) as an off-white solid. LCMS Method C: [M+H]⁺=302.

### Step 4: tert-butyl (4-chloro-5-(4,4-difluoropiperidin-1-yl)pyridin-2-yl)carbamate

4-Chloro-5-(4,4-difluoropiperidin-1-yl)pyridine-2-carbonyl azide (300.0 mg, 0.9 mmol, 1.0 equiv.) was dissolved t-BuOH (3 mL). The resulting solution was heated to 90° C. for 3 hours and then concentrated under vacuum. The residue was purified by flash column chromatography on silica gel, eluting with ethyl acetate/petroleum ether (1:3) to give tert-butyl (4-chloro-5-(4,4-difluoropiperidin-1-yl)pyridin-2-yl)carbamate (250.0 mg) of as an off-white solid. LCMS Method C: [M+H]⁺=348.

### Step 5: 4-chloro-5-(4,4-difluoropiperidin-1-yl)pyridin-2-amine

tert-Butyl [4-chloro-5-(4,4-difluoropiperidin-1-yl)pyridin-2-yl]carbamate (250.0 mg, 0.7 mmol, 1.0 equiv.) was dissolved in BF₃.Et₂O (3.0 mL). The resulting solution was stirred for 3 hours at ambient temperature and then quenched by the addition of water. The resulting solution was adjusted to pH 7 with aqueous NaOH (3 M). The resulting solution was extracted with DCM, washed with brine, dried over anhydrous Na₂SO₄ and concentrated under vacuum to give 4-chloro-5-(4,4-difluoropiperidin-1-yl)pyridin-2-amine (180.0 mg) as an off-white solid. LCMS Method C: [M+H]⁺=248.

### Example 1: Synthesis of Compound 101

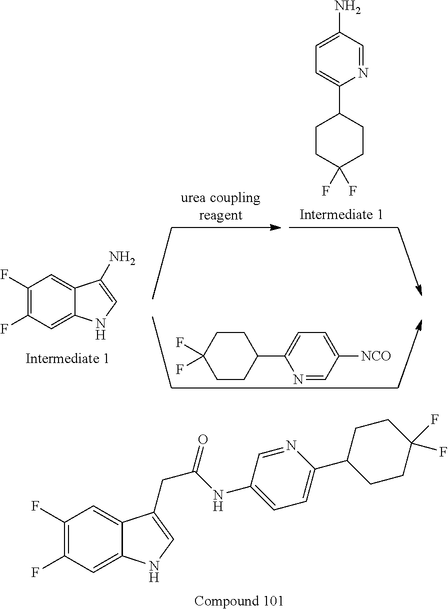

The scheme above illustrates exemplary methods for synthesizing compound 101. Intermediate 1 is treated with a urea coupling agent under basic conditions. Reaction of the resulting intermediate with Intermediate 2 affords compound 101. Alternatively, isocyanate of intermediate 3 prepared by methods well known in the art (e.g., from intermediate 2) is treated with Intermediate 1 (e.g., under basic conditions) to afford compound 101.

Compounds 102-122 are synthesized using methods similar to Example 1, above.

### Example 2: 1-(5-chloro-1H-indol-3-yl)-3-(5-chloro-6-(4,4-difluoropiperidin-1-yl)pyridin-3-yl)urea (Compound 196)

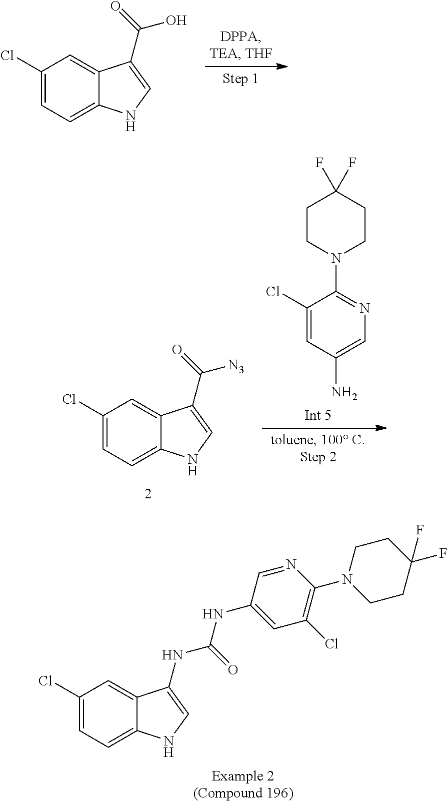 [SMILES: O=C(O)c1c[nH]c2ccc(Cl)cc12 ; [N-]=[N+]=NC(=O)c1c[nH]c2ccc(Cl)cc12 ; Nc1cnc(N2CCC(F)(F)CC2)c(Cl)c1 ; O=C(Nc1cnc(N2CCC(F)(F)CC2)c(Cl)c1)Nc1c[nH]c2ccc(Cl)cc12]

### Step 1: 5-chloro-1H-indole-3-carbonyl azide

5-chloro-1H-indole-3-carboxylic acid (10.0 g, 51.3 mmol, 1.0 equiv.) was dissolved in THE (150 mL), then TEA (15.5 g, 153.9 mmol, 3.0 equiv.) and DPPA (42.3 g, 153.9 mmol, 3.0 equiv.) were added. The reaction mixture was stirred overnight at rt. The reaction was quenched by addition of 200 mL of ice/water. The desired product was precipitated and collected by filtration. This resulted in 5-chloro-1H-indole-3-carbonyl azide as an off-white solid. MS-ESI: 221 [M+H]+.

### Step 2: 1-(5-chloro-1H-indol-3-yl)-3-(5-chloro-6-(4,4-difluoropiperidin-1-yl)pyridin-3-yl)urea

5-chloro-6-(4,4-difluoropiperidin-1-yl)pyridin-3-amine (Int5) (4.0 g, 16.2 mmol, 1.0 equiv.) and 5-chloro-1H-indole-3-carbonyl azide (4.3 g, 19.4 mmol, 1.2 equiv.) were dissolved in toluene (50 mL), then TEA (3.3 g, 32.4 mmol, 2.0 equiv.) was added. The reaction mixture was heated to 90° C. for 16 hours and then cooled to room temperature. The desired product was precipitated and collected by filtration. The crude product was further recrystallized from CH₃CN. 1-(5-chloro-1H-indol-3-yl)-3-(5-chloro-6-(4,4-difluoropiperidin-1-yl)pyridin-3-yl)urea was isolated as a white solid.

MS-ESI: 440 [M+H]+.

¹H-NMR (400 MHz, DMSO-d₆) δ: 11.01 (s, 1H), 8.68 (s, 2H), 8.25 (d, J=2.4 Hz, 1H), 8.16 (d, J=2.4 Hz, 1H), 7.56 (d, J=1.6 Hz, 1H), 7.55 (d, J=2.4 Hz, 1H), 7.37 (d, J=8.4 Hz, 1H), 7.10 (dd, J=8.4, 2.4 Hz, 1H), 3.30-3.27 (m, 4H), 2.16-2.06 (m, 4H). Note: 5-chloro-6-(4,4-difluoropiperidin-1-yl)pyridin-3-amine (Int 5) was obtained using the following steps:

### Step 1: 3-chloro-2-(4,4-difluoropiperidin-1-yl)-5-nitropyridine

2,3-dichloro-5-nitropyridine (5.0 g, 26.1 mmol, 1.0 equiv.), 4,4-difluoropiperidine hydrochloride (4.5 g, 28.7 mmol, 1.1 equiv.) and Cs₂CO₃ (21.3 g, 65.3 mmol, 2.5 equiv.) were dissolved in DMF (70 mL). The reaction mixture was stirred overnight at 90° C. and then quenched by the addition of water. The resulting mixture was extracted with EtOAc, washed with brine, then dried over anhydrous Na₂SO₄ and concentrated under vacuum. This resulted in crude 3-chloro-2-(4,4-difluoropiperidin-1-yl)-5-nitropyridine as a yellow solid. MS-ESI: 278 [M+H]+.

### Step 2: 5-chloro-6-(4,4-difluoropiperidin-1-yl)pyridin-3-amine

3-chloro-2-(4,4-difluoropiperidin-1-yl)-5-nitropyridine (6.9 g, 24.9 mmol, 1.0 equiv.) was dissolved in aq. HBr (40%, 40 mL), then SnCl₂ (14.2 g, 74.7 mmol, 3.0 equiv.) was added. The resulting mixture was heated to 70° C. for 2 h, then cooled to room temperature and quenched by the addition of water. The resulting mixture was extracted with EtOAc, washed with brine, then dried over anhydrous Na₂SO₄ and concentrated under vacuum. The residue was purified by flash column chromatography on silica gel, eluting with ethyl acetate/petroleum ether (1:3) to give 5-chloro-6-(4,4-difluoropiperidin-1-yl)pyridin-3-amine as a dark green solid. MS-ESI: 248 [M+H]+.

The following examples were prepared using the method described for Example 2.

| | Starting materials | | |
|---|---|---|---|
| Example # | Used | Structure | LCMS |
| Example 3 (Compound 123) | 5,6-difluoro-1H- indole-3-carboxylic acid; Intermediate 31 | 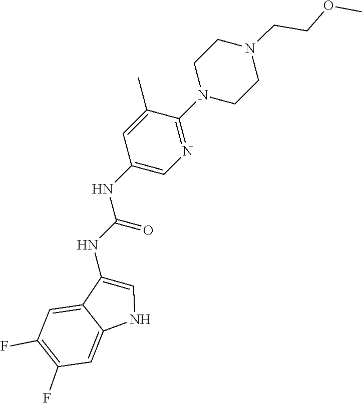 [SMILES: COCCN1CCN(c2ncc(NC(=O)Nc3c[nH]c4cc(F)c(F)cc34)cc2C)CC1] | Method D: MS-ESI: 445 [M + H]⁺. |
| | | 1-(5,6-difluoro-1H-indol- | |
| | | 3-yl)-3-(6-(4-(2- | |
| | | methoxyethyl)piperazin-1- | |
| | | yl)-5-methylpyridin-3- | |
| | | yl)urea | |
| Example 4 Compound 124) | 5-chloro-1H-indole- 3-carboxylic acid; Intermediate 6 | 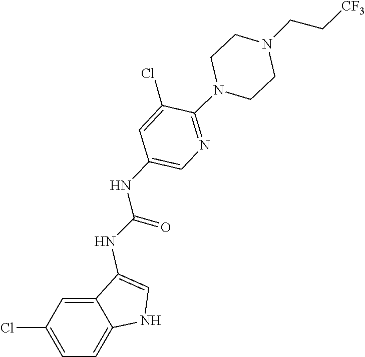 [SMILES: O=C(Nc1cnc(N2CCN(CCC(F)(F)F)CC2)c(Cl)c1)Nc1c[nH]c2ccc(Cl)cc12] | Method D: MS-ESI: 501 [M + H]⁺. |
| | | 1-(5-chloro-1H-indol-3- | |
| | | yl)-3-(5-chloro-6-(4-(3,3,3- | |
| | | trifluoropropyl)piperazin- | |
| | | 1-yl)pyridin-3-yl)urea | |
| Example 5 (Compound 125) | 5,6-difluoro-1H- indole-3-carboxylic acid; Intermediate 56 | 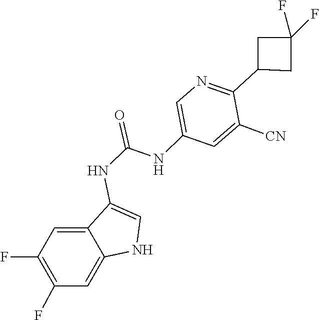 [SMILES: N#Cc1cc(NC(=O)Nc2c[nH]c3cc(F)c(F)cc23)cnc1C1CC(F)(F)C1] | Method D: MS-ESI: 404 [M + H]⁺. |
| | | 1-(5-cyano-6-(3,3- | |
| | | difluorocyclobutyl)pyridin- | |
| | | 3-yl)-3-(5,6-difluoro-1H- | |
| | | indol-3-yl)urea | |
| Example 6 (Compound 126) | 5-chloro-1H-indole- 3-carboxylic acid; Intermediate 7 | 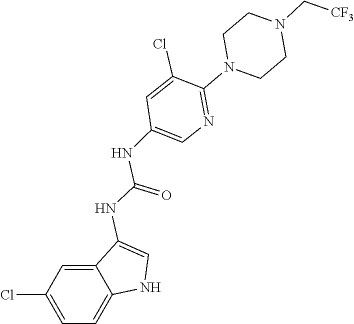 [SMILES: O=C(Nc1cnc(N2CCN(CC(F)(F)F)CC2)c(Cl)c1)Nc1c[nH]c2ccc(Cl)cc12] | Method D: MS-ESI: 487 [M + H]⁺. |
| | | 1-(5-chloro-1H-indol-3- | |
| | | yl)-3-(5-chloro-6-(4-(2,2,2- | |
| | | trifluoroethyl)piperazin-1- | |
| | | yl)pyridin-3-yl)urea | |
| Example 7 (Compound 127) | 5-chloro-1H-indole- 3-carboxylic acid; Intermediate 53 | 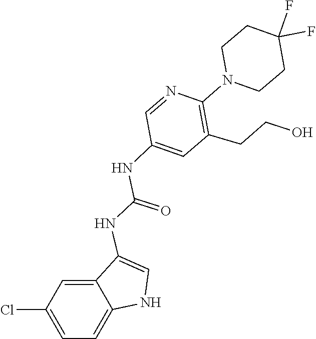 [SMILES: O=C(Nc1cnc(N2CCC(F)(F)CC2)c(CCO)c1)Nc1c[nH]c2ccc(Cl)cc12] | Method C: MS-ESI: 450 [M + H]⁺. |
| | | 1-(5-chloro-1H-indol-3- | |
| | | yl)-3-(6-(4,4- | |
| | | difluoropiperidin-1-yl)-5- | |
| | | (2-hydroxyethyl)pyridin-3- | |
| | | yl)urea | |
| Example 8 (Compound 128) | Intermediate 61; Intermediate 8 | 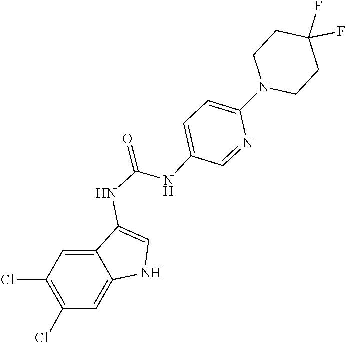 [SMILES: O=C(Nc1ccc(N2CCC(F)(F)CC2)nc1)Nc1c[nH]c2cc(Cl)c(Cl)cc12] | Method C: MS-ESI: 440 [M + H]⁺. |
| | | 1-(5,6-dichloro-1H-indol- | |
| | | 3-yl)-3-(6-(4,4- | |
| | | difluoropiperidin-1- | |
| | | yl)pyridin-3-yl)urea | |
| Example 9 (Compound 129) | 5-chloro-1H-indole- 3-carboxylic acid; Intermediate 9 | 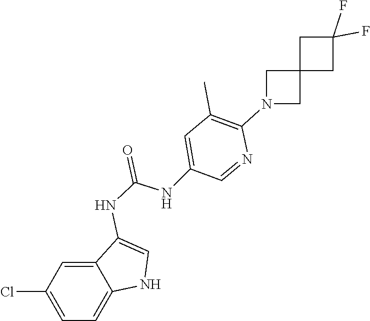 [SMILES: Cc1cc(NC(=O)Nc2c[nH]c3ccc(Cl)cc23)cnc1N1CC2(C1)CC(F)(F)C2] | Method D: MS-ESI: 432 [M + H]⁺. |
| | | 1-(5-chloro-1H-indol-3- | |
| | | yl)-3-(6-(6,6-difluoro-2- | |
| | | azaspiro[3.3]heptan-2-yl)- | |
| | | 5-methylpyridin-3-yl)urea | |
| Example 10 (Compound 130) | 5-chloro-1H-indole- 3-carboxylic acid; Intermediate 43 | 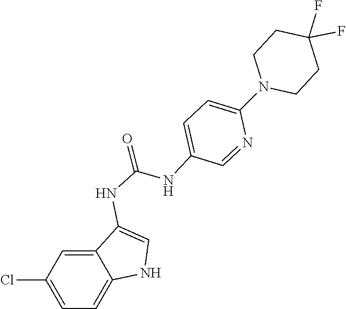 [SMILES: O=C(Nc1ccc(N2CCC(F)(F)CC2)nc1)Nc1c[nH]c2ccc(Cl)cc12] | Method D: MS-ESI: 405 [M + H]⁺. |
| | | 1-(5-chloro-1H-indol-3- | |
| | | yl)-3-(6-(4,4- | |
| | | difluorocyclohexyl)pyridin- | |
| | | 3-yl)urea | |
| Example 11 (Compound 131) | 5-chloro-1H-indole- 3-carboxylic acid; Intermediate 52 | 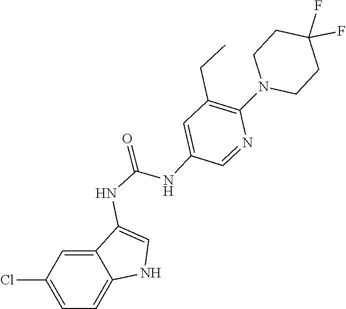 [SMILES: CCc1cc(NC(=O)Nc2c[nH]c3ccc(Cl)cc23)cnc1N1CCC(F)(F)CC1] | Method D: MS-ESI: 434 [M + H]⁺. |
| | | 1-(5-chloro-1H-indol-3- | |
| | | yl)-3-(6-(4,4- | |
| | | difluoropiperidin-1-yl)-5- | |
| | | ethylpyridin-3-yl)urea | |
| Example 12 (Compound 132) | 5-fluoro-1H-indole- 3-carboxylic acid Intermediate 52 | 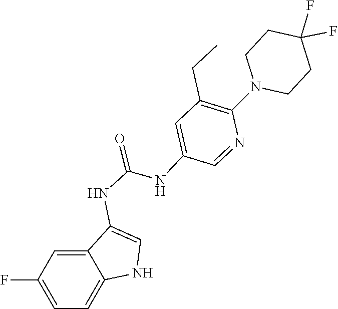 [SMILES: CCc1cc(NC(=O)Nc2c[nH]c3ccc(F)cc23)cnc1N1CCC(F)(F)CC1] | Method D: MS-ESI: 418 [M + H]⁺. |
| | | 1-(6-(4,4- | |
| | | difluoropiperidin-1-yl)-5- | |
| | | ethylpyridin-3-yl)-3-(5- | |
| | | fluoro-1H-indol-3-yl)urea | |
| Example 13 (Compound 133) | 5-chloro-1H-indole- 3-carboxylic acid; Intermediate 46 | 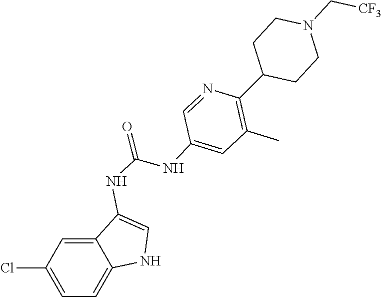 [SMILES: Cc1cc(NC(=O)Nc2c[nH]c3ccc(Cl)cc23)cnc1C1CCN(CC(F)(F)F)CC1] | Method G: MS-ESI: 466 [M + H]⁺. |
| | | 1-(5-chloro-1H-indol-3- | |
| | | yl)-3-(5-methyl-6-(1- | |
| | | (2,2,2- | |
| | | trifluoroethyl)piperidin-4- | |
| | | yl)pyridin-3-yl)urea | |
| Example 14 (Compound 134) | 5,6-difluoro-1H- indole-3-carboxylic acid Intermediate 10 | 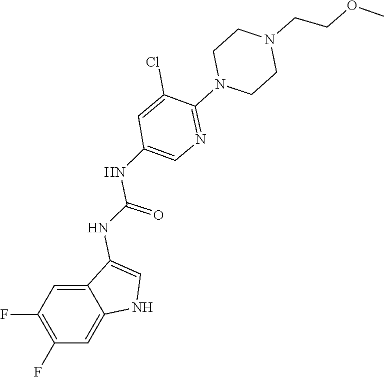 [SMILES: COCCN1CCN(c2ncc(NC(=O)Nc3c[nH]c4cc(F)c(F)cc34)cc2Cl)CC1] | Method D: MS-ESI: 465 [M + H]⁺. |
| | | 1-(5-chloro-6-(4-(2- | |
| | | methoxyethyl)piperazin-1- | |
| | | yl)pyridin-3-yl)-3-(5,6- | |
| | | difluoro-1H-indol-3- | |
| | | yl)urea | |
| Example 15 (Compound 135) | 5-chloro-1H-indole- 3-carboxylic acid; Intermediate 10 | 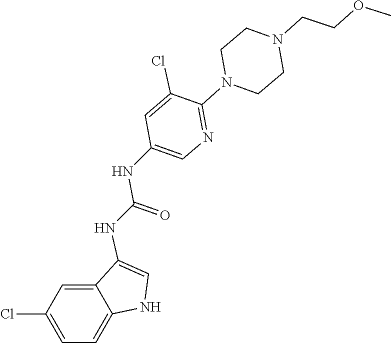 [SMILES: COCCN1CCN(c2ncc(NC(=O)Nc3c[nH]c4ccc(Cl)cc34)cc2Cl)CC1] | Method D: MS-ESI: 463 [M + H]⁺. |
| | | 1-(5-chloro-1H-indol-3- | |
| | | yl)-3-(5-chloro-6-(4-(2- | |
| | | methoxyethyl)piperazin-1- | |
| | | yl)pyridin-3-yl)urea | |
| Example 16 (Compound 136) | 5-chloro-1H-indole- 3-carboxylic acid; Intermediate 54 | 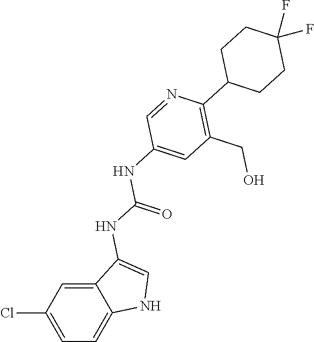 [SMILES: O=C(Nc1cnc(C2CCC(F)(F)CC2)c(CO)c1)Nc1c[nH]c2ccc(Cl)cc12] | Method D: MS-ESI: 435 [M + H]⁺. |
| | | 1-(5-chloro-1H-indol-3- | |
| | | yl)-3-(6-(4,4- | |
| | | difluorocyclohexyl)-5- | |
| | | (hydroxymethyl)pyridin-3- | |
| | | yl)urea | |
| Example 17 (Compound 137) | 5-chloro-1H-indole- 3-carboxylic acid; Intermediate 11 | 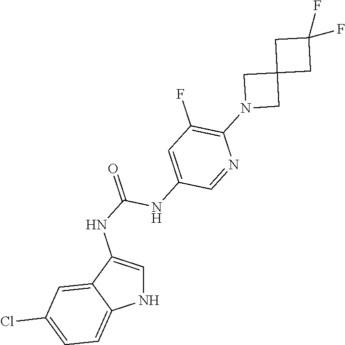 [SMILES: O=C(Nc1cnc(N2CC3(C2)CC(F)(F)C3)c(F)c1)Nc1c[nH]c2ccc(Cl)cc12] | Method D: MS-ESI: 436 [M + H]⁺. |
| | | 1-(5-chloro-1H-indol-3- | |
| | | yl)-3-(6-(6,6-difluoro-2- | |
| | | azaspiro[3.3]heptan-2-yl)- | |
| | | 5-fluoropyridin-3-yl)urea | |
| Example 18 (Compound 138) | 5-chloro-1H-indole- 3-carboxylic acid; Intermediate 12 | 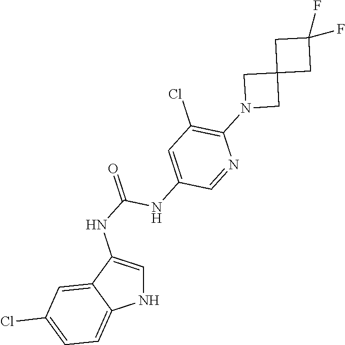 [SMILES: O=C(Nc1cnc(N2CC3(C2)CC(F)(F)C3)c(Cl)c1)Nc1c[nH]c2ccc(Cl)cc12] | Method D: MS-ESI: 452 [M + H]⁺. |
| | | 1-(5-chloro-1H-indol-3- | |
| | | yl)-3-(5-chloro-6-(6,6- | |
| | | difluoro-2- | |
| | | azaspiro[3.3]heptan-2- | |
| | | yl)pyridin-3-yl)urea | |
| Example 19 (Compound 139) | 5-chloro-1H-indole- 3-carboxylic acid; Intermediate 50 | 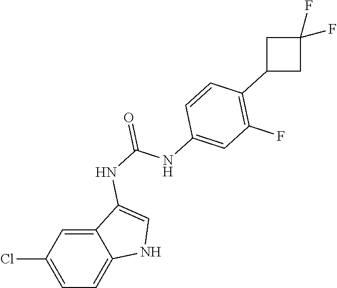 [SMILES: O=C(Nc1ccc(C2CC(F)(F)C2)c(F)c1)Nc1c[nH]c2ccc(Cl)cc12] | Method D: MS-ESI: 394 [M + H]⁺. |
| | | 1-(5-chloro-1H-indol-3- | |
| | | yl)-3-(4-(3,3- | |
| | | difluorocyclobutyl)-3- | |
| | | fluorophenyl)urea | |
| Example 20 (Compound 140) | 6-chloro-1H-indole- 3-carboxylic acid Intermediate 44 | 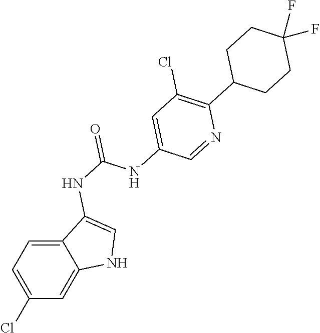 [SMILES: O=C(Nc1cnc(C2CCC(F)(F)CC2)c(Cl)c1)Nc1c[nH]c2cc(Cl)ccc12] | Method D: MS-ESI: 439 [M + H]⁺. |
| | | 1-(6-chloro-1H-indol-3- | |
| | | yl)-3-(5-chloro-6-(4,4- | |
| | | difluorocyclohexyl)pyridin- | |
| | | 3-yl)urea | |
| Example 21 (Compound 141) | 6-fluoro-1H-indole- 3-carboxylic acid Intermediate 44 | 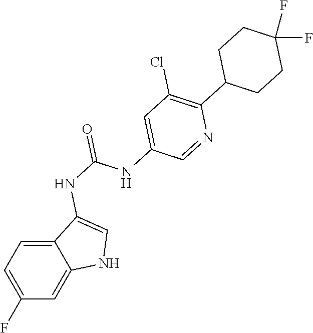 [SMILES: O=C(Nc1cnc(C2CCC(F)(F)CC2)c(Cl)c1)Nc1c[nH]c2cc(F)ccc12] | Method G: MS-ESI: 423 [M + H]⁺. |
| | | 1-(5-chloro-6-(4,4- | |
| | | difluorocyclohexyl)pyridin- | |
| | | 3-yl)-3-(6-fluoro-1H- | |
| | | indol-3-yl)urea | |
| Example 22 (Compound 142) | 5,6-difluoro-1H- indole-3-carboxylic acid Intermediate 13 | 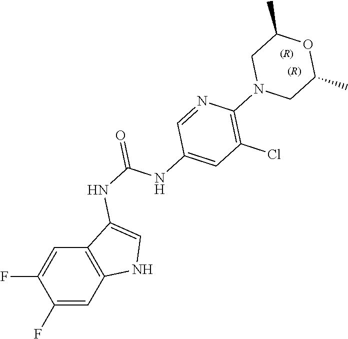 | Method D: MS-ESI: 436 [M + H]⁺. |
| | | 1-(5-chloro-6-((2R,6R)- | |
| | | 2,6- | |
| | | dimethylmorpholino)pyridin- | |
| | | 3-yl)-3-(5,6-difluoro-1H- | |
| | | indol-3-yl)urea | |
| Example 23 (Compound 143) | 5,6-difluoro-1H- indole-3-carboxylic acid Intermediate 53 | 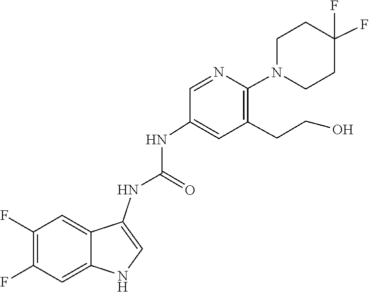 [SMILES: O=C(Nc1cnc(N2CCC(F)(F)CC2)c(CCO)c1)Nc1c[nH]c2cc(F)c(F)cc12] | Method C: MS-ESI: 452 [M + H]⁺. |
| | | 1-(5,6-difluoro-1H-indol- | |
| | | 3-yl)-3-(6-(4,4- | |
| | | difluoropiperidin-1-yl)-5- | |
| | | (2-hydroxyethyl)pyridin-3- | |
| | | yl)urea | |
| Example 24 (Compound 144) | 5-chloro-1H-indole- 3-carboxylic acid Intermediate 51 | 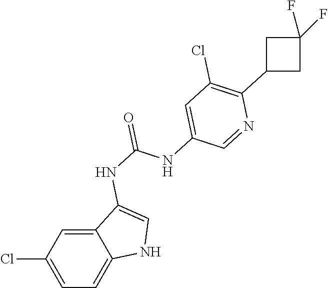 [SMILES: O=C(Nc1cnc(C2CC(F)(F)C2)c(Cl)c1)Nc1c[nH]c2ccc(Cl)cc12] | Method D: MS-ESI: 411 [M + H]⁺. |
| | | 1-(5-chloro-1H-indol-3- | |
| | | yl)-3-(5-chloro-6-(3,3- | |
| | | difluorocyclobutyl)pyridin- | |
| | | 3-yl)urea | |
| Example 25 (Compound 145) | 6-fluoro-1H-indole- 3-carboxylic acid Intermediate 14 | 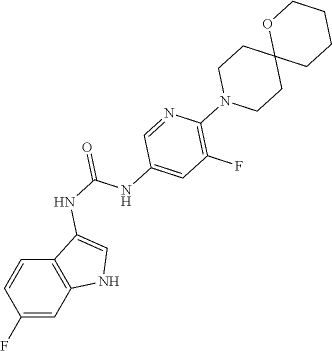 [SMILES: O=C(Nc1cnc(N2CCC3(CCCCO3)CC2)c(F)c1)Nc1c[nH]c2cc(F)ccc12] | Method C: MS-ESI: 442 [M + H]⁺. |
| | | 1-(6-fluoro-1H-indol-3-yl)- | |
| | | 3-(5-fluoro-6-(1-oxa-9- | |
| | | azaspiro[5.5]undecan-9- | |
| | | yl)pyridin-3-yl)urea | |
| Example 26 (Compound 146) | 5,6-difluoro-1H- indole-3-carboxylic acid Intermediate 15 | 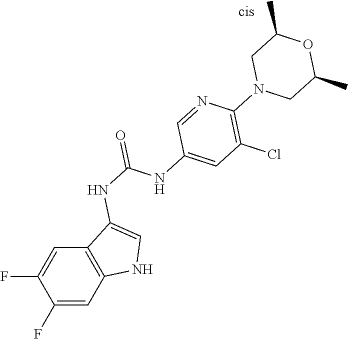 [SMILES: C[C@@H]1CN(c2ncc(NC(=O)Nc3c[nH]c4cc(F)c(F)cc34)cc2Cl)C[C@H](C)O1] | Method C: MS-ESI: 436 [M + H]⁺. |
| | | 1-(5-chloro-6-((2R,6S)- | |
| | | 2,6- | |
| | | dimethylmorpholino)pyridin- | |
| | | 3-yl)-3-(5,6-difluoro-1H- | |
| | | indol-3-yl)urea | |
| Example 27 (Compound 147) | 5,6-difluoro-1H- indole-3-carboxylic acid Intermediate 16 | 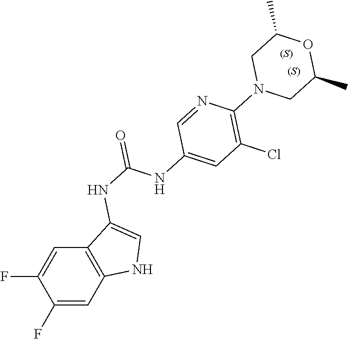 | Method D: MS-ESI: 436 [M + H]⁺. |
| | | 1-(5-chloro-6-((2S,6S)-2,6- | |
| | | dimethylmorpholino)pyridin- | |
| | | 3-yl)-3-(5,6-difluoro-1H- | |
| | | indol-3-yl)urea | |
| Example 28 (Compound 149) | 5,6-difluoro-1H- indole-3-carboxylic acid Intermediate 17 | 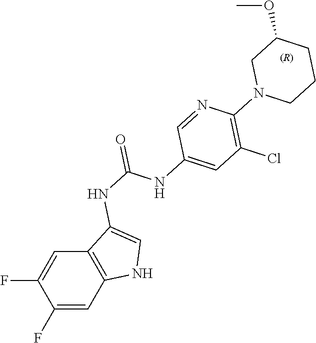 | Method E: MS-ESI: 436 [M + H]⁺. |
| | | (R)-1-(5-chloro-6-(3- | |
| | | methoxypiperidin-1- | |
| | | yl)pyridin-3-yl)-3-(5,6- | |
| | | difluoro-1H-indol-3- | |
| | | yl)urea | |
| Example 29 (Compound 148) | 5,6-difluoro-1H- indole-3-carboxylic acid Intermediate 18 | 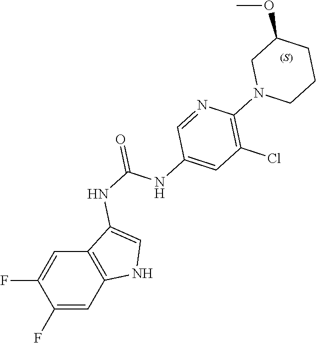 | Method D: MS-ESI: 436 [M + H]⁺. |
| | | (S)-1-(5-chloro-6-(3- | |
| | | methoxypiperidin-1- | |
| | | yl)pyridin-3-yl)-3-(5,6- | |
| | | difluoro-1H-indol-3- | |
| | | yl)urea | |
| Example 30 (Compound 150) | 5,6-difluoro-1H- indole-3-carboxylic acid Intermediate 19 | 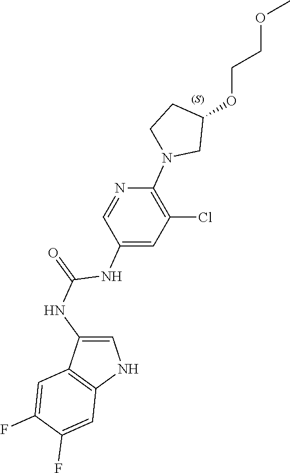 | Method C: MS-ESI: 466 [M + H]⁺. |
| | | (S)-1-(5-chloro-6-(3-(2- | |
| | | methoxyethoxy)pyrrolidin- | |
| | | 1-yl)pyridin-3-yl)-3-(5,6- | |
| | | difluoro-1H-indol-3- | |
| | | yl)urea | |
| Example 31 (Compound 151) | 5,6-difluoro-1H- indole-3-carboxyIic acid Intermediate 20 | 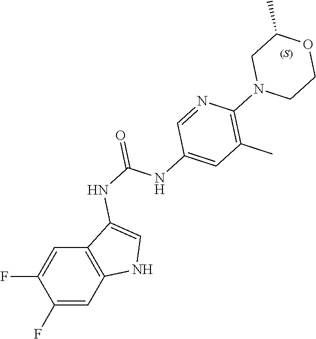 | Method E: MS-ESI: 402 [M + H]⁺. |
| | | (S)-1-(5,6-difluoro-1H- | |
| | | indol-3-yl)-3-(5-methyl-6- | |
| | | (2- | |
| | | methylmorpholino)pyridin- | |
| | | 3-yl)urea | |
| Example 32 (Compound 152) | 5,6-difluoro-1H- indole-3-carboxylic acid Intermediate 21 | 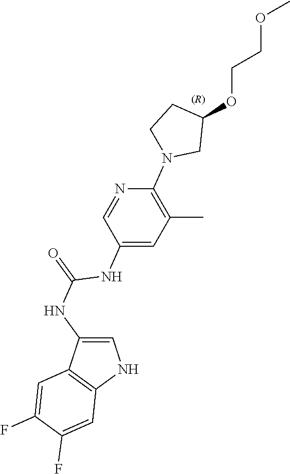 | Method D: MS-ESI: 446 [M + H]⁺. |
| | | (R)-1-(5,6-difluoro-1H- | |
| | | indol-3-yl)-3-(6-(3-(2- | |
| | | methoxyethoxy)pyrrolidin- | |
| | | 1-yl)-5-methylpyridin-3- | |
| | | yl)urea | |
| Example 33 (Compound 153) | 5,6-difluoro-1H- indole-3-carboxylic acid Intermediate 22 | 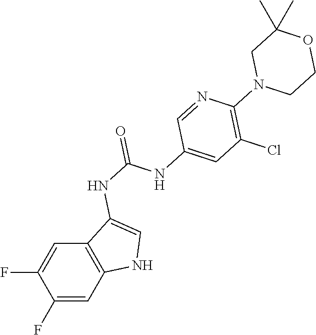 [SMILES: CC1(C)CN(c2ncc(NC(=O)Nc3c[nH]c4cc(F)c(F)cc34)cc2Cl)CCO1] | Method F: MS-ESI: 436 [M + H]⁺. |
| | | 1-(5-chloro-6-(2,2- | |
| | | dimethylmorpholino)pyridin- | |
| | | 3-yl)-3-(5,6-difluoro-1H- | |
| | | indol-3-yl)urea | |
| Example 34 (Compound 154) | 5,6-difluoro-1H- indole-3-carboxylic acid Intermediate 60 | 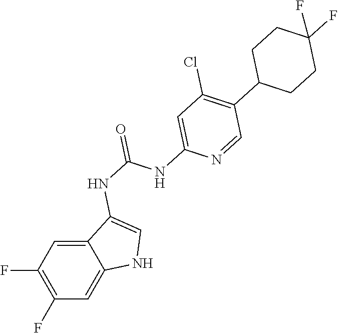 [SMILES: O=C(Nc1cc(Cl)c(C2CCC(F)(F)CC2)cn1)Nc1c[nH]c2cc(F)c(F)cc12] | Method D: MS-ESI: 441 [M + H]⁺. |
| | | 1-(4-chloro-5-(4,4- | |
| | | difluorocyclohexyl)pyridin- | |
| | | 2-yl)-3-(5,6-difluoro-1H- | |
| | | indol-3-yl)urea | |
| Example 35 (Compound 155) | 5-chloro-1H-indole- 3-carboxylic acid Intermediate 23 | 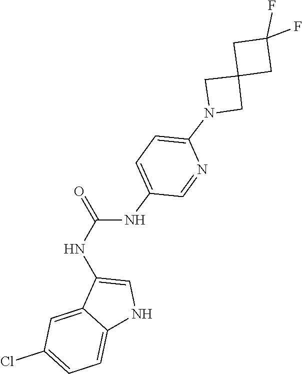 [SMILES: O=C(Nc1ccc(N2CC3(C2)CC(F)(F)C3)nc1)Nc1c[nH]c2ccc(Cl)cc12] | Method D: MS-ESI: 418 [M + H]⁺. |
| | | 1-(5-chloro-1H-indol-3- | |
| | | yl)-3-(6-(6,6-difluoro-2- | |
| | | azaspiro[3.3]heptan-2- | |
| | | yl)pyridin-3-yl)urea | |
| Example 36 (Compound 156) | 5-chloro-1H-indole- 3-carboxylic acid Intermediate 45 | 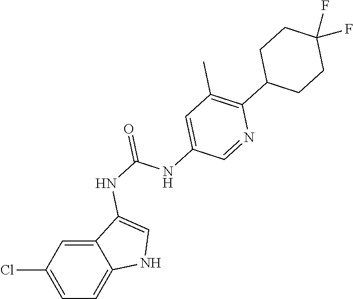 [SMILES: Cc1cc(NC(=O)Nc2c[nH]c3ccc(Cl)cc23)cnc1C1CCC(F)(F)CC1] | Method D: MS-ESI: 419 [M + H]⁺. |
| | | 1-(5-chloro-1H-indol-3- | |
| | | yl)-3-(6-(4,4- | |
| | | difluorocyclohexyl)-5- | |
| | | methylpyridin-3-yl)urea | |
| Example 37 (Compound 157) | 5-chloro-1H-indole- 3-carboxylic acid Intermediate 44 | 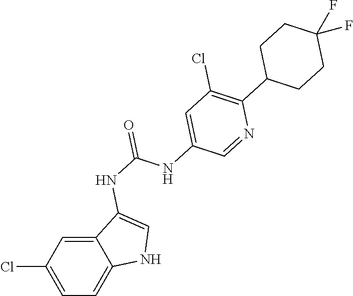 [SMILES: O=C(Nc1cnc(C2CCC(F)(F)CC2)c(Cl)c1)Nc1c[nH]c2ccc(Cl)cc12] | Method D: MS-ESI: 439 [M + H]⁺. |
| | | 1-(5-chloro-1H-indol-3- | |
| | | yl)-3-(5-chloro-6-(4,4- | |
| | | difluorocyclohexyl)pyridin- | |
| | | 3-yl)urea | |
| Example 38 (Compound 158) | 5,6-difluoro-1H- indole-3-carboxylic acid Intermediate 24 | 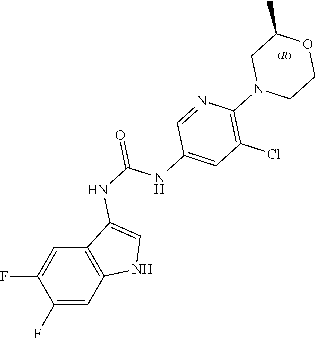 | Method D: MS-ESI: 422 [M + H]⁺. |
| | | (R)-1-(5-chloro-6-(2- | |
| | | methylmorpholino)pyridin- | |
| | | 3-yl)-3-(5,6-difluoro-1H- | |
| | | indol-3-yl)urea | |
| Example 39 (Compound 160) | 5,6-difluoro-1H- indole-3-carboxylic acid Intermediate 25 | 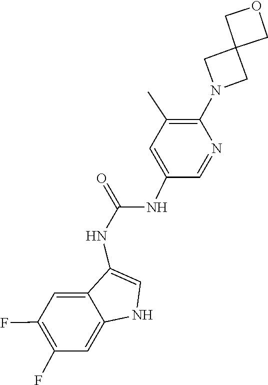 [SMILES: Cc1cc(NC(=O)Nc2c[nH]c3cc(F)c(F)cc23)cnc1N1CC2(COC2)C1] | Method F: MS-ESI: 400 [M + H]⁺. |
| | | 1-(5,6-difluoro-1H-indol- | |
| | | 3-yl)-3-(5-methyl-6-(2- | |
| | | oxa-6-azaspiro[3.3]heptan- | |
| | | 6-yl)pyridin-3-yl)urea | |
| Example 40 (Compound 161) | 5,6-difluoro-1H- indole-3-carboxylic acid Intermediate 26 | 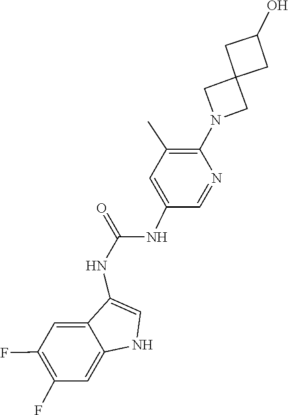 [SMILES: Cc1cc(NC(=O)Nc2c[nH]c3cc(F)c(F)cc23)cnc1N1CC2(CC(O)C2)C1] | Method D: MS-ESI: 414 [M + H]⁺. |
| | | 1-(5,6-difluoro-1H-indol- | |
| | | 3-yl)-3-(6-(6-hydroxy-2- | |
| | | azaspiro[3.3]heptan-2-yl)- | |
| | | 5-methylpyridin-3-yl)urea | |
| Example 41 (Compound 162) | 5,6-difluoro-1H- indole-3-carboxylic acid Intermediate 27 | 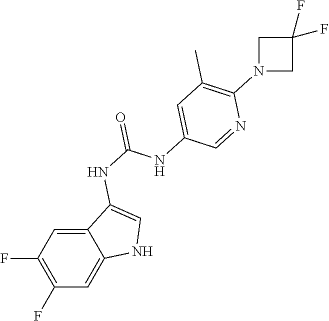 [SMILES: Cc1cc(NC(=O)Nc2c[nH]c3cc(F)c(F)cc23)cnc1N1CC(F)(F)C1] | Method D: MS-ESI: 394 [M + H]⁺. |
| | | 1-(5,6-difluoro-1H-indol- | |
| | | 3-yl)-3-(6-(3,3- | |
| | | difluoroazetidin-1-yl)-5- | |
| | | methylpyridin-3-yl)urea | |
| Example 42 (Compound 163) | 5,6-difluoro-1H- indole-3-carboxylic acid Intermediate 57 | 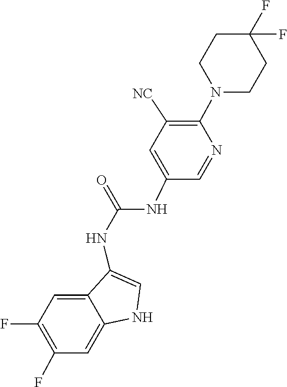 [SMILES: N#Cc1cc(NC(=O)Nc2c[nH]c3cc(F)c(F)cc23)cnc1N1CCC(F)(F)CC1] | Method E: MS-ESI: 433 [M + H]⁺. |
| | | 1-(5-cyano-6-(4,4- | |
| | | difluoropiperidin-1- | |
| | | yl)pyridin-3-yl)-3-(5,6- | |
| | | difluoro-1H-indol-3- | |
| | | yl)urea | |
| Example 43 (Compound 164) | Intermediate 62; Intermediate 28 | 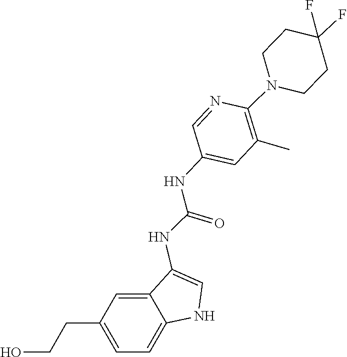 [SMILES: Cc1cc(NC(=O)Nc2c[nH]c3ccc(CCO)cc23)cnc1N1CCC(F)(F)CC1] | Method D: MS-ESI: 430 [M + H]⁺. |
| | | 1-(6-(4,4- | |
| | | difluoropiperidin-1-yl)-5- | |
| | | methylpyridin-3-yl)-3-(5- | |
| | | (2-hydroxyethyl)-1H- | |
| | | indol-3-yl)urea | |
| Example 44 (Compound 165) | 5,6-difluoro-1H- indole-3-carboxylic acid Intermediate 29 | 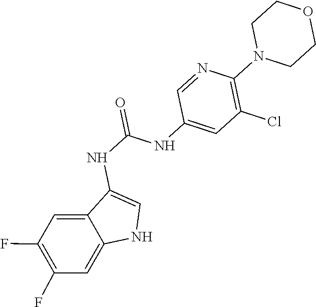 [SMILES: O=C(Nc1cnc(N2CCOCC2)c(Cl)c1)Nc1c[nH]c2cc(F)c(F)cc12] | Method D: MS-ESI: 408 [M + H]⁺. |
| | | 1-(5-chloro-6- | |
| | | morpholinopyridin-3-yl)-3- | |
| | | (5,6-difluoro-1H-indol-3- | |
| | | yl)urea | |
| Example 45 (Compound 166) | 5,6-difluoro-1H- indole-3-carboxylic acid Intermediate 46 | 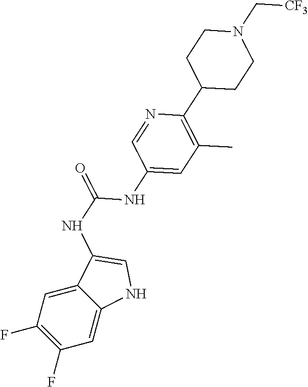 [SMILES: Cc1cc(NC(=O)Nc2c[nH]c3cc(F)c(F)cc23)cnc1C1CCN(CC(F)(F)F)CC1] | Method D: MS-ESI: 468 [M + H]⁺. |
| | | 1-(5,6-difluoro-1H-indol- | |
| | | 3-yl)-3-(5-methyl-6-(1- | |
| | | (2,2,2- | |
| | | trifluoroethyl)piperidin-4- | |
| | | yl)pyridin-3-yl)urea | |
| Example 46 (Compound 167) | 5,6-difluoro-1H- indole-3-carboxylic acid Intermediate 9 | 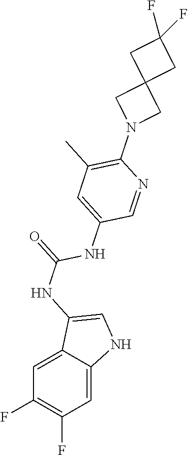 [SMILES: Cc1cc(NC(=O)Nc2c[nH]c3cc(F)c(F)cc23)cnc1N1CC2(C1)CC(F)(F)C2] | Method D: MS-ESI: 434 [M + H]⁺. |
| | | 1-(5,6-difluoro-1H-indol- | |
| | | 3-yl)-3-(6-(6,6-difluoro-2- | |
| | | azaspiro[3.3]heptan-2-yl)- | |
| | | 5-methylpyridin-3-yl)urea | |
| Example 47 (Compound 168) | 5,6-difluoro-1H- indole-3-carboxylic acid Intermediate 30 | 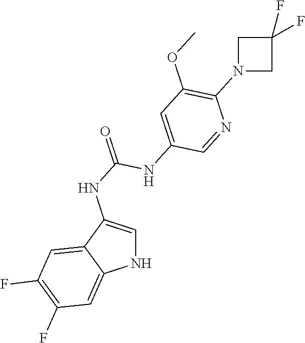 [SMILES: COc1cc(NC(=O)Nc2c[nH]c3cc(F)c(F)cc23)cnc1N1CC(F)(F)C1] | Method E: MS-ESI: 410 [M + H]⁺. |
| | | 1-(5,6-difluoro-1H-indol- | |
| | | 3-yl)-3-(6-(3,3- | |
| | | difluoroazetidin-1-yl)-5- | |
| | | methoxypyridin-3-yl)urea | |
| Example 48 (Compound 169) | 5,6-difluoro-1H- indole-3-carboxylic acid Intermediate 47 | 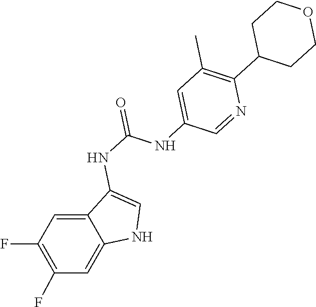 [SMILES: Cc1cc(NC(=O)Nc2c[nH]c3cc(F)c(F)cc23)cnc1C1CCOCC1] | Method D: MS-ESI: 387 [M + H]⁺. |
| | | 1-(5,6-difluoro-1H-indol- | |
| | | 3-yl)-3-(5-methyl-6- | |
| | | (tetrahydro-2H-pyran-4- | |
| | | yl)pyridin-3-yl)urea | |
| Example 49 (Compound 170) | Intermediate 63; Intermediate 5 | 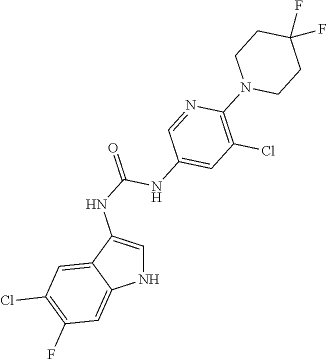 [SMILES: O=C(Nc1cnc(N2CCC(F)(F)CC2)c(Cl)c1)Nc1c[nH]c2cc(F)c(Cl)cc12] | Method D: MS-ESI: 458 [M + H]⁺. |
| | | 1-(5-chloro-6-(4.4- | |
| | | difluoropiperidin-1- | |
| | | yl)pyridin-3-yl)-3-(5- | |
| | | chloro-6-fluoro-1H-indol- | |
| | | 3-yl)urea | |
| Example 50 (Compound 183) | Intermediate 64; Intermediate 5 | 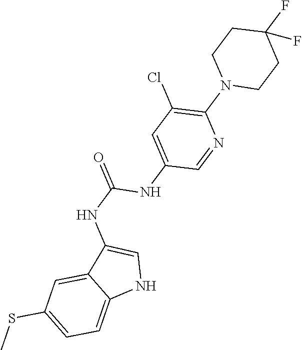 [SMILES: CSc1ccc2[nH]cc(NC(=O)Nc3cnc(N4CCC(F)(F)CC4)c(Cl)c3)c2c1] | Method G: MS-ESI: 452 [M + H]⁺. |
| | | 1-(5-chloro-6-(4,4- | |
| | | difluoropiperidin-1- | |
| | | yl)pyridin-3-yl)-3-(5- | |
| | | (methylthio)-1H-indol-3- | |
| | | yl)urea | |
| Example 51 (Compound 172) | 1H-indole-3- carboxylic acid Intermediate 32 | 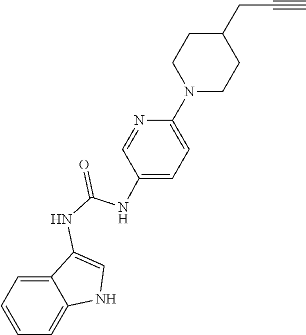 [SMILES: C#CCC1CCN(c2ccc(NC(=O)Nc3c[nH]c4ccccc34)cn2)CC1] | Method D: MS-ESI: 374 [M + H]⁺. |
| | | 1-(1H-indol-3-yl)-3-(6-(4- | |
| | | (prop-2-yn-1-yl)piperidin- | |
| | | 1-yl)pyridin-3-yl)urea | |
| Example 52 (Compound 173) | 5,6-difluoro-1H- indole-3-carboxylic acid Intermediate 51 | 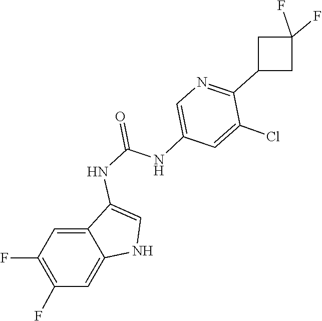 [SMILES: O=C(Nc1cnc(C2CC(F)(F)C2)c(Cl)c1)Nc1c[nH]c2cc(F)c(F)cc12] | Method D: MS-ESI: 413 [M + H]⁺. |
| | | 1-(5-chloro-6-(3,3- | |
| | | difluorocyclobutyl)pyridin- | |
| | | 3-yl)-3-(5,6-difluoro-1H- | |
| | | indol-3-yl)urea | |
| Example 53 (Compound 174) | 5-chloro-1H-indole- 3-carboxylic acid Intermediate 58 | 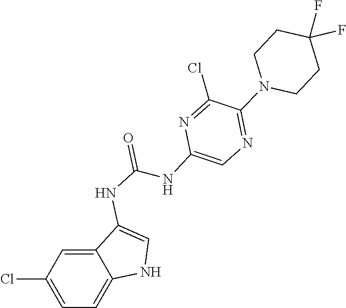 [SMILES: O=C(Nc1cnc(N2CCC(F)(F)CC2)c(Cl)n1)Nc1c[nH]c2ccc(Cl)cc12] | Method D: MS-ESI: 441 [M + H]⁺. |
| | | 1-(5-chloro-1H-indol-3- | |
| | | yl)-3-(6-chloro-5-(4,4- | |
| | | difluoropiperidin-1- | |
| | | yl)pyrazin-2-yl)urea | |
| Example 54 (Compound 175) | 5,6-difluoro-1H- indole-3-carboxylic acid Intermediate 59 | 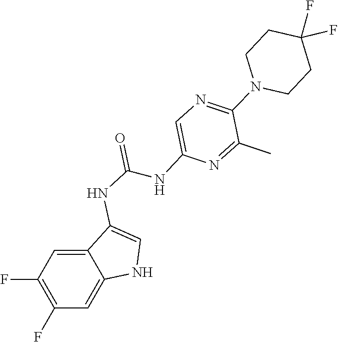 [SMILES: Cc1nc(NC(=O)Nc2c[nH]c3cc(F)c(F)cc23)cnc1N1CCC(F)(F)CC1] | Method F: MS-ESI: 423 [M + H]⁺. |
| | | 1-(5,6-difluoro-1H-indol- | |
| | | 3-yl)-3-(5-(4,4- | |
| | | difluoropiperidin-1-yl)-6- | |
| | | methylpyrazin-2-yl)urea | |
| Example 55 (Compound 176) | 5,6-difluoro-1H- indole-3-carboxylic acid Intermediate 33 | 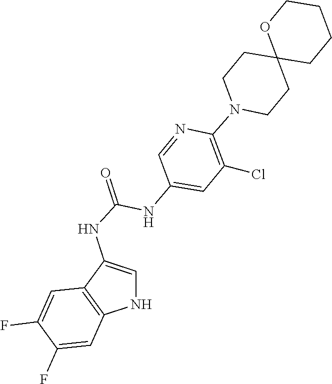 [SMILES: O=C(Nc1cnc(N2CCC3(CCCCO3)CC2)c(Cl)c1)Nc1c[nH]c2cc(F)c(F)cc12] | Method D: MS-ESI: 476 [M + H]⁺. |
| | | 1-(5-chloro-6-(1-oxa-9- | |
| | | azaspiro[5.5]undecan-9- | |
| | | yl)pyridin-3-yl)-3-(5,6- | |
| | | difluoro-1H-indol-3- | |
| | | yl)urea | |
| Example 56 (Compound 177) | 5,6-difluoro-1H- indole-3-carboxylic acid Intermediate 34 | 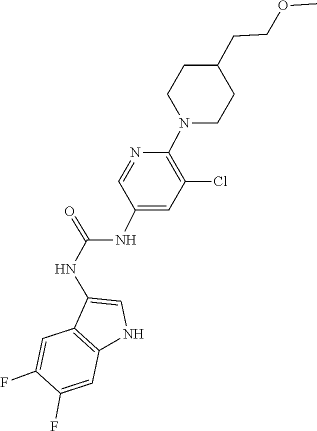 [SMILES: COCCC1CCN(c2ncc(NC(=O)Nc3c[nH]c4cc(F)c(F)cc34)cc2Cl)CC1] | Method D: MS-ESI: 464 [M + H]⁺. |
| | | 1-(5-chloro-6-(4-(2- | |
| | | methoxyethyl)piperidin-1- | |
| | | yl)pyridin-3-yl)-3-(5,6- | |
| | | difluoro-1H-indol-3- | |
| | | yl)urea | |
| Example 57 (Compound 179) | 5,6-difluoro-1H- indole-3-carboxylic acid Intermediate 35 | 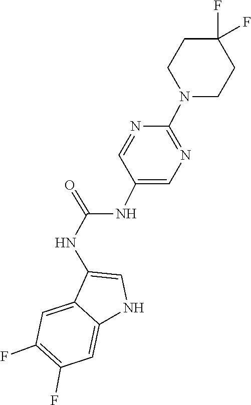 [SMILES: O=C(Nc1cnc(N2CCC(F)(F)CC2)nc1)Nc1c[nH]c2cc(F)c(F)cc12] | Method D: MS-ESI: 409 [M + H]⁺. |
| | | 1-(5,6-difluoro-1H-indol- | |
| | | 3-yl)-3-(2-(4,4- | |
| | | difluoropiperidin-1- | |
| | | yl)pyrimidin-5-yl)urea | |
| Example 58 (Compound 180) | 5-chloro-1H-indole- 3-carboxylic acid Intermediate 35 | 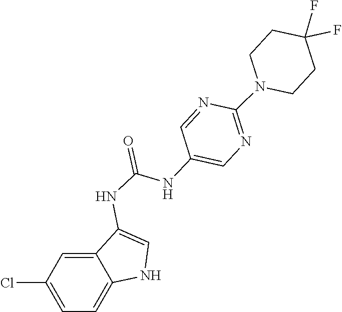 [SMILES: O=C(Nc1cnc(N2CCC(F)(F)CC2)nc1)Nc1c[nH]c2ccc(Cl)cc12] | Method D: MS-ESI: 407 [M + H]⁺. |
| | | 1-(5-choro-1H-indol-3- | |
| | | yl)-3-(2-(4,4- | |
| | | difluoropiperidin-1- | |
| | | yl)pyrimidin-5-yl)urea | |
| Example 59 (Compound 178) | 1H-indole-3- carboxylic acid Intermediate 35 | 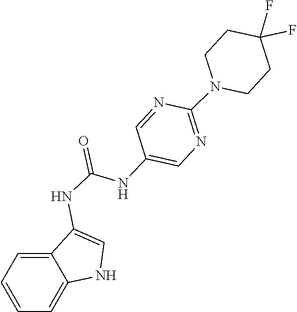 [SMILES: O=C(Nc1cnc(N2CCC(F)(F)CC2)nc1)Nc1c[nH]c2ccccc12] | Method D: MS-ESI: 373 [M + H]⁺. |
| | | 1-(2-(4,4- | |
| | | difluoropiperidin-1- | |
| | | yl)pyrimidin-5-yl)-3-(1H- | |
| | | indol-3-yl)urea | |
| Example 60 (Compound 181) | 5-cyano-1H-indole- 3-carboxylic acid Intermediate 5 | 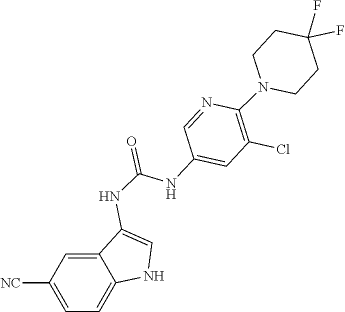 [SMILES: N#Cc1ccc2[nH]cc(NC(=O)Nc3cnc(N4CCC(F)(F)CC4)c(Cl)c3)c2c1] | Method D: MS-ESI: 431 [M + H]⁺. |
| | | 1-(5-chloro-6-(4,4- | |
| | | difluoropiperidin-1- | |
| | | yl)pyridin-3-yl)-3-(5- | |
| | | cyano-1H-indol-3-yl)urea | |
| Example 61 (Compound 182) | 5-fluoro-1H-indole- 3-carboxylic acid Intermediate 55 | 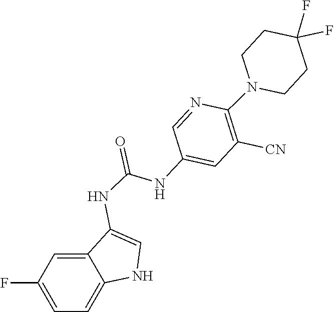 [SMILES: N#Cc1cc(NC(=O)Nc2c[nH]c3ccc(F)cc23)cnc1N1CCC(F)(F)CC1] | Method C: MS-ESI: 414 [M + H]⁺. |
| | | 1-(5-cyano-6-(4,4- | |
| | | difluorocyclohexyl)pyridin- | |
| | | 3-yl)-3-(5-fluoro-1H- | |
| | | indol-3-yl)urea | |
| Example 62 (Compound 185) | 5,6-difluoro-1H- indole-3-carboxylic acid Intermediate 36 | 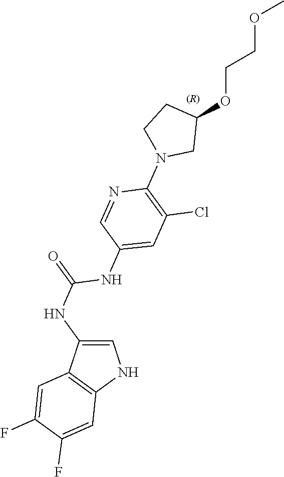 | Method D: MS-ESI: 466 [M + H]⁺. |
| | | (R)-1-(5-chloro-6-(3-(2- | |
| | | methoxyethoxy)pyrrolidin- | |
| | | 1-yl)pyridin-3-yl)-3-(5,6- | |
| | | difluoro-1H-indol-3- | |
| | | yl)urea | |
| Example 63 (Compound 186) | 5,6-difluoro-1H- indole-3-carboxylic acid Intermediate 37 | 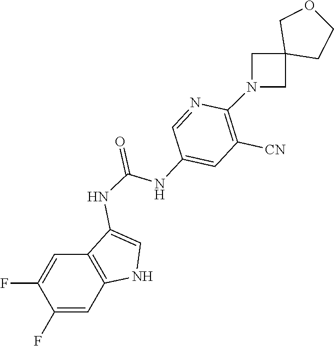 [SMILES: N#Cc1cc(NC(=O)Nc2c[nH]c3cc(F)c(F)cc23)cnc1N1CC2(CCOC2)C1] | Method F: MS-ESI: 434 [M + H]⁺. |
| | | 1-(5-chloro-6-(6-oxa-2- | |
| | | azaspiro[3.4]octan-2- | |
| | | yl)pyridin-3-yl)-3-(5,6- | |
| | | difluoro-1H-indol-3- | |
| | | yl)urea | |
| Example 64 (Compound 187) | 5,6-difluoro-1H- indole-3-carhoxylic acid Intermediate 38 | 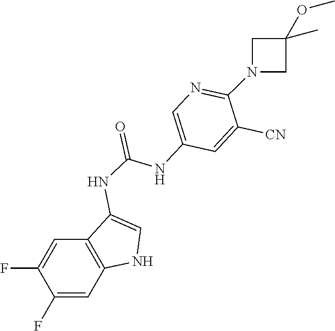 | Method F: MS-ESI: 422 [M + H]⁺. |
| | | 1-(5-chloro-6-(3-methoxy- | |
| | | 3-methylazetidin-1- | |
| | | yl)pyridin-3-yl)-3-(5,6- | |
| | | difluoro-1H-indol-3- | |
| | | yl)urea | |
| Example 65 (Compound 188) | 5,6-difluoro-1H- indole-3-carboxylic acid Intermediate 39 | 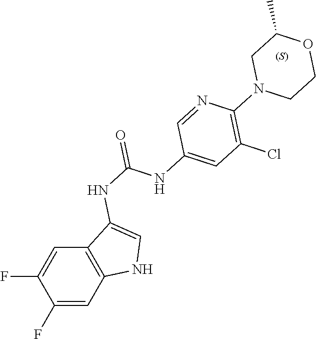 | Method D: MS-ESI: 422 [M + H]⁺. |
| | | (S)-1-(5-chloro-6-(2- | |
| | | methylmorpholino)pyridin- | |
| | | 3-yl)-3-(5,6-difluoro-1H- | |
| | | indol-3-yl)urea | |
| Example 66 (Compound 189) | 5-chloro-1H-indole- 3-carboxylic acid Intermediate 40 | 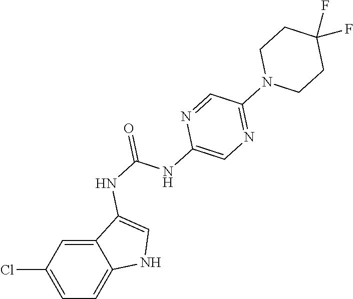 [SMILES: O=C(Nc1cnc(N2CCC(F)(F)CC2)cn1)Nc1c[nH]c2ccc(Cl)cc12] | Method D: MS-ESI: 407 [M + H]⁺. |
| | | 1-(5-chloro-1H-indol-3- | |
| | | yl)-3-(5-(4,4- | |
| | | difluoropiperidin-1- | |
| | | yl)pyrazin-2-yl)urea | |
| Example 67 (Compound 190) | 1H-indole-3- carboxylic acid Intermediate 43 | 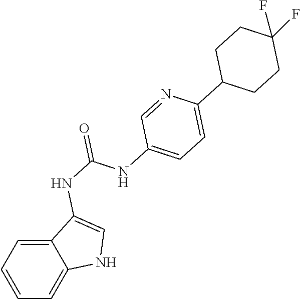 [SMILES: O=C(Nc1ccc(C2CCC(F)(F)CC2)nc1)Nc1c[nH]c2ccccc12] | Method D: MS-ESI: 371 [M + H]⁺. |
| | | 1-(6-(4,4- | |
| | | difluorocyclohexyl)pyridin- | |
| | | 3-yl)-3-(1H-indol-3- | |
| | | yl)urea | |
| Example 68 (Compound 192) | 5-iodo-1H-indole-3- carboxylic acid Intermediate 43 | 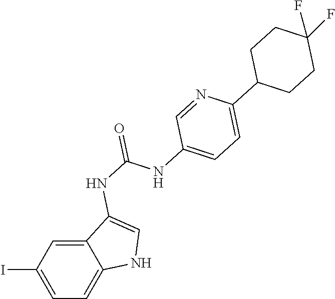 [SMILES: O=C(Nc1ccc(C2CCC(F)(F)CC2)nc1)Nc1c[nH]c2ccc(I)cc12] | Method F: MS-ESI: 497 [M + H]⁺. |
| | | 1-(6-(4,4- | |
| | | difluorocyclohexyl)pyridin- | |
| | | 3-yl)-3-(5-iodo-1H-indol- | |
| | | 3-yl)urea | |
| Example 69 (Compound 193) | 5-fluoro-1H-indole- 3-carboxylic acid Intermediate 5 | 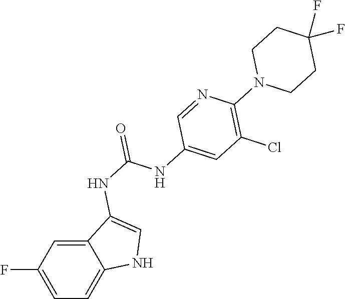 [SMILES: O=C(Nc1cnc(N2CCC(F)(F)CC2)c(Cl)c1)Nc1c[nH]c2ccc(F)cc12] | Method C: MS-ESI: 424 [M + H]⁺. |
| | | 1-(5-chloro-6-(4,4- | |
| | | difluoropiperidin-1- | |
| | | yl)pyridin-3-yl)-3-(5- | |
| | | fluoro-1H-indol-3-yl)urea | |
| Example 70 (Compound 194) | 5,6-difluoro-1H- indole-3-carboxylic acid Intermediate 40 | 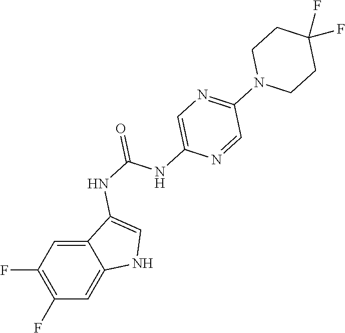 [SMILES: O=C(Nc1cnc(N2CCC(F)(F)CC2)cn1)Nc1c[nH]c2cc(F)c(F)cc12] | Method D: MS-ESI: 409 [M + H]⁺. |
| | | 1-(5,6-difluoro-1H-indol- | |
| | | 3-yl)-3-(5-(4,4- | |
| | | difluoropiperidin-1- | |
| | | yl)pyrazin-2-yl)urea | |
| Example 71 (Compound 195) | 5-fluoro-1H-indole- 3-carboxylic acid Intermediate 28 | 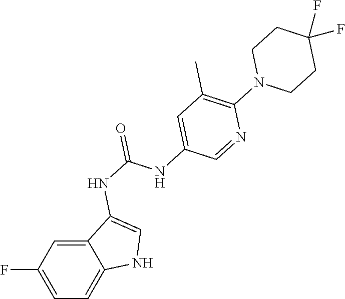 [SMILES: Cc1cc(NC(=O)Nc2c[nH]c3ccc(F)cc23)cnc1N1CCC(F)(F)CC1] | Method D: MS-ESI: 404 [M + H]⁺. |
| | | 1-(6-(4,4- | |
| | | difluoropiperidin-1-yl)-5- | |
| | | methylpyridin-3-yl)-3-(5- | |
| | | fluoro-1H-indol-3-yl)urea | |
| Example 73 (Compound 197) | 5-methyl-1H-indole- 3-carboxylic acid Intermediate 28 | 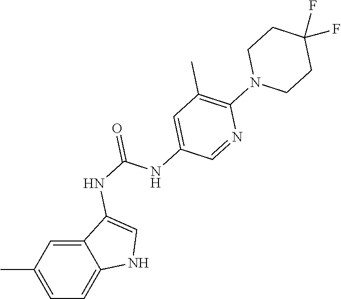 [SMILES: Cc1ccc2[nH]cc(NC(=O)Nc3cnc(N4CCC(F)(F)CC4)c(C)c3)c2c1] | Method D: MS-ESI: 400 [M + H]⁺. |
| | | 1-(6-(4,4- | |
| | | difluoropiperidin-1-yl)-5- | |
| | | methylpyridin-3-yl)-3-(5- | |
| | | methyl-1H-indol-3-yl)urea | |
| Example 74 (Compound 198) | 1H-indole-3- carboxylic acid Intermediate 41 | 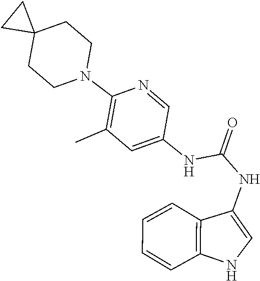 [SMILES: Cc1cc(NC(=O)Nc2c[nH]c3ccccc23)cnc1N1CCC2(CC1)CC2] | Method D: MS-ESI: 376.1. [M + H]⁺. |
| | | 1-(1H-indol-3-yl)-3-(5- | |
| | | methyl-6-(6- | |
| | | azaspiro[2.5]octan-6- | |
| | | yl)pyridin-3-yl)urea | |
| Example 75 (Compound 199) | 5,6-difluoro-1H- indole-3-carboxylic acid Intermediate 5 | 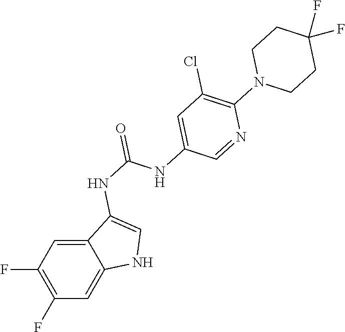 [SMILES: O=C(Nc1cnc(N2CCC(F)(F)CC2)c(Cl)c1)Nc1c[nH]c2cc(F)c(F)cc12] | Method F: MS-ESI: 442 [M + H]⁺. |
| | | 1-(5-chloro-6-(4,4- | |
| | | difluoropiperidin-1- | |
| | | yl)pyridin-3-yl)-3-(5,6- | |
| | | difluoro-1H-indol-3- | |
| | | yl)urea | |
| Example 76 (Compound 201) | 1H-indole-3- carboxylic acid Intermediate 28 | 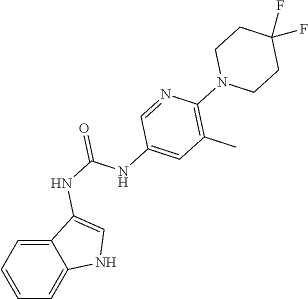 [SMILES: Cc1cc(NC(=O)Nc2c[nH]c3ccccc23)cnc1N1CCC(F)(F)CC1] | Method D: MS-ESI: 386 [M + H]⁺. |
| | | 1-(6-(4,4- | |
| | | difluoropiperidin-1-yl)-5- | |
| | | methylpyridin-3-yl)-3-(1H- | |
| | | indol-3-yl)urea | |
| Example 77 (Compound 200) | 5,6-difluoro-1H- indole-3-carboxylic acid Intermediate 28 | 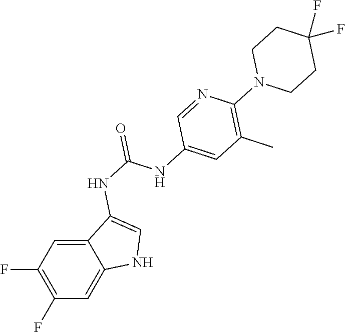 [SMILES: Cc1cc(NC(=O)Nc2c[nH]c3cc(F)c(F)cc23)cnc1N1CCC(F)(F)CC1] | Method G: MS-ESI: 422 [M + H]⁺. |
| | | 1-(5,6-difluoro-1H-indol- | |
| | | 3-yl)-3-(6-(4,4- | |
| | | difluoropiperidin-1-yl)-5- | |
| | | methylpyridin-3-yl)urea | |
| Example 78 (Compound 202) | 1H-indole-3- carboxylic acid Intermediate 42 | 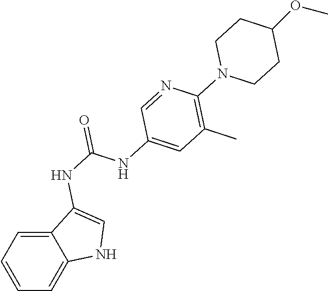 [SMILES: COC1CCN(c2ncc(NC(=O)Nc3c[nH]c4ccccc34)cc2C)CC1] | Method D: MS-ESI: 380 [M + H]⁺. |
| | | 1-(1H-indol-3-yl)-3-(6-(4- | |
| | | methoxypiperidin-1-yl)-5- | |
| | | methylpyridin-3-yl)urea | |
| Example 79 (Compound 203) | 5-bromo-1H-indole- 3-carboxylic acid Intermediate 43 | 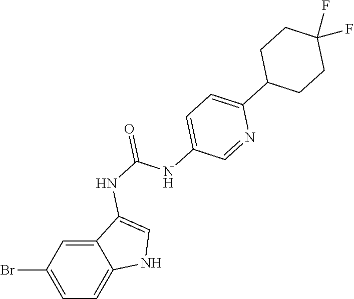 [SMILES: O=C(Nc1ccc(C2CCC(F)(F)CC2)nc1)Nc1c[nH]c2ccc(Br)cc12] | Method D: MS-ESI: 449 [M + H]⁺. |
| | | 1-(5-bromo-1H-indol-3- | |
| | | yl)-3-(6-(4,4- | |
| | | difluorocyclohexyl)pyridin- | |
| | | 3-yl)urea | |
| Example 80 (Compound 204) | 1H-indole-3- carboxylic acid Intermediate 49 | 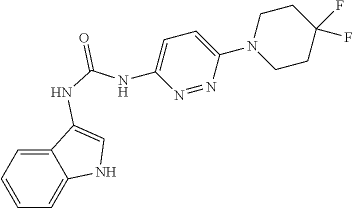 [SMILES: O=C(Nc1ccc(N2CCC(F)(F)CC2)nn1)Nc1c[nH]c2ccccc12] | Method D: MS-ESI: 373 [M + H]⁺. |
| | | 1-(6-(4,4- | |
| | | difluoropiperidin-1- | |
| | | yl)pyridazin-3-yl)-3-(1H- | |
| | | indol-3-yl)urea | |
| Example 81 (Compound 205) | 1H-indole-3- carboxylic acid Intermediate 40 | 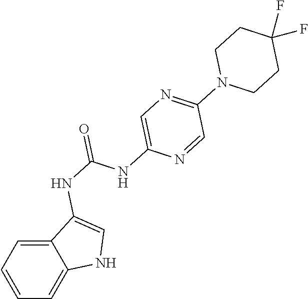 [SMILES: O=C(Nc1cnc(N2CCC(F)(F)CC2)cn1)Nc1c[nH]c2ccccc12] | Method C: MS-ESI: 373 [M + H]⁺. |
| | | 1-(5-(4,4- | |
| | | difluoropiperidin-1- | |
| | | yl)pyrazin-2-yl)-3-(1H- | |
| | | indol-3-yl)urea | |
| Example 82 (Compound 206) | 1H-indole-3- carboxylic acid Intermediate 5 | 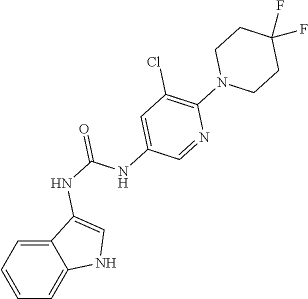 [SMILES: O=C(Nc1cnc(N2CCC(F)(F)CC2)c(Cl)c1)Nc1c[nH]c2ccccc12] | Method D: MS-ESI: 406 [M + H]⁺. |
| | | 1-(5-chloro-6-(4,4- | |
| | | difluoropiperidin-1- | |
| | | yl)pyridin-3-yl)-3-(1H- | |
| | | indol-3-yl)urea | |
| Example 83 (Compound 207) | 1H-indole-3- carboxylic acid Intermediate 48 | 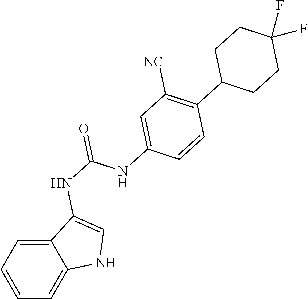 [SMILES: N#Cc1cc(NC(=O)Nc2c[nH]c3ccccc23)ccc1C1CCC(F)(F)CC1] | Method D: MS-ESI: 395 [M + H]⁺. |
| | | 1-(3-cyano-4-(4,4- | |
| | | difluorocyclohexyl)phenyl)- | |
| | | 3-(1H-indol-3-yl)urea | |
| Example 84 (Compound 208) | 5-bromo-1H-indole- 3-carboxylic acid Intermediate 5 | 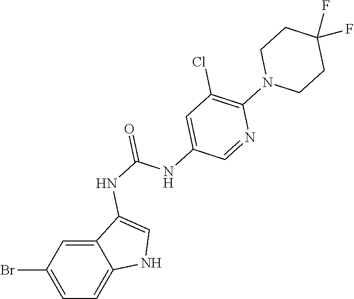 [SMILES: O=C(Nc1cnc(N2CCC(F)(F)CC2)c(Cl)c1)Nc1c[nH]c2ccc(Br)cc12] | Method E: MS-ESI: 484 [M + H]⁺. |
| | | 1-(5-bromo-1H-indol-3- | |
| | | yl)-3-(5-chloro-6-(4,4- | |
| | | difluoropiperidin-1- | |
| | | yl)pyridin-3-yl)urea | |
| Example 85 (Compound 184) | 5-bromo-1H-indole- 3-carboxylic acid Intermediate 28 | 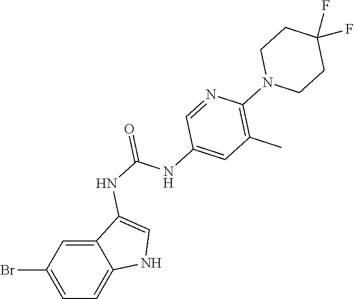 [SMILES: Cc1cc(NC(=O)Nc2c[nH]c3ccc(Br)cc23)cnc1N1CCC(F)(F)CC1] | Method D: MS-ESI: 464 [M + H]⁺. |
| | | 1-(5-bromo-1H-indol-3- | |
| | | yl)-3-(6-(4,4- | |
| | | difluoropiperidin-1-yl)-5- | |
| | | methylpyridin-3-yl)urea | |
| Example 86 (Compound 171) | Intermediate 67; Intermediate 5 | 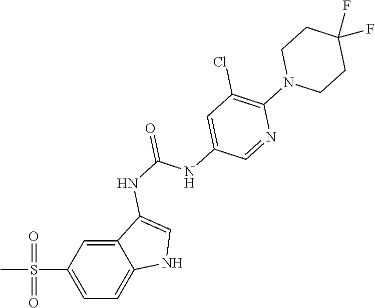 [SMILES: CS(=O)(=O)c1ccc2[nH]cc(NC(=O)Nc3cnc(N4CCC(F)(F)CC4)c(Cl)c3)c2c1] | Method D: MS-ESI: 484 [M + H]⁺. |
| | | 1-(5-chloro-6-(4,4- | |
| | | difluoropiperidin-1- | |
| | | yl)pyridin-3-yl)-3-(5- | |
| | | (methylsulfonyl)-1H-indol- | |
| | | 3-yl)urea | |
| Example 87 (Compound 213) | 5,6-difluoro-1H- indole-3-carboxylic acid; Intermediate 105 | 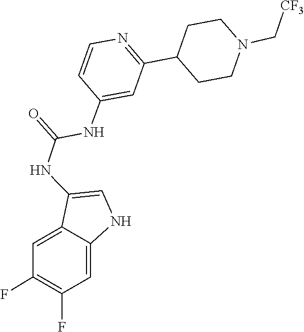 [SMILES: O=C(Nc1ccnc(C2CCN(CC(F)(F)F)CC2)c1)Nc1c[nH]c2cc(F)c(F)cc12] | Method E: MS-ESI: 454 [M + H]⁺. |
| | | 1-(5,6-difluoro-1H-indol- | |
| | | 3-yl)-3-(2-(1-(2,2,2- | |
| | | trifluoroethyl)piperidin-4- | |
| | | yl)pyridin-4-yl)urea | |
| Example 88 (Compound 214) | 5-chloro-1H-indole- 3-carboxylic acid; Intermediate 110 | 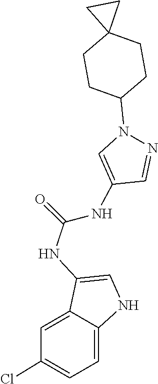 [SMILES: O=C(Nc1cnn(C2CCC3(CC2)CC3)c1)Nc1c[nH]c2ccc(Cl)cc12] | Method I: MS-ESI: 384 [M + H]⁺. |
| | | 1-(5-chloro-1H-indol-3- | |
| | | yl)-3-(1-(spiro[2.5]octan-6- | |
| | | yl)-1H-pyrazol-4-yl)urea | |
| Example 89 (Compound 215) | 5-chloro-1H-indole- 3-carboxylic acid; Intermediate 116 | 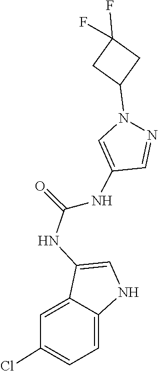 [SMILES: O=C(Nc1cnn(C2CC(F)(F)C2)c1)Nc1c[nH]c2ccc(Cl)cc12] | Method D: MS-ESI: 366 [M + H]+. |
| | | 1-(5-chloro-1H-indol-3- | |
| | | yl)-3-(1-(3,3- | |
| | | difluorocyclobutyl)-1H- | |
| | | pyrazol-4-yl)urea | |
| Example 90 (Compound 216) | 5,6-difluoro-1H- indole-3-carboxylic acid; Intermediate 116 | 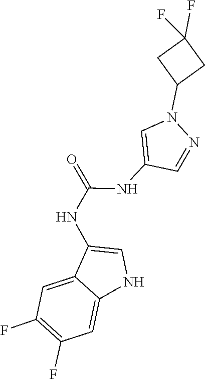 [SMILES: O=C(Nc1cnn(C2CC(F)(F)C2)c1)Nc1c[nH]c2cc(F)c(F)cc12] | Method H: MS-ESI: 368 [M + H]+. |
| | | 1-(5,6-difluoro-1H-indol- | |
| | | 3-yl)-3-(1-(3,3- | |
| | | difluorocyclobutyl)-1H- | |
| | | pyrazol-4-yl)urea | |
| Example 91 (Compound 217) | 5-chloro-1H-indole- 3-carboxylic acid; Intermediate 111 | 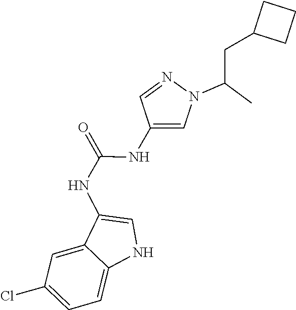 [SMILES: CC(CC1CCC1)n1cc(NC(=O)Nc2c[nH]c3ccc(Cl)cc23)cn1] | Method I: MS-ESI: 372 [M + H]+. |
| | | 1-(5-chloro-1H-indol-3- | |
| | | yl)-3-(1-(1- | |
| | | cyclobutylpropan-2-yl)- | |
| | | 1H-pyrazol-4-yl)urea | |
| Example 92 (Compound 218) | 5-chloro-1H-indole- 3-carboxylic acid; Intermediate 109 | 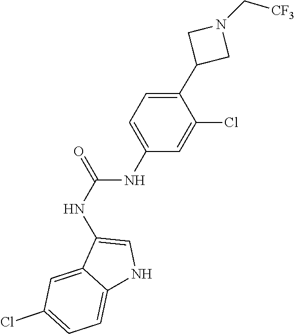 [SMILES: O=C(Nc1ccc(C2CN(CC(F)(F)F)C2)c(Cl)c1)Nc1c[nH]c2ccc(Cl)cc12] | Method I: MS-ESI: 457 [M + H]+. |
| | | 1-(5-chloro-1H-indol-3- | |
| | | yl)-3-(3-chloro-4-(1-(2,2,2- | |
| | | trifluoroethyl)azetidin-3- | |
| | | yl)phenyl)urea | |
| Example 93 (Compound 219) | 5,6-difluoro-1H- indole-3-carboxylic acid; Intermediate 106 | 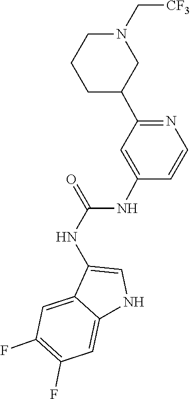 [SMILES: O=C(Nc1ccnc(C2CCCN(CC(F)(F)F)C2)c1)Nc1c[nH]c2cc(F)c(F)cc12] | Method E: MS-ESI: 454 [M + H]+. |
| | | 1-(5,6-difluoro-1H-indol- | |
| | | 3-yl)-3-(2-(1-(2,2,2- | |
| | | trifluoroethyl)piperidin-3- | |
| | | yl)pyridin-4-yl)urea | |
| Example 94 (Compound 220) | 5,6-difluoro-1H- indole-3-carboxylic acid; Intermediate 71 | 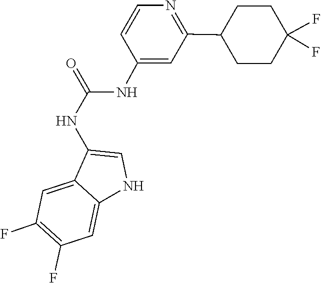 [SMILES: O=C(Nc1ccnc(C2CCC(F)(F)CC2)c1)Nc1c[nH]c2cc(F)c(F)cc12] | Method H: MS-ESI: 407 [M + H]+. |
| | | 1-(5,6-difluoro-1H-indol- | |
| | | 3-yl)-3-(2-(4,4- | |
| | | difluorocyclohexyl)pyridin- | |
| | | 4-yl)urea | |
| Example 95 (Compound 221) | 5-chloro-1H-indole- 3-carboxylic acid; Intermediate 72 | 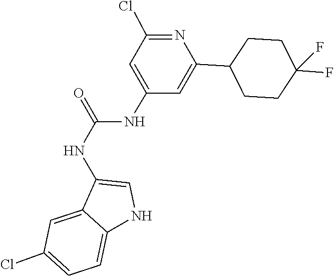 [SMILES: O=C(Nc1cc(Cl)nc(C2CCC(F)(F)CC2)c1)Nc1c[nH]c2ccc(Cl)cc12] | Method I: MS-ESI: 439 [M + H]+. |
| | | 1-(5-chloro-1H-indol-3- | |
| | | yl)-3-(2-chloro-6-(4,4- | |
| | | difluorocyclohexyl)pyridin- | |
| | | 4-yl)urea | |
| Example 96 (Compound 222) | 5-chloro-1H-indole- 3-carboxylic acid; Intermediate 112 | 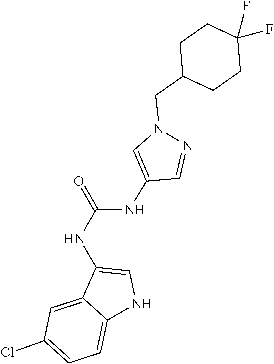 [SMILES: O=C(Nc1cnn(CC2CCC(F)(F)CC2)c1)Nc1c[nH]c2ccc(Cl)cc12] | Method I: MS-ESI: 408 [M + H]+. |
| | | 1-(5-chloro-1H-indol-3- | |
| | | yl)-3-(1-((4,4- | |
| | | difluorocyclohexyl)methyl)- | |
| | | 1H-pyrazol-4-yl)urea | |
| Example 97 (Compound 223) | 5-chloro-1H-indole- 3-carboxylic acid; Intermediate 117 | 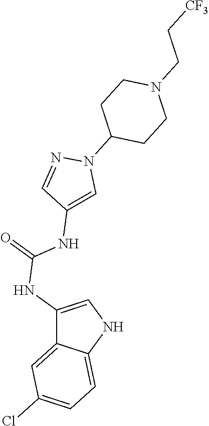 [SMILES: O=C(Nc1cnn(C2CCN(CCC(F)(F)F)CC2)c1)Nc1c[nH]c2ccc(Cl)cc12] | Method I: MS-ESI: 455 [M + H]+. |
| | | 1-(5-chloro-1H-indol-3- | |
| | | yl)-3-(1-(1-(3,3,3- | |
| | | trifluoropropyl)piperidin-4- | |
| | | yl)-1H-pyrazol-4-yl)urea | |
| Example 98 (Compound 224) | 5-chloro-1H-indole- 3-carboxylic acid; Intermediate 118 | 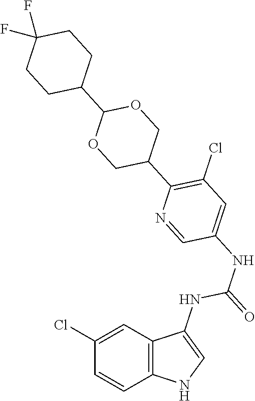 [SMILES: O=C(Nc1cnc(C2COC(C3CCC(F)(F)CC3)OC2)c(Cl)c1)Nc1c[nH]c2ccc(Cl)cc12] | Method E: MS-ESI: 510 [M + H]+. |
| | | 1-(5-chloro-1H-indol-3- | |
| | | yl)-3-(5-chloro-6-(9,9- | |
| | | difluoro-1,5- | |
| | | dioxaspiro[5.5]undecan-3- | |
| | | yl)pyridin-3-yl)urea | |
| Example 99 (Compound 225) | 5,6-difluoro-1H- indole-3-carboxylic acid; Intermediate 124 | 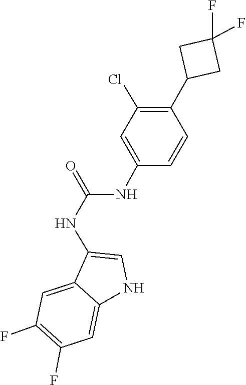 [SMILES: O=C(Nc1ccc(C2CC(F)(F)C2)c(Cl)c1)Nc1c[nH]c2cc(F)c(F)cc12] | Method I: MS-ESI: 412 [M + H]+. |
| | | 1-(3-chloro-4-(3,3- | |
| | | difluorocyclobutyl)phenyl)- | |
| | | 3-(5,6-difluoro-1H-indol- | |
| | | 3-yl)urea | |
| Example 100 (Compound 226) | 5-chloro-1H-indole- 3-carboxylic acid; Intermediate 124 | 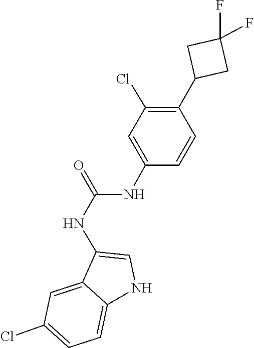 [SMILES: O=C(Nc1ccc(C2CC(F)(F)C2)c(Cl)c1)Nc1c[nH]c2ccc(Cl)cc12] | Method I: MS-ESI: 410 [M + H]+. |
| | | 1-(5-chloro-1H-indol-3- | |
| | | yl)-3-(3-chloro-4-(3,3- | |
| | | difluorocyclobutyl)phenyl) | |
| | | urea | |
| Example 101 (Compound 227) | 5-chloro-1H-indole- 3-carboxylic acid; Intermediate 125 | 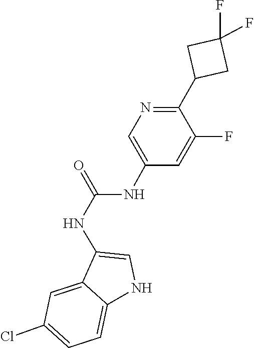 [SMILES: O=C(Nc1cnc(C2CC(F)(F)C2)c(F)c1)Nc1c[nH]c2ccc(Cl)cc12] | Method E: MS-ESI: 395 [M + H]+. |
| | | 1-(5-chloro-1H-indol-3- | |
| | | yl)-3-(6-(3,3- | |
| | | difluorocyclobutyl)-5- | |
| | | fluoropyridin-3-yl)urea | |
| Example 102 (Compound 228) | 5-chloro-1H-indole- 3-carboxylic acid; Intermediate 121 | 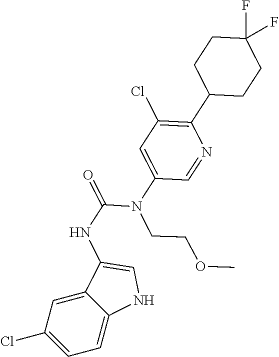 [SMILES: COCCN(C(=O)Nc1c[nH]c2ccc(Cl)cc12)c1cnc(C2CCC(F)(F)CC2)c(Cl)c1] | Method I: MS-ESI: 497 [M + H]+. |
| | | 3-(5-chloro-1H-indol-3- | |
| | | yl)-1-(5-chloro-6-(4,4- | |
| | | difluorocyclohexyl)pyridin- | |
| | | 3-yl)-1-(2- | |
| | | methoxyethyl)urea | |
| Example 103 (Compound 229) | 5-chloro-1H-indole- 3-carboxylic acid; Intermediate 122 | 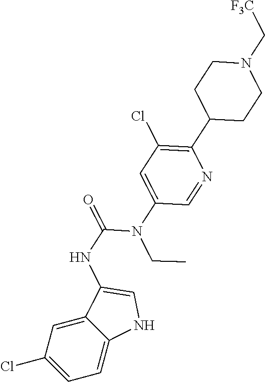 [SMILES: CCN(C(=O)Nc1c[nH]c2ccc(Cl)cc12)c1cnc(C2CCN(CC(F)(F)F)CC2)c(Cl)c1] | Method I: MS-ESI: 514 [M + H]+. |
| | | 3-(5-chloro-1H-indol-3- | |
| | | yl)-1-(5-chloro-6-(1-(2,2,2- | |
| | | trifluoroethyl)piperidin-4- | |
| | | yl)pyridin-3-yl)-1- | |
| | | ethylurea | |
| Example 104 (Compound 230) | Intermediate 100; Intermediate 113 | 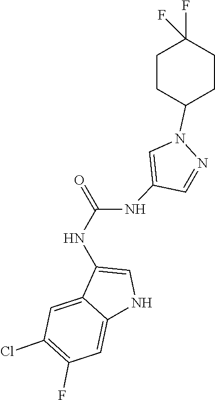 [SMILES: O=C(Nc1cnn(C2CCC(F)(F)CC2)c1)Nc1c[nH]c2cc(F)c(Cl)cc12] | Method E: MS-ESI: 412 [M + H]+. |
| | | 1-(5-chloro-6-fluoro-1H- | |
| | | indol-3-yl)-3-(1-(4,4- | |
| | | difluorocyclohexyl)-1H- | |
| | | pyrazol-4-yl)urea | |
| Example 105 (Compound 231) | 5-chloro-1H-indole- 3-carboxylic acid; Intermediate 73 | 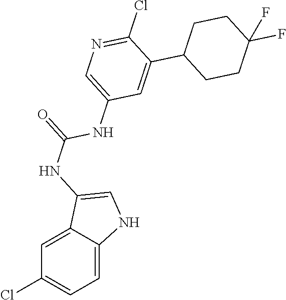 [SMILES: O=C(Nc1cnc(Cl)c(C2CCC(F)(F)CC2)c1)Nc1c[nH]c2ccc(Cl)cc12] | Method I: MS-ESI: 439 [M + H]+. |
| | | 1-(5-chloro-1H-indol-3- | |
| | | yl)-3-(6-chloro-5-(4,4- | |
| | | difluorocyclohexyl)pyridin- | |
| | | 3-yl)urea | |
| Example 106 (Compound 232) | Intermediate 101; Intermediate 5 | 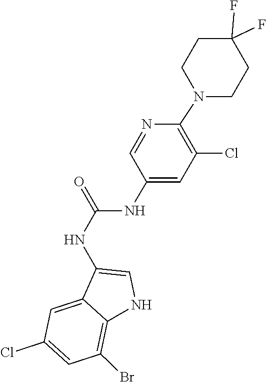 [SMILES: O=C(Nc1cnc(N2CCC(F)(F)CC2)c(Cl)c1)Nc1c[nH]c2c(Br)cc(Cl)cc12] | Method I: MS-ESI: 518 [M + H]+. |
| | | 1-(7-bromo-5-chloro-1H- | |
| | | indol-3-yl)-3-(5-chloro-6- | |
| | | (4,4-difluoropiperidin-1- | |
| | | yl)pyridin-3-yl)urea | |
| Example 107 (Compound 233) | 5-chloro-1H-indole- 3-carboxylic acid; Intermediate 79 | 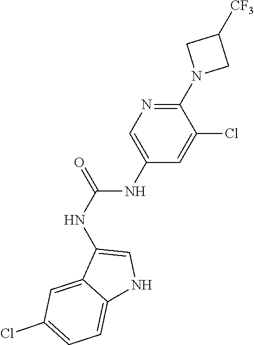 [SMILES: O=C(Nc1cnc(N2CC(C(F)(F)F)C2)c(Cl)c1)Nc1c[nH]c2ccc(Cl)cc12] | Method I: MS-ESI: 444 [M + H]+. |
| | | 1-(5-chloro-1H-indol-3- | |
| | | yl)-3-(5-chloro-6-(3- | |
| | | (trifluoromethyl)azetidin- | |
| | | 1-yl)pyridin-3-yl)urea | |
| Example 108 (Compound 234) | 5-chloro-1H-indole- 3-carboxylic acid; Intermediate 114 | 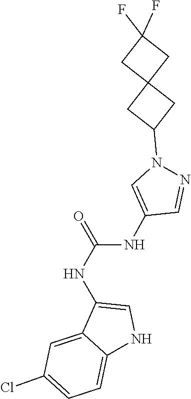 [SMILES: O=C(Nc1cnn(C2CC3(C2)CC(F)(F)C3)c1)Nc1c[nH]c2ccc(Cl)cc12] | Method H: MS-ESI: 406 [M + H]+. |
| | | 1-(5-chloro-1H-indol-3- | |
| | | yl)-3-(1-(6,6- | |
| | | difluorospiro[3.3]heptan-2- | |
| | | yl)-1H-pyrazol-4-yl)urea | |
| Example 109 (Compound 235) | 5-chloro-1H-indole- 3-carboxylic acid; Intermediate 115 | 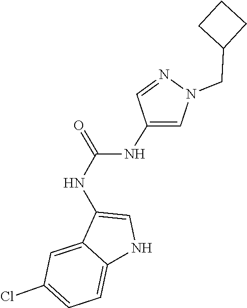 [SMILES: O=C(Nc1cnn(CC2CCC2)c1)Nc1c[nH]c2ccc(Cl)cc12] | Method E: MS-ESI: 344 [M + H]+. |
| | | 1-(5-chloro-1H-indol-3- | |
| | | yl)-3-(1- | |
| | | (cyclobutylmethyl)-1H- | |
| | | pyrazol-4-yl)urea | |
| Example 110 (Compound 236) | 5,6-difluoro-1H- indole-3-carboxylic acid; Intermediate 80 | 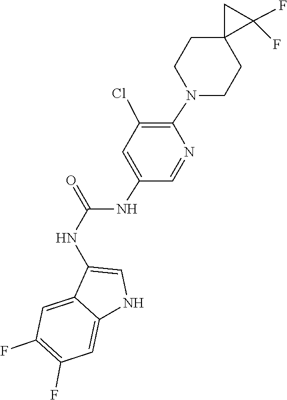 [SMILES: O=C(Nc1cnc(N2CCC3(CC2)CC3(F)F)c(Cl)c1)Nc1c[nH]c2cc(F)c(F)cc12] | Method D: MS-ESI: 468 [M + H]+. |
| | | 1-(5-chloro-6-(1,1- | |
| | | difluoro-6- | |
| | | azaspiro[2.5]octan-6- | |
| | | yl)pyridin-3-yl)-3-(5,6- | |
| | | difluoro-1H-indol-3- | |
| | | yl)urea | |
| Example 111 (Compound 237) | 5-chloro-1H-indole- 3-carboxylic acid; Intermediate 80 | 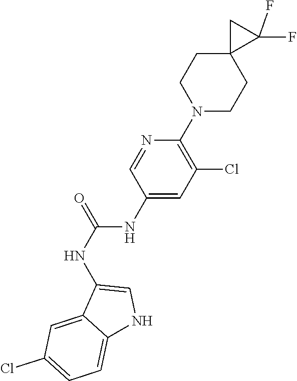 [SMILES: O=C(Nc1cnc(N2CCC3(CC2)CC3(F)F)c(Cl)c1)Nc1c[nH]c2ccc(Cl)cc12] | Method D: MS-ESI: 466 [M + H]+. |
| | | 1-(5-chloro-1H-indol-3- | |
| | | yl)-3-(5-chloro-6-(1,1- | |
| | | difluoro-6- | |
| | | azaspiro[2.5]octan-6- | |
| | | yl)pyridin-3-yl)urea | |
| Example 112 (Compound 238) | 5-chloro-1H-indole- 3-carboxylic acid; Intermediate 107 | 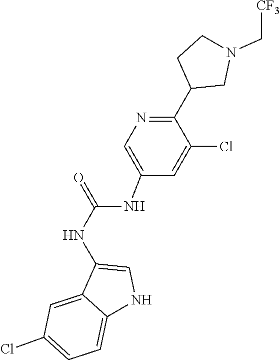 [SMILES: O=C(Nc1cnc(C2CCN(CC(F)(F)F)C2)c(Cl)c1)Nc1c[nH]c2ccc(Cl)cc12] | Method D: MS-ESI: 472 [M + H]+. |
| | | 1-(5-chloro-1H-indol-3- | |
| | | yl)-3-(5-chloro-6-(1-(2,2,2- | |
| | | trifluoroethyl)pyrrolidin-3- | |
| | | yl)pyridin-3-yl)urea | |
| Example 113 (Compound 240) | 5-chloro-1H-indole- 3-carboxylic acid; Intermediate 81 | 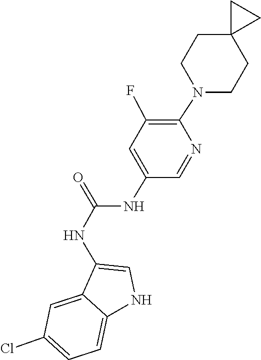 [SMILES: O=C(Nc1cnc(N2CCC3(CC2)CC3)c(F)c1)Nc1c[nH]c2ccc(Cl)cc12] | Method D: MS-ESI: 414 [M + H]+. |
| | | 1-(5-chloro-1H-indol-3- | |
| | | yl)-3-(5-fluoro-6-(6- | |
| | | azaspiro[2.5]octan-6- | |
| | | yl)pyridin-3-yl)urea | |
| Example 114 (Compound 242) | 5-chloro-1H-indole- 3-carboxylic acid; Intermediate 108 | 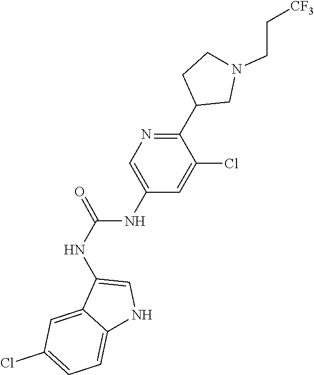 [SMILES: O=C(Nc1cnc(C2CCN(CCC(F)(F)F)C2)c(Cl)c1)Nc1c[nH]c2ccc(Cl)cc12] | Method D: MS-ESI: 486 [M + H]+. |
| | | 1-(5-chloro-1H-indol-3- | |
| | | yl)-3-(5-chloro-6-(1-(3,3,3- | |
| | | trifluoropropyl)pyrrolidin- | |
| | | 3-yl)pyridin-3-yl)urea | |
| Example 115 (Compound 243) | 5-chloro-1H-indole- 3-carboxylic acid; Intermediate 82 | 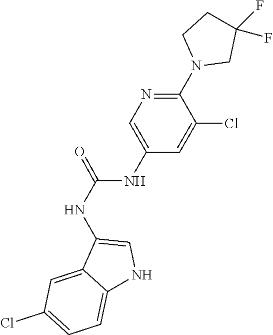 [SMILES: O=C(Nc1cnc(N2CCC(F)(F)C2)c(Cl)c1)Nc1c[nH]c2ccc(Cl)cc12] | Method D: MS-ESI: 426 [M + H]+. |
| | | 1-(5-chloro-1H-indol-3- | |
| | | yl)-3-(5-chloro-6-(3,3- | |
| | | difluoropyrrolidin-1- | |
| | | yl)pyridin-3-yl)urea | |
| Example 116 (Compound 247) | Intermediate 101; Intermediate 44 | 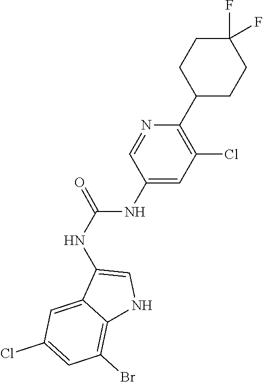 [SMILES: O=C(Nc1cnc(C2CCC(F)(F)CC2)c(Cl)c1)Nc1c[nH]c2c(Br)cc(Cl)cc12] | Method D: MS-ESI: 517 [M + H]+. |
| | | 1-(7-bromo-5-chloro-1H- | |
| | | indol-3-yl)-3-(5-chloro-6- | |
| | | (4,4- | |
| | | difluorocyclohexyl)pyridin- | |
| | | 3-yl)urea | |
| Example 117 (Compound 244) | Intermediate 101; Intermediate 12 | 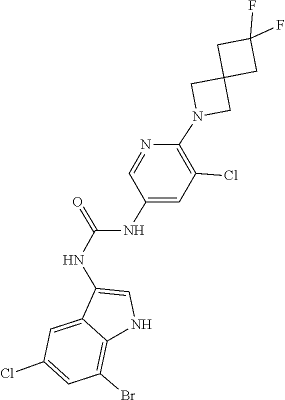 [SMILES: O=C(Nc1cnc(N2CC3(C2)CC(F)(F)C3)c(Cl)c1)Nc1c[nH]c2c(Br)cc(Cl)cc12] | Method D: MS-ESI: 530 [M + H]+. |
| | | 1-(7-bromo-5-chloro-1H- | |
| | | indol-3-yl)-3-(5-chloro-6- | |
| | | (6,6-difluoro-2- | |
| | | azaspiro[3.3]heptan-2- | |
| | | yl)pyridin-3-yl)urea | |
| Example 118 (Compound 245) | 5-fluoro-1H-indole- 3-carboxylic acid; Intermediate 6 | 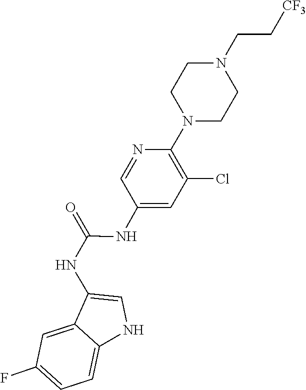 [SMILES: O=C(Nc1cnc(N2CCN(CCC(F)(F)F)CC2)c(Cl)c1)Nc1c[nH]c2ccc(F)cc12] | Method D: MS-ESI: 485 [M + H]+. |
| | | 1-(5-chloro-6-(4-(3,3,3- | |
| | | trifluoropropyl)piperazin- | |
| | | 1-yl)pyridin-3-yl)-3-(5- | |
| | | fluoro-1H-indol-3-yl)urea | |
| Example 119 (Compound 246) | 1H-indole-3- carboxylic acid; Intermediate 124 | 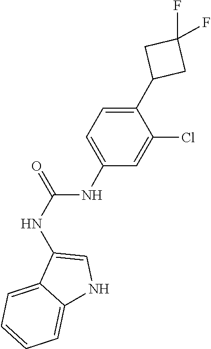 [SMILES: O=C(Nc1ccc(C2CC(F)(F)C2)c(Cl)c1)Nc1c[nH]c2ccccc12] | Method D: MS-ESI: 376 [M + H]+. |
| | | 1-(3-chloro-4-(3,3- | |
| | | difluorocyclobutyl)phenyl)- | |
| | | 3-(1H-indol-3-yl)urea | |
| Example 120 (Compound 248) | 1H-indole-3- carboxylic acid; Intermediate 44 | 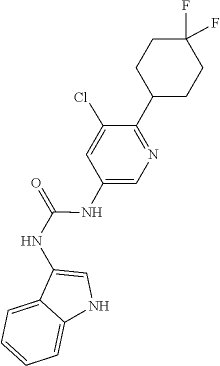 [SMILES: O=C(Nc1cnc(C2CCC(F)(F)CC2)c(Cl)c1)Nc1c[nH]c2ccccc12] | Method D: MS-ESI: 405 [M + H]+. |
| | | 1-(5-chloro-6-(4,4- | |
| | | difluorocyclohexyl)pyridin- | |
| | | 3-yl)-3-(1H-indol-3- | |
| | | yl)urea | |
| Example 121 (Compound 249) | 5-chloro-1H-indole- 3-carboxylic acid; Intermediate 83 | 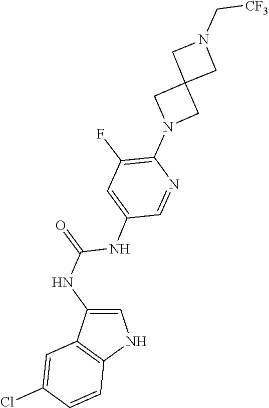 [SMILES: O=C(Nc1cnc(N2CC3(CN(CC(F)(F)F)C3)C2)c(F)c1)Nc1c[nH]c2ccc(Cl)cc12] | Method E: MS-ESI: 483 [M + H]+. |
| | | 1-(5-chloro-1H-indol-3- | |
| | | yl)-3-(5-fluoro-6-(6-(2,2,2- | |
| | | trifluoroethyl)-2,6- | |
| | | diazaspiro[3.3]heptan-2- | |
| | | yl)pyridin-3-yl)urea | |
| Example 122 (Compound 250) | 1H-indole-3- carboxylic acid; Intermediate 88 | 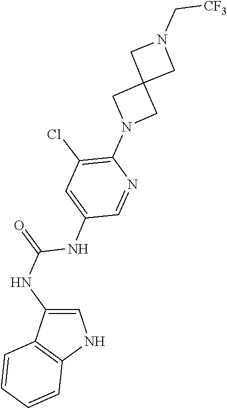 [SMILES: O=C(Nc1cnc(N2CC3(CN(CC(F)(F)F)C3)C2)c(Cl)c1)Nc1c[nH]c2ccccc12] | Method D: MS-ESI: 465 [M + H]+. |
| | | 1-(5-chloro-6-(6-(2,2,2- | |
| | | trifluoroethyl)-2,6- | |
| | | diazaspiro[3.3]heptan-2- | |
| | | yl)pyridin-3-yl)-3-(1H- | |
| | | indol-3-yl)urea | |
| Example 123 (Compound 251) | 5,6-difluoro-1H- indole-3-carboxylic acid; Intermediate 84 | 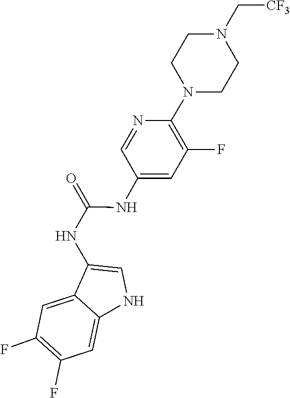 [SMILES: O=C(Nc1cnc(N2CCN(CC(F)(F)F)CC2)c(F)c1)Nc1c[nH]c2cc(F)c(F)cc12] | Method D: MS-ESI: 473 [M + H]+. |
| | | 1-(5,6-difluoro-1H-indol- | |
| | | 3-yl)-3-(5-fluoro-6-(4- | |
| | | (2,2,2- | |
| | | trifluoroethyl)piperazin-1- | |
| | | yl)pyridin-3-yl)urea | |
| Example 124 (Compound 252) | 5-fluoro-1H-indole- 3-carboxylic acid; Intermediate 127 | 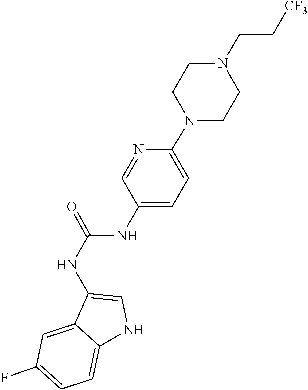 [SMILES: O=C(Nc1ccc(N2CCN(CCC(F)(F)F)CC2)nc1)Nc1c[nH]c2ccc(F)cc12] | Method D: MS-ESI: 451 [M + H]+. |
| | | 1-(5-fluoro-1H-indol-3-yl)- | |
| | | 3-(6-(4-(3,3,3- | |
| | | trifluoropropyl)piperazin- | |
| | | 1-yl)pyridin-3-yl)urea | |
| Example 125 (Compound 253) | 5-fluoro-1H-indole- 3-carboxylic acid; Intermediate 85 | 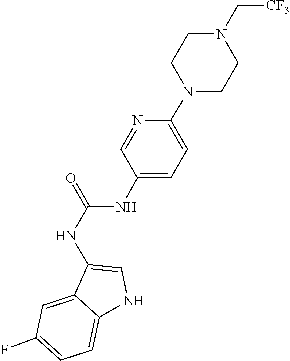 [SMILES: O=C(Nc1ccc(N2CCN(CC(F)(F)F)CC2)nc1)Nc1c[nH]c2ccc(F)cc12] | Method D: MS-ESI: 437 [M + H]+. |
| | | 1-(5-chloro-1H-indol-3-yl)- | |
| | | 3-(6-(4-(2,2,2- | |
| | | trifluoroethyl)piperazin-1- | |
| | | yl)pyridin-3-yl)urea | |
| Example 126 (Compound 254) | 1H-indole-3- carboxylic acid; Intermediate 71 | 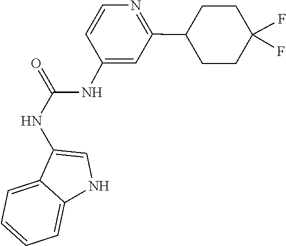 [SMILES: O=C(Nc1ccnc(C2CCC(F)(F)CC2)c1)Nc1c[nH]c2ccccc12] | Method D: MS-ESI: 371 [M + H]+. |
| | | 1-(2-(4,4- | |
| | | difluorocyclohexyl)pyridin- | |
| | | 4-yl)-3-(1H-indol | |
| | | yl)urea | |
| Example 127 (Compound 255) | 5-chloro-1H-indole- 3-carboxylic acid; Intermediate 71 | 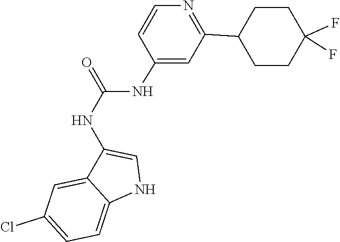 [SMILES: O=C(Nc1ccnc(C2CCC(F)(F)CC2)c1)Nc1c[nH]c2ccc(Cl)cc12] | Method D: MS-ESI: 405 [M + H]+. |
| | | 1-(5-chloro-1H-indol-3- | |
| | | yl)-3-(2-(4,4- | |
| | | difluorocyclohexyl)pyridin- | |
| | | 4-yl)urea | |
| Example 128 (Compound 256) | 5-chloro-1H-indole- 3-carboxylic acid; Intermediate 74 | 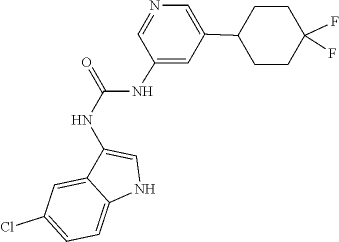 [SMILES: O=C(Nc1cncc(C2CCC(F)(F)CC2)c1)Nc1c[nH]c2ccc(Cl)cc12] | Method D: MS-ESI: 405 [M + H]+. |
| | | 1-(5-chloro-1H-indol-3- | |
| | | yl)-3-(5-(4,4- | |
| | | difluorocyclohexyl)pyridin- | |
| | | 3-yl)urea | |
| Example 129 (Compound 257) | 5-chloro-1H-indole- 3-carboxylic acid; Intermediate 56 | 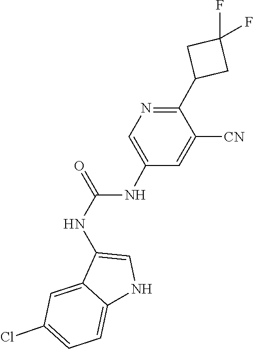 [SMILES: N#Cc1cc(NC(=O)Nc2c[nH]c3ccc(Cl)cc23)cnc1C1CC(F)(F)C1] | Method D: MS-ESI: 402 [M + H]+. |
| | | 1-(5-chloro-1H-indol-3- | |
| | | yl)-3-(5-cyano-6-(3,3- | |
| | | difluorocyclobutyl)pyridin- | |
| | | 3-yl)urea | |
| Example 130 (Compound 258) | 5-chloro-1H-indole- 3-carhoxylic acid; Intermediate 123 | 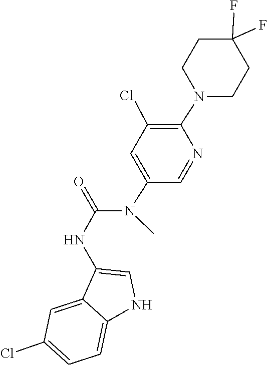 [SMILES: CN(C(=O)Nc1c[nH]c2ccc(Cl)cc12)c1cnc(N2CCC(F)(F)CC2)c(Cl)c1] | Method D: MS-ESI: 454 [M + H]+. |
| | | 3-(5-chloro-1H-indol-3- | |
| | | yl)-1-(5-chloro-6-(4,4- | |
| | | difluoropiperidin-1- | |
| | | yl)pyridin-3-yl)-1- | |
| | | methylurea | |
| Example 131 (Compound 260) | 5-chloro-1H-indole- 3-carboxylic acid; Intermediate 86 | 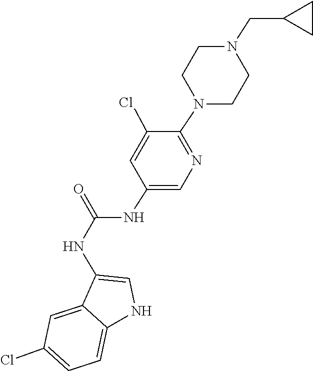 [SMILES: O=C(Nc1cnc(N2CCN(CC3CC3)CC2)c(Cl)c1)Nc1c[nH]c2ccc(Cl)cc12] | Method E: MS-ESI: 459 [M + H]+. |
| | | 1-(5-chloro-1H-indol-3- | |
| | | yl)-3-(5-chloro-6-(4- | |
| | | (cyclopropylmethyl)piperazin- | |
| | | 1-yl)pyridin-3-yl)urea | |
| Example 132 (Compound 261) | 5-chloro-1H-indole- 3-carboxylic acid; Intermediate 87 | 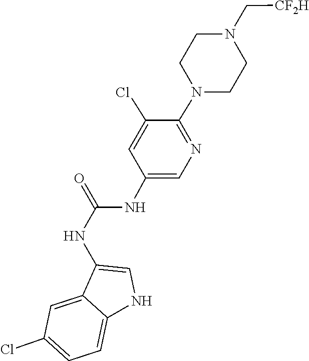 [SMILES: O=C(Nc1cnc(N2CCN(CC(F)F)CC2)c(Cl)c1)Nc1c[nH]c2ccc(Cl)cc12] | Method D: MS-ESI: 469 [M + H]+. |
| | | 1-(5-chloro-1H-indol-3- | |
| | | yl)-3-(5-chloro-6-(4-(2,2- | |
| | | difluoroethyl)piperazin-1- | |
| | | yl)pyridin-3-yl)urea | |
| Example 133 (Compound 262) | 5-chloro-1H-indole- 3-carboxylic acid; Intermediate 88 | 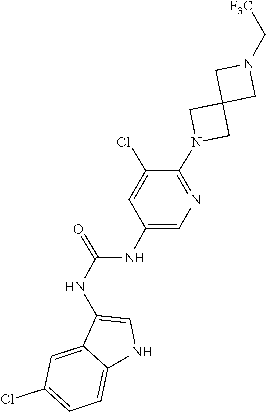 [SMILES: O=C(Nc1cnc(N2CC3(CN(CC(F)(F)F)C3)C2)c(Cl)c1)Nc1c[nH]c2ccc(Cl)cc12] | Method D: MS-ESI: 499 [M + H]+. |
| | | 1-(5-chloro-1H-indol-3- | |
| | | yl)-3-(5-chloro-6-(6-(2,2,2- | |
| | | trifluoroethyl)-2,6- | |
| | | diazaspiro[3.3]heptan-2- | |
| | | yl)pyridin-3-yl)urea | |
| Example 134 (Compound 263) | 1H-indole-3- carboxylic acid; Intermediate 119 | 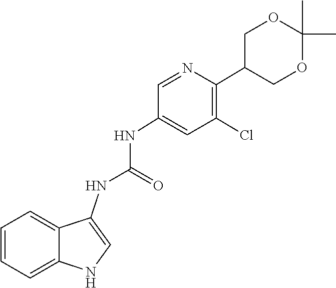 [SMILES: CC1(C)OCC(c2ncc(NC(=O)Nc3c[nH]c4ccccc34)cc2Cl)CO1] | Method D: MS-ESI: 401 [M + H]+. |
| | | 1-(5-chloro-6-(2,2- | |
| | | dimethyl-1,3-dioxan-5- | |
| | | yl)pyridin-3-yl)-3-(1H- | |
| | | indol-3-yl)urea | |
| Example 135 (Compound 264) | 5-chloro-1H-indole- 3-carboxylic acid; Intermediate 119 | 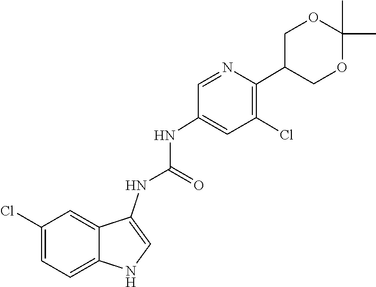 [SMILES: CC1(C)OCC(c2ncc(NC(=O)Nc3c[nH]c4ccc(Cl)cc34)cc2Cl)CO1] | Method F: MS-ESI: 435 [M + H]+. |
| | | 1-(5-chloro-1H-indol-3- | |
| | | yl)-3-(5-chloro-6-(2,2- | |
| | | dimethyl-1,3-dioxan-5- | |
| | | yl)pyridin-3-yl)urea | |
| Example 136 (Compound 266) | 5-fluoro-1H-indole- 3-carboxylic acid; Intermediate 7 | 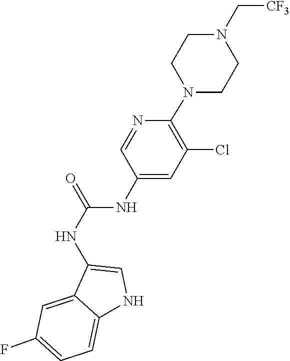 [SMILES: O=C(Nc1cnc(N2CCN(CC(F)(F)F)CC2)c(Cl)c1)Nc1c[nH]c2ccc(F)cc12] | Method D: MS-ESI: 471 [M + H]+. |
| | | 1-(5-chloro-6-(4-(2,2,2- | |
| | | trifluoroethyl)piperazin-1- | |
| | | yl)pyridin-3-yl)-3-(5- | |
| | | fluoro-1H-indol-3-yl)urea | |
| Example 137 (Compound 267) | 5-fluoro-1H-indole- 3-carboxylic acid; Intermediate 89 | 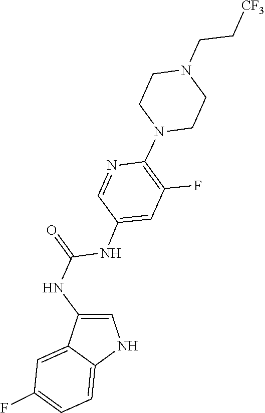 [SMILES: O=C(Nc1cnc(N2CCN(CCC(F)(F)F)CC2)c(F)c1)Nc1c[nH]c2ccc(F)cc12] | Method D: MS-ESI: 469 [M + H]+. |
| | | 1-(5-fluoro-1H-indol-3-yl)- | |
| | | 3-(5-fluoro-6-(4-(3,3,3- | |
| | | trifluoropropyl)piperazin- | |
| | | 1-yl)pyridin-3-yl)urea | |
| Example 138 (Compound 269) | 5,6-difluoro-1H- indole-3-carboxylic acid; Intermediate 7 | 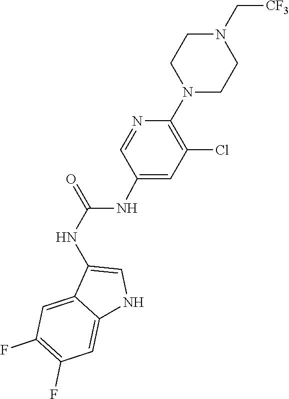 [SMILES: O=C(Nc1cnc(N2CCN(CC(F)(F)F)CC2)c(Cl)c1)Nc1c[nH]c2cc(F)c(F)cc12] | Method D: MS-ESI: 489 [M + H]+. |
| | | 1-(5-chloro-6-(4-(2,2,2- | |
| | | trifluoroethyl)piperazin-1- | |
| | | yl)pyridin-3-yl)-3-(5,6- | |
| | | difluoro-1H-indol-3- | |
| | | yl)urea | |
| Example 139 (Compound 270) | 5-fluoro-1H-indole- 3-carboxylic acid; Intermediate 75 | 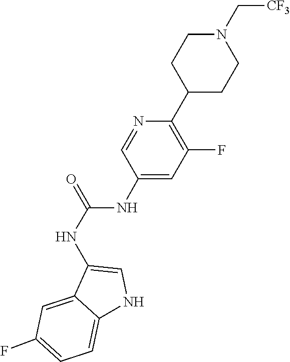 [SMILES: O=C(Nc1cnc(C2CCN(CC(F)(F)F)CC2)c(F)c1)Nc1c[nH]c2ccc(F)cc12] | Method D: MS-ESI: 454 [M + H]+. |
| | | 1-(5-fluoro-1H-indol-3-yl)- | |
| | | 3-(5-fluoro-6-(1-(2,2,2- | |
| | | trifluoroethyl)piperidin-4- | |
| | | yl)pyridin-3-yl)urea | |
| Example 140 (Compound 271) | 1H-indole-3- carboxylic acid; Intermediate 75 | 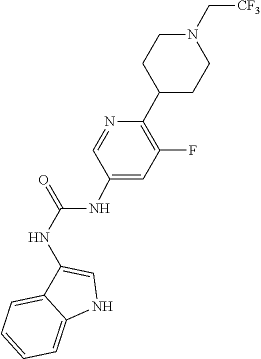 [SMILES: O=C(Nc1cnc(C2CCN(CC(F)(F)F)CC2)c(F)c1)Nc1c[nH]c2ccccc12] | Method D: MS-ESI: 436 [M + H]+. |
| | | 1-(5-fluoro-6-(1-(2,2,2- | |
| | | trifluoroethyl)piperidin-4- | |
| | | yl)pyridin-3-yl)-3-(1H- | |
| | | indol-3-yl)urea | |
| Example 141 (Compound 272) | 5-fluoro-1H-indole- 3-carboxylic acid; Intermediate 50 | 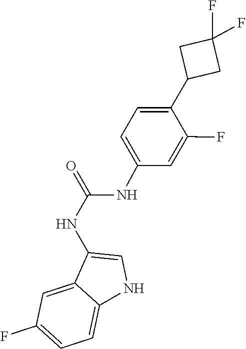 [SMILES: O=C(Nc1ccc(C2CC(F)(F)C2)c(F)c1)Nc1c[nH]c2ccc(F)cc12] | Method D: MS-ESI: 378 [M + H]+. |
| | | 1-(4-(3,3- | |
| | | difluorocyclobutyl)-3- | |
| | | fluorophenyl)-3-(5-fluoro- | |
| | | 1H-indol-3-yl)urea | |
| Example 142 (Compound 273) | 5-fluoro-1H-indole- 3-carboxylic acid; Intermediate 8 | 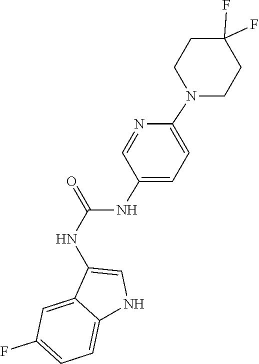 [SMILES: O=C(Nc1ccc(N2CCC(F)(F)CC2)nc1)Nc1c[nH]c2ccc(F)cc12] | Method D: MS-ESI: 390 [M + H]+. |
| | | 1-(6-(4,4- | |
| | | difluoropiperidin-1- | |
| | | yl)pyridin-3-yl)-3-(5- | |
| | | fluoro-1H-indol-3-yl)urea | |
| Example 143 (Compound 274) | 5-fluoro-1H-indole- 3-carboxylic acid; Intermediate 90 | 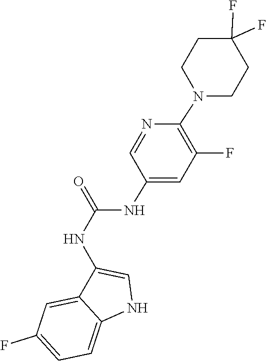 [SMILES: O=C(Nc1cnc(N2CCC(F)(F)CC2)c(F)c1)Nc1c[nH]c2ccc(F)cc12] | Method D: MS-ESI: 408 [M + H]+. |
| | | 1-(6-(4,4- | |
| | | difluoropiperidin-1-yl)-5- | |
| | | fluoropyridin-3-yl)-3-(5- | |
| | | fluoro-1H-indol-3-yl)urea | |
| Example 144 (Compound 275) | 5-chloro-1H-indole- 3-carboxylic acid; Intermediate 91 | 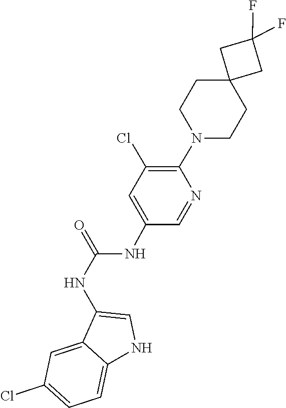 [SMILES: O=C(Nc1cnc(N2CCC3(CC2)CC(F)(F)C3)c(Cl)c1)Nc1c[nH]c2ccc(Cl)cc12] | Method D: MS-ESI: 480 [M + H]+. |
| | | 1-(5-chloro-1H-indol-3- | |
| | | yl)-3-(5-chloro-6-(2,2- | |
| | | difluoro-7- | |
| | | azaspiro[3.5]nonan-7- | |
| | | yl)pyridin-3-yl)urea | |
| Example 145 (Compound 277) | 5-fluoro-1H-indole- 3-carboxylic acid; Intermediate 51 | 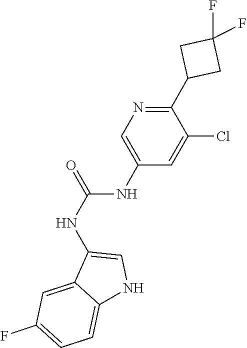 [SMILES: O=C(Nc1cnc(C2CC(F)(F)C2)c(Cl)c1)Nc1c[nH]c2ccc(F)cc12] | Method D: MS-ESI: 395 [M + H]+. |
| | | 1-(5-chloro-6-(3,3- | |
| | | difluorocyclobutyl)pyridin- | |
| | | 3-yl)-3-(5-fluoro-1H-indol- | |
| | | 3-yl)urea | |
| Example 146 (Compound 278) | 1H-indole-3- carboxylic acid; Intermediate 84 | 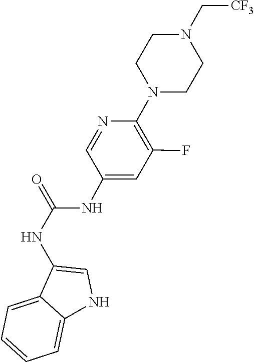 [SMILES: O=C(Nc1cnc(N2CCN(CC(F)(F)F)CC2)c(F)c1)Nc1c[nH]c2ccccc12] | Method D: MS-ESI: 437 [M + H]+. |
| | | 1-(5-fluoro-6-(4-(2,2,2- | |
| | | trifluoroethyl)piperazin-1- | |
| | | yl)pyridin-3-yl)-3-(1H- | |
| | | indol-3-yl)urea | |
| Example 147 (Compound 279) | 1H-indole-3- carboxylic acid; Intermediate 76 | 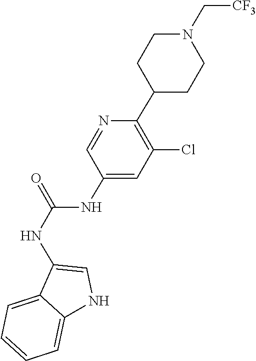 [SMILES: O=C(Nc1cnc(C2CCN(CC(F)(F)F)CC2)c(Cl)c1)Nc1c[nH]c2ccccc12] | Method D: MS-ESI: 452 [M + H]+. |
| | | 1-(5-chloro-6-(1-(2,2,2- | |
| | | trifluoroethyl)piperidin-4- | |
| | | yl)pyridin-3-yl)-3-(1H- | |
| | | indol-3-yl)urea | |
| Example 148 (Compound 280) | 1H-indole-3- carboxylic acid; Intermediate 85 | 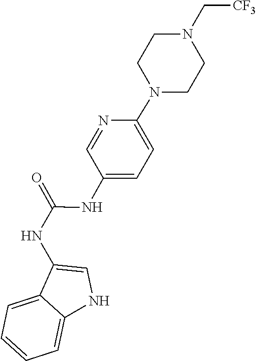 [SMILES: O=C(Nc1ccc(N2CCN(CC(F)(F)F)CC2)nc1)Nc1c[nH]c2ccccc12] | Method D: MS-ESI: 419 [M + H]+. |
| | | 1-(1H-indol-3-yl)-3-(6-(4- | |
| | | (2,2,2- | |
| | | trifluoroethyl)piperazin-1- | |
| | | yl)pyridin-3-yl)urea | |
| Example 149 (Compound 281) | 1H-indole-3- carboxylic acid; Intermediate 33 | 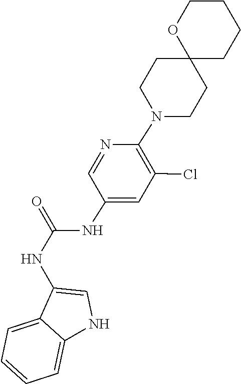 [SMILES: O=C(Nc1cnc(N2CCC3(CCCCO3)CC2)c(Cl)c1)Nc1c[nH]c2ccccc12] | Method D: MS-ESI: 440 [M + H]+. |
| | | 1-(5-chloro-6-(1-oxa-9- | |
| | | azaspiro[5.5]undecan-9- | |
| | | yl)pyridin-3-yl)-3-(1H- | |
| | | indol-3-yl)urea | |
| Example 150 (Compound 282) | 1H-indole-3- carboxylic acid; Intermediate 50 | 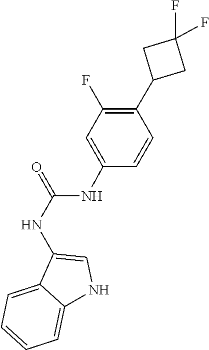 [SMILES: O=C(Nc1ccc(C2CC(F)(F)C2)c(F)c1)Nc1c[nH]c2ccccc12] | Method D: MS-ESI: 360 [M + H]+. |
| | | 1-(4-(3,3- | |
| | | difluorocyclobutyl)-3- | |
| | | fluorophenyl)-3-(1H-indol- | |
| | | 3-yl)urea | |
| Example 151 (Compound 283) | 1H-indole-3- carboxylic acid; Intermediate 51 | 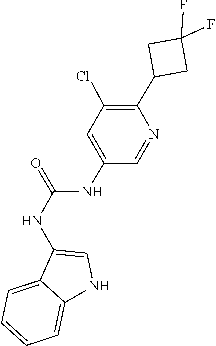 [SMILES: O=C(Nc1cnc(C2CC(F)(F)C2)c(Cl)c1)Nc1c[nH]c2ccccc12] | Method D: MS-ESI: 377 [M + H]+. |
| | | 1-(5-chloro-6-(3,3- | |
| | | difluorocyclobutyl)pyridin- | |
| | | 3-yl)-3-(1H-indol-3-yl)urea | |
| Example 152 (Compound 284) | 5-chloro-1H-indole- 3-carboxylic acid; Intermediate 92 | 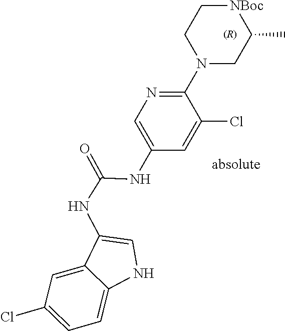 | Method D: MS-ESI: 519 [M + H]+. |
| | | tert-butyl (R)-4-(3-chloro- | |
| | | 5-(3-(5-chloro-1H-indol-3- | |
| | | yl)ureido)pyridin-2-yl)-2- | |
| | | methylpiperazine-1- | |
| | | carboxylate | |
| Example 153 (Compound 285) | 5-chloro-1H-indole- 3-carboxylic acid; Intermediate 93 | 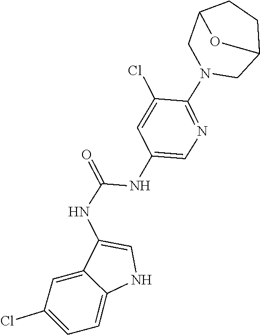 [SMILES: O=C(Nc1cnc(N2CC3CCC(C2)O3)c(Cl)c1)Nc1c[nH]c2ccc(Cl)cc12] | Method D: MS-ESI: 432 [M + H]+. |
| | | 1-(6-(8-oxa-3- | |
| | | azabicyclo[3.2.1]octan-3- | |
| | | yl)-5-chloropyridin-3-yl)- | |
| | | 3-(5-chloro-1H-indol-3- | |
| | | yl)urea | |
| Example 154 (Compound 286) | 5-chloro-1H-indole- 3-carboxylic acid; Intermediate 16 | 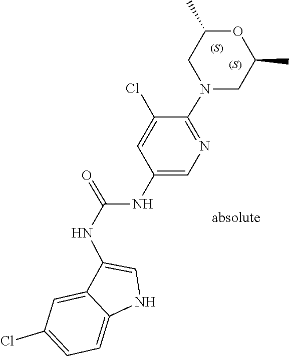 | Method D: MS-ESI: 434 [M + H]+. |
| | | 1-(5-chloro-1H-indol-3- | |
| | | yl)-3-(5-chloro-6-((2S,6S)- | |
| | | 2,6- | |
| | | dimethylmorpholino)pyridin- | |
| | | 3-yl)urea | |
| Example 155 (Compound 287) | 5-fluoro-1H-indole- 3-carboxylic acid; Intermediate 60 | 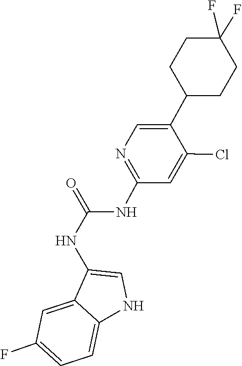 [SMILES: O=C(Nc1cc(Cl)c(C2CCC(F)(F)CC2)cn1)Nc1c[nH]c2ccc(F)cc12] | Method D: MS-ESI: 423 [M + H]+. |
| | | 1-(4-chloro-5-(4,4- | |
| | | difluorocyclohexyl)pyridin- | |
| | | 2-yl)-3-(5-fluoro-1H- | |
| | | indol-3-yl)urea | |
| Example 156 (Compound 290) | 5,6-difluoro-1H- indole-3-carboxylic acid; Intermediate 95 | 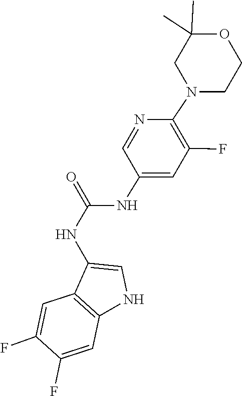 [SMILES: CC1(C)CN(c2ncc(NC(=O)Nc3c[nH]c4cc(F)c(F)cc34)cc2F)CCO1] | Method D: MS-ESI: 420 [M + H]+. |
| | | 1-(5,6-difluoro-1H-indol- | |
| | | 3-yl)-3-(6-(2,2- | |
| | | dimethylmorpholino)-5- | |
| | | fluoropyridin-3-yl)urea | |
| Example 157 (Compound 292) | 5-chloro-1H-indole- 3-carboxylic acid; Intermediate 90 | 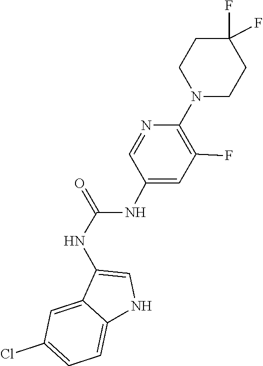 [SMILES: O=C(Nc1cnc(N2CCC(F)(F)CC2)c(F)c1)Nc1c[nH]c2ccc(Cl)cc12] | Method D: MS-ESI: 424 [M + H]+. |
| | | 1-(5-chloro-1H-indol-3- | |
| | | yl)-3-(6-(4,4- | |
| | | difluoropiperidin-1-yl)-5- | |
| | | fluoropyridin-3-yl)urea | |
| Example 158 (Compound 293) | 5-chloro-1H-indole- 3-carboxylic acid; Intermediate 15 | 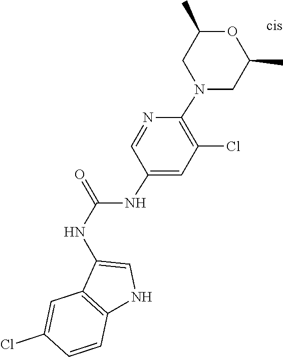 [SMILES: C[C@@H]1CN(c2ncc(NC(=O)Nc3c[nH]c4ccc(Cl)cc34)cc2Cl)C[C@H](C)O1] | Method D: MS-ESI: 434 [M + H]+. |
| | | 1-(5-chloro-1H-indol-3- | |
| | | yl)-3-(5-chloro-6-((2S,6R)- | |
| | | 2,6- | |
| | | dimethylmorpholino)pyridin- | |
| | | 3-yl)urea | |
| Example 159 (Compound 294) | 5-chloro-1H-indole- 3-carboxylic acid; Intermediate 120 | 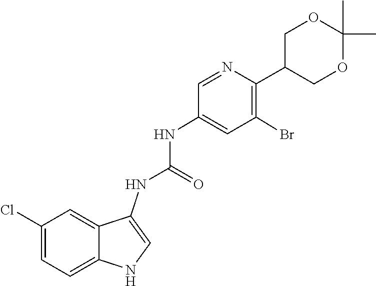 [SMILES: CC1(C)OCC(c2ncc(NC(=O)Nc3c[nH]c4ccc(Cl)cc34)cc2Br)CO1] | Method D: MS-ESI: 479 [M + H]+. |
| | | 1-(5-bromo-6-(2,2- | |
| | | dimethyl-1,3-dioxan-5- | |
| | | yl)pyridin-3-yl)-3-(5- | |
| | | chloro-1H-indol-3-yl)urea | |
| Example 160 (Compound 297) | 5-chloro-1H-indole- 3-carboxylic acid; Intermediate 76 | 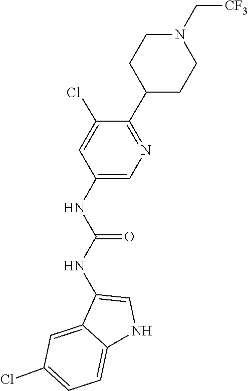 [SMILES: O=C(Nc1cnc(C2CCN(CC(F)(F)F)CC2)c(Cl)c1)Nc1c[nH]c2ccc(Cl)cc12] | Method D: MS-ESI: 486 [M + H]+. |
| | | 1-(5-chloro-1H-indol-3- | |
| | | yl)-3-(5-chloro-6-(1-(2,2,2- | |
| | | trifluoroethyl)piperidin-4- | |
| | | yl)pyridin-3-yl)urea | |
| Example 161 (Compound 298) | 5-chloro-1H-indole- 3-carboxylic acid; Intermediate 22 | 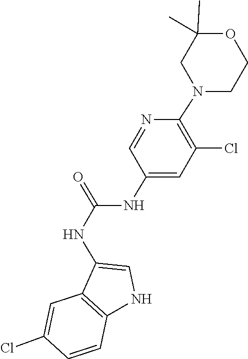 [SMILES: CC1(C)CN(c2ncc(NC(=O)Nc3c[nH]c4ccc(Cl)cc34)cc2Cl)CCO1] | Method D: MS-ESI: 434 [M + H]+. |
| | | 1-(5-chloro-1H-indol-3- | |
| | | yl)-3-(5-chloro-6-(2,2- | |
| | | dimethylmorpholino)pyridin- | |
| | | 3-yl)urea | |
| Example 162 (Compound 239) | 5-fluoro-1H-indole- 3-carboxylic acid; Intermediate 96 | 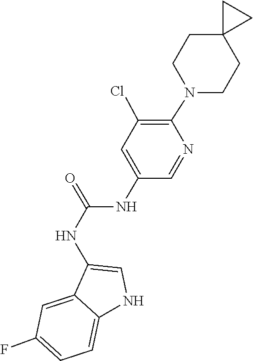 [SMILES: O=C(Nc1cnc(N2CCC3(CC2)CC3)c(Cl)c1)Nc1c[nH]c2ccc(F)cc12] | Method F: MS-ESI: 414 [M + H]+. |
| | | 1-(5-chloro-6-(6- | |
| | | azaspiro[2.5]octan-6- | |
| | | yl)pyridin-3-yl)-3-(5- | |
| | | fluoro-1H-indol-3-yl)urea | |
| Example 163 (Compound 241) | 5,6-difluoro-1H- indole-3-carboxylic acid; Intermediate 96 | 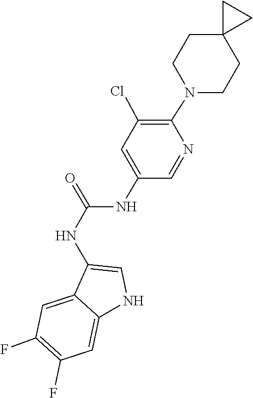 [SMILES: O=C(Nc1cnc(N2CCC3(CC2)CC3)c(Cl)c1)Nc1c[nH]c2cc(F)c(F)cc12] | Method D: MS-ESI: 432 [M + H]+. |
| | | 1-(5-chloro-6-(6- | |
| | | azaspiro[2.5]octan-6- | |
| | | yl)pyridin-3-yl)-3-(5,6- | |
| | | difluoro-1H-indol-3- | |
| | | yl)urea | |
| Example 164 (Compound 265) | 1H-indole-3- carboxylic acid; Intermediate 78 | 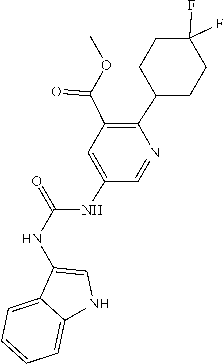 [SMILES: COC(=O)c1cc(NC(=O)Nc2c[nH]c3ccccc23)cnc1C1CCC(F)(F)CC1] | Method E: MS-ESI: 429 [M + H]+. |
| | | methyl 5-(3-(1H-indol-3- | |
| | | yl)ureido)-2-(4,4- | |
| | | difluorocyclohexyl) | |
| | | nicotinate | |
| Example 165 (Compound 268) | 5-fluoro-1H-indole- 3-carboxylic acid; Intermediate 76 | 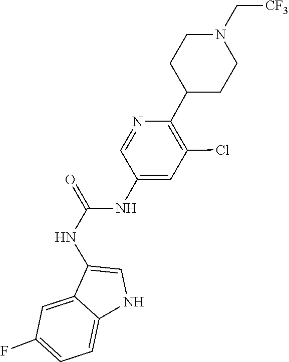 [SMILES: O=C(Nc1cnc(C2CCN(CC(F)(F)F)CC2)c(Cl)c1)Nc1c[nH]c2ccc(F)cc12] | Method D: MS-ESI: 470 [M + H]+. |
| | | 1-(5-chloro-6-(1-(2,2,2- | |
| | | trifluoroethyl)piperidin-4- | |
| | | yl)pyridin-3-yl)-3-(5- | |
| | | fluoro-1H-indol-3-yl)urea | |
| Example 166 (Compound 299) | 5,7-dichloro-1H- indole-3-carboxylic acid; Intermediate 28 | 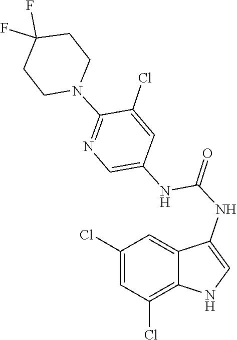 [SMILES: O=C(Nc1cnc(N2CCC(F)(F)CC2)c(Cl)c1)Nc1c[nH]c2c(Cl)cc(Cl)cc12] | Method D: MS-ESI: 473 [M + H]+. |
| | | 1-(5-chloro-6-(4,4- | |
| | | difluoropiperidin-1- | |
| | | yl)pyridin-3-yl)-3-(5,7- | |
| | | dichloro-1H-indol-3- | |
| | | yl)urea | |

NMR Data for Example 33 (Compound 153): ¹H NMR (300 MHz, DMSO-d₆) δ 10.97 (brs, 1H), 8.68 (s, 1H), 8.60 (s, 1H), 8.24 (d, J=2.4 Hz, 1H), 8.13 (d, J=2.4 Hz, 1H), 7.52 (d, J=2.1 Hz, 1H), 7.46-7.33 (m, 2H), 3.77 (t, J=4.8 Hz, 2H), 3.06 (t, J=4.8 Hz, 2H), 2.93 (s, 2H), 1.26 (s, 6H).

NMR Data for Example 34 (Compound 154): ¹H NMR (400 MHz, DMSO-d₆) δ 11.01 (brs, 1H), 9.98 (s, 1H), 9.49 (s, 1H), 8.37 (s, 1H), 7.67 (s, 1H), 7.61 (d, J=2.4 Hz, 1H), 7.47-7.36 (m, 2H), 3.05-2.99 (m, 1H), 2.15-2.06 (m, 2H), 2.03-2.00 (m, 2H), 1.97-1.90 (m, 2H), 1.82-1.74 (m, 2H).

NMR Data for Example 26 (Compound 146): ¹H NMR (300 MHz, DMSO-d₆) δ 10.98 (brs, 1H), 8.69 (s, 1H), 8.61 (s, 1H), 8.25 (d, J=2.4 Hz, 1H), 8.13 (d, J=2.4 Hz, 1H), 7.53 (d, J=2.4 Hz, 1H), 7.48-7.34 (m, 2H), 3.80-3.71 (m, 2H), 3.48-3.44 (m, 2H), 2.51-2.45 (m, 2H), 1.14 (d, J=6.0 Hz, 6H).

NMR Data for Example 24 (Compound 144): ¹H NMR (400 MHz, DMSO-d₆) δ 11.04 (brs, 1H), 8.92 (s, 1H), 8.77 (s, 1H), 8.52 (d, J=2.0 Hz, 1H), 8.23 (d, J=2.0 Hz, 1H), 7.56 (d, J=2.0 Hz, 2H), 7.38 (d, J=8.8 Hz, 2H), 7.12-7.09 (m, 1H), 3.75-3.70 (m, 1H), 2.98-2.89 (m, 4H).

### Example 167: 1-(5-chloro-1H-indol-3-yl)-3-(6-(4,4-difluorocyclohexyl)-5-fluoropyridin-3-yl)urea (Compound 295)

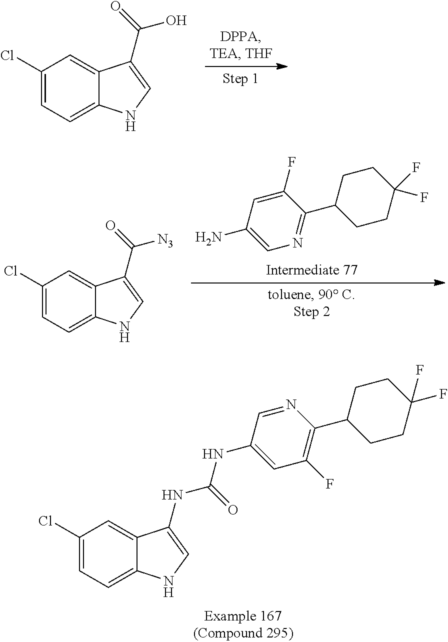 [SMILES: O=C(O)c1c[nH]c2ccc(Cl)cc12 ; [N-]=[N+]=NC(=O)c1c[nH]c2ccc(Cl)cc12 ; Nc1cnc(C2CCC(F)(F)CC2)c(F)c1 ; O=C(Nc1cnc(C2CCC(F)(F)CC2)c(F)c1)Nc1c[nH]c2ccc(Cl)cc12]

### Step 1: 5-chloro-1H-indole-3-carbonyl azide

5-Chloro-1H-indole-3-carboxylic acid (10.0 g, 51.1 mmol, 1.0 equiv.) was dissolved in THE (200.0 mL) and cooled to 0° C. DPPA (28.1 g, 102.3 mmol, 2.0 equiv.) and TEA (14.1 mL, 102.3 mmol, 2.0 equiv.) were added. The resulting mixture was stirred overnight at ambient temperature and quenched by the addition of water. The solid was collected by filtration and dried to give 5-chloro-1H-indole-3-carbonyl azide as a yellow solid, which was used to next step directly. LCMS Method A: [M+H]⁺=221.

### Step 2: 1-(5-chloro-1H-indol-3-yl)-3-(6-(4,4-difluorocyclohexyl)-5-fluoropyridin-3-yl)urea

5-Chloro-1H-indole-3-carbonyl azide (10.0 g, 45.3 mmol, 1.0 equiv.) was dissolved in toluene (500 mL), and then 6-(4,4-difluorocyclohexyl)-5-fluoropyridin-3-amine (11.5 g, 49.9 mmol, 1.1 equiv.) was added. The reaction mixture was heated to 90° C. for 6 hours, then cooled to ambient temperature and precipitated solid was collected by filtration, recrystallized twice from acetonitrile to give 3-(5-chloro-1H-indol-3-yl)-1-[6-(4,4-difluorocyclohexyl)-5-fluoropyridin-3-yl]urea as an off-white solid. LCMS Method I: [M+H]⁺=423. ¹HNMR (400 MHz, DMSO-d₆): δ 11.03 (s, 1H), 8.90 (s, 1H), 8.73 (s, 1H), 8.34 (d, 1H), 7.98-7.94 (m, 1H), 7.56 (s, 2H), 7.38 (d, 1H), 7.12-7.09 (m, 1H), 3.15-3.09 (m, 1H), 2.13-1.96 (m, 4H), 1.85-1.82 (m, 4H).

### Example 168: 1-(5-chloro-6-(4,4-difluorocyclohexyl)pyridin-3-yl)-3-(5-hydroxy-1H-indol-3-yl)urea (Compound 191)

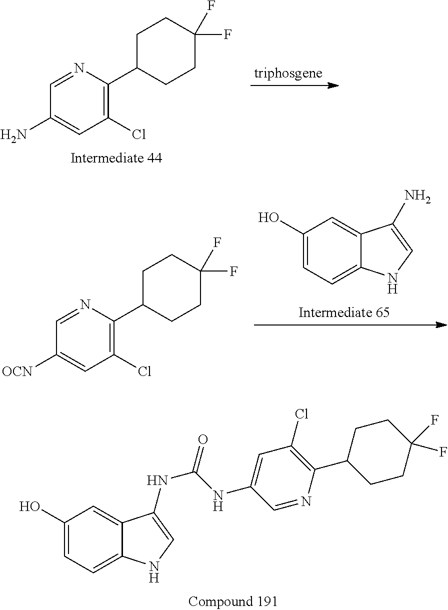

### Step 1: 3-chloro-2-(4,4-difluorocyclohexyl)-5-isocyanatopyridine

5-Chloro-6-(4,4-difluorocyclohexyl)pyridin-3-amine (300.0 mg, 1.2 mmol, 1.0 equiv.) was dissolved in THE (10 mL) and cooled to 0° C., then triphosgene (508.2 mg, 0.6 mmol, 0.5 equiv.) was added at 0° C. The resulting solution was heated to 70° C. for 2 hours, then cooled to ambient temperature and concentrated under vacuum to give 3-chloro-2-(4,4-difluorocyclohexyl)-5-isocyanatopyridine as a brown yellow solid.

### Step 2: 1-(5-chloro-6-(4,4-difluorocyclohexyl)pyridin-3-yl)-3-(5-hydroxy-1H-indol-3-yl)urea

3-Amino-1H-indol-5-ol (50.0 mg, 0.3 mmol, 1.0 equiv.) and TEA (0.6 mL, 0.4 mmol, 1.2 equiv.) were dissolved in THE (20 mL), then a solution of 3-chloro-2-(4,4-difluorocyclohexyl)-5-isocyanatopyridine (101.2 mg, 0.4 mmol, 1.1 equiv.) in THE (2 mL) was added dropwise. The reaction mixture was stirred for 30 min at ambient temperature and then quenched by the addition of water. The resulting solution was extracted with ethyl acetate and concentrated under vacuum. The residue was purified by flash column chromatography on silica gel, eluting with ethyl acetate/petroleum ether (1:3) to give the crude product, which was further purified by Prep-HPLC with the following conditions: Column, YMC-Actus Triart C18, 20*250 mm, 5 m; mobile phase, Water (10 mM NH₄HCO₃) and ACN (33% Phase B up to 63% in 10 min); Detector, UV 254/220 nm. This resulted in 1-[5-chloro-6-(4,4-difluorocyclohexyl)pyridin-3-yl]-3-(5-hydroxy-1H-indol-3-yl)urea as a white solid. LCMS Method E: [M+H]⁺=421. ¹HNMR (400 MHz, DMSO-d₆): δ 10.49 (s, 1H), 8.91 (s, 1H), 8.73 (s, 1H), 8.44-8.42 (m, 2H), 8.21 (d, 1H), 7.39 (d, 1H), 7.14 (d, 1H), 6.81 (d, 1H), 6.64-6.62 (m, 1H), 3.24-3.27 (m, 1H), 2.14-1.95 (m, 4H), 1.86-1.82 (m, 4H).

The following compound was prepared using the method described for Example 168.

| Compound | Starting materials Used | Structure | LCMS data |
|---|---|---|---|
| Example 169 (Compound 159) | Intermediate 66 Intermediate 44 | 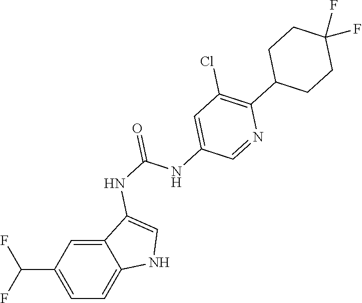 [SMILES: O=C(Nc1cnc(C2CCC(F)(F)CC2)c(Cl)c1)Nc1c[nH]c2ccc(C(F)F)cc12] 1-(5-chloro-6-(4,4- difluorocyclohexyl)pyridin- 3-yl)-3-(5- (difluoromethyl)-1H-indol- 3-yl)urea | Method D: MS-ESI: 455 [M + H]⁺. |

### Example 170: Synthesis of 1-(5-chloro-1H-indol-3-yl)-3-(1-(4,4-difluorocyclohexyl)-1H-pyrazol-4-yl)urea (Compound 209)

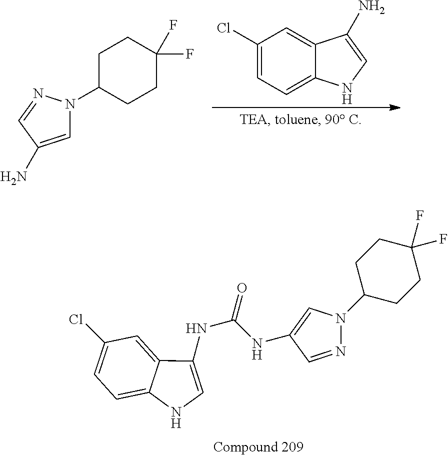 [SMILES: Nc1cnn(C2CCC(F)(F)CC2)c1 ; Nc1c[nH]c2ccc(Cl)cc12 ; O=C(Nc1cnn(C2CCC(F)(F)CC2)c1)Nc1c[nH]c2ccc(Cl)cc12]

### Step 1: 1-(5-chloro-1H-indol-3-yl)-3-(1-(4,4-difluorocyclohexyl)-1H-pyrazol-4-yl)urea

1-(4,4-difluorocyclohexyl)pyrazol-4-amine (200.0 mg, 1.0 mmol, 1.0 equiv.) was dissolved toluene (10 mL), then 5-chloro-1H-indole-3-carbonyl azide (219.3 mg, 1.0 mmol, 1.0 equiv.) was added. The resulting solution was heated to 90° C. for 2 hours, then cooled to room temperature and concentrated under vacuum. The crude product was purified by Prep-HPLC with the following conditions: Column, Xselect CSH OBD Column 30*150 mm 5 um; Mobile Phase A: Water (0.1% FA), Mobile Phase B: ACN; Flow rate: 60 mL/min; Gradient: 41% to 51% in 8 min; RT1: 7.75; Detector, UV 220/254 nm. This resulted in 1-(5-chloro-1H-indol-3-yl)-3-[1-(4,4-difluorocyclohexyl)pyrazol-4-yl]urea as an off-white solid. MS-ESI: 394 [M+H]+. ¹H NMR (400 MHz, DMSO-d₆) δ 10.93 (s, 1H), 8.39 (brs, 1H), 8.13 (brs, 1H), 7.79 (s, 1H), 7.52-7.50 (m, 2H), 7.43 (s, 1H), 7.35 (d, 1H), 7.09-7.06 (m, 1H), 4.35-4.32 (m, 1H), 2.13-1.95 (m, 8H).

### Example 171: Synthesis of 3-(5-chloro-1H-indol-3-yl)-1-[1-(4,4-difluorocyclohexyl)imidazol-4-yl]urea (Compound 212)

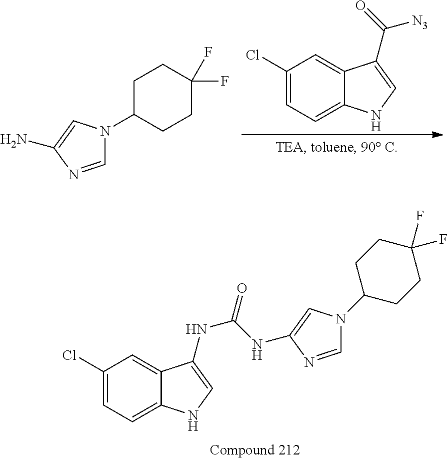

### Step 1: 3-(5-chloro-1H-indol-3-yl)-1-[1-(4,4-difluorocyclohexyl)imidazol-4-yl]urea

1-(4,4-difluorocyclohexyl)imidazol-4-amine (300.0 mg, 1.5 mmol, 1.0 equiv.) and 5-chloro-1H-indole-3-carbonyl azide (493.4 mg, 2.2 mmol, 1.5 equiv.) were dissolved in toluene (10 mL), then TEA (226.3 mg, 2.2 mmol, 1.5 equiv.) was added. The reaction mixture heated to 90° C. overnight and then quenched by the addition of water. The resulting mixture was extracted with EtOAc, washed with brine, dried over anhydrous Na₂SO₄ and concentrated under vacuum. The crude product was purified by Prep-HPLC with the following conditions (Column: YMC-Actus Triart C18 ExRS, 30*250, Sum; Mobile Phase A: Water (10 mM NH₄HCO₃+0.1% NH₄OH), Mobile Phase B: ACN; Flow rate: 60 mL/min; Gradient: 30% B to 60% B in 7 min, 254/210 nm; RT1: 5.87) to afford 3-(5-chloro-1H-indol-3-yl)-1-[1-(4,4-difluorocyclohexyl)imidazol-4-yl]urea as a white solid. MS-ESI: 394 [M+H]⁺. ¹H NMR (400 MHz, DMSO-d₆) δ 10.92 (s, 1H), 8.78 (brs, 1H), 8.56-8.53 (m, 1H), 7.55-7.53 (m, 2H), 7.47 (d, 1H), 7.36 (d, 1H), 7.11-7.08 (m, 1H), 6.99 (s, 1H), 4.22 (t, 1H), 2.10-2.06 (m, 4H), 1.99-1.88 (m, 4H).

### Example 172: Synthesis of 1-(1-(4,4-difluorocyclohexyl)-1H-pyrazol-3-yl)-3-(5-fluoro-1H-indol-3-yl)urea (Compound 211)

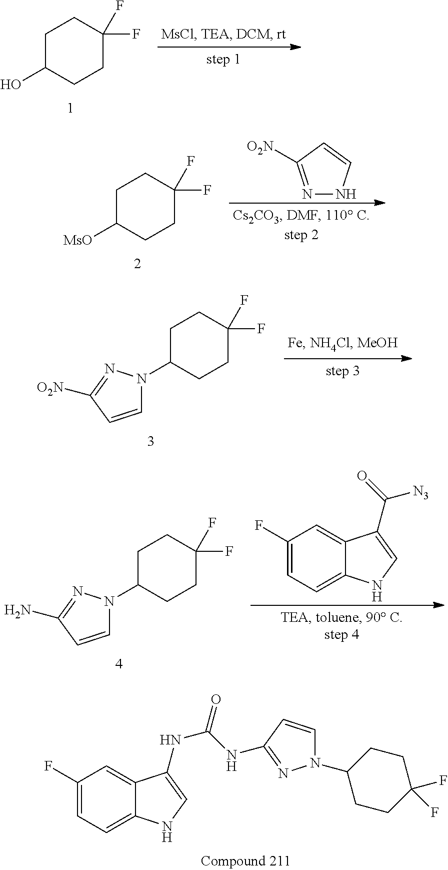

### Step 1: 4,4-difluorocyclohexyl methanesulfonate

4,4-difluorocyclohexan-1-ol (5.0 g, 36.7 mmol, 1.0 equiv.) and TEA (7.6 mL, 75.7 mmol, 1.5 equiv.) was dissolved in DCM (150 mL) and cooled to 0° C., then MsCl (4.2 mL, 37.2 mmol, 1.5 equiv.) was added dropwise. The reaction mixture was stirred overnight at room temperature and then quenched by the addition of water. The resulting mixture was extracted with DCM, washed with brine, dried over anhydrous Na₂SO₄ and concentrated under vacuum. This resulted in 4,4-difluorocyclohexyl methanesulfonate as a yellow crude oil.

### Step 2: 1-(4,4-difluorocyclohexyl)-3-nitro-1H-pyrazole

3-nitro-1H-pyrazole (500.0 mg, 4.4 mmol, 1.0 equiv.) was dissolved in THE (5 mL) and cooled to 0° C., NaH (60% wt., 264.2 mg, 49.0 mmol, 1.5 equiv.) was added under atmosphere of nitrogen. After 10 min at 0° C., 4,4-difluorocyclohexyl methanesulfonate (941.6 mg, 4.4 mmol, 1.0 equiv.) was added. The resulting mixture was heated to 110° C. overnight, then cooled to room temperature and quenched by the addition of water. The resulting mixture was extracted with EtOAc, dried over anhydrous Na₂SO₄ and concentrated under vacuum. The residue was purified by flash column chromatography on silica gel, eluting with ethyl acetate/petroleum ether (1:1) to give 1-(4,4-difluorocyclohexyl)-3-nitroimidazole as a yellow solid. ¹H NMR (400 MHz, DMSO-d₆): δ 8.16 (d, 1H), 7.08 (d, 1H), 4.64-4.54 (m, 1H), 2.18-1.93 (m, 8H).

### Step 3: 1-(4,4-difluorocyclohexyl)-1H-pyrazol-3-amine

1-(4,4-difluorocyclohexyl)-3-nitroimidazole (400.0 mg, 1.7 mmol, 1.0 equiv.) was dissolved in HBr aqueous (40%, 8 mL), then SnCl₂ (1.6 g, 8.7 mmol, 5.0 equiv.) was added. The reaction mixture was heated to 70° C. for 1 hour, and quenched by the addition of water. The resulting mixture was extracted with EtOAc, dried over anhydrous Na₂SO₄ and concentrated under vacuum. This resulted in crude 1-(4,4-difluorocyclohexyl)imidazol-3-amine as an off-white solid. MS-ESI: 202 [M+H]⁺. ¹HNMR (400 MHz, DMSO-d₆): δ 7.32 (d, 1H), 5.35 (d, 1H), 4.53 (s, 2H), 4.08-4.00 (m, 1H), 2.11-1.83 (m, 8H).

### Step 4: 1-(1-(4,4-difluorocyclohexyl)-1H-pyrazol-3-yl)-3-(5-fluoro-1H-indol-3-yl)urea

1-(4,4-difluorocyclohexyl)pyrazol-3-amine (201.0 mg, 1.0 mmol, 1.0 equiv.) and 5-fluoro-1H-indole-3-carbonyl azide (244.8 mg, 1.2 mmol, 1.2 equiv.) were dissolved in toluene (4 mL), then TEA (201.2 mg, 2.0 mmol, 2.0 equiv.) was added. The reaction mixture was heated to 90° C. for 8 hours and then concentrated under vacuum. The crude product was purified by Prep-HPLC with the following conditions (Column: XBridge Prep OBD C18 Column, 30×150 mm Sum; Mobile Phase A:Water (10 mM NH₄HCO₃+0.1% NH₄OH), Mobile Phase B: ACN; Flow rate: 60 mL/min; Gradient: 30 B to 60 B in 8 min, 254/220 nm; RT1: 6.5) to afford 3-[1-(4,4-difluorocyclohexyl)pyrazol-3-yl]-1-(5-fluoro-1H-indol-3-yl)urea as a white solid. MS-ESI: 378 [M+H]⁺. ¹H NMR (300 MHz, DMSO-d₆) δ 10.86 (s, 1H), 8.96 (s, 1H), 8.84 (brs, 1H), 7.65 (d, 1H), 7.56 (d, 1H), 7.37-7.32 (m, 1H), 7.16-7.12 (m, 1H), 6.96-6.93 (m, 1H), 6.17 (d, 1H), 4.32-4.29 (m, 1H), 2.15-2.00 (m, 8H).

### Example 173: Synthesis of 1-[1-(4,4-difluorocyclohexyl)imidazol-4-yl]-3-(5-fluoro-1H-indol-3-yl)urea (Compound 210)

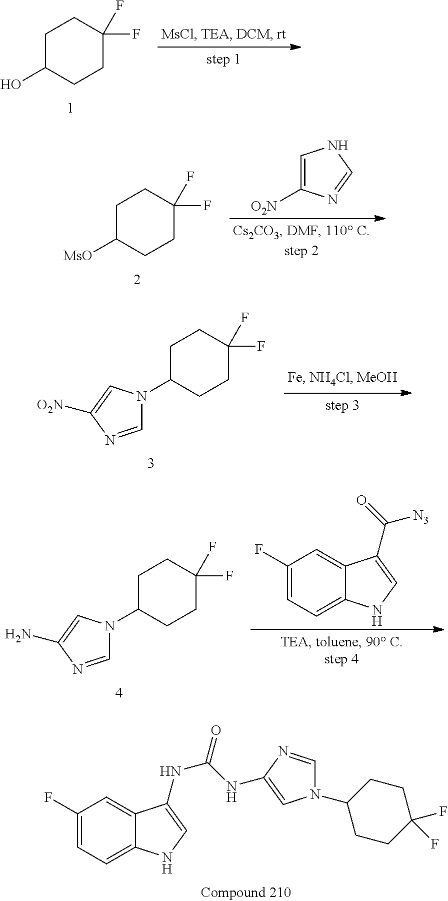

### Step 1: 4,4-difluorocyclohexyl methanesulfonate

4,4-difluorocyclohexan-1-ol (5.0 g, 36.7 mmol, 1.0 equiv.) and TEA (7.6 mL, 75.7 mmol, 1.5 equiv.) was dissolved in DCM (150 mL) and cooled to 0° C., then MsC (4.2 mL, 37.2 mmol, 1.5 equiv.) was added dropwise. The reaction mixture was stirred overnight at room temperature and then quenched by the addition of water. The resulting mixture was extracted with DCM, washed with brine, dried over anhydrous Na₂SO₄ and concentrated under vacuum. This resulted in 4,4-difluorocyclohexyl methanesulfonate as a yellow crude oil.

### Step 2: 1-(4,4-difluorocyclohexyl)-4-nitroimidazole

4-nitroimidazole (5.5 g, 49.0 mmol, 1.5 equiv.) was dissolved in THE (35 mL) and cooled to 0° C., NaH (60% wt., 1.9 g, 49.0 mmol, 1.5 equiv.) was added under atmosphere of nitrogen. After 10 min at 0° C., 4,4-difluorocyclohexyl methanesulfonate (7.0 g, 32.7 mmol, 1.0 equiv.) was added. The resulting mixture was heated to 80° C. overnight, then cooled to room temperature and quenched by the addition of water. The resulting mixture was extracted with EtOAc, dried over anhydrous Na₂SO₄ and concentrated under vacuum. The residue was purified by flash column chromatography on silica gel, eluting with ethyl acetate/petroleum ether (1:1) to give 1-(4,4-difluorocyclohexyl)-4-nitroimidazole as an orange oil. ¹HNMR (400 MHz, DMSO-d₆): δ 8.57 (d, 1H), 7.99 (d, 1H), 4.43-4.39 (m, 1H), 2.24-2.08 (m, 4H), 2.09-1.92 (m, 4H).

### Step 3: 1-(4,4-difluorocyclohexyl)imidazol-4-amine

1-(4,4-difluorocyclohexyl)-4-nitroimidazole (500.0 mg, 2.2 mmol, 1.0 equiv.) was dissolved in MeOH (5 mL), then Fe (241.9 mg, 4.3 mmol, 2.0 equiv.) and NH4C1 (aq.) (1.0 mL) were added. The reaction mixture was stirred overnight at room temperature and quenched by the addition of water. The resulting mixture was extracted with EtOAc, dried over anhydrous Na₂SO₄ and concentrated under vacuum. This resulted in crude 1-(4,4-difluorocyclohexyl)imidazol-4-amine as a brown oil. MS-ESI: 202 [M+H]*.

### Step 4: 1-[1-(4,4-difluorocyclohexyl)imidazol-4-yl]-3-(5-fluoro-1H-indol-3-yl)urea

1-(4,4-difluorocyclohexyl)imidazol-4-amine (300.0 mg, 1.5 mmol, 1.0 equiv.) and 5-fluoro-1H-indole-3-carbonyl azide (456.5 mg, 2.2 mmol, 1.5 equiv.) were dissolved in toluene (10 mL), then TEA (301.7 mg, 3.0 mmol, 2.0 equiv.) was added. The reaction mixture was heated to 90° C. overnight, then cooled to room temperature and concentrated under vacuum. The residue was diluted with water, extracted with EtOAc, washed with brine, dried over anhydrous Na₂SO₄ and concentrated under vacuum. The residue was purified by Prep-HPLC with the following conditions (Column: YMC-Actus Triart C18 ExRS, 30 mm×150 mm, Sum; Mobile Phase A: Water (10 mM NH₄HCO₃), Mobile Phase B: ACN; Flow rate: 60 mL/min; Gradient: 20% B to 50% B in 9 min, 254/220 nm) to afford 1-[1-(4,4-difluorocyclohexyl)imidazol-4-yl]-3-(5-fluoro-1H-indol-3-yl)urea as a yellow solid. MS-ESI: 378 [M+H]⁺. ¹H NMR (400 MHz, DMSO-d₆) δ 10.81 (s, 1H), 8.72 (brs, 1H), 8.59 (s, 1H), 7.54-7.52 (m, 2H), 7.35-7.31 (m, 1H), 7.16-7.13 (m, 1H), 6.98 (s, 1H), 6.96-6.91 (m, 1H), 4.22 (t, 1H), 2.13-1.88 (m, 8H).

### Example 174: Synthesis of 1-(1H-indol-3-yl)-3-(5-methyl-6-(1-(2,2,2-trifluoroethyl)piperidin-4-yl)pyridin-3-yl)urea (Compound 276)

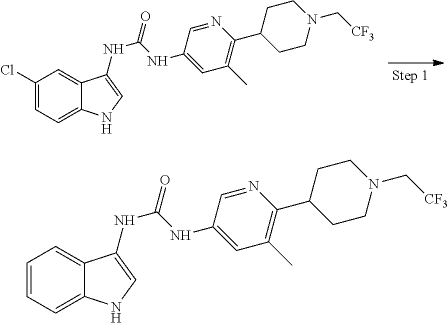 [SMILES: Cc1cc(NC(=O)Nc2c[nH]c3ccc(Cl)cc23)cnc1C1CCN(CC(F)(F)F)CC1 ; Cc1cc(NC(=O)Nc2c[nH]c3ccccc23)cnc1C1CCN(CC(F)(F)F)CC1]

3-(5-Chloro-1H-indol-3-yl)-1-[5-methyl-6-[1-(2,2,2-trifluoroethyl)piperidin-4-yl]pyridin-3-yl]urea (100.0 mg, 0.2 mmol, 1.0 equiv.) was dissolved in MeOH (5 mL), then Pd/C (10% wt., 5.0 mg) was added. The mixture was sparged with nitrogen, placed under an atmosphere of hydrogen gas (balloon), then stirred for 4 hours at ambient temperature. The solids were removed by filtration and the filtrate was concentrated under vacuum. The residue was purified by reverse flash chromatography with following conditions: column, C18 silica gel; mobile phase A, MeCN; mobile phase B, water, 30% B to 60% B gradient in 30 min; detector, UV 254 nm. This resulted in 3-(1H-indol-3-yl)-1-[5-methyl-6-[1-(2,2,2-trifluoroethyl)piperidin-4-yl]pyridin-3-yl]urea as a white solid. LCMS Method D: [M+H]⁺=432. ¹HNMR (300 MHz, DMSO-d₆): δ 10.74 (s, 1H), 8.58-8.55 (m, 1H), 8.54 (s, 1H), 8.36 (d, 1H), 7.76 (d, 1H), 7.53-7.49 (m, 2H), 7.35-7.32 (m, 1H), 7.13-7.07 (m, 1H), 7.04-6.99 (m, 1H), 3.24-3.14 (m, 3H), 3.03-2.99 (m, 2H), 2.82-2.73 (m, 1H), 2.30 (s, 3H), 1.90-1.78 (m, 2H), 1.65-1.60 (m, 2H).

### Example 175: Synthesis of 1-(5-chloro-1H-indol-3-yl)-3-(5-chloro-6-(cis-3,5-dimethylpiperazin-1-yl)pyridin-3-yl)urea (Compound 288)

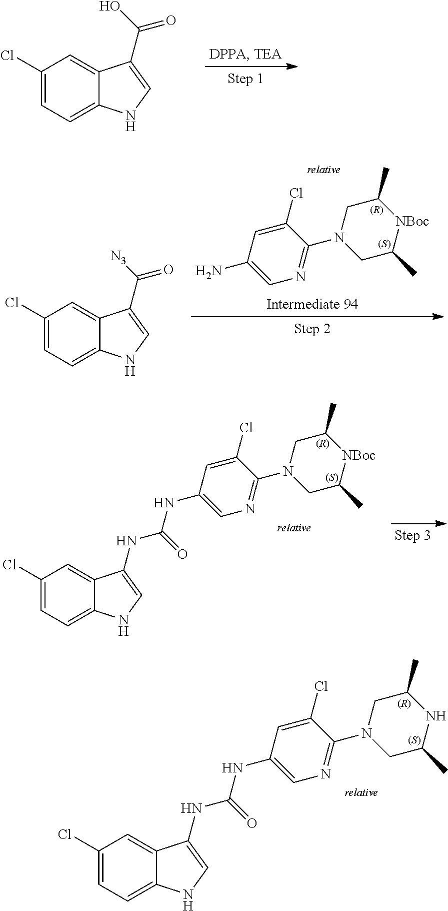

### Step 1 and Step 2: tert-butyl cis-4-(3-chloro-5-(3-(5-chloro-1H-indol-3-yl)ureido)pyridin-2-yl)-2,6-dimethylpiperazine-1-carboxylate

The title compound was prepared using the same methods described for Example 2 with intermediate 94 and 5-chloro-1H-indole-3-carboxylic acid.

### Step 3: 1-(5-chloro-1H-indol-3-yl)-3-(5-chloro-6-(cis-3,5-dimethylpiperazin-1-yl)pyridin-3-yl)urea

tert-Butyl cis-4-(3-chloro-5-[[(5-chloro-1H-indol-3-yl)carbamoyl]amino]pyridin-2-yl)-2,6-dimethylpiperazine-1-carboxylate (300.0 mg, 0.6 mmol, 1.0 equiv.) was dissolved in DCM (5 mL), TFA (5 mL) was added. The reaction mixture was stirred for 4 hours at ambient temperature and then concentrated under vacuum. The residue was purified by Prep-HPLC with following conditions: Column: XBridge Prep OBD C18 Column, 30×150 mm 5 um; Mobile Phase A: Water (10 mM NH₄HCO₃), Mobile Phase B: ACN; Flow rate: 60 mL/min; Gradient: 22 B to 52 B in 7 min; 254 nm. This resulted in 1-(5-chloro-1H-indol-3-yl)-3-(5-chloro-6-(cis-3,5-dimethylpiperazin-1-yl)pyridin-3-yl)urea as an off-white solid. LCMS Method D: [M+H]⁺=433. ¹HNMR (300 MHz, DMSO-d₆): δ 10.99 (s, 1H), 8.70 (d, 2H), 8.22 (d, 1H), 8.10 (d, 1H), 7.57-7.54 (m, 2H), 7.38-7.35 (m, 1H), 7.11-7.07 (m, 1H), 3.43-3.39 (m, 3H), 2.94-2.88 (m, 2H), 2.31-2.24 (m, 2H), 0.99 (d, 6H).

The following compounds were prepared using the method described for Example 175.

| Compound | Starting materials | Structure | LCMS data |
|---|---|---|---|
| Example 176 (Compound 289) | 5-chloro-1H-indol- 3-amine Intermediate 97 | 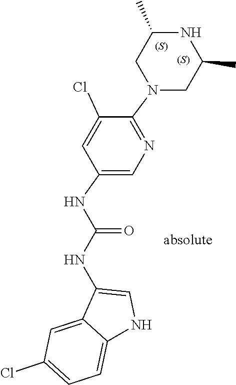 1-(5-chloro-1H-indol-3-yl)-3- (5-chloro-6-((3S,5S)-3,5- dimethylpiperazin-1- yl)pyridin-3-yl)urea | Method D: MS-ESI: 433 [M + H]⁺. |
| Example 177 (Compound 291) | 5-chloro-1H-indol- 3-amine Intermediate 98 | 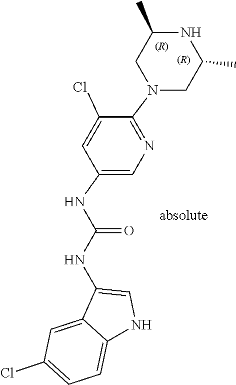 1-(5-chloro-1H-indol-3-yl)-3- (5-chloro-6-((3R,5R)-3,5- dimethylpiperazin-1- yl)pyridin-3-yl)urea | Method D: MS-ESI: 433 [M + H]⁺. |
| Example 178 (Compound 296) | 5-chloro-1H-indol- 3-amine Intermediate 99 | 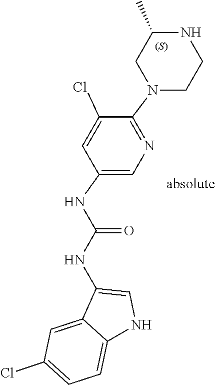 (S)-1-(5-chloro-1H-indol-3- yl)-3-(5-chloro-6-(3- methylpiperazin-1-yl)pyridin- 3-yl)urea | Method D: MS-ESI: 419 [M + H]⁺. |

### Biological Assays

STING pathway activation by the compounds described herein is measured using THP1-Dual™ cells (KO-IFNAR2).

THP1-Dual™ KO-IFNAR2 Cells (obtained from invivogen) are maintained in RPMI, 10% FCS, 5 ml P/S, 2 mM L-glut, 10 mM Hepes, and 1 mM sodium pyruvate.

Compounds are spotted in empty 384 well tissue culture plates (Greiner 781182) by Echo for a final concentration of 0.0017-100 μM. Cells are plated into the TC plates at 40 μL per well, 2×10E6 cells/mL. For activation with STING ligand, 2′3′cGAMP (MW 718.38, obtained from Invivogen), is prepared in Optimem media.

The following solutions are prepared for each 1×384 plate:
Solution A: 2 mL Optimem with one of the following stimuli:
   60 uL of 10 mM 2′3′cGAMP->150 μM stock
Solution B: 2 mL Optimem with 60 μL Lipofectamine 2000->Incubate 5 min at RT

2 mL of solution A and 2 ml Solution B is mixed and incubated for 20 min at room temperature (RT). 20 uL of transfection solution (A+B) is added on top of the plated cells, with a final 2′3′cGAMP concentration of 15 μM. The plates are then centrifuged immediately at 340 g for 1 minute, after which they are incubated at 37° C., 5% CO₂, >98% humidity for 24 h. Luciferase reporter activity is then measured. EC₅₀ values are calculated by using standard methods known in the art.

Luciferase reporter assay: 10 μL of supernatant from the assay is transferred to white 384-plate with flat bottom and squared wells. One pouch of QUANTI-Luc™ Plus us dissolved in 25 mL of water. 100 μL of QLC Stabilizer per 25 mL of QUANTI-Luc™ Plus solution is added. 50 μL of QUANTI-Luc™ Plus/QLC solution per well is then added. Luminescence is measured on a Platereader (e.g., Spectramax I3X (Molecular Devices GF3637001)).

Luciferase reporter activity is then measured. EC₅₀ values are calculated by using standard methods known in the art.

Table BA shows the activity of compounds in STING reporter assay: <0.008 μM=“++++++”; >0.008 and <0.04 μM=“+++++”; >0.04 and <0.2 μM=“++++”; >0.2 and <1 μM=“+++”; >1 and <5 μM=“++”; >5 and <100 μM=

TABLE BA

| Compound No. | hSTING EC₅₀ |
|---|---|
| 123 | + |
| 124 | ++++ |
| 125 | +++ |
| 126 | ++++ |
| 127 | +++ |
| 128 | ++ |
| 129 | ++++ |
| 130 | ++++ |
| 131 | ++++ |
| 132 | ++++ |
| 133 | ++++ |
| 134 | ++ |
| 135 | +++ |
| 136 | +++ |
| 137 | ++++ |
| 138 | ++++ |
| 139 | ++++ |
| 140 | ++ |
| 141 | +++ |
| 142 | ++++ |
| 143 | +++ |
| 144 | ++++ |
| 145 | +++ |
| 146 | ++++ |
| 147 | ++++ |
| 148 | ++++ |
| 149 | +++ |
| 150 | +++ |
| 151 | +++ |
| 152 | ++ |
| 153 | ++++ |
| 154 | ++++ |
| 155 | ++++ |
| 156 | ++++ |
| 157 | ++++ |
| 158 | +++ |
| 159 | ++++ |
| 160 | >30.0 |
| 161 | + |
| 162 | +++ |
| 163 | ++++ |
| 164 | ++ |
| 165 | +++ |
| 166 | +++ |
| 167 | +++ |
| 168 | +++ |
| 169 | +++ |
| 170 | ++++ |
| 171 | + |
| 172 | +++ |
| 173 | +++ |
| 174 | ++++ |
| 175 | +++ |
| 176 | +++ |
| 177 | ++++ |
| 178 | ++ |
| 179 | +++ |
| 180 | ++++ |
| 181 | ++++ |
| 182 | ++++ |
| 183 | ++++ |
| 184 | ++++ |
| 185 | +++ |
| 186 | +++ |
| 187 | +++ |
| 188 | +++ |
| 189 | ++++ |
| 190 | +++ |
| 191 | +++ |
| 192 | ++++ |
| 193 | ++++ |
| 194 | +++ |
| 195 | ++++ |
| 196 | ++++ |
| 197 | ++++ |
| 198 | ++++ |
| 199 | ++++ |
| 200 | ++++ |
| 201 | +++ |
| 202 | ++ |
| 203 | ++++ |
| 204 | ++ |
| 205 | +++ |
| 206 | ++++ |
| 207 | ++++ |
| 208 | ++ |
| 209 | +++ |
| 210 | ++ |
| 211 | ++ |
| 212 | ++ |
| 213 | ++ |
| 214 | +++ |
| 215 | +++ |
| 216 | ++ |
| 217 | +++ |
| 218 | ++++ |
| 219 | ++ |
| 220 | +++ |
| 221 | ++++ |
| 222 | +++ |
| 223 | ++ |
| 224 | ++++ |
| 225 | +++ |
| 226 | ++++ |
| 227 | ++++ |
| 228 | +++ |
| 229 | +++ |
| 230 | +++ |
| 231 | ++++ |
| 232 | +++ |
| 233 | ++++ |
| 234 | ++++ |
| 235 | +++ |
| 236 | ++++ |
| 237 | ++++ |
| 238 | ++++ |
| 239 | ++++ |
| 240 | ++++ |
| 241 | ++++ |
| 242 | ++++ |
| 243 | ++++ |
| 244 | ++ |
| 245 | ++++ |
| 246 | ++++ |
| 247 | +++ |
| 248 | ++++ |
| 249 | +++ |
| 250 | +++ |
| 251 | ++++ |
| 252 | +++ |
| 253 | +++ |
| 254 | +++ |
| 255 | +++ |
| 256 | +++ |
| 257 | ++++ |
| 258 | +++ |
| 260 | ++++ |
| 261 | ++++ |
| 262 | ++++ |
| 263 | +++ |
| 264 | ++++ |
| 265 | ++++ |
| 266 | ++++ |
| 267 | ++++ |
| 268 | ++++ |
| 269 | ++++ |
| 270 | ++++ |
| 271 | +++ |
| 272 | ++++ |
| 273 | +++ |
| 274 | ++++ |
| 275 | ++++ |
| 276 | +++ |
| 277 | ++++ |
| 278 | +++ |
| 279 | ++++ |
| 280 | +++ |
| 281 | ++++ |
| 282 | +++ |
| 283 | ++++ |
| 284 | ++++ |
| 285 | ++++ |
| 286 | ++++ |
| 287 | ++++ |
| 288 | + |
| 289 | + |
| 290 | ++++ |
| 291 | ++ |
| 292 | ++++ |
| 293 | ++++ |
| 294 | ++++ |
| 295 | ++++ |
| 296 | ++ |
| 297 | ++++ |
| 298 | ++++ |
| 300 | ++++ |

### Numbered Clauses

The compounds, compositions, methods, and other subject matter described herein are further described in the following numbered clauses:

1. A compound of Formula I:

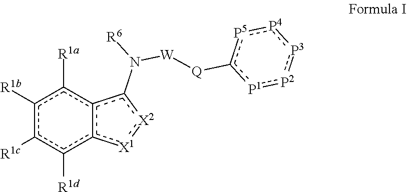

or a pharmaceutically acceptable salt thereof or a tautomer thereof, wherein:

X¹ is selected from the group consisting of O, S, N, NR², and CR¹;

X² is selected from the group consisting of O, S, N, NR⁴, and CR⁵;

each [symbol] is independently a single bond or a double bond, provided that:

the five-membered ring comprising X¹ and X² is heteroaryl;

the 6-membered ring

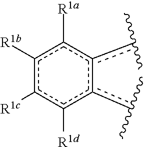

is aromatic; and

and the ring comprising P¹, P², P³, P⁴, and P⁵ is aromatic;

P¹, P², P³, P⁴, and P⁵ are defined according to (AA) or (BB):

### AA

each of P¹, P², P³, P⁴, and P⁵ is independently selected from the group consisting of: N, CH, CR⁷, and CR^{c}, provided that 1-2 of P¹, P², P³, P⁴, and P⁵ is an independently selected CR⁷; or

### BB

P¹ is absent, thereby providing a 5-membered ring,

each of P², P³, P⁴, and P⁵ is independently selected from the group consisting of O, S, N, NH, NR^{d}, NR⁷, CH, CR⁷, and CR^{c}, provided that 1-3 of P², P³, P⁴, and P⁵ is O, S, N, NH, NR^{d}, or NR⁷; and 1-2 of P², P³, P⁴, and P⁵ is an independently selected NR⁷ or CR⁷;

each R⁷ is independently selected from the group consisting of: —R⁸ and -L³-R⁹

R⁸ and R⁹ are independently selected from the group consisting of:

(a) C₃₋₁₂ cycloalkyl or C₃₋₁₂ cycloalkenyl, each of which is optionally substituted with 1-4 independently selected R⁷′;

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R⁷′;

(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R⁷′; and

(d) C₆₋₁₀ aryl optionally substituted with 1-4 independently selected R⁷′;

-L³ is selected from the group consisting of —O—, —C₁₋₄ alkylene, —S—, —NH—, S(O)₁₋₂, C(═O)NH, NHC(═O), C(═O)O, OC(═O), C(═O), NHS(O)₂, and S(O)₂NH;

each occurrence of R⁷′ is independently selected from the group consisting of: halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),

W is selected from the group consisting of:

(i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);

Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents point of attachment to W;

each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);

each occurrence of R² is independently selected from the group consisting of:

(i) H;

(ii) C₁₋₆ alkyl, which is optionally substituted with 1-3 independently selected R^{a};

(iii) —C(O)(C₁₋₆ alkyl) optionally substituted with 1-3 independently selected R^{a};

(iv) —C(O)O(C₁₋₄ alkyl) optionally substituted with 1-3 independently R^{a};

(v) —CON(R′)(R″);

(vi) —S(O)₁₋₂(NR′R″);

(vii) —S(O)₁₋₂(C₁₋₄ alkyl) optionally substituted with 1-3 independently selected R^{a};

(viii) —OH;

(ix) C₁₋₄ alkoxy; and

(x) -L⁴-L⁵-Rⁱ;

R⁴ is selected from the group consisting of H and C₁₋₆ alkyl optionally substituted with 1-3 independently selected R^{a};

R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;

each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;

each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; —C(═O)N(R′)(R″); and -L¹-L²-R^{h};

R^{d} is selected from the group consisting of: C₁₋₆ alkyl optionally substituted with 1-3 substituents each independently selected from the group consisting of: halo, C₁₋₃ alkoxy, C₁₋₃ haloalkoxy, OH, and C₃₋₆ cycloalkyl; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂N(R′)(R″); —S(O)₁₋₂(C₁₋₄ alkyl); —OH; and C₁₋₄ alkoxy;

each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂N(R′)(R″); —S(O)₁₋₂(C₁₋₄ alkyl); —OH; and C₁₋₄ alkoxy; or

R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene; -L² is —O—, —N(H)—, —S(O)₀₋₂—, or a bond;

R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;

-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};

-L⁵- is selected from the group consisting of a bond and C₁₋₄ alkylene;

Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and

each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl;

or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S;

provided that:

(a) when X¹ is NR²; X² is CH; each of R^{1a}, R^{1b}, R^{1c}, R^{1d}, and R⁶ is H; W is C(═O); Q is NH; and P¹, P², P³, P⁴, and P⁵ are defined according to (AA); then:
R² cannot be CH₂CH₂OCH₃, CH₃, CH₂CH₃, or SO₂-(p-tolyl) when the

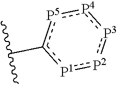

moiety is

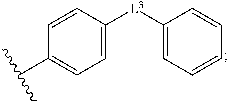

and -L³ is —O—, —NH—, or C(═O), and
R² cannot be CH₂CH₂CH₂N(CH₃)₂ or CH₂CH₂CH₂N(CH₂CH₃)₂ when the

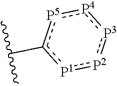

moiety is pyrimidinyl or pyridyl each substituted with one R⁷, wherein R⁷ is R⁸, and R⁸ is unsubstituted phenyl; and

(b) the compound is not:

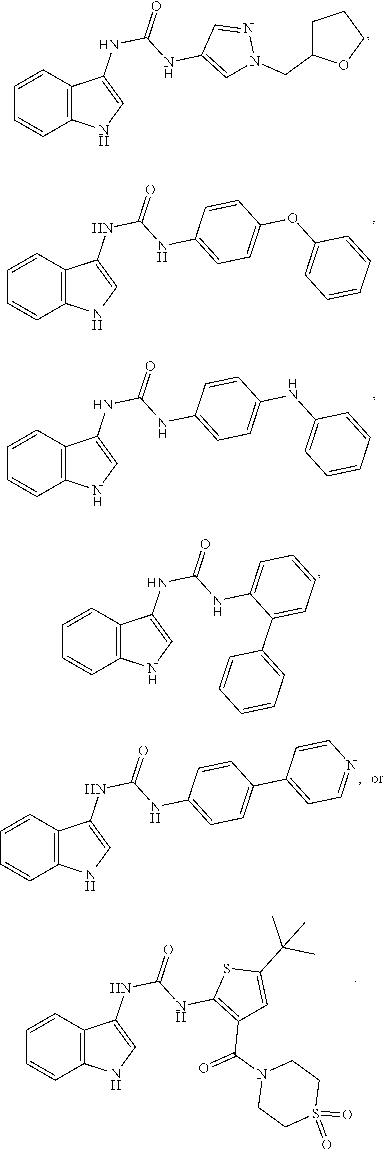

2. The compound of clause 1, wherein P¹, P², P³, P⁴, and P⁵ are defined according to (AA).

3. The compound of clauses 1 or 2, wherein one of P¹, P², P³, P⁴, and P⁵ is N.

4. The compound of clauses 1 or 2, wherein two of P¹, P², P³, P⁴, and P⁵ are N.

5. The compound of clauses 1 or 2, wherein each one of P¹, P², P³, P⁴, and P⁵ is independently selected from the group consisting of CH, CR⁷, and, CR^{c}.

6. The compound of any one of clauses 1-5, wherein one of P¹, P², P³, P⁴, and P⁵ is CR⁷.

7. The compound of any one of clauses 1-6, wherein P³ is CR⁷.

8. The compound of any one of clauses 1-4 or 6-7, wherein P⁴ is N.

9. The compound of any one of clauses 7-8, wherein each of P¹, P², and P⁵ is independently selected from the group consisting of CH and CR^{c}.

10. The compound of any one of clauses 7-8, wherein one of P¹, P², and P⁵ is N; and each remaining of P¹, P², and P⁵ is independently selected from the group consisting of CH and CR^{c}.

11. The compound of any one of clauses 1-4 or 6-7, wherein P¹ is N.

12. The compound of any one of clauses 7 or 11, wherein each of P², P⁴, and P⁵ is independently selected from the group consisting of CH and CR^{c}.

13. The compound of any one of clauses 7 or 11, wherein one of P², P⁴, and P⁵ is N; and each remaining of P², P⁴, and P⁵ is independently selected from the group consisting of CH and CR^{c}.

14. The compound of clauses 1 or 2, wherein P³ is CR⁷; P⁴ is N; and each of P¹, P², and P⁵ is independently selected from the group consisting of CH and CR^{c}.

15. The compound of clauses 1 or 2, wherein P³ is CR⁷; P⁴ is N; P¹ is N; and each of P² and P⁵ is independently selected from the group consisting of CH and CR^{c}.

16. The compound of clauses 1 or 2, wherein P³ is CR⁷; P⁴ is N; P⁵ is N; and each of P² and P¹ is independently selected from the group consisting of CH and CR^{c}; or

wherein P³ is CR⁷; P⁴ and P² are N; and each of P¹ and P⁵ is independently selected from the group consisting of CH and CR^{c}.

17. The compound of clauses 1 or 2, wherein P³ is CR⁷; and each of P¹, P², P⁴ and P⁵ is independently selected from the group consisting of CH and CR^{c}.

18. The compound of clauses 1 or 2, wherein P³ is CR⁷; P¹ is N; and each of P², P⁴, and P⁵ is independently selected from the group consisting of CH and CR^{c}.

19. The compound of any one of clauses 1-6, wherein P⁴ is CR⁷.

20. The compound of clause 19, wherein each of P¹, P², P³, and P⁵ is independently selected from the group consisting of N, CH, and CR^{c}.

21. The compound of clauses 19 or 20, wherein each of P¹, P², P³, and P⁵ is independently selected from the group consisting of CH and CR^{c}.

22. The compound of clauses 19 or 20, wherein one of P¹, P², P³, and P⁵ is N; and each remaining of P¹, P², P³, and P⁵ is independently selected from the group consisting of CH and CR^{c}.

23. The compound of any one of clauses 1-2, 19-20 or 22, wherein P⁴ is CR⁷, P³ is N; and each of P¹, P², and P⁵ is independently selected from the group consisting of CH and CR^{c}.

24. The compound of any one of clauses 1-2, 19-20 or 22, wherein P⁴ is CR⁷, P² is N; and each of P¹, P³, and P⁵ is independently selected from the group consisting of CH and CR^{c}.

25. The compound of clauses 1 or 2, wherein the

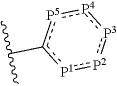

moiety has the formula:

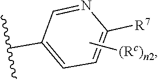

wherein n2 is 0, 1, or 2.

26. The compound of any one of clauses 1-2 or 25, wherein the

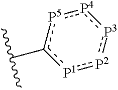

moiety has the formula:

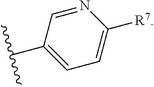

27. The compound of any one of clauses 1-2 or 25, wherein the

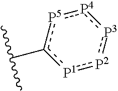

moiety has the formula:

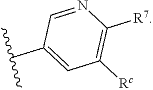

28. The compound of clauses 1 or 2, wherein the

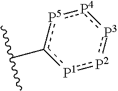

moiety has the formula:

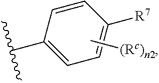

wherein n2 is 0, 1, or 2.

29. The compound of any one of clauses 1-2 or 28, wherein the

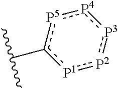

moiety has the formula:

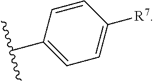

30. The compound of any one of clauses 1-2 or 28, wherein the

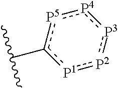

moiety has the formula:

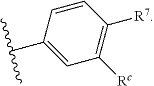

31. The compound of clauses 1 or 2, wherein the

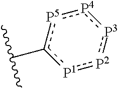

moiety has the formula:

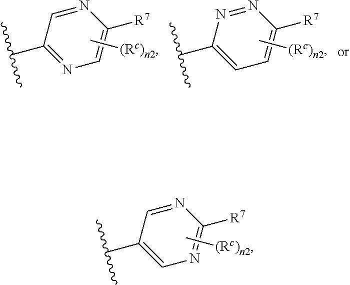

wherein n2 is 0, 1, or 2.

32. The compound of clauses 1 or 2, wherein the

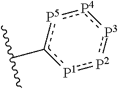

moiety has the formula:

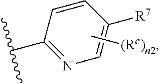

wherein n2 is 0, 1, or 2.

33. The compound of any one of clauses 1-2 or 32, wherein the

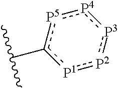

moiety has the formula:

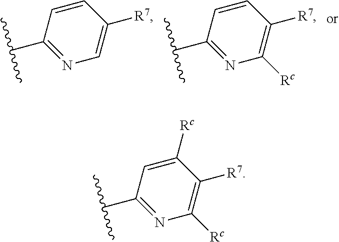

34. The compound of clauses 1 or 2, wherein the

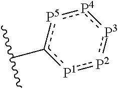

moiety has the formula:

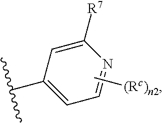

wherein n2 is 0, 1, or 2.

35. The compound of any one of clauses 1-2 or 34, wherein the

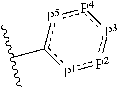

moiety has the formula:

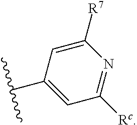

36. The compound of any one of clauses 1-2 or 34, wherein the

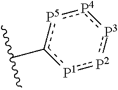

moiety has the formula:

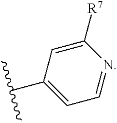

37. The compound of clauses 1 or 2, wherein the

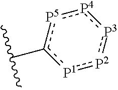

moiety has the formula:

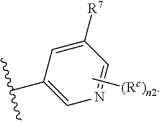

38. The compound of any one of clauses 1-2 or 37, wherein the

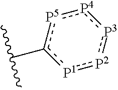

moiety has the formula:

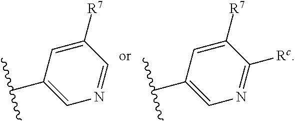

39. The compound of clause 1, wherein P¹, P², P³, P⁴, and P⁵ are defined according to (BB).

40. The compound of clauses 1 or 39, wherein P³ is CR⁷ or NR⁷; and each of P², P⁴, and P⁵ is independently selected from the group consisting of: O, S, N, NH, NR^{d}, CH, and CR^{c}, provided that 1-3 of P², P³, P⁴, and P⁵ is O, S, N, NH, NR^{d}, or NR⁷.

41. The compound of any one of clauses 1 or 39-40, wherein P³ is NR⁷; and each of P², P⁴, and P⁵ is independently selected from the group consisting of: O, S, N, NH, NR^{d}, CH, and CR^{c}.

42. The compound of any one of clauses 1 or 39-41, wherein P³ is NR⁷; and each of P², P⁴, and P⁵ is independently selected from the group consisting of: N, CH, and CR^{c}.

43. The compound of any one of clauses 1 or 39-42, wherein P³ is NR⁷; P² is CH or CR^{c}, such as CH; P⁴ is N; and P⁵ is CH or CR^{c}, such as CH.

44. The compound of any one of clauses 1 or 39-42, wherein P³ is NR⁷; P² is N; P⁴ is CH or CR^{c}, such as CH; and P⁵ is CH or CR^{c}, such as CH.

45. The compound of any one of clauses 1 or 39-42, wherein P³ is NR⁷; P² is CH or CR^{c}, such as C; P⁴ is CH or CR^{c}, such as CH; and P⁵ is N.

46. The compound of clauses 1 or 39, wherein the

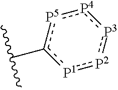

moiety has the formula:

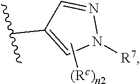

wherein n2 is 0 or 1, such as 0.

47. The compound of clauses 1 or 39, wherein the

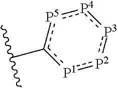

moiety has the formula:

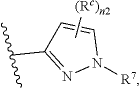

wherein n2 is 0 or 1, such as 0.

48. The compound of clauses 1 or 39, wherein the

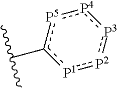

moiety has the formula:

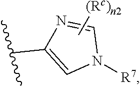

wherein n2 is 0 or 1, such as 0.

49. The compound of any one of clauses 1-48, wherein R⁷ is R⁸.

50. The compound of any one of clauses 1-49, wherein R⁸ is selected from the group consisting of:

(a) C₃₋₁₂ cycloalkyl or C₃₋₁₂ cycloalkenyl, each of which is optionally substituted with 1-4 independently selected R⁷′; and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′}.

51. The compound of any one of clauses 1-50, wherein R⁸ is selected from the group consisting of:

(a) C₃₋₁₂ cycloalkyl or C₃₋₁₂ cycloalkenyl, each of which is substituted with 1-4 independently selected R⁷′; and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

52. The compound of any one of clauses 1-51, wherein R⁸ is C₃₋₁₂ cycloalkyl or C₃₋₁₂ cycloalkenyl, each of which is substituted with 1-4 independently selected R^{7′}.

53. The compound of any one of clauses 1-52, wherein R⁸ is C₄₋₁₀ cycloalkyl or C₄₋₁₀ cycloalkenyl, each of which is substituted with 1-4 independently selected R^{7′}.

54. The compound of any one of clauses 1-53, wherein R⁸ is C₄₋₈ cycloalkyl or C₄₋₈ cycloalkenyl, each of which is substituted with 1-4 independently selected R^{7′}

55. The compound of any one of clauses 1-54, wherein R⁸ is C₄₋₈ cycloalkyl which is substituted with 1-4 independently selected R^{7′}.

56. The compound of any one of clauses 1-55, wherein R⁸ is C₄₋₈ cycloalkyl which is substituted with 1-3 independently selected R^{7′}.

57. The compound of any one of clauses 1-56, wherein R⁸ is cyclohexyl which is substituted with 1-3 (e.g., 1 or 2) R⁷′; or wherein R⁸ is cyclobutyl which is substituted with 1-3 (e.g., 1 or 2) R⁷′.

58. The compound of any one of clauses 1-57, wherein R⁸ is

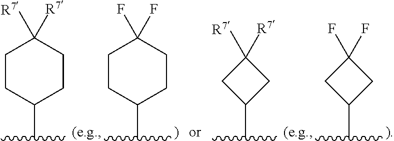

59. The compound of any one of clauses 1-57, wherein R⁸ is

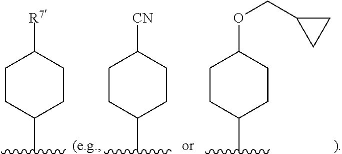

60. The compound of any one of clauses 1-52, wherein R⁸ is spirocyclic C₆₋₁₂ cycloalkyl which is substituted with 1-4 independently selected R⁷′.

61. The compound of any one of clauses 1-52 or 60, wherein R⁸ is

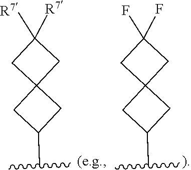

62. The compound of any one of clauses 1-51, wherein R⁸ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

63. The compound of any one of clauses 1-51 or 62, wherein R⁸ is heterocyclyl or heterocycloalkenyl of 4-10 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

64. The compound of any one of clauses 1-51 or 62-63, wherein R⁸ is heterocyclyl or heterocycloalkenyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

65. The compound of any one of clauses 1-51 or 62-64, wherein R⁸ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R^{7′}.

66. The compound of any one of clauses 1-51 or 62-65, wherein R⁸ is heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 1-3 independently selected R^{7′}.

67. The compound of any one of clauses 1-51 or 62-66, wherein R⁸ is selected from the group consisting of azetidinyl, oxetanyl, pyrrolidinyl, tetrahydrofuranyl, dioxanyl (e.g., 1,3-dioxanyl), piperidinyl, piperazinyl, morpholinyl, and tetrahydropyranyl, each of which is substituted with 1-3 (e.g., 1 or 2) independently selected R^{7′}.

68. The compound of any one of clauses 1-51 or 62-67, wherein R⁸ is selected from the group consisting of azetidinyl, pyrrolidinyl, morpholinyl, and piperidinyl, each of which is substituted with 1-3 (e.g., 1 or 2) independently selected R^{7′}.

69. The compound of any one of clauses 1-51 or 62-68, wherein R⁸ is selected from the group consisting of:

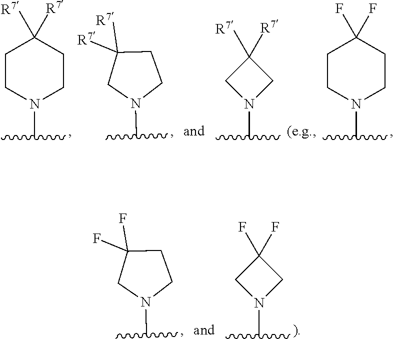

70. The compound of any one of clauses 1-51 or 62-69, wherein R⁸ is selected from the group consisting of:

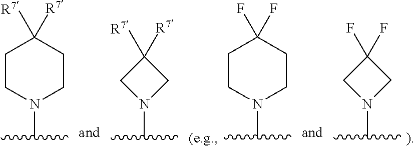

71. The compound of any one of clauses 1-51 or 62-68, wherein R⁸ is selected from the group consisting of:

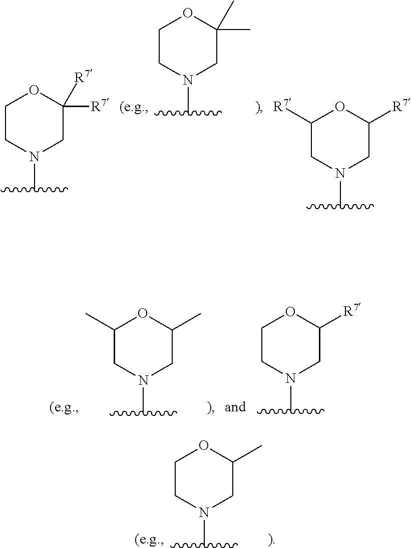

72. The compound of any of clauses 1-51 or 62-68, wherein R⁸ is selected from the group consisting of:

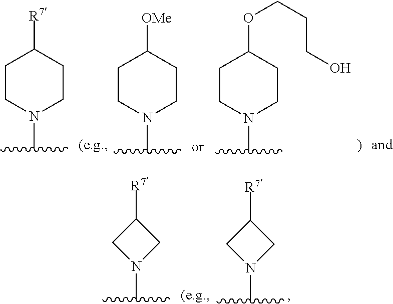

wherein R⁷′ is C₁₋₄ haloalkyl, such as —CF₃).

73. The compound of any one of clauses 1-51 or 62-67, wherein R⁸ is

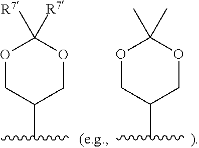

74. The compound of any one of clauses 1-51 or 62-67, wherein R⁸ is selected from the group consisting of:

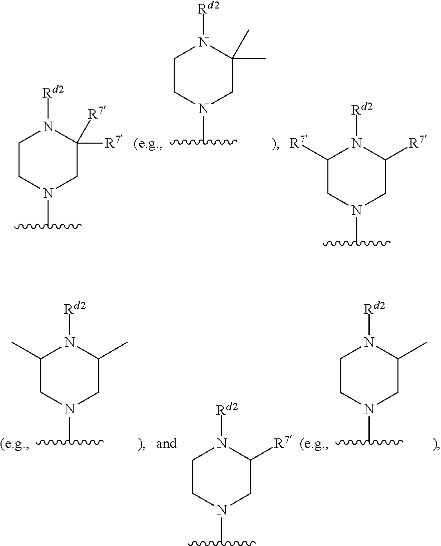

wherein R^{d2} is H or R^{d}.

75. The compound of any one of clauses 1-50, wherein R⁸ is spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R^{7′}.

76. The compound of any one of clauses 1-50 or 75, wherein R⁸ is selected from the group consisting of: 2-azaspiro[3.3]heptanyl, 1-oxa-9-azaspiro[5.5]undecanyl, 6-azaspiro[2.5]octanyl, 1,5-dioxaspiro[5.5]undecanyl, 7-azaspiro[3.5]nonanyl, and 2,6-diazaspiro[3.3]heptanyl, each of which is optionally substituted with 1-4 independently selected R⁷′ at one or more ring carbon atoms, wherein a ring nitrogen is optionally substituted with R^{d}.

77. The compound of any one of clauses 1-50 or 75-76, wherein R⁸ is selected from the group consisting of: 2-azaspiro[3.3]heptanyl, 1-oxa-9-azaspiro[5.5]undecanyl, and 6-azaspiro[2.5]octanyl, each of which is optionally substituted with 1-4 independently selected R⁷′ at one or more ring carbon atoms.

78. The compound of any one of clauses 1-51 or 75-77, wherein R⁸ is

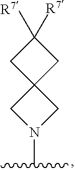

such as:

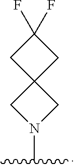 [SMILES: *N1CC2(C1)CC(F)(F)C2]

79. The compound of any one of clauses 1-51 or 75-76, wherein R⁸ is selected from the group consisting of:

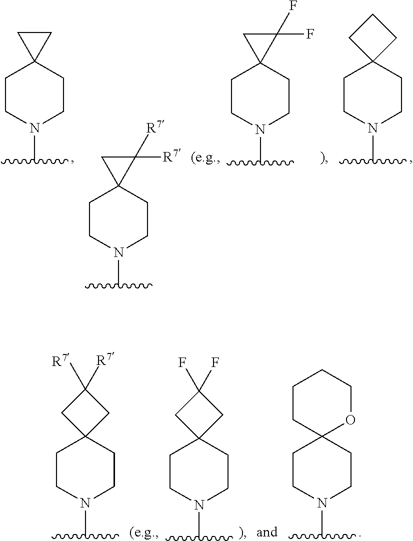

80. The compound of any one of clauses 1-51 or 75-76, wherein R⁸ is

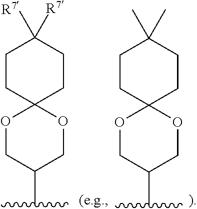

81. The compound of any one of clauses 1-51 or 75-76, wherein R⁸ is

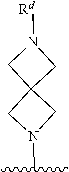

optionally wherein R^{d} is C₁₋₆ alkyl optionally substituted with 1-3 substituents each independently selected from the group consisting of halo, C₁₋₃ alkoxy, and C₁₋₃ haloalkoxy, such as wherein R^{d} is C₂₋₄ alkyl substituted with 1-3 independently selected halo

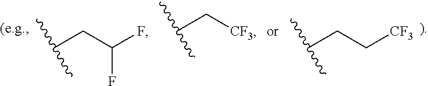

82. The compound of any one of clauses 1-50, wherein R⁸ is bridged heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′, such as wherein R⁸ is

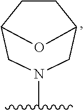 [SMILES: *N1CC2CCC(C1)O2]

which is optionally substituted with 1-2 R⁷′ at one or more ring carbon atoms.

83. The compound of any one of clauses 1-50, wherein R⁸ is C₃₋₁₂ cycloalkyl or C₃₋₁₂ cycloalkenyl which is unsubstituted.

84. The compound of clause 83, wherein R⁸ is C₃₋₈ (e.g., C₃₋₅ or C₇₋₈) monocyclic cycloalkyl which is unsubstituted.

85. The compound of clause 84, wherein R⁸ is C₄₋₆ monocyclic cycloalkyl which is unsubstituted.

86. The compound of any one of clauses 1-50 or 85, wherein R⁸ is cyclobutyl or cyclopentyl.

87. The compound of any one of clauses 1-50 or 85, wherein R⁸ is cyclohexyl.

88. The compound of any one of clauses 1-50, wherein R⁸ is C₇₋₁₂ bicyclic cycloalkyl which is unsubstituted.

89. The compound of any one of clauses 1-50 or 88, wherein R⁸ is C₇₋₁₂ spirocyclic cycloalkyl which is unsubstituted.

90. The compound of any one of clauses 1-50 or 88-89, wherein R⁸ is

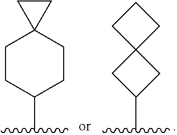 [SMILES: *C1CCC2(CC1)CC2 ; *C1CC2(CCC2)C1]

91. The compound of any one of clauses 1-50 or 88, wherein R⁸ is C₇₋₁₂ bridged cycloalkyl which is unsubstituted.

92. The compound of any one of clauses 1-50, 88 or 91, wherein R⁸ is

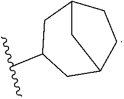 [SMILES: *C1CC2CCC(C1)C2]

93. The compound of any one of clauses 1-50, wherein R⁸ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂.

94. The compound of any one of clauses 1-50 or 93, wherein R⁸ is monocyclic heterocyclyl of 3-8 ring atoms, wherein 1-2 ring atoms are heteroatoms, each independently selected from the group consisting of N, N(H), N(R^{d}), O, and S(O)₀₋₂.

95. The compound of any one of clauses 1-50 or 93-94, wherein R⁸ is selected from the group consisting of: azetidinyl, oxetanyl, pyrrolidinyl, tetrahydrofuranyl, tetrahydropyranyl, piperidinyl, piperazinyl, morpholinyl, azepinyl, and oxepanyl, wherein a ring nitrogen atom is optionally substituted with R^{d}.

96. The compound of any one of clauses 1-50 or 93-95, wherein R⁸ is azetidinyl, pyrrolidinyl, piperidinyl, piperazinyl, morpholinyl, or oxepanyl, wherein a ring nitrogen atom is optionally substituted with R^{d}, such as wherein R⁸ is pyrrolidinyl, piperidinyl, or piperazinyl, wherein a ring nitrogen atom is substituted with R^{d}.

97. The compound of any one of clauses 1-50 or 93-96, wherein R⁸ is azetidinyl

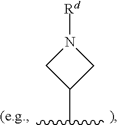

pyrrolidinyl

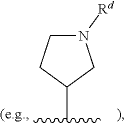

piperidinyl

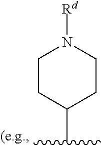

such as

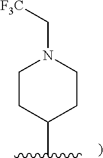 [SMILES: *C1CCN(CC(F)(F)F)CC1]

or piperazinyl

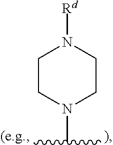

wherein a ring nitrogen atom is substituted with R^{d},

optionally wherein R^{d} is C₁₋₆ alkyl optionally substituted with 1-3 substituents each independently selected from the group consisting of halo, C₁₋₃ alkoxy, and C₁₋₃ haloalkoxy, such as wherein R^{d} is C₂₋₄ alkyl substituted with 1-3 independently selected halo

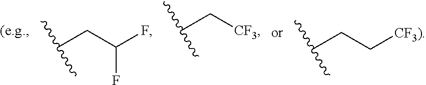

98. The compound of any one of clauses 1-50 or 93-97, wherein R⁸ is piperidinyl

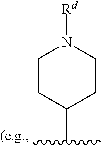

such as

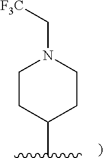 [SMILES: *C1CCN(CC(F)(F)F)CC1]

or piperazinyl

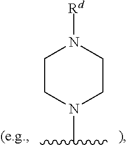

wherein a ring nitrogen atom is substituted with R^{d},

optionally wherein R^{d} is C₁₋₆ alkyl optionally substituted with 1-3 substituents each independently selected from the group consisting of halo, C₁₋₃ alkoxy, and C₁₋₃ haloalkoxy, such as wherein R^{d} is C₂₋₄ alkyl substituted with 1-3 independently selected halo

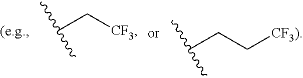

99. The compound of any one of clauses 1-50, wherein R⁸ is bicyclic or polycyclic heterocyclyl or heterocycloalkenyl of 7-12 ring atoms, wherein 1-3 ring atoms are heteroatoms, each independently selected from the group consisting of N, N(H), N(R^{d}), O, and S(O)₀₋₂.

100. The compound of any one of clauses 1-50 or 99, wherein R⁸ is bicyclic or polycyclic heterocyclyl of 7-12 ring atoms, wherein 1-3 ring atoms are heteroatoms, each independently selected from the group consisting of N, N(H), N(R^{d}), O, and S(O)₀₋₂, such as wherein R⁸ is

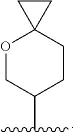 [SMILES: *C1CCC2(CC2)OC1]

101. The compound of any one of clauses 1-50, wherein R⁸ is selected from the group consisting of:

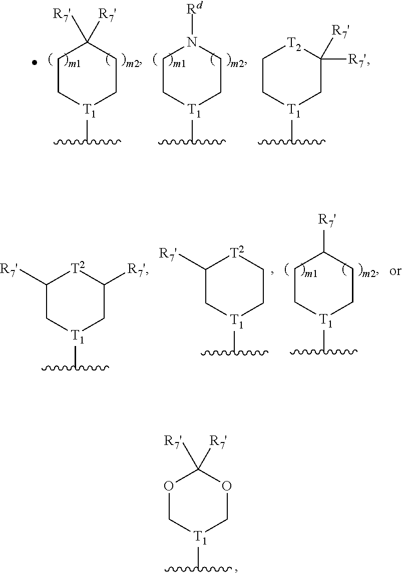

wherein m1 and m2 are independently 0, 1, or 2; T¹ is CH or N; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′,

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F; and

optionally wherein R^{d} is C₁₋₆ alkyl, such as C₂₋₄ alkyl, optionally substituted with 1-3 independently selected halo, such as —F.

102. The compound of any one of clauses 1-50 or 101, wherein R⁸ is selected from the group consisting of:

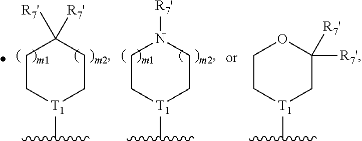

wherein m1 and m2 are independently 0, 1, or 2, and T¹ is CH or N; and
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′, such as:

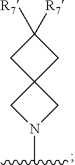

optionally wherein each R⁷′ is independently selected from the group consisting of C₁₋₃ alkyl and halo, such as wherein each R⁷′ is independently selected from the group consisting of methyl and —F; and

optionally wherein R^{d} is C₁₋₆ alkyl, such as C₂₋₄ alkyl, optionally substituted with 1-3 independently selected halo, such as —F.

103. The compound of any one of clauses 1-50 or 101-102, wherein R⁸ is

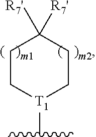

wherein m1 and m2 are independently 0, 1, or 2, and T¹ is CH or N, such as:

wherein R⁸ is selected from the group consisting of:

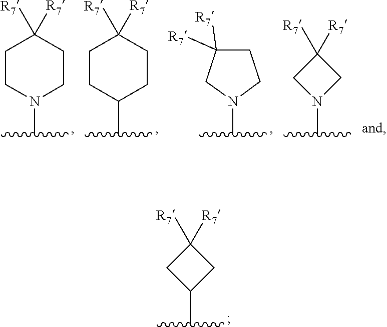

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F, such as wherein each R⁷′ is an independently selected halo, such as —F.

104. The compound any one of clauses 1-50 or 101-102, wherein R⁸ is

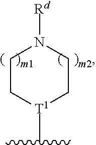

wherein m1 and m2 are independently 0, 1, or 2, and T is CH or N, such as:

wherein R⁸ is selected from the group consisting of:

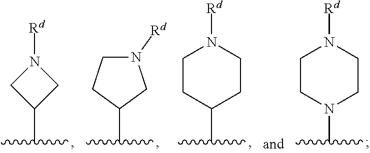

optionally wherein R^{d} is C₁₋₆ alkyl, such as C₂₋₄ alkyl, optionally substituted with 1-3 independently selected halo, such as —F.

105. The compound of any one of clauses 1-50 or 101, wherein R⁸ is selected from the group consisting of:

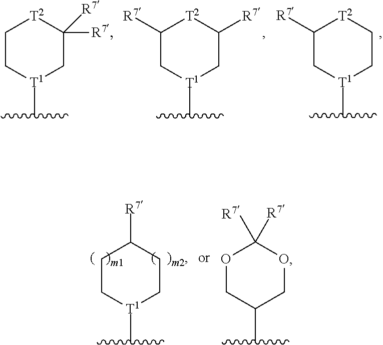

wherein m1 and m2 are independently 0, 1, or 2; T¹ is CH or N; and T² is CH₂, NH, NR^{d}, or O; such as: wherein R⁸ is selected from the group consisting of:

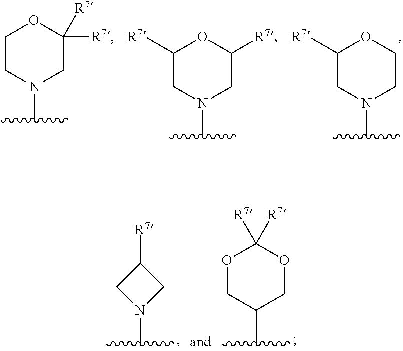

optionally wherein each R⁷′ is independently selected from the group consisting of C₁₋₃ alkyl and C₁₋₃ haloalkyl.

106. The compound of any one of clauses 1-50 or 101, wherein R⁸ is selected from the group consisting of:
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′;

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F.

107. The compound of any one of clauses 1-50, 101, or 106, wherein R⁸ is

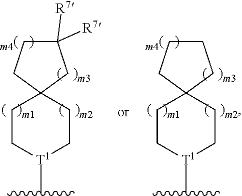

wherein m1, m2, m3, and m4 are independently 0, 1, or 2, provided that m1+m2+m3+m4≤6, and T¹ is CH or N, such as:

wherein R⁸ is selected from the group consisting of:

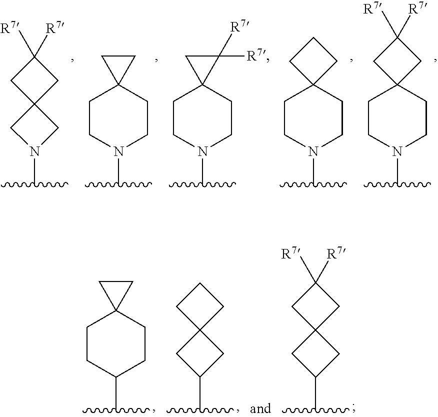

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F, such as: wherein each R⁷′ is an independently selected halo, such as —F.

108. The compound of any one of clauses 1-50, 101-102, or 106, wherein R⁸ is

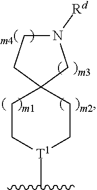

wherein m1, m2, m3, and m4 are independently 0, 1, or 2, provided that m1+m2+m3+m4≤6, T¹ is CH or N, such as: wherein R⁸ is

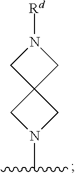

optionally wherein R^{d} is C₁₋₆ alkyl, such as C₂₋₄ alkyl, optionally substituted with 1-3 independently selected halo, such as —F.

109. The compound of any one of clauses 1-50, 101-102, or 106, wherein R⁸ is

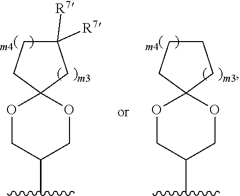

wherein m3 and m4 are independently 0, 1, or 2, provided that m3+m4≤4, such as: wherein R⁸ is

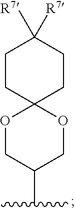

optionally wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F, such as: wherein each R⁷′ is an independently selected halo, such as —F.

110. The compound of any one of clauses 1-49, wherein R⁸ is heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}.

111. The compound of any one of clauses 1-49 or 110, wherein R⁸ is heteroaryl of 5-6 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R^{7′}.

112. The compound of any one of clauses 1-49 or 110-111, wherein R⁸ is heteroaryl of 5 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R^{7′}.

113. The compound of any one clauses 1-49 or 110-112, wherein R⁸ is pyrazolyl, imidazolyl, thiazolyl, oxazolyl, triazolyl, each of which is optionally substituted with 1-2 independently selected R⁷′ at one or more ring carbon atoms and optionally substituted with one R^{d} at a ring nitrogen atom.

114. The compound of any one of clauses 1-49 or 110-113, wherein R⁸ is thiazolyl optionally substituted with 1-2 independently selected R⁷′

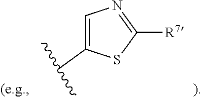

115. The compound of any one of clauses 1-49 or 110, wherein R⁸ is bicyclic heteroaryl of 7-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-2 independently selected R^{7′}.

116. The compound of any one of clauses 1-49, 110, or 115, wherein R⁸ is

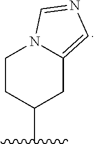 [SMILES: *C1CCn2cncc2C1]

117. The compound of any one of clauses 1-49, wherein R⁸ is C₆₋₁₀ aryl optionally substituted with 1-4 independently selected R^{7′}.

118. The compound of clause 117, wherein R⁸ is phenyl optionally substituted with 1-2 independently selected R⁷′ (e.g., unsubstituted phenyl).

119. The compound of any one of clauses 1-48, wherein R⁷ is -L³-R⁹.

120. The compound of any one of clauses 1-48 or 119, wherein -L³ is —O—.

121. The compound of any one of clauses 1-48 or 119, wherein -L³ is —NH—.

122. The compound of any one of clauses 1-48 or 119, wherein -L³ is —S— or S(O)₁₋₂.

123. The compound of any one of clauses 1-48 or 119, wherein -L³ is selected from the group consisting of: C(═O)NH, NHC(═O), C(═O)O, OC(═O), C(═O), NHS(O)₂, and S(O)₂NH; or wherein -L³ is C₁₋₄ alkylene, such as CH₂ or

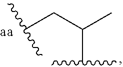

wherein aa is the point of attachment to R⁹.

124. The compound of any one of clauses 1-48 or 119-123, wherein R⁹ is selected from the group consisting of:

(a) C₃₋₁₂ cycloalkyl or C₃₋₁₂ cycloalkenyl, each of which is optionally substituted with 1-4 independently selected R⁷′, and

(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′}.

125. The compound of any one of clauses 1-48 or 119-124, wherein R⁹ is C₃₋₁₂ cycloalkyl or C₃₋₁₂ cycloalkenyl, each of which is optionally substituted with 1-4 independently selected R^{7′}.

126. The compound of any one of clauses 1-48 or 119-125, wherein R⁹ is C₄₋₈ cycloalkyl which is optionally substituted with 1-2 R^{7′}.

127. The compound of any one of clauses 1-48 or 119-126, wherein R⁹ is cyclobutyl, cyclopentyl, cyclohexyl, or spiro[3.3]heptanyl, each of which is optionally substituted with 1-2 R⁷′ (e.g., unsubstituted).

128. The compound of any one of clauses 1-48 or 119-124, wherein R⁹ is heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′}.

129. The compound of any one of clauses 1-48, 119-124, or 128, wherein R⁹ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

130. The compound of any one of clauses 1-48, 119-124, or 128-129, wherein R⁹ is selected from the group consisting of azetidinyl, oxetanyl, pyrrolidinyl, tetrahydrofuranyl, piperidinyl, piperazinyl, morpholinyl, and azepinyl, each of which is optionally substituted with 1-2 independently selected R⁷′ (e.g., unsubstituted).

131. The compound of any one of clauses 1-48, wherein R⁷ is L³-R⁹; L³ is —O— or —NH—; and R⁹ is selected from the group consisting:

C₄₋₈ cycloalkyl which is optionally substituted with 1-2 R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

132. The compound of clause 131, wherein R⁷ is L³-R⁹; L³ is —O— or —NH—; and R⁹ is selected from the group consisting of cyclobutyl, cyclopentyl, cyclohexyl, and oxetanyl, each of which is optionally substituted with 1-2 independently selected R⁷′ (e.g., unsubstituted).

133. The compound of clauses 131 or 132, wherein L³ is —O—.

134. The compound of any one of clauses 131-133, wherein R⁷ is

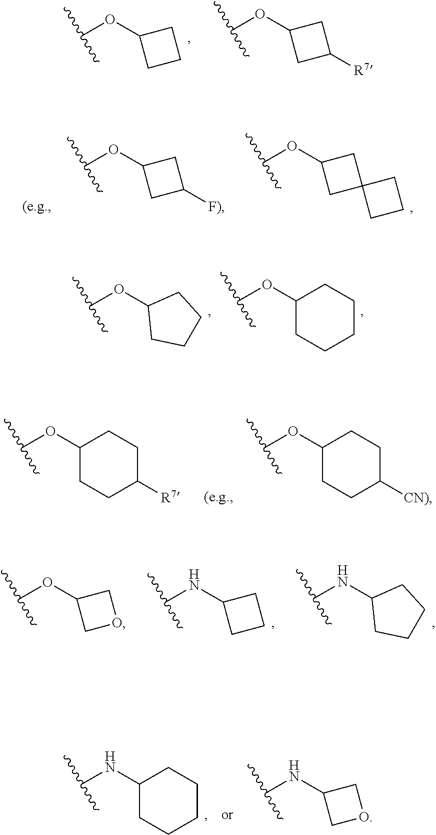

135. The compound of clauses 1 or 2, wherein the

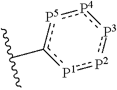

moiety has the formula:

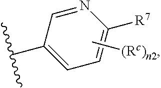

wherein n2 is 0, 1, or 2; and R⁷ is R⁸, wherein R⁸ is selected from the group consisting of:

C₄₋₈ cycloalkyl which is optionally substituted with 1-4 independently selected R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′.

136. The compound of clauses 1 or 2, wherein the

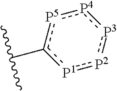

moiety has the formula:

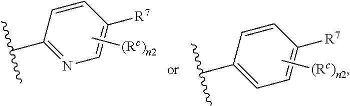

wherein n2 is 0, 1, or 2; and R⁷ is R⁸, wherein R⁸ is selected from the group consisting of:

C₄₋₈ cycloalkyl which is optionally substituted with 1-4 independently selected R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′.

137. The compound of clauses 1 or 2, wherein the

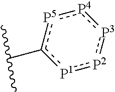

moiety has the formula:

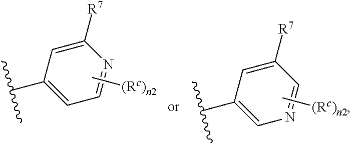

wherein n2 is 0, 1, or 2; and R⁷ is R⁸, wherein R⁸ is selected from the group consisting of:

C₄₋₈ cycloalkyl which is optionally substituted with 1-4 independently selected R^{7′}; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′.

138. The compound of clauses 1 or 39, wherein the

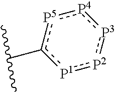

Moiety has the formula:

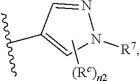

wherein n2 is 0 or 1; and R⁷ is R⁸, wherein R⁸ is selected from the group consisting of:

C₄₋₈ cycloalkyl which is optionally substituted with 1-4 independently selected R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R^{7′}.

139. The compound of any one of clauses 135-138, wherein n2 is 0.

140. The compound of any one of clauses 135-138, wherein n2 is 1.

141. The compound of any one of clauses 135-140, wherein R^{e} is located ortho to R⁷.

142. The compound of any one of clauses 135-140, wherein R^{c} is located meta to R⁷.

143. The compound of any one of clauses 135-142, wherein R⁷ is R⁸; and R⁸ is C₄₋₈ cycloalkyl which is substituted with 1-3 R^{7′}.

144. The compound of any one of clauses 135-143, wherein R⁸ is cyclohexyl which is substituted with 1-3 R⁷′, such as

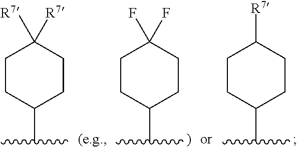

or wherein R⁸ is cyclobutyl which is substituted with 1-3 R⁷′, such as

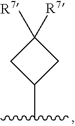

such as

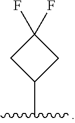 [SMILES: *C1CC(F)(F)C1]

145. The compound of any one of clauses 135-142, wherein R⁷ is R⁸; and R⁸ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is substituted with 1-4 independently selected R⁷′, such as:

wherein R⁸ is heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 1-3 independently selected R^{7′}.

146. The compound of any one of clauses 135-142 or 145, wherein R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 1-3 (e.g., 1 or 2) independently selected R⁷′ at one or more ring carbon atoms.

147. The compound of any one of clauses 135-142 or 145-146, wherein R⁸ is selected from the group consisting of azetidinyl, pyrrolidinyl, morpholinyl, and piperidinyl, each of which is substituted with 2-4 (e.g., 2) independently selected R⁷′ at one or more ring carbon atoms, such as wherein R⁸ is selected from the group consisting of:

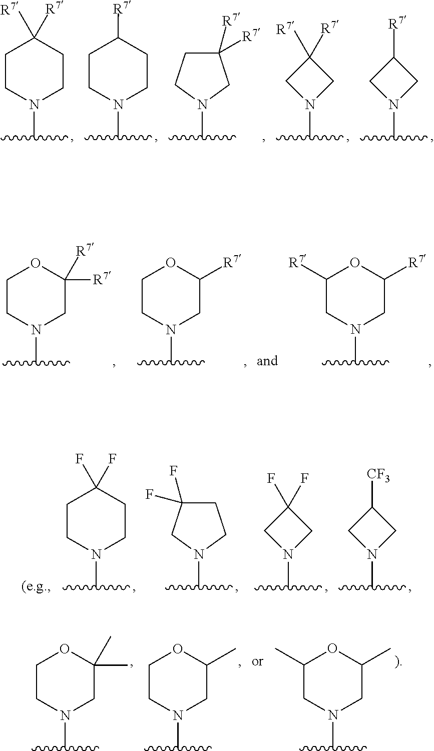

148. The compound of any one of clauses 135-142, wherein R⁸ is spirocyclic heterocyclyl of 6-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′, such as:

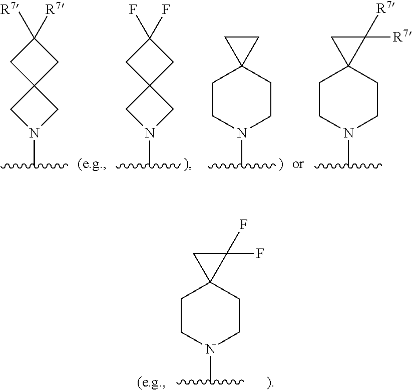

149. The compound of any one of clauses 135-142, wherein R⁸ is heterocyclyl of 4-8 ring atoms, wherein 1-3 ring atoms are heteroatoms, each independently selected from the group consisting of N, N(H), N(R^{d}), O, and S(O)₀₋₂, provided that R⁸ contains a ring N(R^{d}) group.

150. The compound of any one of clauses 135-142 or 149, wherein R⁸ is selected from the group consisting of: azetidinyl, pyrrolidinyl, piperidinyl, piperazinyl, and 2,6-diazaspiro[3.3]heptanyl, wherein a ring nitrogen atom is substituted with R^{d}, such as wherein R⁸ is

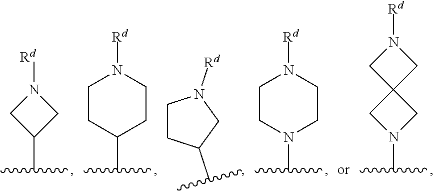

optionally wherein R^{d} is C₁₋₆ alkyl optionally substituted with 1-3 substituents each independently selected from the group consisting of halo, C₁₋₃ alkoxy, and C₁₋₃ haloalkoxy, such as wherein R^{d} is C₂₋₄ alkyl substituted with 1-3 independently selected halo

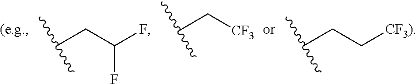

151. The compound of any one of clauses 135-142, wherein R⁸ is C₄₋₆ monocyclic cycloalkyl which is unsubstituted (e.g., cyclopentyl, cyclobutyl, or cyclohexyl); or R⁸ is C₇₋₈ bicyclic (e.g., spirocyclic) cycloalkyl which is unsubstituted

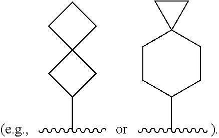 [SMILES: *C1CC2(CCC2)C1 ; *C1CCC2(CC1)CC2]

152. The compound of clauses 1 or 2, wherein the

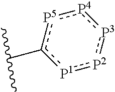

moiety has the formula:

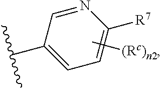

wherein n2 is 0, 1, or 2; and R⁷ is -L³-R⁹, wherein:

L³ is —NH— or —O—; and R⁹ is selected from the group consisting:

C₄₋₈ cycloalkyl which is optionally substituted with 1-2 R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R⁷′.

153. The compound of clauses 1 or 2, wherein the

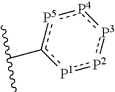

moiety has the formula:

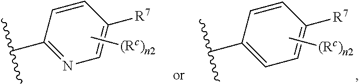

wherein n2 is 0, 1, or 2; and R⁷ is -L³-R⁹, wherein:

L³ is —NH— or —O—; and R⁹ is selected from the group consisting:

C₄₋₈ cycloalkyl which is optionally substituted with 1-2 R⁷′; and

heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

154. The compound of clauses 152 or 153, wherein R⁷ is L³-R⁹; L³ is —O— or —NH—; and R⁹ is selected from the group consisting of cyclobutyl, cyclopentyl, cyclohexyl, and oxetanyl, each of which is optionally substituted with 1-2 independently selected R⁷′ (e.g., unsubstituted).

155. The compound of any one of clauses 152-154, wherein L³ is —O—.

156. The compound of any one of clauses 152-155, wherein R⁷ is

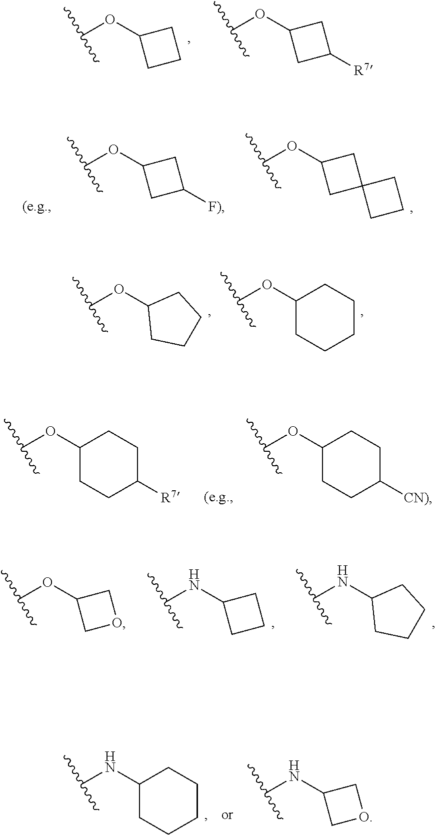

157. The compound of any one of clauses 1-156, wherein each R⁷′ when present is independently selected from the group consisting of: halo, —CN, —OH, —C₁₋₄ alkyl optionally substituted with R^{a}, —C₁₋₄ haloalkyl, —C₁₋₆ alkoxy optionally substituted with R^{a}, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″).

158. The compound of any one of clauses 1-157, wherein each R⁷′ when present is independently selected from the group consisting of: halo, —CN, —C₁₋₄ alkyl optionally substituted with R^{a}, —C₁₋₄ haloalkyl, —C₁₋₆ alkoxy optionally substituted with R^{a}, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″).

159. The compound of any one of clauses 1-158, wherein each R⁷′ when present is an independently selected halo, such as F.

160. The compound of any one of clauses 1-158, wherein each R⁷′ when present is an independently selected C₁₋₃ alkyl, such as methyl.

161. The compound of any one of clauses 1-158, wherein each R⁷′ when present is an independently selected C₁₋₃ haloalkyl, such as —CF₃.

162. The compound of any one of clauses 1-158, wherein one occurrence of R⁷′ is —C₁₋₄ alkyl optionally substituted with R^{a}, such as unsubstituted C₁₋₄ alkyl (e.g., methyl, ethyl, n-propyl) or R⁷′ is —C₁₋₄ alkyl substituted with R^{a} (e.g., —C₁₋₄ alkyl substituted with OH or C₃₋₆ cycloalkyl).

163. The compound of any one of clauses 1-158, wherein one occurrence of R⁷′ is —CN.

164. The compound of any one of clauses 1-158, wherein one occurrence of R⁷′ is C₁₋₆ alkoxy optionally substituted with R^{a}, such as unsubstituted C₁₋₆ alkoxy (e.g., methoxy); or C₁₋₆ alkoxy substituted with R^{a} (e.g., —C₁₋₄ alkoxy substituted with OH or C₃₋₆ cycloalkyl).

165. The compound of any one of clauses 162-164, wherein each remaining occurrence of R⁷′ when present is an independently selected halo (e.g., —F).

166. The compound of any one of clauses 1-165, wherein each R^{c} when present is independently selected from the group consisting of: halo; cyano; C₁₋₁₀ alkyl which is optionally substituted with 1-6 independently selected R^{a}; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; and —C(═O)N(R′)(R″).

167. The compound of any one of clauses 1-166, wherein each R^{c} when present is independently selected from the group consisting of: halo; cyano; C₁₋₁₀ alkyl optionally substituted with 1-6 independently selected —F or —Cl; C₁₋₄ alkoxy; C₁₋₄ haloalkoxy; —S(O)₁₋₂(C₁₋₄ alkyl); and —C(═O)(C₁₋₁₀ alkyl), such as wherein each R^{c} is independently halo (e.g., —F or —Cl), C₁₋₄ alkyl (e.g., CH₃), or CF₃.

168. The compound of any one of clauses 1-167, wherein Q is NH.

169. The compound of any one of clauses 1-167, wherein Q is N(C₁₋₃ alkyl), wherein the C₁₋₃ alkyl is optionally substituted with 1-2 independently selected R^{a} (e.g., Q is NMe or NCH₂CH₂CH₂OH).

170. The compound of any one of clauses 1-167, wherein Q is *—NH—(C₁₋₃ alkylene)-, wherein the asterisk represents point of attachment to W.

171. The compound of any one of clauses 1-170, wherein W is C(═O).

172. The compound of any one of clauses 1-170, wherein W is S(O)₂, C(═S), or C(═NR^{d}).

173. The compound of any one of clauses 1-170, wherein W is C(═C—NO₂) or C(═N—CN).

174. The compound of any one of clauses 1-173, wherein X¹ is NR².

175. The compound of any one of clauses 1-174, wherein X¹ is NH.

176. The compound of any one of clauses 1-175, wherein X² is CR⁵.

177. The compound of any one of clauses 1-176, wherein X² is CH.

178. The compound of any one of clauses 1-173, wherein X¹ is NR²; and X² is CR⁵.

179. The compound of any one of clauses 1-173 or 178, wherein X¹ is NH; and X² is CH.

180. The compound of any one of clauses 1-179, wherein each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); and —C(═O)N(R′)(R″).

181. The compound of any one of clauses 1-180, wherein each of R^{1a}, R^{1b}, R^{1c}, and R^{1d} is H.

182. The compound of any one of clauses 1-180, wherein 1-2 of R^{1a}, R^{1b}, R^{1c}, and R^{1d} is other than H; and each remaining of R^{1a}, R^{1b}, R^{1c}, and R^{1d} is H.

183. The compound of any one of clauses 1-180 or 182, wherein one of R^{1a}, R^{1b}, R^{1c}, and R^{1d} is other than H; and each remaining of R^{1a}, R^{1b}, R^{1c}, and R^{1d} is H.

184. The compound of any one of clauses 1-180 or 182, wherein two of R^{1a}, R^{1b}, R^{1c}, and R^{1d} are other than H; and each remaining of R^{1a}, R^{1b}, R^{1c}, and R^{1d} is H.

185. The compound of any one of clauses 1-184, wherein R^{1a} is H or halo, such as R^{1a} is H.

186. The compound of any one of clauses 1-185, wherein R^{1d} is H or halo, such as R^{1d} is H.

187. The compound of any one of clauses 1-186, wherein R^{1b} is a independently selected substituent that is other than H, optionally wherein each of R^{1a}, R^{1c}, and R^{1d} is H.

188. The compound of any one of clauses 1-186, wherein each of R^{1b} and R^{1c} is an independently selected substituent that is other than H; and optionally wherein each of R^{1a} and R^{1d} is H.

189. The compound of any one of clauses 1-188, wherein R^{1b} is halo, such as —F, —Cl, or —Br.

190. The compound of any one of clauses 1-189, wherein R^{1b} is —F or —Cl (e.g., —F).

191. The compound of any one of clauses 1-188, wherein R^{1b} is C₁₋₆ alkyl optionally substituted with 1-2 R^{a}, such as unsubstituted C₁₋₆ alkyl.

192. The compound of any one of clauses 1-188, wherein R^{1b} is C₁₋₄ haloalkyl, such as —CF₃ or —CHF₂.

193. The compound of any one of clauses 1-188, wherein R^{1b} is —CN.

194. The compound of any one of clauses 1-188, wherein R^{1b} is —SF₅.

195. The compound of any one of clauses 1-188, wherein R^{1b} is C₁₋₄ thioalkoxy (e.g., SMe).

196. The compound of any one of clauses 1-188, wherein R^{1b} is S(O)₂(C₁₋₄ alkyl) (e.g., S(O)₂Me).

197. The compound of any one of clauses 1-188, wherein R^{1b} is C₁₋₄ alkoxy or C₁₋₄ haloalkoxy (e.g., OCHF₂).

198. The compound of any one of clauses 1-186 or 188-197, wherein R^{1c} is halo (e.g., —F).

199. The compound of any one of clauses 1-186 or 188-197, wherein R^{1c} is selected from the group consisting of C₁₋₆ alkyl and C₁₋₄ haloalkyl.

200. The compound of any one of clauses 1-186 or 188-197, wherein R^{1c} is selected from the group consisting of: C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me).

201. The compound of any one of clauses 1-180, wherein each of R^{1b} and R^{1c} is an independently selected halo; and each of R^{1a} and R^{1d} is H.

202. The compound of clause 201, wherein each of R^{1b} and R^{1c} is —F.

203. The compound of any one of clauses 1-180, wherein R^{1c} is halo, such as —F; R^{1b} is selected from the group consisting of: C₁₋₆ alkyl, C₁₋₄ haloalkyl, C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me); and each of R^{1a} and R^{1d} is H.

204. The compound of any one of clauses 1-180, wherein R^{1c} is H; and R^{1b} is halo, such as —F or —Cl, such as —Cl; and each of R^{1a} and R^{1d} is H.

205. The compound of any one of clauses 1-180, wherein R^{c} is H; R^{1b} is selected from the group consisting of: C₁₋₆ alkyl, C₁₋₄ haloalkyl, C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me); and each of R^{1a} and R^{1d} is H.

206. The compound of any one of clauses 1-205, wherein R² is H.

207. The compound of any one of clauses 1-205, wherein R² is selected from the group consisting of:

(iii) —C(O)(C₁₋₆ alkyl) optionally substituted with 1-3 independently selected R^{a};

(iv) —C(O)O(C₁₋₄ alkyl) optionally substituted with 1-3 independently R^{a};

(v) —CON(R′)(R″);

(vi) —S(O)₁₋₂(NR′R″); and

(vii) —S(O)₁₋₂(C₁₋₄ alkyl) optionally substituted with 1-3 independently selected R^{a}.

208. The compound of clause 207, wherein R² is —C(O)(C₁₋₆ alkyl) optionally substituted with 1-3 independently selected R^{a}.

209. The compound of clause 208, wherein each R^{a} substituent of R² is independently —F, —Cl, —OH, or —NR^{e}R^{f}.

210. The compound of clauses 208 or 209, wherein R² is selected from the group consisting of:

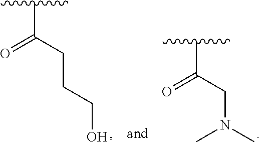

211. The compound of clause 207, wherein R² is —S(O)₁₋₂(C₁₋₄ alkyl) optionally substituted with 1-3 independently selected R^{a} (e.g., S(O)₂Me).

212. The compound of any one of clauses 1-205, wherein R² is -L⁴-L⁵-Rⁱ.

213. The compound of clause 212, wherein -L⁴ is a bond.

214. The compound of clause 212, wherein -L⁴ is C(═O).

215. The compound of clause 212, wherein -L⁴ is S(O)₂.

216. The compound of any one of clauses 212-215, wherein -L⁵ is a bond.

217. The compound of any one of clauses 212-215, wherein -L⁵ is C₁₋₄ alkylene (e.g., C₁₋₂ alkylene).

218. The compound of any one of clauses 212-217, wherein Rⁱ is selected from the group consisting of: (a) C₃₋₈ cycloalkyl, optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is

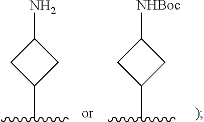 [SMILES: *C1CC(N)C1 ; *C1CC(NC(=O)OC(C)(C)C)C1]

and

(b) heterocyclyl, wherein the heterocyclyl has 3-8 ring atoms, wherein 1-3 ring atoms are heteroatoms, each independently selected from the group consisting of N, N(H), N(R^{d}), O, and S(O)₀₋₂, wherein the heterocyclyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is

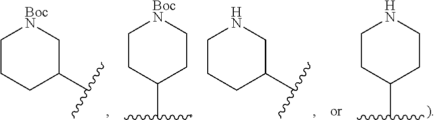

219. The compound of any one of clauses 212-217, wherein Rⁱ is selected from the group consisting of: (a) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g., Rⁱ is pyridyl, pyrimidyl, or pyrazolyl, each optionally substituted with 1-2 substituents independently selected from the group consisting of: halo; C₁₋₄ alkyl; C₁₋₄ haloalkyl; cyano; C₁₋₄ alkoxy; and C₁₋₄ haloalkoxy); and

(b) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

220. The compound of clause 212, wherein R² is -L⁴-L⁵-Rⁱ; L⁴ is a bond; L⁵ is a bond or C₁₋₄ alkylene; and Rⁱ is selected from the group consisting of:

(a) C₃₋₈ cycloalkyl, optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy

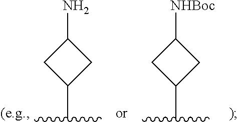 [SMILES: *C1CC(N)C1 ; *C1CC(NC(=O)OC(C)(C)C)C1]

(b) heterocyclyl, wherein the heterocyclyl has 3-8 ring atoms, wherein 1-3 ring atoms are heteroatoms, each independently selected from the group consisting of N, N(H), N(R^{d}), O, and S(O)₀₋₂, wherein the heterocyclyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy (e.g.,

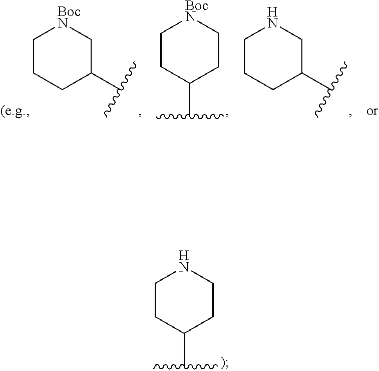

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl, each optionally substituted with 1-2 substituents independently selected from the group consisting of: halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

221. The compound of clause 212, wherein R² is -L⁴-L⁵-Rⁱ; L⁴ is C(═O) or S(O)₂; L⁵ is a bond or C₁₋₄ alkylene; and Rⁱ is selected from the group consisting of:

(c) heteroaryl of 5-6 ring atoms, wherein 1-2 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., pyridyl, pyrimidyl, or pyrazolyl, each optionally substituted with 1-2 substituents independently selected from halo; C₁₋₄ alkyl; C₁₋₄ haloalkyl; cyano; C₁₋₄ alkoxy; and C₁₋₄ haloalkoxy); and

(d) C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy (e.g., phenyl optionally substituted with 1-2 substituents independently selected from halo; C₁₋₄ alkyl; C₁₋₄ haloalkyl; cyano; C₁₋₄ alkoxy; and C₁₋₄ haloalkoxy).

222. The compound of clause 221, wherein R² is selected from the group consisting of:

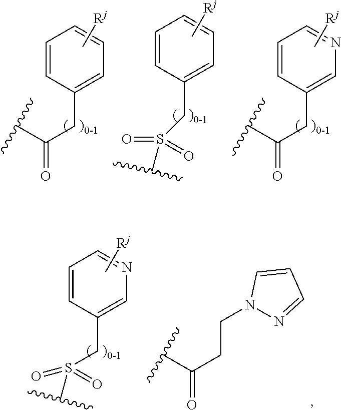

wherein R^{j} is H; halo; C₁₋₄ alkyl; C₁₋₄ haloalkyl; cyano; C₁₋₄ alkoxy; or C₁₋₄ haloalkoxy.

223. The compound of any one of clauses 1-222, wherein R⁵ is H.

224. The compound of clause 1, wherein the compound is a compound of Formula (I-1):

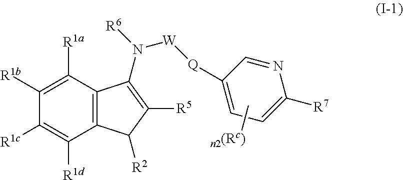

or a pharmaceutically acceptable salt thereof, wherein: n2 is 0, 1, or 2.

225. The compound of clause 1, wherein the compound is a compound of Formula (I-2):

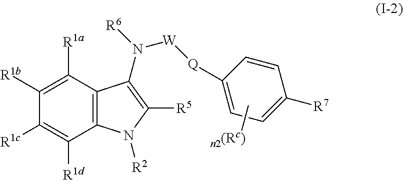

or a pharmaceutically acceptable salt thereof, wherein: n2 is 0, 1, or 2.

226. The compound of clause 1, wherein the compound is a compound of Formula (I-3):

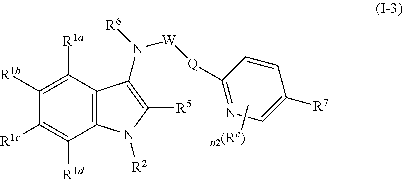

or a pharmaceutically acceptable salt thereof, wherein: n2 is 0, 1, or 2.

227. The compound of clause 1, wherein the compound is a compound of Formula (I-4):

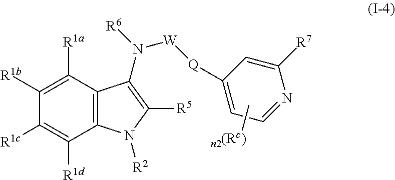

or a pharmaceutically acceptable salt thereof, wherein: n2 is 0, 1, or 2.

228. The compound of clause 1, wherein the compound is a compound of Formula (I-5):

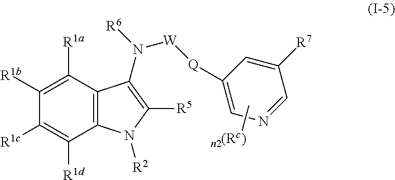

or a pharmaceutically acceptable salt thereof, wherein: n2 is 0, 1, or 2.

229. The compound of clause 1, wherein the compound is a compound of Formula (I-6):

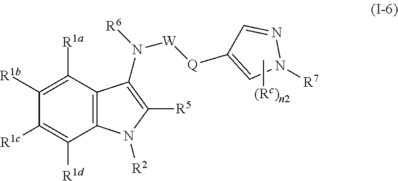

or a pharmaceutically acceptable salt thereof, wherein: n2 is 0 or 1.

230. The compound of any one of clauses 224-229, wherein R⁷ is —R⁸.

231. The compound of any one of clauses 224-230, wherein R⁸ is C₃₋₁₂ cycloalkyl or C₃₋₁₂ cycloalkenyl, each of which is optionally substituted with 1-4 independently selected R^{7′}.

232. The compound of any one of clauses 224-231, wherein R⁸ is C₄₋₈ cycloalkyl which is substituted with 1-3 R^{7′}.

233. The compound of clause 224-232, wherein R⁸ is cyclohexyl which is substituted with 1-3 R⁷′; or wherein R⁸ is cyclobutyl which is substituted with 1-3 R⁷′.

234. The compound of any one of clauses 224-233, wherein R⁸ is

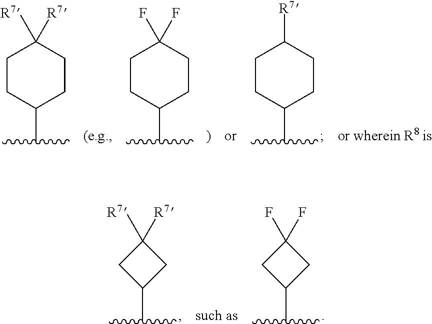

235. The compound of any one of clauses 224-231, wherein R⁸ is C₄₋₆ monocyclic cycloalkyl which is unsubstituted (e.g., cyclopentyl, cyclobutyl, or cyclohexyl); or R⁸ is C₇₋₈ bicyclic (e.g., spirocyclic) cycloalkyl which is unsubstituted

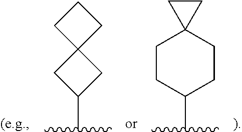 [SMILES: *C1CC2(CCC2)C1 ; *C1CCC2(CC1)CC2]

236. The compound of any one of clauses 224-230, wherein R⁸ is heterocyclyl or heterocycloalkenyl of 4-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′}.

237. The compound of any one of clauses 224-230 or 236, wherein R⁸ is heterocyclyl of 4-8 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 1-3 independently selected R⁷′, such as:

wherein R⁸ is selected from the group consisting of azetidinyl, oxetanyl, pyrrolidinyl, tetrahydrofuranyl, piperidinyl, piperazinyl, morpholinyl, and tetrahydropyranyl, each of which is substituted with 1-3 (e.g., 2) independently selected R⁷′ at one or more ring carbon atoms (e.g., R⁸ is selected from the group consisting of:

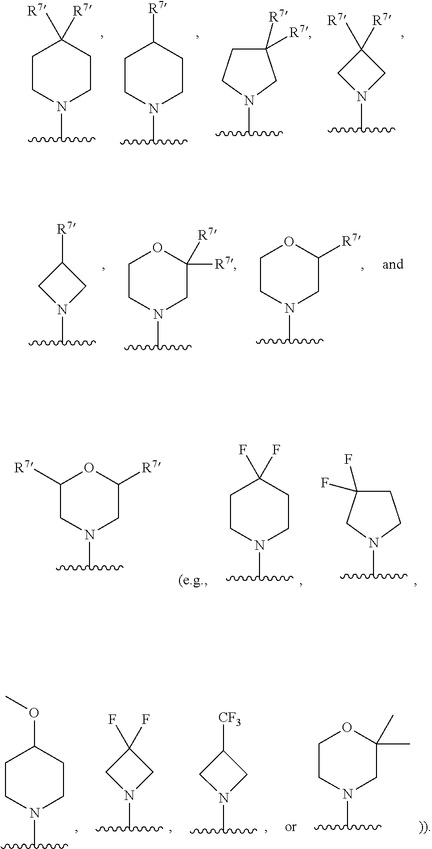

238. The compound of any one of clauses 224-230, wherein R⁸ is spirocyclic heterocyclyl of 6-12, such as 6-8, ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′, such as:

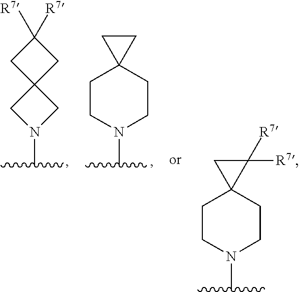

optionally wherein each R⁷′ is an independently selected halo, such as —F.

239. The compound of any one of clauses 224-230, wherein R⁸ is monocyclic heterocyclyl of 3-8 ring atoms, wherein 1-2 ring atoms are heteroatoms, each independently selected from the group consisting of N, N(H), N(R^{d}), O, and S(O)₀₋₂, optionally wherein R⁸ contains a ring N(R^{d}) group.

240. The compound of any one of clauses 224-230 or 239, wherein R⁸ is azetidinyl

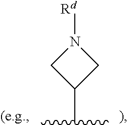

oxetanyl, pyrrolidinyl

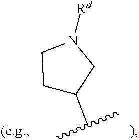

tetrahydrofuranyl tetrahydropyranyl, piperidinyl

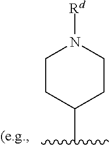

such as

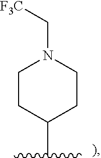 [SMILES: *C1CCN(CC(F)(F)F)CC1]

piperazinyl

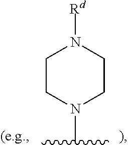

morpholinyl, azepinyl, and 2,6-diazaspiro[3.3]heptanyl

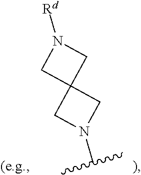

wherein a ring nitrogen atom is substituted with R^{d},

optionally wherein R^{d} is C₁₋₆ alkyl optionally substituted with 1-3 substituents each independently selected from the group consisting of halo, C₁₋₃ alkoxy, and C₁₋₃ haloalkoxy, such as wherein R^{d} is C₂₋₄ alkyl substituted with 1-3 independently selected halo

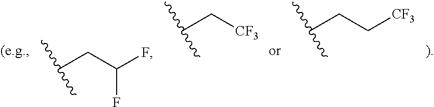

241. The compound of any one of clauses 224-230, wherein R⁷ is -L³-R⁹.

242. The compound of any one of clauses 224-230 or 241, wherein L³ is —O—.

243. The compound of any one of clauses 224-230 and 241, wherein L³ is —NH—.

244. The compound of any one of clauses 241-243, wherein R⁹ is C₃₋₁₂ cycloalkyl or C₃₋₁₂ cycloalkenyl, each of which is optionally substituted with 1-4 independently selected R^{7′}.

245. The compound of clause 244, wherein R⁹ is C₄₋₈ cycloalkyl which is optionally substituted with 1-2 independently selected R^{7′}.

246. The compound of clause 245, wherein R⁹ is cyclobutyl, cyclopentyl, cyclohexyl, or spiro[3.3]heptanyl, each of which is optionally substituted with 1-2 independently selected R⁷′ (e.g., unsubstituted).

247. The compound of any one of clauses 241-243, wherein R⁹ is heterocyclyl of 4-8 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-2 independently selected R^{7′}.

248. The compound of clause 247, wherein R⁹ is selected from the group consisting of azetidinyl, oxetanyl, pyrrolidinyl, tetrahydrofuranyl, piperidinyl, piperazinyl, morpholinyl, and azepinyl, each of which is optionally substituted with 1-2 independently selected R⁷′ (e.g., unsubstituted).

249. The compound of clause 241, wherein R⁷ is

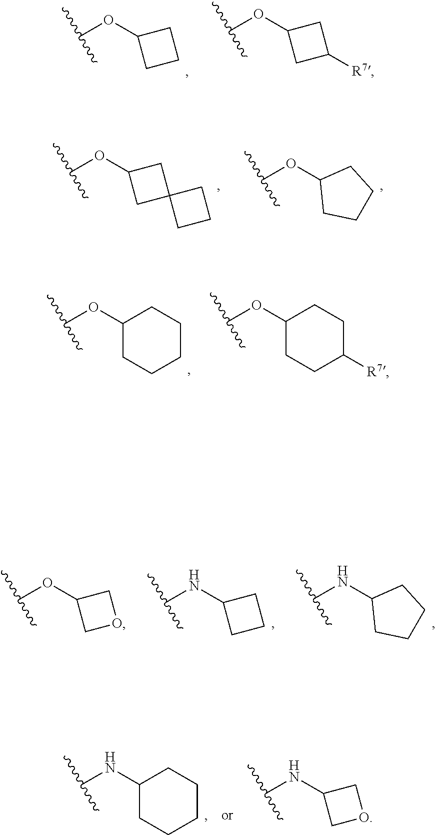

250. The compound of any one of clauses 224-249, wherein each R⁷′ when present is independently selected from the group consisting of: halo, —CN, —OH, —C₁₋₄ alkyl optionally substituted with R^{a}, —C₁₋₄ haloalkyl, —C₁₋₆ alkoxy optionally substituted with R^{a}, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), —C(═O)OH, and —C(═O)N(R′)(R″).

251. The compound of any one of clauses 224-250, wherein each R⁷′ when present is independently selected from the group consisting of: halo, —CN, —C₁₋₄ alkyl optionally substituted with R^{a}, —C₁₋₄ haloalkyl, —C₁₋₆ alkoxy optionally substituted with R^{a}, —C₁₋₆ haloalkoxy, S(O)₁₋₂(C₁₋₄ alkyl), —NR′R″, —S(O)₁₋₂(NR′R″), —C₁₋₄ thioalkoxy, —C(═O)(C₁₋₄ alkyl), —C(═O)O(C₁₋₄ alkyl), and —C(═O)N(R′)(R″), such as wherein each R⁷′ when present is independently halo or C₁₋₃ alkyl, such as —F or methyl

252. The compound of any one of clauses 224-251, wherein each R⁷′ when present is —F.

253. The compound of any one of clauses 224-251, wherein each R⁷′ when present is an independently selected C₁₋₃ alkyl such as methyl; or wherein each R⁷′ when present is an independently selected C₁₋₃ haloalkyl, such as —CF₃.

254. The compound of any one of clauses 224-251, wherein one occurrence of R⁷′ is selected from the group consisting of: —C₁₋₄ alkyl optionally substituted with R^{a}, such as unsubstituted C₁₋₄ alkyl (e.g., methyl, ethyl, n-propyl); —C₁₋₄ alkyl substituted with R^{a} (e.g., —C₁₋₄ alkyl substituted with OH or C₃₋₆ cycloalkyl); —CN; —C₁₋₆ alkoxy optionally substituted with R^{a}, such as unsubstituted C₁₋₆ alkoxy (e.g., methoxy); and C₁₋₆ alkoxy substituted with R^{a} (e.g., —C₁₋₄ alkoxy substituted with OH or C₃₋₆ cycloalkyl); and each remaining R⁷′ when present is independently halo (e.g., —F).

255. The compound of any one of clauses 224-254, wherein n2 is 0.

256. The compound of any one of clauses 224-254, wherein n2 is 1 or 2.

257. The compound of clause 256, wherein n2 is 1, optionally wherein R^{c} is ortho to R⁷.

258. The compound of any one of clauses 224-254 or 256-257, wherein each R^{c} when present is independently selected from the group consisting of: halo; cyano; C₁₋₁₀ alkyl; C₁₋₄ alkoxy; C₁₋₄ haloalkoxy; —S(O)₁₋₂(C₁₋₄ alkyl); —C(═O)(C₁₋₁₀ alkyl); and —C(═O)O(C₁₋₄ alkyl).

259. The compound of any one of clauses 224-254 or 256-258, wherein each R^{e} when present is halo (e.g., —F, —Br, or —Cl) or cyano.

260. The compound of any one of clauses 224-259, wherein Q is NH.

261. The compound of any one of clauses 224-259, wherein Q is N(C₁₋₃ alkyl), wherein the C₁₋₃ alkyl is optionally substituted with R^{a}.

262. The compound of any one of clauses 224-259, wherein Q is *—NH—(C₁₋₃ alkylene), wherein the asterisk represents point of attachment to W.

263. The compound of any one of clauses 224-262, wherein W is C(═O).

264. The compound of any one of clauses 224-262, wherein W is C(═C—NO₂) or C(═N—CN).

265. The compound of any one of clauses 224-262, wherein W is S(O)₂, C(═S), or C(═NR^{d}).

266. The compound of any one of clauses 224-260, wherein Q is NH; and W is C(═O).

267. The compound of any one of clauses 224-266, wherein each of R^{1a}, R^{1b}, R^{1c}, and R^{1d} is independently selected from the group consisting of H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); and —C(═O)N(R′)(R″).

268. The compound of any one of clauses 224-267, wherein each of R^{1a}, R^{1b}, R^{1c}, and R^{1d} is H.

269. The compound of any one of clauses 224-267, wherein 1-2 of R^{1a}, R^{1b}, R^{1c}, and R^{1d} is other than H; and each remaining of R^{1a}, R^{1b}, R^{1c}, and R^{1d} is H.

270. The compound of any one of clauses 224-267 or 269, wherein each of R^{1a} and R^{1d} is independently selected from the group consisting of H and halo.

271. The compound of any one of clauses 224-267 or 269-270, wherein each of R^{1a} and R^{1d} is H.

272. The compound of any one of clauses 224-267 or 269-270, wherein R^{1b} is an independently selected substituent other than H; each of R^{1a}, R^{1c}, and R^{1d} is H.

273. The compound of clause 272, wherein R^{1b} is halo (e.g., —F or —Cl (e.g., —F)).

274. The compound of clause 272, wherein R^{1b} is selected from the group consisting of: C₁₋₆ alkyl, C₁₋₄ haloalkyl (e.g., —CHF₂), C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me).

275. The compound of any one of clauses 224-267 or 269-270, wherein each of R^{1b} and R^{1c} is other than H; and each of R^{1a} and R^{1d} is H.

276. The compound of clause 275, wherein R^{1c} is halo (e.g., —F); R^{1b} is selected from the group consisting of: C₁₋₆ alkyl, C₁₋₄ haloalkyl (e.g., —CHF₂), C₁₋₄ alkoxy, C₁₋₄ haloalkoxy (e.g., OCHF₂), —CN, —SF₅, C₁₋₄ thioalkoxy (e.g., SMe), and S(O)₂(C₁₋₄ alkyl) (e.g., S(O)₂Me).

277. The compound of clause 275, wherein each of R^{1b} and R^{1c} is an independently selected halo.

278. The compound of clause 277, wherein each of R^{1b} and R^{1c} is —F.

279. The compound of any one of clauses 224-278, wherein R² is H; and optionally R⁵ is H.

280. The compound of any one of clauses 224-278, wherein R² is —C(O)(C₁₋₆ alkyl) optionally substituted with 1-3 independently selected R^{a}; or —S(O)₁₋₂(C₁₋₄ alkyl) optionally substituted with 1-3 independently selected R^{a} (e.g., S(O)₂Me).

281. The compound of clause 280, wherein R² is selected from the group consisting of: C(═O)Me, S(O)₂Me,

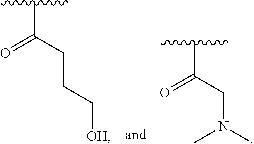

282. The compound of clause 1, wherein the compound is a compound of Formula (I-1a), (I-2a), (I-3a), (I-4a), (I-5a), or (I-6a):

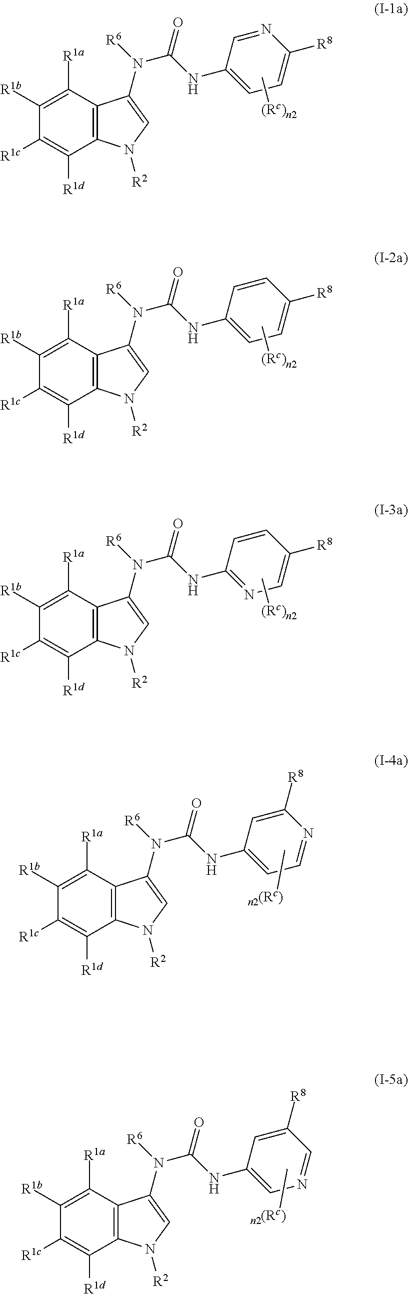

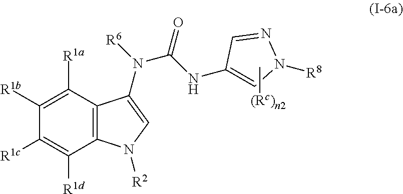

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

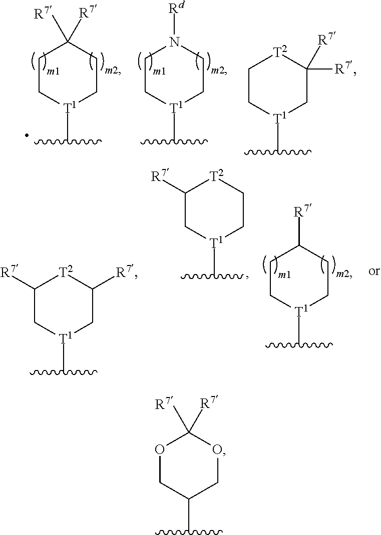

wherein m1 and m2 are independently 0, 1, or 2; T¹ is CH or N; and T² is CH₂, NH, NR d, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R^{7′}.

283. The compound of clauses 1 or 282, wherein the compound is a compound of Formula (I-1a), (I-2a), or (I-3a):

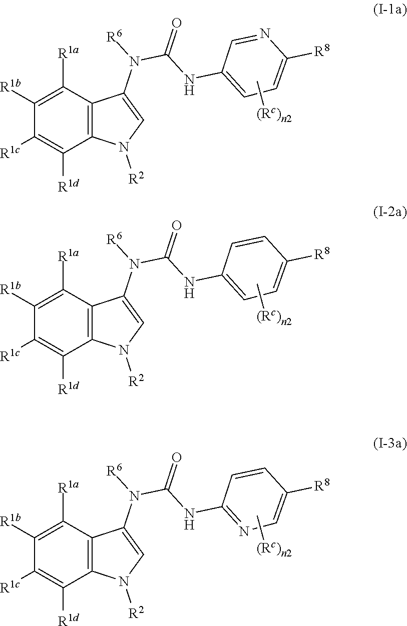

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

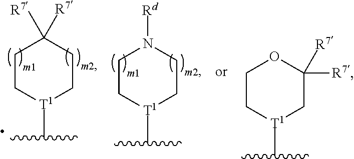

wherein m1 and m2 are independently 0, 1, or 2, and T¹ is CH or N; and
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R^{7′}.

284. The compound of clauses 282 or 283, wherein R² is H.

285. The compound of any one of clauses 282-284, wherein n2 is 1; and R^{c} is ortho to R⁸, optionally wherein R^{c} is halo such as —F or —Cl; or wherein R^{c} is C₁₋₃ alkyl such as methyl.

286. The compound of any one of clauses 282-285, wherein R^{1a} and R^{1d} are H; and R^{1c} is H or halo.

287. The compound of any one of clauses 282-286, wherein R^{1b} is halo, such as —F or —Cl; or wherein R^{1b} is C₁₋₆ alkyl or C₁₋₄ haloalkyl, such as methyl or —CHF₂.

288. The compound of any one of clauses 282-287, wherein R⁸ is

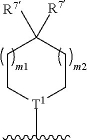

wherein m1 and m2 are independently 0, 1, or 2, and T¹ is CH or N; such as: wherein R⁸ is selected from the group consisting of:

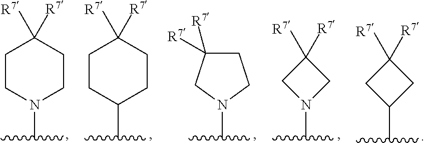

289. The compound of any one of clauses 282-287, wherein R⁸ is

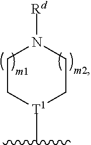

wherein m1 and m2 are independently 0, 1, or 2, and T¹ is CH or N; such as: wherein R⁸ is selected from the group consisting of:

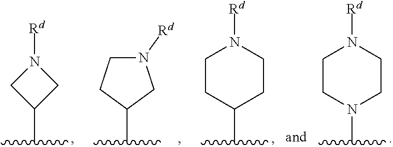

290. The compound of any one of clauses 282-287, wherein R⁸ is selected from the group consisting of:

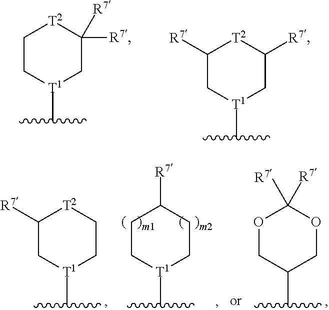

wherein m1 and m2 are independently 0, 1, or 2; T¹ is CH or N; and T² is CH₂, NH, NR^{d}, or O; such as: wherein R⁸ is selected from the group consisting of:

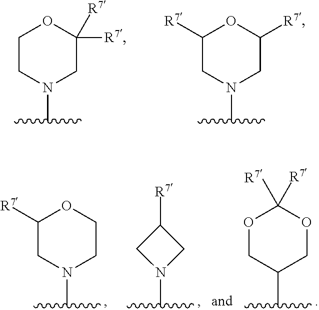

291. The compound of any one of clauses 282-287, wherein R⁸ is

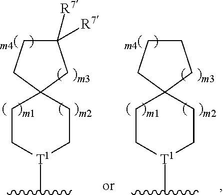

wherein m1, m2, m3, and m4 are independently 0, 1, or 2, provided that m1+m2+m3+m4≤6, and T¹ is CH or N; such as: wherein R⁸ is selected from the group consisting of:

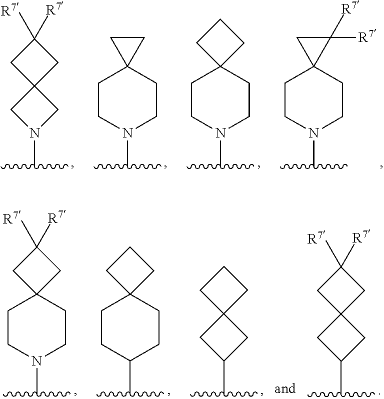

292. The compound of any one of clauses 282-287, wherein R⁸ is

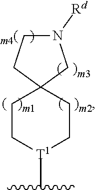

wherein m1, m2, m3, and m4 are independently 0, 1, or 2, provided that m1+m2+m3+m4≤6, and T¹ is CH or N, such as: wherein R⁸ is

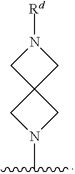

293. The compound of any one of clauses 282-287, wherein R⁸ is selected from the group consisting of:

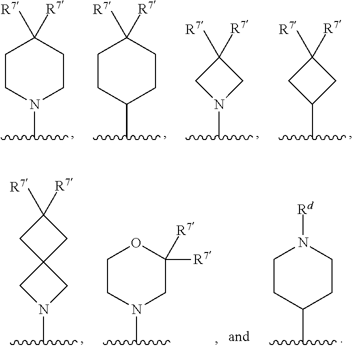

294. The compound of any one of clauses 282-293, wherein each R⁷′ is independently selected from the group consisting of C₁₋₃ alkyl; C₁₋₃ haloalkyl; and halo, such as wherein each R⁷′ is independently selected from the group consisting of methyl, CF₃, and —F; and R^{d} is C₁₋₆ alkyl, such as C₂₋₄ alkyl, optionally substituted with 1-3 independently selected halo, such as —F.

such as: wherein each R⁷′ is independently selected from the group consisting of C₁₋₃ alkyl and halo, such as methyl and —F; and R^{d} is C₁₋₆ alkyl, such as C₂₋₄ alkyl, optionally substituted with 1-3 independently selected halo, such as —F.

295. The compound of clause 1, wherein the compound is a compound of Formula (I-3a):

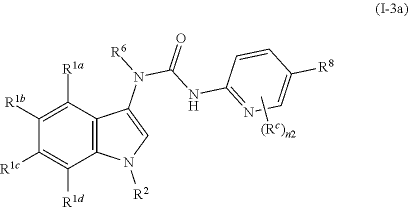

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

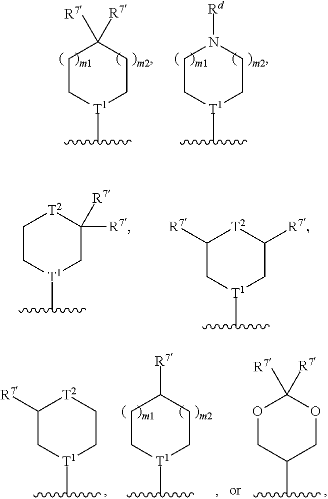

wherein m1 and m2 are independently 0, 1, or 2; T¹ is CH or N; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

296. The compound of clause 295, wherein R⁸ is

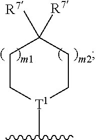

and optionally wherein each R⁷′ is an independently selected halo, such as —F.

297. The compound of clauses 295 or 296, wherein R⁸ is selected from the group consisting of:

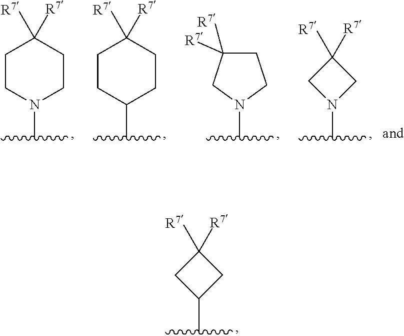

and optionally wherein each R⁷′ is —F; such as wherein R⁸ is

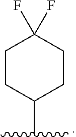 [SMILES: *C1CCC(F)(F)CC1]

298. The compound of any one of clauses 295-297, wherein R^{1a} and R^{1d} are H; R^{1b} is halo, such as —F; R^{1c} is —H or halo, such as —H or —F; and R² is H.

299. The compound of any one of clauses 295-298, wherein the compound has Formula (I-3a-1):

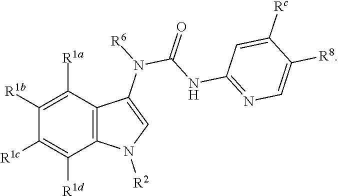

300. The compound of any one of clauses 295-299, wherein R^{c} is halo, such as —F or —Cl.

301. The compound of clause 1, wherein the compound is a compound of Formula (I-2a):

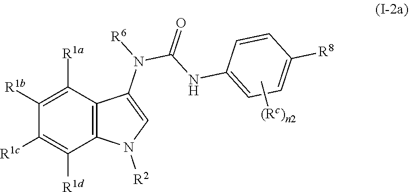

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

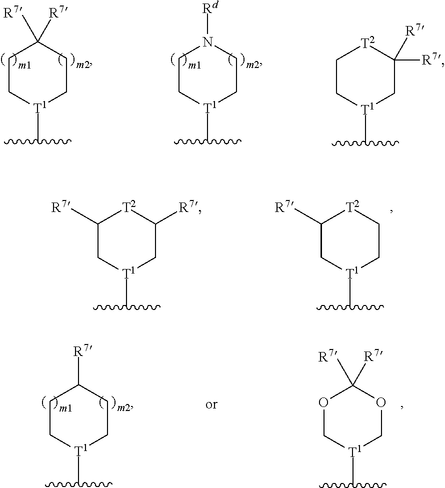

wherein m1 and m2 are independently 0, 1, or 2; T¹ is CH or N; and T² is CH₂, NH, NR^{d}, or 0;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

302. The compound of clause 301, wherein R⁸ is

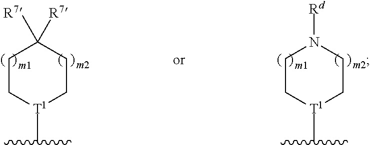

and optionally wherein each R⁷′ is an independently selected halo, such as —F; and optionally wherein R^{d} is C₂₋₄ alkyl which is substituted with 1-3 independently selected halo, such as —F.

303. The compound of clauses 301 or 302, wherein R⁸ is selected from the group consisting of:

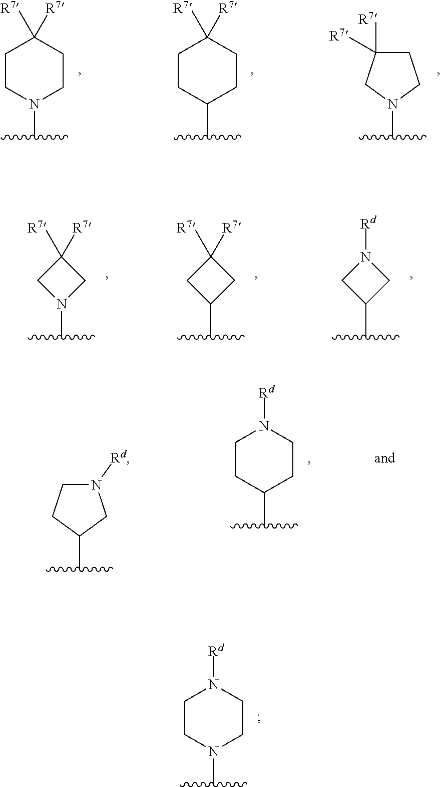

and optionally wherein each R⁷′ is —F; and optionally wherein R^{d} is C₂₋₄ alkyl which is substituted with 1-3 —F, such as wherein R⁸ is

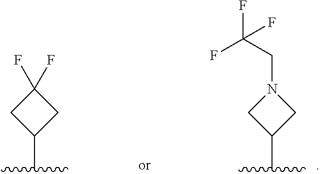 [SMILES: *C1CC(F)(F)C1 ; *C1CN(CC(F)(F)F)C1]

304. The compound of any one of clauses 301-303, wherein R^{1a}, R^{1d}, and R^{1c} are each H; R^{1b} is —H or halo, such as —H, —Cl, or —F; and R² is H.

305. The compound of any one of clauses 301-304, wherein the compound has Formula (I-2a-1):

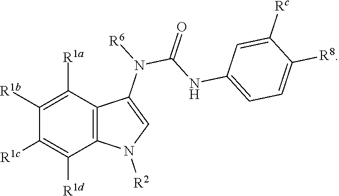

306. The compound of any one of clauses 301-305, wherein R^{c} is -halo.

307. The compound of clause 1, wherein the compound is a compound of Formula (I-7a):

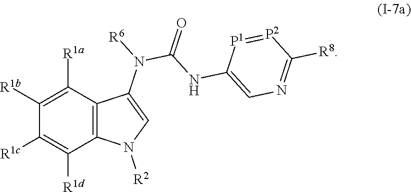

or a pharmaceutically acceptable salt thereof, wherein:

one of P¹ and P² is N; and the other of P¹ and P² is CH;

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

R⁸ is selected from the group consisting of:

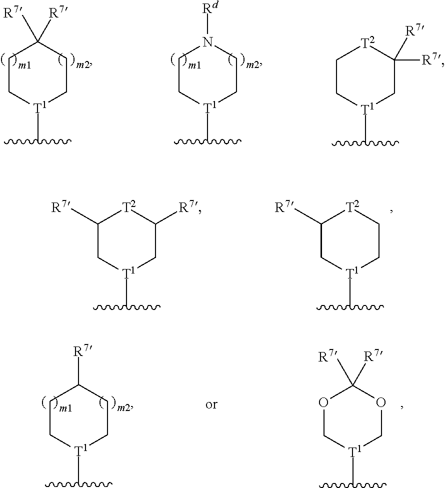

wherein m1 and m2 are independently 0, 1, or 2; T¹ is CH or N; and T² is CH₂, NH, NR^{d}, or 0;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

308. The compound of clause 307, wherein R⁸ is

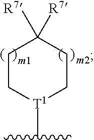

and optionally wherein each R⁷′ is an independently selected halo, such as —F.

309. The compound of clauses 307 or 308, wherein R⁸ is selected from the group consisting of:

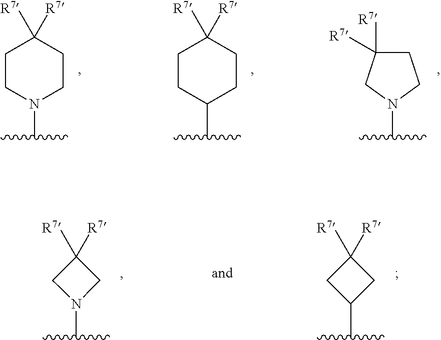

and optionally wherein each R⁷′ is —F, optionally wherein R⁸ is

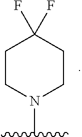 [SMILES: *N1CCC(F)(F)CC1]

310. The compound of any one of clauses 307-309, wherein R^{1a}, R^{1d}, and R^{1c} are H; R^{1b} is halo, such as —Cl; and R² is H.

311. The compound of clause 1, wherein the compound is a compound of Formula (I-1a):

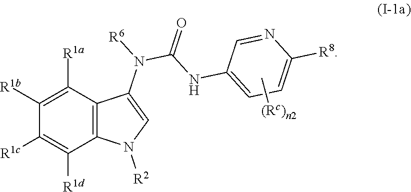

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

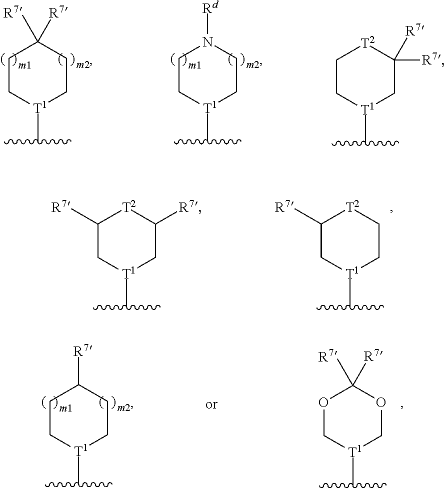

wherein m1 and m2 are independently 0, 1, or 2; T¹ is CH or N; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

312. The compound of clause 311, wherein R⁸ is

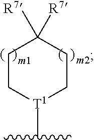

and optionally wherein each R⁷′ is an independently selected halo, such as —F.

313. The compound of clauses 311 or 312, wherein R⁸ is selected from the group consisting of:

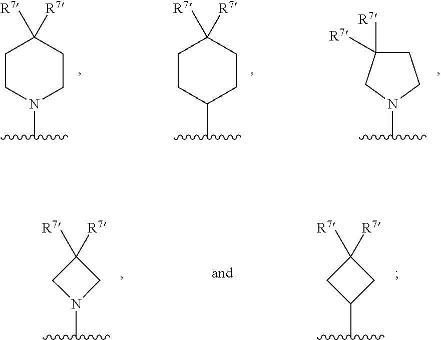

and optionally wherein each R⁷′ is —F, such as wherein R⁸ is selected from the group consisting of:

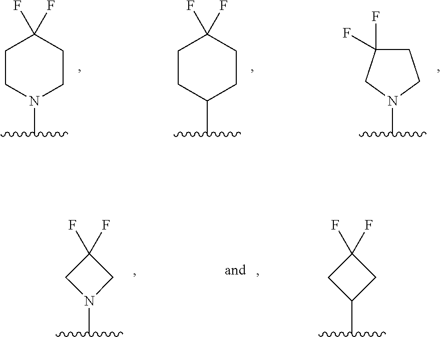 [SMILES: *N1CCC(F)(F)CC1 ; *C1CCC(F)(F)CC1 ; *N1CCC(F)(F)C1 ; *N1CC(F)(F)C1 ; *C1CC(F)(F)C1]

314. The compound of clause 311, wherein R⁸ is

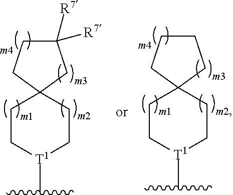

wherein: m1, m2, m3, and m4 are independently 0, 1, or 2, provided that m1+m2+m3+m4≤6; T¹ is CH or N; and

each R⁷′ is independently selected from the group consisting of C₁₋₃ alkyl; C₁₋₃ haloalkyl; and halo, such as methyl, CF₃, and —F.

315. The compound of clauses 311 or 314, wherein R⁸ is selected from the group consisting of:

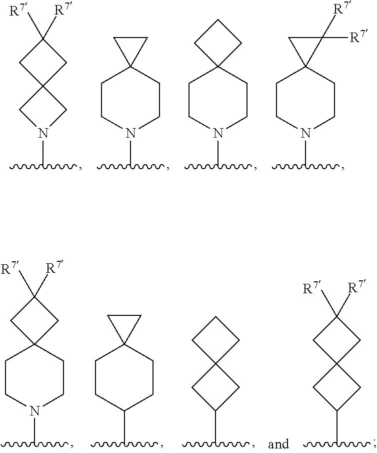

and optionally wherein each R⁷′ is —F, such as wherein R⁸ is selected from the group consisting of:

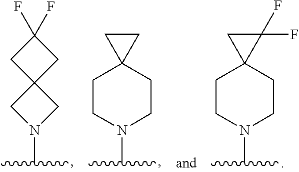

316. The compound of any one of clauses 311-315, wherein R^{1a} and R^{1d} are H; R^{1b} is halo, such as —F or —Cl; R^{1c} is —H or halo, such as —H, —F, or —Cl; and R² is H.

317. The compound of any one of clauses 311-316, wherein the compound has Formula (I-1a-1):

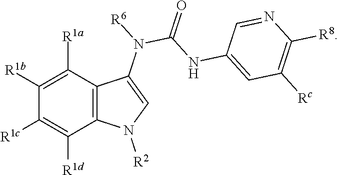

318. The compound of any one of clauses 311-317, wherein R^{c} is halo, such as —F or —Cl.

319. The compound of clause 1, wherein the compound is a compound of Formula (I-6a):

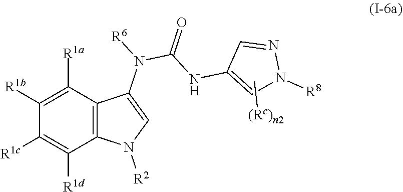

or a pharmaceutically acceptable salt thereof, wherein:

each of R¹, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

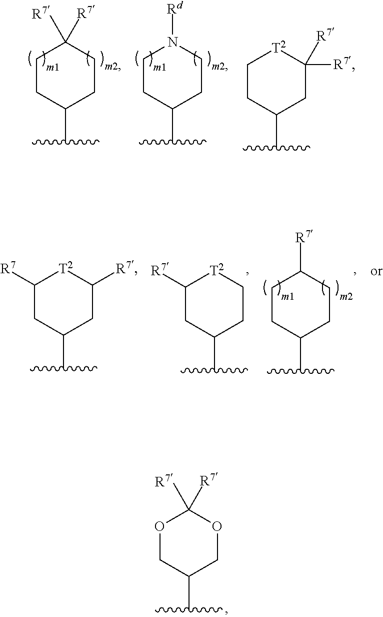

wherein m1 and m2 are independently 0, 1, or 2; and T² is CH₂, NH, NR^{d}, or O;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

320. The compound of clause 319, wherein R⁸ is

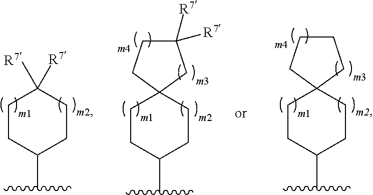

wherein: m1, m2, m3, and m4 are independently 0, 1, or 2, provided that m1+m2+m3+m4≤6; and

each R⁷′ is independently selected from the group consisting of C₁₋₃ alkyl; C₁₋₃ haloalkyl; and halo, such as methyl, CF₃, and —F.

321. The compound of clauses 319 or 320, wherein R⁸ is

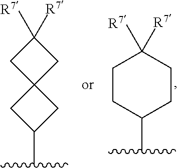

such as:

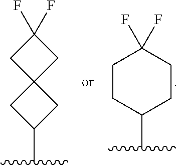 [SMILES: *C1CC2(C1)CC(F)(F)C2 ; *C1CCC(F)(F)CC1]

322. The compound of any one of clauses 319-321, wherein R^{1a}, R^{1d}, and R^{1c} are H; R^{1b} is halo, such as —Cl; and R² is H.

323. The compound of any one of clauses 319-322, wherein n2 is 0.

324. The compound of clause 1, wherein the compound is a compound of Formula (I-4a):

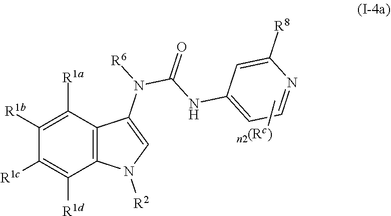

or a pharmaceutically acceptable salt thereof, wherein:

each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;

n2 is 0, 1, or 2;

each R^{c} when present is independently selected from the group consisting of: halo, cyano, C₁₋₃ alkyl, and C₁₋₃ alkoxy;

R⁸ is selected from the group consisting of:

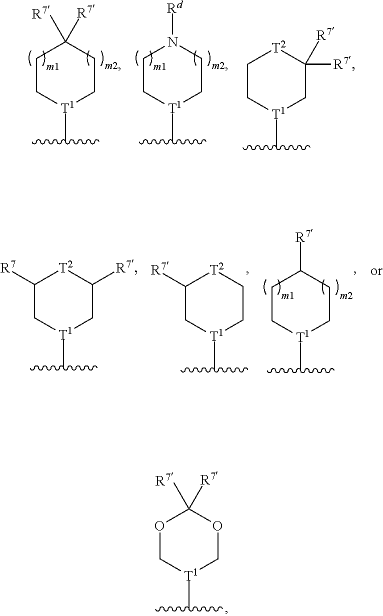

wherein m1 and m2 are independently 0, 1, or 2; T¹ is CH or N; and T² is CH₂, NH, NR^{d}, or 0;
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′; and
spirocyclic C₆₋₁₂ cycloalkyl which is optionally substituted with 1-4 independently selected R⁷′.

325. The compound of clause 324, wherein R⁸ is

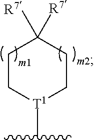

and optionally wherein each R⁷′ is an independently selected halo, such as —F.

326. The compound of clauses 324 or 325, wherein R⁸ is selected from the group consisting of:

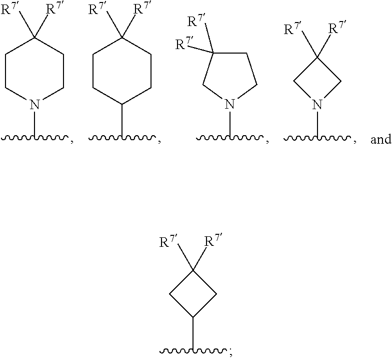

and optionally wherein each R⁷′ is —F, such as wherein R⁸ is:

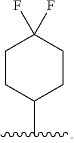 [SMILES: *C1CCC(F)(F)CC1]

327. The compound of any one of clauses 324-326, wherein R^{1a} and R^{1d} are H; R^{1b} is halo, such as —F or —Cl; R^{1c} is H or halo, such as —H or —F; and R² is H.

328. The compound of any one of clauses 324-327, wherein n2 is 1; and the compound has Formula (I-4a-1):

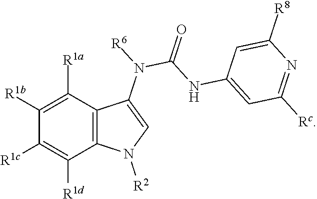

329. The compound of any one of clauses 324-328, wherein R^{c} is -halo.

330. The compound of any one of clauses 324-327, wherein n2 is 0.

331. The compound of any one of clauses 1-330, wherein R⁶ is H.

332. The compound of clause 1, wherein the compound is selected from the group consisting of the compounds delineated in Table C1, or a pharmaceutically acceptable salt thereof.

333. The compound of clause 1, wherein the compound is selected from the group consisting of the following:

| Compound # | Name |
|---|---|
| 196 | 1-(5-chloro-1H-indol-3-yl)-3-(5-chloro-6-(4,4-difluoropiperidin-1-yl)pyridin-3- |
| | yl)urea |
| 123 | 1-(5,6-difluoro-1H-indol-3-yl)-3-(6-(4-(2-methoxyethyl)piperazin-1-yl)-5- |
| | methylpyridin-3-yl)urea |
| 124 | 1-(5-chloro-1H-indol-3-yl)-3-(5-chloro-6-(4-(3,3,3-trifluoropropyl)piperazin-1- |
| | yl)pyridin-3-yl)urea |
| 125 | 1-(5-cyano-6-(3,3-difluorocyclobutyl)pyridin-3-yl)-3-(5,6-difluoro-1H-indol-3- |
| | yl)urea |
| 126 | 1-(5-chloro-1H-indol-3-yl)-3-(5-chloro-6-(4-(2,2,2-trifluoroethyl)piperazin-1- |
| | yl)pyridin-3-yl)urea |
| 127 | 1-(5-chloro-1H-indol-3-yl)-3-(6-(4,4-difluoropiperidin-1-yl)-5-(2- |
| | hydroxyethyl)pyridin-3-yl)urea |
| 128 | 1-(5,6-dichloro-1H-indol-3-yl)-3-(6-(4,4-difluoropiperidin-1-yl)pyridin-3-yl)urea |
| 129 | 1-(5-chloro-1H-indol-3-yl)-3-(6-(6,6-difluoro-2-azaspiro[3.3]heptan-2-yl)-5- |
| | methylpyridin-3-yl)urea |
| 130 | 1-(5-chloro-1H-indol-3-yl)-3-(6-(4,4-difluorocyclohexyl)pyridin-3-yl)urea |
| 131 | 1-(5-chloro-1H-indol-3-yl)-3-(6-(4,4-difluoropiperidin-1-yl)-5-ethylpyridin-3-yl)urea |
| 132 | 1-(6-(4,4-difluoropiperidin-1-yl)-5-ethylpyridin-3-yl)-3-(5-fluoro-1H-indol-3-yl)urea |
| 133 | 1-(5-chloro-1H-indol-3-yl)-3-(5-methyl-6-(1-(2,2,2-trifluoroethyl)piperidin-4- |
| | yl)pyridin-3-yl)urea |
| 134 | 1-(5-chloro-6-(4-(2-methoxyethyl)piperazin-1-yl)pyridin-3-yl)-3-(5,6-difluoro-1H- |
| | indol-3-yl)urea |
| 135 | 1-(5-chloro-1H-indol-3-yl)-3-(5-chloro-6-(4-(2-methoxyethyl)piperazin-1-yl)pyridin- |
| | 3-yl)urea |
| 136 | 1-(5-chloro-1H-indol-3-yl)-3-(6-(4,4-difluorocyclohexyl)-5-(hydroxymethyl)pyridin- |
| | 3-yl)urea |
| 137 | 1-(5-chloro-1H-indol-3-yl)-3-(6-(6,6-difluoro-2-azaspiro[3.3]heptan-2-yl)-5- |
| | fluoropyridin-3-yl)urea |
| 138 | 1-(5-chloro-1H-indol-3-yl)-3-(5-chloro-6-(6,6-difluoro-2-azaspiro[3.3]heptan-2- |
| | yl)pyridin-3-yl)urea |
| 139 | 1-(5-chloro-1H-indol-3-yl)-3-(4-(3,3-difluorocyclobutyl)-3-fluorophenyl)urea |
| 140 | 1-(6-chloro-1H-indol-3-yl)-3-(5-chloro-6-(4,4-difluorocyclohexyl)pyridin-3-yl)urea |
| 141 | 1-(5-chloro-6-(4,4-difluorocyclohexyl)pyridin-3-yl)-3-(6-fluoro-1H-indol-3-yl)urea |
| 142 | 1-(5-chloro-6-((2R,6R)-2,6-dimethylmorpholino)pyridin-3-yl)-3-(5,6-difluoro-1H- |
| | indol-3-yl)urea |
| 143 | 1-(5,6-difluoro-1H-indol-3-yl)-3-(6-(4,4-difluoropiperidin-1-yl)-5-(2- |
| | hydroxyethyl)pyridin-3-yl)urea |
| 144 | 1-(5-chloro-1H-indol-3-yl)-3-(5-chloro-6-(3,3-difluorocyclobutyl)pyridin-3-yl)urea |
| 145 | 1-(6-fluoro-1H-indol-3-yl)-3-(5-fluoro-6-(1-oxa-9-azaspiro[5.5]undecan-9- |
| | yl)pyridin-3-yl)urea |
| 146 | 1-(5-chloro-6-((2R,6S)-2,6-dimethylmorpholino)pyridin-3-yl)-3-(5,6-difluoro-1H- |
| | indol-3-yl)urea |
| 147 | 1-(5-chloro-6-((2S,6S)-2,6-dimethylmorpholino)pyridin-3-yl)-3-(5,6-difluoro-1H- |
| | indol-3-yl)urea |
| 149 | (R)-1-(5-chloro-6-(3-methoxypiperidin-1-yl)pyridin-3-yl)-3-(5,6-difluoro-1H-indol- |
| | 3-yl)urea |
| 148 | (S)-1-(5-chloro-6-(3-methoxypiperidin-1-yl)pyridin-3-yl)-3-(5,6-difluoro-1H-indol- |
| | 3-yl)urea |
| 150 | (S)-1-(5-chloro-6-(3-(2-methoxyethoxy)pyrrolidin-1-yl)pyridin-3-yl)-3-(5,6-difluoro- |
| | 1H-indol-3-yl)urea |
| 151 | (S)-1-(5,6-difluoro-1H-indol-3-yl)-3-(5-methyl-6-(2-methylmorpholino)pyridin-3- |
| | yl)urea |
| 152 | (R)-1-(5,6-difluoro-1H-indol-3-yl)-3-(6-(3-(2-methoxyethoxy)pyrrolidin-1-yl)-5- |
| | methylpyridin-3-yl)urea |
| 153 | 1-(5-chloro-6-(2,2-dimethylmorpholino)pyridin-3-yl)-3-(5,6-difluoro-1H-indol-3- |
| | yl)urea |
| 154 | 1-(4-chloro-5-(4,4-difluorocyclohexyl)pyridin-2-yl)-3-(5,6-difluoro-1H-indol-3- |
| | yl)urea |
| 155 | 1-(5-chloro-1H-indol-3-yl)-3-(6-(6,6-difluoro-2-azaspiro[3.3]heptan-2-yl)pyridin-3- |
| | yl)urea |
| 156 | 1-(5-chloro-1H-indol-3-yl)-3-(6-(4,4-difluorocyclohexyl)-5-methylpyridin-3-yl)urea |
| 157 | 1-(5-chloro-1H-indol-3-yl)-3-(5-chloro-6-(4,4-difluorocyclohexyl)pyridin-3-yl)urea |
| 158 | (R)-1-(5-chloro-6-(2-methylmorpholino)pyridin-3-yl)-3-(5,6-difluoro-1H-indol-3- |
| | yl)urea |
| 160 | 1-(5,6-difluoro-1H-indol-3-yl)-3-(5-methyl-6-(2-oxa-6-azaspiro[3.3]heptan-6- |
| | yl)pyridin-3-yl)urea |
| 161 | 1-(5,6-difluoro-1H-indol-3-yl)-3-(6-(6-hydroxy-2-azaspiro[3.3]heptan-2-yl)-5- |
| | methylpyridin-3-yl)urea |
| 162 | 1-(5,6-difluoro-1H-indol-3-yl)-3-(6-(3,3-difluoroazetidin-1-yl)-5-methylpyridin-3- |
| | yl)urea |
| 163 | 1-(5-cyano-6-(4,4-difluoropiperidin-1-yl)pyridin-3-yl)-3-(5,6-difluoro-1H-indol-3- |
| | yl)urea |
| 164 | 1-(6-(4,4-difluoropiperidin-1-yl)-5-methylpyridin-3-yl)-3-(5-(2-hydroxyethyl)-1H- |
| | indol-3-yl)urea |
| 165 | 1-(5-chloro-6-morpholinopyridin-3-yl)-3-(5,6-difluoro-1H-indol-3-yl)urea |
| 166 | 1-(5,6-difluoro-1H-indol-3-yl)-3-(5-methyl-6-(1-(2,2,2-trifluoroethyl)piperidin-4- |
| | yl)pyridin-3-yl)urea |
| 167 | 1-(5,6-difluoro-1H-indol-3-yl)-3-(6-(6,6-difluoro-2-azaspiro[3.3]heptan-2-yl)-5- |
| | methylpyridin-3-yl)urea |
| 168 | 1-(5,6-difluoro-1H-indol-3-yl)-3-(6-(3,3-difluoroazetidin-1-yl)-5-methoxypyridin-3- |
| | yl)urea |
| 169 | 1-(5,6-difluoro-1H-indol-3-yl)-3-(5-methyl-6-(tetrahydro-2H-pyran-4-yl)pyridin-3- |
| | yl)urea |
| 170 | 1-(5-chloro-6-(4,4-difluoropiperidin-1-yl)pyridin-3-yl)-3-(5-chloro-6-fluoro-1H- |
| | indol-3-yl)urea |
| 183 | 1-(5-chloro-6-(4,4-difluoropiperidin-1-yl)pyridin-3-yl)-3-(5-(methylthio)-1H-indol- |
| | 3-yl)urea |
| 172 | 1-(1H-indol-3-yl)-3-(6-(4-(prop-2-yn-1-yl)piperidin-1-yl)pyridin-3-yl)urea |
| 173 | 1-(5-chloro-6-(3,3-difluorocyclobutyl)pyridin-3-yl)-3-(5,6-difluoro-1H-indol-3- |
| | yl)urea |
| 174 | 1-(5-chloro-1H-indol-3-yl)-3-(6-chloro-5-(4,4-difluoropiperidin-1-yl)pyrazin-2- |
| | yl)urea |
| 175 | 1-(5,6-difluoro-1H-indol-3-yl)-3-(5-(4,4-difluoropiperidin-1-yl)-6-methylpyrazin-2- |
| | yl)urea |
| 176 | 1-(5-chloro-6-(1-oxa-9-azaspiro[5.5]undecan-9-yl)pyridin-3-yl)-3-(5,6-difluoro-1H- |
| | indol-3-yl)urea |
| 177 | 1-(5-chloro-6-(4-(2-methoxyethyl)piperidin-1-yl)pyridin-3-yl)-3-(5,6-difluoro-1H- |
| | indol-3-yl)urea |
| 179 | 1-(5,6-difluoro-1H-indol-3-yl)-3-(2-(4,4-difluoropiperidin-1-yl)pyrimidin-5-yl)urea |
| 180 | 1-(5-chloro-1H-indol-3-yl)-3-(2-(4,4-difluoropiperidin-1-yl)pyrimidin-5-yl)urea |
| 178 | 1-(2-(4,4-difluoropiperidin-1-yl)pyrimidin-5-yl)-3-(1H-indol-3-yl)urea |
| 181 | 1-(5-chloro-6-(4,4-difluoropiperidin-1-yl)pyridin-3-yl)-3-(5-cyano-1H-indol-3- |
| | yl)urea |
| 182 | 1-(5-cyano-6-(4,4-difluorocyclohexyl)pyridin-3-yl)-3-(5-fluoro-1H-indol-3-yl)urea |
| 185 | (R)-1-(5-chloro-6-(3-(2-methoxyethoxy)pyrrolidin-1-yl)pyridin-3-yl)-3-(5,6- |
| | difluoro-1H-indol-3-yl)urea |
| 186 | 1-(5-chloro-6-(6-oxa-2-azaspiro[3.4]octan-2-yl)pyridin-3-yl)-3-(5,6-difluoro-1H- |
| | indol-3-yl)urea |
| 187 | 1-(5-chloro-6-(3-methoxy-3-methylazetidin-1-yl)pyridin-3-yl)-3-(5,6-difluoro-1H- |
| | indol-3-yl)urea |
| 188 | (S)-1-(5-chloro-6-(2-methylmorpholino)pyridin-3-yl)-3-(5,6-difluoro-1H-indol-3- |
| | yl)urea |
| 189 | 1-(5-chloro-1H-indol-3-yl)-3-(5-(4,4-difluoropiperidin-1-yl)pyrazin-2-yl)urea |
| 190 | 1-(6-(4,4-difluorocyclohexyl)pyridin-3-yl)-3-(1H-indol-3-yl)urea |
| 192 | 1-(6-(4,4-difluorocyclohexyl)pyridin-3-yl)-3-(5-iodo-1H-indol-3-yl)urea |
| 193 | 1-(5-chloro-6-(4,4-difluoropiperidin-1-yl)pyridin-3-yl)-3-(5-fluoro-1H-indol-3- |
| | yl)urea |
| 194 | 1-(5,6-difluoro-1H-indol-3-yl)-3-(5-(4,4-difluoropiperidin-1-yl)pyrazin-2-yl)urea |
| 195 | 1-(6-(4,4-difluoropiperidin-1-yl)-5-methylpyridin-3-yl)-3-(5-fluoro-1H-indol-3- |
| | yl)urea |
| 197 | 1-(6-(4,4-difluoropiperidin-1-yl)-5-methylpyridin-3-yl)-3-(5-methyl-1H-indol-3- |
| | yl)urea |
| 198 | 1-(1H-indol-3-yl)-3-(5-methyl-6-(6-azaspiro[2.5]octan-6-yl)pyridin-3-yl)urea |
| 199 | 1-(5-chloro-6-(4,4-difluoropiperidin-1-yl)pyridin-3-yl)-3-(5,6-difluoro-1H-indol-3- |
| | yl)urea |
| 201 | 1-(6-(4,4-difluoropiperidin-1-yl)-5-methylpyridin-3-yl)-3-(1H-indol-3-yl)urea |
| 200 | 1-(5,6-difluoro-1H-indol-3-yl)-3-(6-(4,4-difluoropiperidin-1-yl)-5-methylpyridin-3- |
| | yl)urea |
| 202 | 1-(1H-indol-3-yl)-3-(6-(4-methoxypiperidin-1-yl)-5-methylpyridin-3-yl)urea |
| 203 | 1-(5-bromo-1H-indol-3-yl)-3-(6-(4,4-difluorocyclohexyl)pyridin-3-yl)urea |
| 204 | 1-(6-(4,4-difluoropiperidin-1-yl)pyridazin-3-yl)-3-(1H-indol-3-yl)urea |
| 205 | 1-(5-(4,4-difluoropiperidin-1-yl)pyrazin-2-yl)-3-(1H-indol-3-yl)urea |
| 206 | 1-(5-chloro-6-(4,4-difluoropiperidin-1-yl)pyridin-3-yl)-3-(1H-indol-3-yl)urea |
| 207 | 1-(3-cyano-4-(4,4-difluorocyclohexyl)phenyl)-3-(1H-indol-3-yl)urea |
| 208 | 1-(5-bromo-1H-indol-3-yl)-3-(5-chloro-6-(4,4-difluoropiperidin-1-yl)pyridin-3- |
| | yl)urea |
| 184 | 1-(5-bromo-1H-indol-3-yl)-3-(6-(4,4-difluoropiperidin-1-yl)-5-methylpyridin-3- |
| | yl)urea |
| 171 | 1-(5-chloro-6-(4,4-difluoropiperidin-1-yl)pyridin-3-yl)-3-(5-(methylsulfonyl)-1H- |
| | indol-3-yl)urea |
| 213 | 1-(5,6-difluoro-1H-indol-3-yl)-3-(2-(1-(2,2,2-trifluoroethyl)piperidin-4-yl)pyridin-4- |
| | yl)urea |
| 214 | 1-(5-chloro-1H-indol-3-yl)-3-(1-(spiro[2.5]octan-6-yl)-1H-pyrazol-4-yl)urea |
| 215 | 1-(5-chloro-1H-indol-3-yl)-3-(1-(3,3-difluorocyclobutyl)-1H-pyrazol-4-yl)urea |
| 216 | 1-(5,6-difluoro-1H-indol-3-yl)-3-(1-(3,3-difluorocyclobutyl)-1H-pyrazol-4-yl)urea |
| 217 | 1-(5-chloro-1H-indol-3-yl)-3-(1-(1-cyclobutylpropan-2-yl)-1H-pyrazol-4-yl)urea |
| 218 | 1-(5-chloro-1H-indol-3-yl)-3-(3-chloro-4-(1-(2,2,2-trifluoroethyl)azetidin-3- |
| | yl)phenyl)urea |
| 219 | 1-(5,6-difluoro-1H-indol-3-yl)-3-(2-(1-(2,2,2-trifluoroethyl)piperidin-3-yl)pyridin-4- |
| | yl)urea |
| 220 | 1-(5,6-difluoro-1H-indol-3-yl)-3-(2-(4,4-difluorocyclohexyl)pyridin-4-yl)urea |
| 221 | 1-(5-chloro-1H-indol-3-yl)-3-(2-chloro-6-(4,4-difluorocyclohexyl)pyridin-4-yl)urea |
| 222 | 1-(5-chloro-1H-indol-3-yl)-3-(1-((4,4-difluorocyclohexyl)methyl)-1H-pyrazo1-4- |
| | yl)urea |
| 223 | 1-(5-chloro-1H-indol-3-yl)-3-(1-(1-(3,3,3-trifluoropropyl)piperidin-4-yl)-1H- |
| | pyrazo1-4-yl)urea |
| 224 | 1-(5-chloro-1H-indol-3-yl)-3-(5-chloro-6-(9,9-difluoro-1,5-dioxaspiro[5.5]undecan- |
| | 3-yl)pyridin-3-yl)urea |
| 225 | 1-(3-chloro-4-(3,3-difluorocyclobutyl)phenyl)-3-(5,6-difluoro-1H-indol-3-yl)urea |
| 226 | 1-(5-chloro-1H-indol-3-yl)-3-(3-chloro-4-(3,3-difluorocyclobutyl)phenyl)urea |
| 227 | 1-(5-chloro-1H-indol-3-yl)-3-(6-(3,3-difluorocyclobutyl)-5-fluoropyridin-3-yl)urea |
| 228 | 3-(5-chloro-1H-indol-3-yl)-1-(5-chloro-6-(4,4-difluorocyclohexyl)pyridin-3-yl)-1-(2- |
| | methoxyethyl)urea |
| 229 | 3-(5-chloro-1H-indol-3-yl)-1-(5-chloro-6-(1-(2,2,2-trifluoroethyl)piperidin-4- |
| | yl)pyridin-3-yl)-1-ethylurea |
| 230 | 1-(5-chloro-6-fluoro-1H-indol-3-yl)-3-(1-(4,4-difluorocyclohexyl)-1H-pyrazol-4- |
| | yl)urea |
| 231 | 1-(5-chloro-1H-indol-3-yl)-3-(6-chloro-5-(4,4-difluorocyclohexyl)pyridin-3-yl)urea |
| 232 | 1-(7-bromo-5-chloro-1H-indol-3-yl)-3-(5-chloro-6-(4,4-difluoropiperidin-1- |
| | yl)pyridin-3-yl)urea |
| 233 | 1-(5-chloro-1H-indol-3-yl)-3-(5-chloro-6-(3-(trifluoromethyl)azetidin-1-yl)pyridin- |
| | 3-yl)urea |
| 234 | 1-(5-chloro-1H-indol-3-yl)-3-(1-(6,6-difluorospiro[3.3]heptan-2-yl)-1H-pyrazo1-4- |
| | yl)urea |
| 235 | 1-(5-chloro-1H-indol-3-yl)-3-(1-(cyclobutylmethyl)-1H-pyrazol-4-yl)urea |
| 236 | 1-(5-chloro-6-(1,1-difluoro-6-azaspiro[2.5]octan-6-yl)pyridin-3-yl)-3-(5,6-difluoro- |
| | 1H-indol-3-yl)urea |
| 237 | 1-(5-chloro-1H-indol-3-yl)-3-(5-chloro-6-(1,1-difluoro-6-azaspiro[2.5]octan-6- |
| | yl)pyridin-3-yl)urea |
| 238 | 1-(5-chloro-1H-indol-3-yl)-3-(5-chloro-6-(1-(2,2,2-trifluoroethyl)pyrrolidin-3- |
| | yl)pyridin-3-yl)urea |
| 240 | 1-(5-chloro-1H-indol-3-yl)-3-(5-fluoro-6-(6-azaspiro[2.5]octan-6-yl)pyridin-3- |
| | yl)urea |
| 242 | 1-(5-chloro-1H-indol-3-yl)-3-(5-chloro-6-(1-(3,3,3-trifluoropropyl)pyrrolidin-3- |
| | yl)pyridin-3-yl)urea |
| 243 | 1-(5-chloro-1H-indol-3-yl)-3-(5-chloro-6-(3,3-difluoropyrrolidin-1-yl)pyridin-3- |
| | yl)urea |
| 247 | 1-(7-bromo-5-chloro-1H-indol-3-yl)-3-(5-chloro-6-(4,4-difluorocyclohexyl)pyridin- |
| | 3-yl)urea |
| 244 | 1-(7-bromo-5-chloro-1H-indol-3-yl)-3-(5-chloro-6-(6,6-difluoro-2- |
| | azaspiro[3.3]heptan-2-yl)pyridin-3-yl)urea |
| 245 | 1-(5-chloro-6-(4-(3,3,3-trifluoropropyl)piperazin-1-yl)pyridin-3-yl)-3-(5-fluoro-1H- |
| | indol-3-yl)urea |
| 246 | 1-(3-chloro-4-(3,3-difluorocyclobutyl)phenyl)-3-(1H-indol-3-yl)urea |
| 248 | 1-(5-chloro-6-(4,4-difluorocyclohexyl)pyridin-3-yl)-3-(1H-indol-3-yl)urea |
| 249 | 1-(5-chloro-1H-indol-3-yl)-3-(5-fluoro-6-(6-(2,2,2-trifluoroethyl)-2,6- |
| | diazaspiro[3.3]heptan-2-yl)pyridin-3-yl)urea |
| 250 | 1-(5-chloro-6-(6-(2,2,2-trifluoroethyl)-2,6-diazaspiro[3.3]heptan-2-yl)pyridin-3-yl)- |
| | 3-(1H-indol-3-yl)urea |
| 251 | 1-(5,6-difluoro-1H-indol-3-yl)-3-(5-fluoro-6-(4-(2,2,2-trifluoroethyl)piperazin-1- |
| | yl)pyridin-3-yl)urea |
| 252 | 1-(5-fluoro-1H-indol-3-yl)-3-(6-(4-(3,3,3-trifluoropropyl)piperazin-1-yl)pyridin-3- |
| | yl)urea |
| 253 | 1-(5-fluoro-1H-indol-3-yl)-3-(6-(4-(2,2,2-trifluoroethyl)piperazin-1-yl)pyridin-3- |
| | yl)urea |
| 254 | 1-(2-(4,4-difluorocyclohexyl)pyridin-4-yl)-3-(1H-indol-3-yl)urea |
| 255 | 1-(5-chloro-1H-indol-3-yl)-3-(2-(4,4-difluorocyclohexyl)pyridin-4-yl)urea |
| 256 | 1-(5-chloro-1H-indol-3-yl)-3-(5-(4,4-difluorocyclohexyl)pyridin-3-yl)urea |
| 257 | 1-(5-chloro-1H-indol-3-yl)-3-(5-cyano-6-(3,3-difluorocyclobutyl)pyridin-3-yl)urea |
| 258 | 3-(5-chloro-1H-indol-3-yl)-1-(5-chloro-6-(4,4-difluoropiperidin-1-yl)pyridin-3-yl)-1- |
| | methylurea |
| 260 | 1-(5-chloro-1H-indol-3-yl)-3-(5-chloro-6-(4-(cyclopropylmethyl)piperazin-1- |
| | yl)pyridin-3-yl)urea |
| 261 | 1-(5-chloro-1H-indol-3-yl)-3-(5-chloro-6-(4-(2,2-difluoroethyl)piperazin-1- |
| | yl)pyridin-3-yl)urea |
| 262 | 1-(5-chloro-1H-indol-3-yl)-3-(5-chloro-6-(6-(2,2,2-trifluoroethyl)-2,6- |
| | diazaspiro[3.3]heptan-2-yl)pyridin-3-yl)urea |
| 263 | 1-(5-chloro-6-(2,2-dimethyl-1,3-dioxan-5-yl)pyridin-3-yl)-3-(1H-indol-3-yl)urea |
| 264 | 1-(5-chloro-1H-indol-3-yl)-3-(5-chloro-6-(2,2-dimethyl-1,3-dioxan-5-yl)pyridin-3- |
| | yl)urea |
| 266 | 1-(5-chloro-6-(4-(2,2,2-trifluoroethyl)piperazin-1-yl)pyridin-3-yl)-3-(5-fluoro-1H- |
| | indol-3-yl)urea |
| 267 | 1-(5-fluoro-1H-indol-3-yl)-3-(5-fluoro-6-(4-(3,3,3-trifluoropropyl)piperazin-1- |
| | yl)pyridin-3-yl)urea |
| 269 | 1-(5-chloro-6-(4-(2,2,2-trifluoroethyl)piperazin-1-yl)pyridin-3-yl)-3-(5,6-difluoro- |
| | 1H-indol-3-yl)urea |
| 270 | 1-(5-fluoro-1H-indol-3-yl)-3-(5-fluoro-6-(1-(2,2,2-trifluoroethyl)piperidin-4- |
| | yl)pyridin-3-yl)urea |
| 271 | 1-(5-fluoro-6-(1-(2,2,2-trifluoroethyl)piperidin-4-yl)pyridin-3-yl)-3-(1H-indol-3- |
| | yl)urea |
| 272 | 1-(4-(3,3-difluorocyclobutyl)-3-fluorophenyl)-3-(5-fluoro-1H-indol-3-yl)urea |
| 273 | 1-(6-(4,4-difluoropiperidin-1-yl)pyridin-3-yl)-3-(5-fluoro-1H-indol-3-yl)urea |
| 274 | 1-(6-(4,4-difluoropiperidin-1-yl)-5-fluoropyridin-3-yl)-3-(5-fluoro-1H-indol-3- |
| | yl)urea |
| 275 | 1-(5-chloro-1H-indol-3-yl)-3-(5-chloro-6-(2,2-difluoro-7-azaspiro[3.5]nonan-7- |
| | yl)pyridin-3-yl)urea |
| 277 | 1-(5-chloro-6-(3,3-difluorocyclobutyl)pyridin-3-yl)-3-(5-fluoro-1H-indol-3-yl)urea |
| 278 | 1-(5-fluoro-6-(4-(2,2,2-trifluoroethyl)piperazin-1-yl)pyridin-3-yl)-3-(1H-indol-3- |
| | yl)urea |
| 279 | 1-(5-chloro-6-(1-(2,2,2-trifluoroethyl)piperidin-4-yl)pyridin-3-yl)-3-(1H-indol-3- |
| | yl)urea |
| 280 | 1-(1H-indol-3-yl)-3-(6-(4-(2,2,2-trifluoroethyl)piperazin-1-yl)pyridin-3-yl)urea |
| 281 | 1-(5-chloro-6-(1-oxa-9-azaspiro[5.5]undecan-9-yl)pyridin-3-yl)-3-(1H-indol-3- |
| | yl)urea |
| 282 | 1-(4-(3,3-difluorocyclobutyl)-3-fluorophenyl)-3-(1H-indol-3-yl)urea |
| 283 | 1-(5-chloro-6-(3,3-difluorocyclobutyl)pyridin-3-yl)-3-(1H-indol-3-yl)urea |
| 284 | tert-butyl(R)-4-(3-chloro-5-(3-(5-chloro-1H-indol-3-yl)ureido)pyridin-2-yl)-2- |
| | methylpiperazine-1-carboxylate |
| 285 | 1-(6-(8-oxa-3-azabicyclo[3.2.1]octan-3-yl)-5-chloropyridin-3-yl)-3-(5-chloro-1H- |
| | indol-3-yl)urea |
| 286 | 1-(5-chloro-1H-indol-3-yl)-3-(5-chloro-6-((2S,6S)-2,6-dimethylmorpholino)pyridin- |
| | 3-yl)urea |
| 287 | 1-(4-chloro-5-(4,4-difluorocyclohexyl)pyridin-2-yl)-3-(5-fluoro-1H-indol-3-yl)urea |
| 290 | 1-(5,6-difluoro-1H-indol-3-yl)-3-(6-(2,2-dimethylmorpholino)-5-fluoropyridin-3- |
| | yl)urea |
| 292 | 1-(5-chloro-1H-indol-3-yl)-3-(6-(4,4-difluoropiperidin-1-yl)-5-fluoropyridin-3- |
| | yl)urea |
| 293 | 1-(5-chloro-1H-indol-3-yl)-3-(5-chloro-6-((2S,6R)-2,6-dimethylmorpholino)pyridin- |
| | 3-yl)urea |
| 294 | 1-(5-bromo-6-(2,2-dimethyl-1,3-dioxan-5-yl)pyridin-3-yl)-3-(5-chloro-1H-indol-3- |
| | yl)urea |
| 297 | 1-(5-chloro-1H-indol-3-yl)-3-(5-chloro-6-(1-(2,2,2-trifluoroethyl)piperidin-4- |
| | yl)pyridin-3-yl)urea |
| 298 | 1-(5-chloro-1H-indol-3-yl)-3-(5-chloro-6-(2,2-dimethylmorpholino)pyridin-3-yl)urea |
| 239 | 1-(5-chloro-6-(6-azaspiro[2.5]octan-6-yl)pyridin-3-yl)-3-(5-fluoro-1H-indol-3- |
| | yl)urea |
| 241 | 1-(5-chloro-6-(6-azaspiro[2.5]octan-6-yl)pyridin-3-yl)-3-(5,6-difluoro-1H-indol-3- |
| | yl)urea |
| 265 | methyl 5-(3-(1H-indol-3-yl)ureido)-2-(4,4-difluorocyclohexyl)nicotinate |
| 268 | 1-(5-chloro-6-(1-(2,2,2-trifluoroethyl)piperidin-4-yl)pyridin-3-yl)-3-(5-fluoro-1H- |
| | indol-3-yl)urea |
| 299 | 1-(5-chloro-6-(4,4-difluoropiperidin-1-yl)pyridin-3-yl)-3-(5,7-dichloro-1H-indol-3- |
| | yl)urea |
| 295 | 1-(5-chloro-1H-indol-3-yl)-3-(6-(4,4-difluorocyclohexyl)-5-fluoropyridin-3-yl)urea |
| 191 | 1-(5-chloro-6-(4,4-difluorocyclohexyl)pyridin-3-yl)-3-(5-hydroxy-1H-indol-3- |
| | yl)urea |
| 159 | 1-(5-chloro-6-(4,4-difluorocyclohexyl)pyridin-3-yl)-3-(5-(difluoromethyl)-1H-indol- |
| | 3-yl)urea |
| 209 | 1-(5-chloro-1H-indol-3-yl)-3-(1-(4,4-difluorocyclohexyl)-1H-pyrazol-4-yl)urea |
| 212 | 3-(5-chloro-1H-indol-3-yl)-1-[1-(4,4-difluorocyclohexyl)imidazol-4-yl]urea |
| 211 | 1-(1-(4,4-difluorocyclohexyl)-1H-pyrazol-3-yl)-3-(5-fluoro-1H-indol-3-yl)urea |
| 210 | 1-[1-(4,4-difluorocyclohexyl)imidazol-4-yl]-3-(5-fluoro-1H-indol-3-yl)urea |
| 276 | 1-(1H-indol-3-yl)-3-(5-methyl-6-(1-(2,2,2-trifluoroethyl)piperidin-4-yl)pyridin-3- |
| | yl)urea |
| 288 | 1-(5-chloro-1H-indol-3-yl)-3-(5-chloro-6-(cis-3,5-dimethylpiperazin-1-yl)pyridin-3- |
| | yl)urea |
| 289 | 1-(5-chloro-1H-indol-3-yl)-3-(5-chloro-6-((3S,5S)-3,5-dimethylpiperazin-1- |
| | yl)pyridin-3-yl)urea |
| 291 | 1-(5-chloro-1H-indol-3-yl)-3-(5-chloro-6-((3R,5R)-3,5-dimethylpiperazin-1- |
| | yl)pyridin-3-yl)urea |
| 296 | (S)-1-(5-chloro-1H-indol-3-yl)-3-(5-chloro-6-(3-methylpiperazin-1-yl)pyridin-3- |
| | yl)urea |

334. The compound of clause 1, wherein the compound is selected from the group consisting of the following:

| Compound # | Name |
|---|---|
| 101 | 1-(5,6-difluoro-1H-indol-3-yl)-3-(6-(4,4-difluorocyclohexyl)pyridin-3-yl)urea |
| 102 | 1-(5,6-difluoro-1H-indol-3-yl)-3-(6-(4,4-difluoropiperidin-1-yl)-5-fluoropyridin- |
| | 3-yl)urea |
| 103 | 1-(5-fluoro-1H-indol-3-yl)-3-(6-(spiro[2.5]octan-6-yl)pyridin-3-yl)urea |
| 104 | 1-(5-fluoro-1H-indol-3-yl)-3-(5-(spiro[2.5]octan-6-yl)pyrazin-2-yl)urea |
| 105 | 1-(5-chloro-1H-indol-3-yl)-3-(5-fluoro-6-(1-(2,2,2-trifluoroethyl)piperidin-4- |
| | yl)pyridin-3-yl)urea |
| 106 | 1-(4-fluoro-5-(trifluoromethyl)-1H-indol-3-yl)-3-(5-(1-(2,2,2- |
| | trifluoroethyl)piperidin-4-yl)pyrazin-2-yl)urea |
| 107 | 1-(1H-indol-3-yl)-3-(1-((1-(2,2,2-trifluoroethyl)piperidin-4-yl)methyl)-1H- |
| | pyrazo1-4-yl)urea |
| 108 | 1-(5-bromo-1H-indol-3-yl)-3-(4-(4- |
| | (cyclopropylmethoxy)cyclohexyl)phenyl)urea |
| 109 | 1-(6-((4-cyanocyclohexyl)oxy)pyridin-3-yl)-3-(5-(difluoromethoxy)-1H-indol-3- |
| | yl)urea |
| 110 | 1-(6-((4-cyanocyclohexyl)oxy)pyridazin-3-yl)-3-(5-ethyl-6-fluoro-1H-indol-3- |
| | yl)urea |
| 111 | 1-(6-(bicyclo[3.2.1]octan-3-yl)-5-methylpyridin-3-yl)-3-(5,6-difluoro-1H-indol- |
| | 3-yl)urea |
| 112 | 1-(1-(dimethylglycyl)-5,6-difluoro-1H-indol-3-yl)-3-(6-(7,7-dimethyloxepan-4- |
| | yl)-5-methylpyridin-3-yl)urea |
| 113 | 1-(1-acetyl-5,6-difluoro-1H-indol-3-yl)-3-(6-(4,4-difluoropiperidin-1-yl)-5- |
| | fluoropyridin-3-yl)urea |
| 114 | 1-(5,6-difluoro-1-(methylsulfonyl)-1H-indol-3-yl)-3-(5-(4-methoxypiperidin-1- |
| | yl)pyridin-2-yl)urea |
| 115 | 1-(5-(4-methoxypiperidin-1-yl)pyridin-2-yl)-1-methyl-3-(5-methyl-1H-indol-3- |
| | yl)urea |
| 116 | (E)-2-cyano-1-(5-(4-methoxypiperidin-1-yl)pyridin-2-yl)-3-(5-methyl-1H-indol- |
| | 3-yl)guanidine |
| 117 | (E)-N-(5-(4-methoxypiperidin-1-yl)pyridin-2-yl)-N′-(5-methyl-1H-indol-3-yl)-2- |
| | nitroethene-1,1-diamine |
| 118 | 1-(4-ethyl-6-fluoro-5-(4-methoxypiperidin-1-yl)pyridin-2-yl)-3-(5-methyl-1H- |
| | indol-3-yl)urea |
| 119 | 1-(6-fluoro-5-(4-methoxypiperidin-1-yl)pyridin-2-yl)-1-(3-hydroxypropyl)-3-(5- |
| | (pentafluoro-l6-sulfanyl)-1H-indol-3-yl)urea |
| 120 | 1-(6-fluoro-5-(4-(3-hydroxypropyl)piperidin-1-yl)pyridin-2-yl)-3-(5- |
| | (methylthio)-1H-indol-3-yl)urea |
| 121 | 1-(2-(6-(3-fluorocyclobutoxy)pyridin-3-yl)ethyl)-3-(5-(methylthio)-1H-indol-3- |
| | yl)urea |
| 122 | 1-(5-(methylsulfonyl)-1H-indol-3-yl)-3-(4-(spiro[3.3]heptan-2- |
| | yloxy)phenethyl)urea |
| 300 | 1-(5-chloro-1H-indol-3-yl)-3-(4-cyclobutylphenyl)urea |

335. A pharmaceutical composition comprising a compound of clauses 1-334 and one or more pharmaceutically acceptable excipients.

336. A method for inhibiting STING activity, the method comprising contacting STING with a compound as defined in any one of clauses 1-334.

337. The method of clause 336, wherein the inhibiting comprises antagonizing STING.

338. The method of clause 336 or 337, which is carried out in vitro.

339. The method of clause 338, wherein the method comprises contacting a sample comprising one or more cells comprising STING with the compound.

340. The method of clause 338 or 339, wherein the one or more cells are one or more cancer cells.

341. The method of clause 339 or 340 wherein the sample further comprises one or more cancer cells, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

342. The method of clause 336 or 337, which is carried out in vivo.

343. The method of clause 342, wherein the method comprises administering the compound to a subject having a disease in which increased (e.g., excessive) STING signaling contributes to the pathology and/or symptoms and/or progression of the disease.

344. The method of clause 343, wherein the subject is a human.

345. The method of clause 344, wherein the disease is cancer.

346. The method of clause 345, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

347. The method of clause 345 or 346, wherein the cancer is a refractory cancer.

348. The method of clause 343, wherein the compound is administered in combination with one or more additional cancer therapies.

349. The method of clause 348, wherein the one or more additional cancer therapies comprises surgery, radiotherapy, chemotherapy, toxin therapy, immunotherapy, cryotherapy or gene therapy, or a combination thereof.

350. The method of clause 349, wherein chemotherapy comprises administering one or more additional chemotherapeutic agents.

351. The method of clause 350, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

352. The method of any one of clauses 343-351, wherein the compound is administered intratumorally.

353. A method of treating cancer, comprising administering to a subject in need of such treatment an effective amount of a compound as defined in any one of clauses 1-334, or a pharmaceutical composition as defined in clause 335.

354. The method of clause 353, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

355. The method of clause 353 or 354, wherein the cancer is a refractory cancer.

356. The method of clause 353, wherein the compound is administered in combination with one or more additional cancer therapies.

357. The method of clause 356, wherein the one or more additional cancer therapies comprises surgery, radiotherapy, chemotherapy, toxin therapy, immunotherapy, cryotherapy or gene therapy, or a combination thereof.

358. The method of clause 357, wherein chemotherapy comprises administering one or more additional chemotherapeutic agents.

359. The method of clause 357, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

360. The method of any one of clauses 353-359, wherein the compound is administered intratumorally.

361. A method of inducing an immune response in a subject in need thereof, the method comprising administering to the subject an effective amount of a compound as defined in any one of clauses 1-334, or a pharmaceutical composition as defined in clause 335.

362. The method of clause 361, wherein the subject has cancer.

363. The method of clause 362, wherein the subject has undergone and/or is undergoing and/or will undergo one or more cancer therapies.

364. The method of clause 362, wherein the cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

365. The method of clause any one of clauses 362-364, wherein the cancer is a refractory cancer.

366. The method of clause 361, wherein the immune response is an innate immune response.

367. The method of clause 363, wherein the at least one or more cancer therapies comprises surgery, radiotherapy, chemotherapy, toxin therapy, immunotherapy, cryotherapy or gene therapy, or a combination thereof.

368. The method of clause 367, wherein chemotherapy comprises administering one or more additional chemotherapeutic agents.

369. The method of clause 368, wherein the one or more additional chemotherapeutic agents is selected from alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

370. A method of treatment of a disease in which increased (e.g., excessive) STING signaling contributes to the pathology and/or symptoms and/or progression of the disease, comprising administering to a subject in need of such treatment an effective amount of a compound as defined in any one of clauses 1-334, or a pharmaceutical composition as defined in clause 335.

371. A method of treatment comprising administering to a subject having a disease in which increased (e.g., excessive) STING signaling contributes to the pathology and/or symptoms and/or progression of the disease an effective amount of a compound as defined in any one of clauses 1-334, or a pharmaceutical composition as defined in clause 335.

372. A method of treatment comprising administering to a subject a compound as defined in any one of clauses 1-334, or a pharmaceutical composition as defined in clause 335, wherein the compound or composition is administered in an amount effective to treat a disease in which increased (e.g., excessive) STING signaling contributes to the pathology and/or symptoms and/or progression of the disease, thereby treating the disease.

373. The method of any one of clauses 370-372, wherein the disease is cancer.

374. The method of clause 373, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

375. The method of clause 373 or 374, wherein the cancer is a refractory cancer.

376. The method of any one of clauses 373-375, wherein the compound is administered in combination with one or more additional cancer therapies.

377. The method of clause 376, wherein the one or more additional cancer therapies comprises surgery, radiotherapy, chemotherapy, toxin therapy, immunotherapy, cryotherapy or gene therapy, or a combination thereof.

378. The method of clause 377, wherein chemotherapy comprises administering one or more additional chemotherapeutic agents.

379. The method of clause 378, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

380. The method of any one of clauses 370-379, wherein the compound is administered intratumorally.

381. A method of treatment of a disease, disorder, or condition associated with STING, comprising administering to a subject in need of such treatment an effective amount of a compound as defined in any one of clauses 1-334, or a pharmaceutical composition as defined in clause 335.

382. The method of clause 381, wherein the disease, disorder, or condition is selected from type I interferonopathies, Aicardi-Goutières Syndrome (AGS), genetic forms of lupus, inflammation-associated disorders, and rheumatoid arthritis.

383. The method of clause 382, wherein the disease, disorder, or condition is a type I interferonopathy (e.g., STING-associated vasculopathy with onset in infancy (SAVI)).

384. The method of clause 383, wherein the type I interferonopathy is STING-associated vasculopathy with onset in infancy (SAVI)).

385. The method of clause 382, wherein the disease, disorder, or condition is Aicardi-Goutières Syndrome (AGS).

386. The method of clause 382, wherein the disease, disorder, or condition is a genetic form of lupus.

387. The method of clause 382, wherein the disease, disorder, or condition is inflammation-associated disorder.

388. The method of clause 387, wherein the inflammation-associated disorder is systemic lupus erythematosus.

389. The method of any one of clauses 336-388, wherein the method further comprises identifying the subject.

390. A combination comprising a compounds defined in any one of clauses 1-334 or a pharmaceutically acceptable salt or tautomer thereof, and one or more therapeutically active agents.

391. A compound defined in any one of clauses 1-334 or a pharmaceutically acceptable salt or tautomer thereof, or a pharmaceutical composition defined in clause 335, for use as a medicament.

392. A compound defined in any one of clauses 1-334 or a pharmaceutically acceptable salt or tautomer thereof, or a pharmaceutical composition defined in clause 335, for use in the treatment of a disease, condition or disorder modulated by STING inhibition.

393. A compound defined in any one of clauses 1-334 or a pharmaceutically acceptable salt or tautomer thereof, or the pharmaceutical composition defined in clause

335, for use in the treatment of a disease mentioned in any one of clauses 336 to 389 (e.g., any one of clauses 341, 345-347, 354-355, 362, 364, 365, 370-375, or 381-388).

394. Use of a compound defined in any one of clauses 1-334 or a pharmaceutically acceptable salt or tautomer thereof, or a pharmaceutical composition defined in clause 335, in the manufacture of a medicament for the treatment of a disease mentioned in in any one of clauses 336 to 389 (e.g., any one of clauses 341, 345-347, 354-355, 362, 364, 365, 370-375, or 381-388).

What is claimed is:

1. A compound of Formula I: 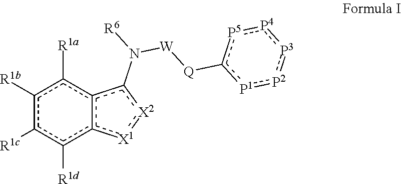
or a pharmaceutically acceptable salt thereof or a tautomer thereof, wherein:
X¹ is selected from the group consisting of O, S, N, NR², and CR¹;
X² is selected from the group consisting of O, S, N, NR⁴, and CR⁵;
each [symbol] is independently a single bond or a double bond, provided that:
the five-membered ring comprising X¹ and X² is heteroaryl;
the 6-membered ring 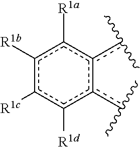
is aromatic; and
and the ring comprising P¹, P², P³, P⁴, and P⁵ is aromatic;
P¹, P², P³, P⁴, and P⁵ are defined according to (AA) or (BB):
AA
each of P¹, P², P³, P⁴, and P⁵ is independently selected from the group consisting of: N, CH, CR⁷, and CR^{c}, provided that 1-2 of P¹, P², P³, P⁴, and P⁵ is an independently selected CR⁷; or
BB
P¹ is absent, thereby providing a 5-membered ring,
each of P², P³, P⁴, and P⁵ is independently selected from the group consisting of O, S, N, NH, NR^{d}, NR⁷, CH, CR⁷, and CR^{c}, provided that 1-3 of P², P³, P⁴, and P⁵ is O, S, N, NH, NR^{d}, or NR⁷; and 1-2 of P², P³, P⁴, and P⁵ is an independently selected NR⁷ or CR⁷;
each R⁷ is independently selected from the group consisting of: —R⁸ and -L³-R⁹;
R⁸ and R⁹ are independently selected from the group consisting of:
(a) C₃₋₁₂ cycloalkyl or C₃₋₁₂ cycloalkenyl, each of which is optionally substituted with 1-4 independently selected R⁷′;
(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R⁷′;
(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R⁷′; and
(d) C₆₋₁₀ aryl optionally substituted with 1-4 independently selected R⁷′;
-L³ is selected from the group consisting of —O—, —C₁₋₄ alkylene, —S—, —NH—, S(O)₁₋₂, C(═O)NH, NHC(═O), C(═O)O, OC(═O), C(═O), NHS(O)₂, and S(O)₂NH;
each occurrence of R⁷′ is independently selected from the group consisting of:
halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),
W is selected from the group consisting of:
(i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);
Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents point of attachment to W;
each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);
each occurrence of R² is independently selected from the group consisting of:
(i) H;
(ii) C₁₋₆ alkyl, which is optionally substituted with 1-3 independently selected R^{a};
(iii) —C(O)(C₁₋₆ alkyl) optionally substituted with 1-3 independently selected R^{a};
(iv) —C(O)O(C₁₋₄ alkyl) optionally substituted with 1-3 independently R^{a};
(v) —CON(R′)(R″);
(vi) —S(O)₁₋₂(NR′R″);
(vii) —S(O)₁₋₂(C₁₋₄ alkyl) optionally substituted with 1-3 independently selected R^{a};
(viii) —OH;
(ix) C₁₋₄ alkoxy; and
(x) -L⁴-L⁵-Rⁱ;
R⁴ is selected from the group consisting of H and C₁₋₆ alkyl optionally substituted with 1-3 independently selected R^{a};
R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;
each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};
each occurrence of R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; —C(═O)N(R′)(R″); and -L¹-L²-R^{h};
R^{d} is selected from the group consisting of: C₁₋₆ alkyl optionally substituted with 1-3 substituents each independently selected from the group consisting of halo, C₁₋₃ alkoxy, C₁₋₃ haloalkoxy, OH, and C₃₋₆ cycloalkyl; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or
R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;
-L¹ is a bond or C₁₋₃ alkylene; -L² is —O—, —N(H)—, —S(O)₀₋₂—, or a bond;
R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};
-L⁵- is selected from the group consisting of a bond and C₁₋₄ alkylene;
Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy; and
each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl;
or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S;
provided that:
(a) when X¹ is NR²; X² is CH; each of R^{1a}, R^{1b}, R^{1c}, R^{1d}, and R⁶ is H; W is C(═O); Q is NH; and P¹, P², P³, P⁴, and P⁵ are defined according to (AA); then:
R² cannot be CH₂CH₂OCH₃, CH₃, CH₂CH₃, or SO₂-(p-tolyl) when the 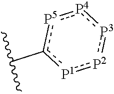
moiety is 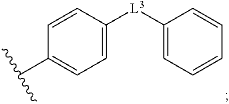
and -L³ is —O—, —NH—, or C(═O), and
R² cannot be CH₂CH₂CH₂N(CH₃)₂ or CH₂CH₂CH₂N(CH₂CH₃)₂ when the 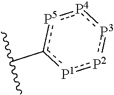
moiety is pyrimidinyl or pyridyl each substituted with one R⁷, wherein R⁷ is R⁸, and R⁸ is unsubstituted phenyl; and
(b) the compound is not:

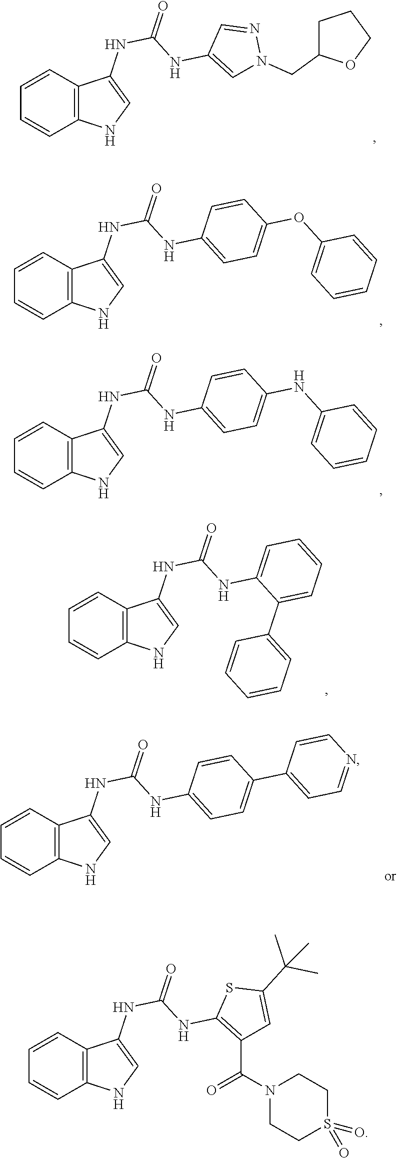 [SMILES: O=C(Nc1cnn(CC2CCCO2)c1)Nc1c[nH]c2ccccc12 ; O=C(Nc1ccc(Oc2ccccc2)cc1)Nc1c[nH]c2ccccc12 ; O=C(Nc1ccc(Nc2ccccc2)cc1)Nc1c[nH]c2ccccc12 ; O=C(Nc1ccccc1-c1ccccc1)Nc1c[nH]c2ccccc12 ; O=C(Nc1ccc(-c2ccncc2)cc1)Nc1c[nH]c2ccccc12 ; CC(C)(C)c1cc(C(=O)N2CCS(=O)(=O)CC2)c(NC(=O)Nc2c[nH]c3ccccc23)s1]

2. The compound of claim 1, wherein P¹, P², P³, P⁴, and P⁵ are defined according to (AA).

3. The compound of claim 2, wherein one or two of P¹, P², P³, P⁴, and P⁵ is N, or one of P¹, P², P³, P⁴, and P⁵ is N.

4. The compound of claim 2 or 3, wherein the 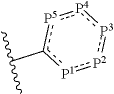
moiety has the formula: 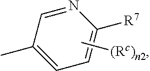
wherein n2 is 0, 1, or 2.

5. The compound of any one of claims 2 to 4, wherein the 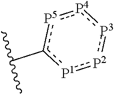
moiety has the formula

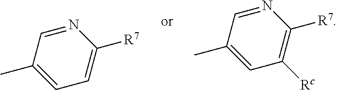

6. The compound of claim 2 or 3, wherein the 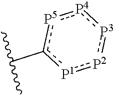
moiety has the formula: 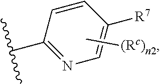
wherein n2 is 0, 1, or 2; or
wherein the 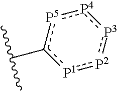
moiety has the formula: 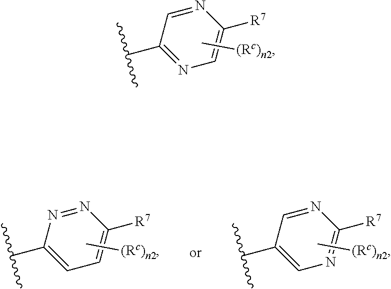
wherein n2 is 0, 1, or 2.

7. The compound of claim 2, 3 or 6, wherein the 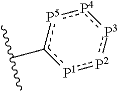
moiety has the formula:

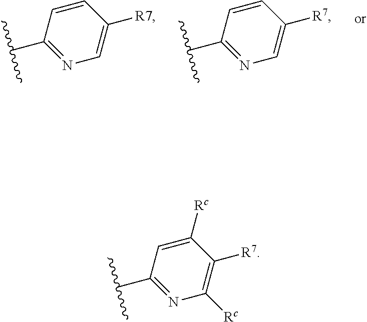

8. The compound of claim 2, wherein each of P¹, P², P³, P⁴, and P⁵ is independently selected from the group consisting of CH, CR⁷, and CR^{c}.

9. The compound of claim 2 or 8, wherein the 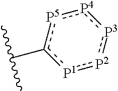
moiety has the formula: 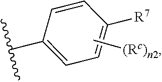
wherein n2 is 0, 1, or 2

10. The compound of claim 2, 8, or 9, wherein the 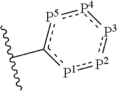
moiety has the formula:

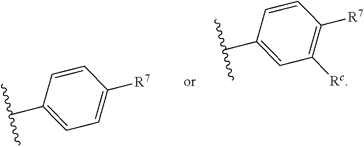

11. The compound of any one of claims 1-10, wherein R⁷ is R⁸.

12. The compound of any one of claims 1-11, wherein R⁸ is
i) C₃₋₁₂ cycloalkyl or C₃₋₁₂ cycloalkenyl, each of which is substituted with 1-4 independently selected R⁷′;
ii) C₄₋₈ cycloalkyl which is substituted with 1-4 independently selected R⁷′;
iii) cyclohexyl or cyclobutyl, each of which is substituted with 1-4 independently selected R⁷′;
or
iv) 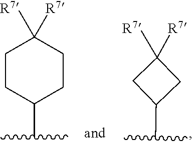
wherein each R⁷′ is independently halo.

13. The compound of any one of claims 1-11, wherein R⁸ is
i) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R⁷′;
ii) heterocyclyl of 4-6 ring atoms, wherein 1-2 ring atoms are heteroatoms, each independently selected from the group consisting of N, N(H), N(R^{d}), O, and S(O)₀₋₂, and wherein one or more ring carbon atoms of the heterocyclyl ring is substituted with 1-3 independently selected R⁷′;
iii) 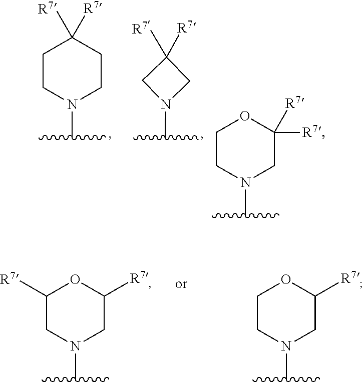
iv) spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′;
v) 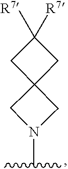
or
vi) an R⁸ of any one of i) to v) wherein each R⁷′ is independently halo or C₁₋₃ alkyl.

14. The compound of any one of claims 1-13, wherein each R^{c} is an independently selected halo.

15. The compound of any one of claims 1-14, wherein Q is NH; and W is C(═O), and optionally wherein R⁶ is H.

16. The compound of any one of claims 1-15, wherein X¹ is NR²; and X² is CR⁵, or wherein X¹ is NH; and X² is CH.

17. The compound of any one of claims 1-16, wherein
i) 1-2 of R^{1a}, R^{1b}, R^{1c}, and R^{1d} is other than H; and each remaining of R^{1a}, R^{1b}, R^{1c}, and R^{1d} is H;
ii) each of R^{1b} and R^{1c} is other than H; and each of R^{1a} and R^{1d} is H;
iii) each of R^{1b} and R^{1c} is an independently selected halo, and each of R^{1a} and R^{1d} is H;
iv) R^{1b} is other than H; and each of R^{1a}, R^{1c}, and R^{1d} is H;
v) R^{1b} is selected from the group consisting of halo; C₁₋₆ alkyl optionally substituted with 1-2 R^{a}; C₁₋₄ haloalkyl; —CN; —SF₅; C₁₋₄ thioalkoxy; S(O)₂(C₁₋₄ alkyl); and C₁₋₄ alkoxy or C₁₋₄ haloalkoxy; and each of R^{1a}, R^{1c}, and R^{1d} is H;
or
vi) R^{1b} is halo; and each of R^{1a}, R^{1c}, and R^{1d} is H.

18. The compound of claim 1, wherein the compound is a compound of Formula (I-1a), (I-2a), or (I-3a): 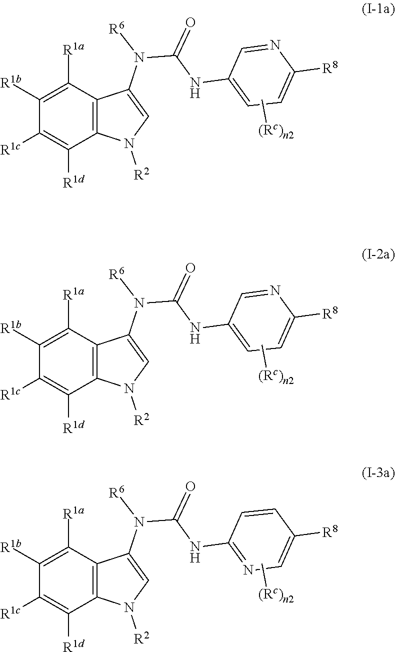
or a pharmaceutically acceptable salt thereof, wherein:
each of R^{1a}, R^{1b}, R^{1c}, and R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;
n2 is 0, 1, or 2;
each R^{c} when present is independently selected from the group consisting of: halo, cyano, C₁₋₃ alkyl, and C₁₋₃ alkoxy;
R⁸ is selected from the group consisting of: 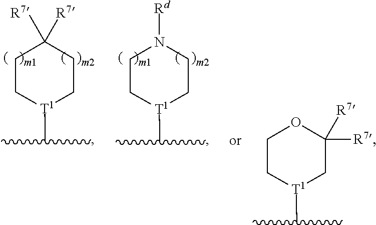
wherein m1 and m2 are independently 0, 1, or 2, and T¹ is CH or N; and
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R⁷′,
optionally wherein each R⁷′ is independently halo or C₁₋₃ alkyl, and
optionally wherein R^{d} is C₁₋₆ alkyl which is optionally substituted with 1-3 independently selected halo.

19. The compound of claim 18, wherein R⁸ is selected from the group consisting of: 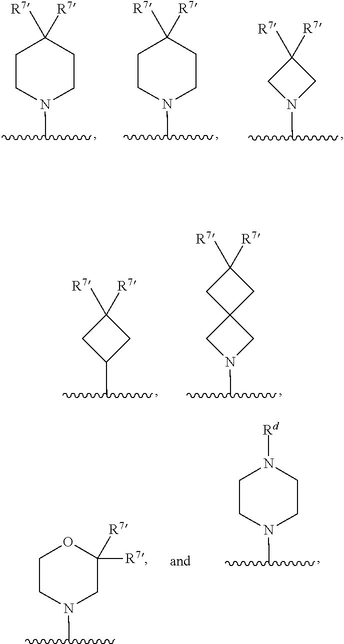
optionally wherein each R⁷′ is independently halo or C₁₋₃ alkyl, such as —F or methyl, and
optionally wherein R^{d} is C₁₋₆ alkyl which is optionally substituted with 1-3 independently selected halo, such as C₂₋₄ alkyl optionally substituted with 1-3 —F.

20. The compound of claim 18 or 19, wherein each R⁷′ is independently halo or C₁₋₃ alkyl, and wherein R^{d} is C₁₋₆ alkyl which is optionally substituted with 1-3 independently selected halo.

21. The compound of claim 1, wherein the compound is selected from the group consisting of the compounds delineated in Table C1, or a pharmaceutically acceptable salt thereof.

22. A pharmaceutical composition comprising a compound of claims 1-21 and one or more pharmaceutically acceptable excipients.

23. A method for inhibiting STING activity, the method comprising contacting STING with a compound as claimed in any one of claims 1-21, or a pharmaceutically acceptable salt thereof; or a pharmaceutical composition as claimed in claim 22.

24. A method of inducing an immune response in a subject in need thereof, the method comprising administering to the subject an effective amount of a compound as claimed in any one of claims 1-21, or a pharmaceutically acceptable salt thereof; or a pharmaceutical composition as claimed in claim 22.

25. A method of treatment of disease, disorder, or condition associated with STING, such as a disease, disorder, or condition, in which increased STING signaling, such as excessive STING signaling, contributes to the pathology and/or symptoms and/or progression of the disease comprising administering to a subject in need of such treatment an effective amount of a compound as claimed in any one of claims 1-21, or a pharmaceutically acceptable salt thereof, or a pharmaceutical composition as claimed in claim 22.
